(19) United States
(12) Patent Application Publication
(10) Pub. No.: US 2024/0299533 A1
(43) Pub. Date: Sep. 12, 2024

(54) DEOPTIMIZED SARS-COV-2 VARIANTS AND METHODS AND USES THEREOF

(71) Applicants: Codagenix Inc., Farmingdale, NY (US); Serum Institute of India Private Limited, Hadapsar, Pune (IN)

(72) Inventors: Steffen Mueller, Great Neck, NY (US); John Robert Coleman, Blauvelt, NY (US); Ying Wang, South Setauket, NY (US); Chen Yang, Plainview, NY (US); Yutong Song, Stony Brook, NY (US)

(73) Assignees: Codagenix Inc., Farmingdale, NY (US); Serum Institute of India Private Limited, Hadapsar, Pune (IN)

(21) Appl. No.: 18/574,973

(22) PCT Filed: Jun. 30, 2022
(86) PCT No.: PCT/US22/35824

§ 371 Date: Dec. 28, 2023

Related U.S. Application Data
(60) Provisional application No. 63/219,263, filed on Jul. 7, 2021.

(51) Int. Cl. A61K 39/215; A61K 9/00; A61K 39/00; A61P 31/14; C07K 14/005; C12N 7/00

(52) U.S. Cl. CPC A61K 39/215; A61K 9/0043; A61P 31/14; C07K 14/005; C12N 7/00; A61K 2039/5254; A61K 2039/543; A61K 2039/545; C12N 2770/20021; C12N 2770/20022; C12N 2770/20034

(57) ABSTRACT

Described herein are modified SARS-CoV-2 variants. These viruses have been recoded, for example, codon deoptimized or codon pair bias deoptimized and are useful for reducing the likelihood or severity of a SARS-CoV-2 variant infection, preventing a SARS-CoV-2 variant infection, eliciting and immune response, or treating a SARS-CoV-2 variant infection.

### CROSS-REFERENCE TO RELATED APPLICATIONS

This application includes a claim of priority to U.S. provisional patent application No. 63/219,263, filed Jul. 7, 2021, the entirety of which is hereby incorporated by reference.

### REFERENCE TO SEQUENCE LISTING

This application contains a Sequence Listing submitted as an electronic text file named “SequenceListing_064955_000051WO00_ST25”, having a size in bytes of 277,718 bytes, and created on Jun. 30, 2022. The information contained in this electronic file is hereby incorporated by reference in its entirety.

### FIELD OF INVENTION

This invention relates to modified SARS-CoV-2 coronavirus variants, compositions for eliciting an immune response and vaccines for providing protective immunity, prevention and treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

An outbreak of a novel coronavirus was identified during mid-December 2019 in the city of Wuhan in central China. A new strain of coronavirus, now designated as SARS-CoV-2, was identified. The deadly coronavirus has been declared by the WHO as pandemic. The public health crisis of this virus rapidly grew from claiming the lives of dozens of people and infecting over a thousand as of the end of January 2020, to claiming the lives of over 4 million people and infecting over 185 million people as of the beginning of July 2021, to claiming the lives of over 6.3 million as of June 2022.

Since the outbreak, emergence of SARS-CoV-2 variants have been particularly troublesome and hampering vaccine efforts to provide immunity to everyone. Accordingly, prophylactic and therapeutic treatments that are effective against the SARS-CoV-2 variants remain exceedingly and urgently needed.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a polynucleotide comprising a polynucleotide encoding one or more viral proteins or one or more fragments thereof of a parent SARS-CoV-2 variant, wherein the polynucleotide is recoded compared to its parent SARS-CoV-2 variant polynucleotide, and wherein the amino acid sequence of the one or more viral proteins, or one or more fragments thereof of the parent SARS-CoV-2 variant encoded by the polynucleotide remains the same, or wherein the amino acid sequence of the one or more viral proteins or one or more fragments thereof of the parent SARS-CoV-2 variant encoded by the polynucleotide comprises up to 20 amino acid substitutions, additions, or deletions, and wherein the one or more viral proteins or one or more fragments thereof comprises spike protein or a fragment thereof.

In various embodiments, the parent SARS-CoV-2 variant comprises SEQ ID NO:1, or the parent SARS-CoV-2 variant can comprise SEQ ID NO:1 wherein nt 9469 is changed from to A to G and nt 26222 changed from T to G, or the parent SARS-CoV-2 variant can comprise SEQ ID NO:1 wherein there is one or more mutations in SEQ ID NO: 1; and wherein a spike protein coding sequence in SEQ ID NO:1, SEQ ID NO:1 wherein nt 9469 is changed from to A to G and nt 26222 changed from T to G, or SEQ ID NO:1 wherein there is one or more mutations, is replaced with a recoded spike protein coding sequence from a SARS-CoV-2 variant.

In various embodiments, the SARS-CoV-2 variant can be selected from the group consisting of U.K. variant, South Africa variant, Brazil variant, Delta variant, and Omicron variant.

In various embodiments, the polynucleotide can be recoded by reducing codon-pair bias (CPB) or reducing codon usage bias compared to its parent SARS-CoV-2 variant polynucleotide. In various embodiments, the polynucleotide can be recoded by increasing the number of CpG or UpA di-nucleotides compared to its parent SARS-CoV-2 variant polynucleotide. In various embodiments, each of the recoded one or more viral proteins, or each of the recoded one or more fragments thereof can have a codon pair bias less than, −0.05, less than −0.1, less than −0.2, less than −0.3, or less than −0.4. In various embodiments, the polynucleotide can be CPB deoptimized compared to its parent SARS-CoV-2 variant polynucleotide. In various embodiments, the polynucleotide can be codon deoptimized compared to its parent SARS-CoV-2 variant polynucleotide. In various embodiments, the codon-deoptimized or CPB deoptimized can be based on frequently used codons or CPB in humans. In various embodiments, the codon-deoptimized or CPB deoptimized can be based on frequently used codons or CPB in a coronavirus. In various embodiments, the codon-deoptimized or CPB deoptimized can be based on frequently used codons or CPB in a wild-type SARS-CoV-2 coronavirus. In various embodiments, a furin cleavage site can be eliminated.

Various embodiments provide for a vector comprising a polynucleotide of the present invention described herein.

Various embodiments provide for a cell comprising a polynucleotide of the present invention described herein or a vector of the present invention described herein. In various embodiments, the cell can be Vero cell or baby hamster kidney (BHK) cell.

Various embodiments provide for a polypeptide encoded by a polynucleotide of the present invention described herein.

Various embodiments provide for a modified SARS-CoV-2 variant comprising a polynucleotide of the present invention described herein. Various embodiments provide for a modified SARS-CoV-2 variant comprising a polypeptide encoded by a polynucleotide of the present invention described herein. Various embodiments provide for a modified SARS-CoV-2 variant of the invention as described herein, wherein expression of one or more of its viral proteins can be reduced compared to its parent SARS-CoV-2 variant. Various embodiments provide for a modified SARS-CoV-2 variant of the invention as described herein, wherein the reduction in the expression of one or more of its viral proteins can be reduced as the result of recoding a spike protein or a fragment thereof.

Various embodiments provide for an immune composition or vaccine composition for inducing an immune response in a subject, comprising: one or more modified SARS-CoV-2 variant of the invention as described herein. In various embodiments, the immune composition or vaccine composition of the invention as described herein, can further comprise a pharmaceutically acceptable carrier or excipient.

Various embodiments provide for a multivalent immune composition or vaccine composition for inducing an immune response in a subject, comprising: one or more modified SARS-CoV-2 variant of the invention as described herein. In various embodiments, the multivalent immune composition of the invention as described herein or multivalent vaccine composition of the invention as described herein, further comprises a modified SARS-CoV-2 coronavirus comprising a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the multivalent immune composition of the invention as described herein or multivalent vaccine composition of the invention as described herein, can further comprise a modified SARS-CoV-2 coronavirus comprising polypeptide encoded by the polynucleotide having SEQ ID NO: 1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant. In various embodiments, the multivalent immune composition of the invention as described herein or multivalent vaccine composition of the invention as described herein, can further comprise a pharmaceutically acceptable carrier or excipient.

Various embodiments provide for a method of eliciting an immune response in a subject, comprising: administering to the subject a dose of a modified SARS-CoV-2 variant of the invention as described herein.

Various embodiments provide for a method of eliciting an immune response in a subject, comprising: administering to the subject a dose of an immune composition of the invention as described herein.

Various embodiments provide for a method of eliciting an immune response in a subject, comprising: administering to the subject a dose of a vaccine composition of the invention as described herein.

Various embodiments provide for a method of eliciting an immune response in a subject, comprising: administering to the subject a dose of a multivalent immune composition or multivalent vaccine composition of the invention as described herein.

Various embodiments provide for a method of eliciting an immune response in a subject, comprising: administering to the subject a prime dose of a modified SARS-CoV-2 coronavirus of the invention as described herein, or a vaccine composition of the invention as described herein, or an immune composition of the invention as described herein, or a multivalent immune or vaccine composition as described herein; and administering to the subject one or more boost doses of a modified SARS-CoV-2 coronavirus of the invention as described herein, or a vaccine composition of the invention as described herein, or an immune composition of the invention as described herein, or a multivalent immune or vaccine composition as described herein.

In various embodiments, the immune response can be a protective immune response. In various embodiments, the dose is a prophylactically effective or therapeutically effective dose.

In various embodiments, administering can be via a nasal route. In various embodiments, administering can be via nasal drop. In various embodiments, administering can be via nasal spray.

In various embodiments, the dose can be about 10⁴-10⁶ PFU, or the prime dose is about 10⁴-10⁶ PFU and the one or more boost dose can be about 10⁴-10⁶ PFU.

Various embodiments provide for a method of making a deoptimized SARS-CoV-2 variant, comprising: obtaining a nucleotide sequence encoding one or more proteins of a parent SARS-CoV-2 variant or one or more fragments thereof, recoding the nucleotide sequence to reduce protein expression of the one or more proteins, or the one or more fragments thereof, and substituting a nucleic acid having the recoded nucleotide sequence into the parent SARS-CoV-2 variant genome to make the deoptimized SARS-CoV-2 variant genome, wherein expression of the recoded nucleotide sequence is reduced compared to the parent virus. In various embodiments, the deoptimized SARS-CoV-2 variant can be a deoptimized SARS-CoV-2 variant as described herein.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A depicts CDX-005 (deoptimized SARS-CoV-2 “Washington Isolate” virus) is temperature sensitive, i.e. it is more attenuated at higher temperatures. The same amount of CDX-005 virus (such as a 100,000/5 log dilution, or 10,000/4 log dilution will form plaques at 37° C., but not at 40° C. At 40° C. it takes much more virus (i.e., a lower dilution, such as 100/2 log dilution, to see any plaques, and those are very small. The wt virus on the other hand, does not appear to be temperature sensitive; it performs equally well at both temperatures.

FIG. 1B depicts plaque formation for CDX-005, CDX-005.1 (Beta Variant). First row depicts the CDX-005 3 days incubation at 37° C. Second row depicts the CDX-005 3 days incubation at 40° C. Third row depicts the CDX-005.1 (Beta Variant) 3 days incubation at 37° C. Second row depicts the CDX-005.1 (Beta Variant) 3 days incubation at 40° C. This figure also shows that CDX-005 and CDX-005.1 are temperature sensitive, i.e. it is more attenuated at higher temperatures. The same amount of CDX-005 and CDX-005.1 virus (such as a 100,000/5 log dilution, or 10,000/4 log dilution will form plaques at 37 C, but not at 40 C. At 40 C it takes much more virus (i.e., a lower dilution, such as 100/2 log dilution, to see any plaques, and those are very small. This shows that CDX-005.1 is severely temperature sensitive at 40° C. Plaque assays were performed in parallel on the same batch of Vero E6 cell monolayers in 12-well clusters and stained with Crystal Violet after 3 days of incubation at 37° C. or 40° C. CDX-005 (top two rows) and CDX-005.1 (lower two row) samples from the same virus dilution series were used to perform plaque assays at either 37° C. or 40° C. Whereas both CDX-005 and CDX-005.1 form plaques similarly at 37° C., CDX-005 μlaques at 40° C. are significantly smaller, irregularly shaped, and much reduced in number (approximately 1,000-fold). CDX-005.1 was even more severely temperature restricted than CDX-005 and formed no visible plaques at 40° C. at any dilution. Temperature sensitive phenotypes at elevated temperature (human equivalent of fever) are generally regarded as a positive safety feature of live attenuated virus vaccines. With reference to the wt virus in FIG. 1A, on the other hand, does not appear to be temperature sensitive; it performs equally well at both temperatures. A temperature sensitive phenotype is a good and desirable feature of a live attenuated vaccine. It can serve as a “safety valve”. The vaccine replicates well enough at the lower temperature in the vaccine recipient (37° C. (normal body temp)) to induce an immune response. If things do not perform as planned, such as in a particular individual who is hyper sensitive to the vaccine, that individual may develop a fever, which will reduce the virus replication/activity to prevent things from becoming worse.

FIG. 2 depicts body weight changes after dosing of wild-type SARS-COV-2 and CDX-005 in Syrian Gold hamsters.

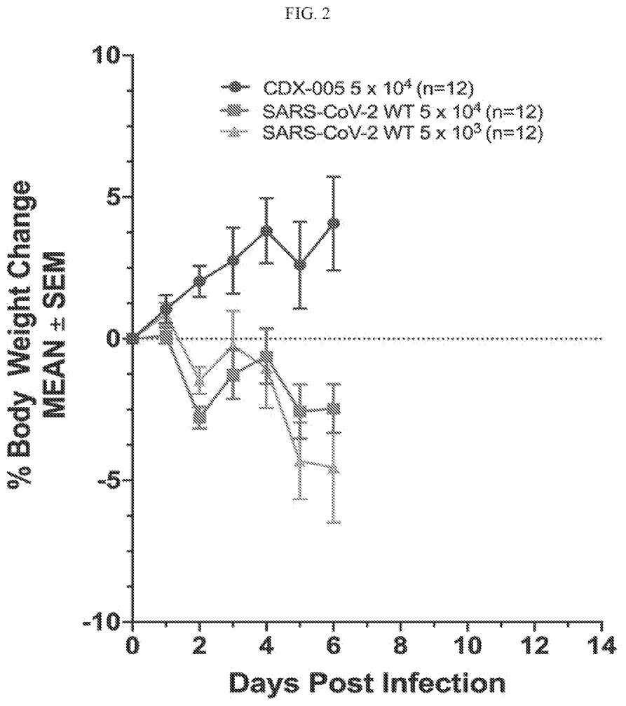

FIG. 3 depicts growth of wt WA1 and CDX-005 in Vero cells. Vero cells were infected with the 0.01MOI of wt WA1 or CDX-005 and cultured for up to 96 hrs at 33° C. or 37° C. Supernatants were collected to recover virus. Titers were determined by plaque forming assays and reported as log of PFU/ml culture medium.

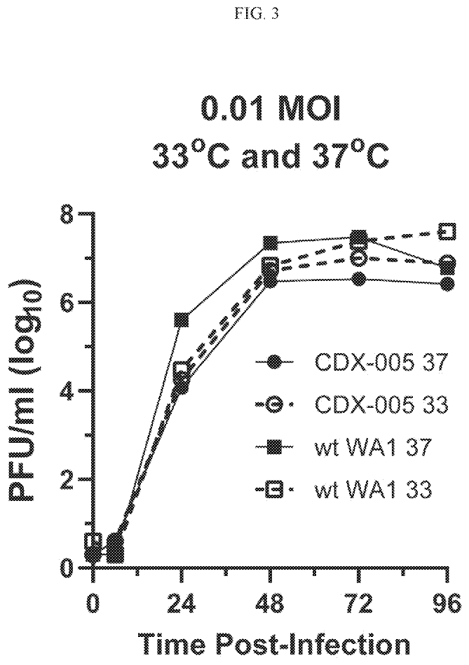

FIGS. 4A-4D depict in vivo attenuation of CDX-005 in hamsters. Hamsters were inoculated with 5×10⁴ or 5×10³ PFU/ml of wt WA1, 5×10⁴ PFU/ml CDX-005. Viral RNA was measured by qPCR at Days 2 and 4 PI in the 4A) olfactory bulb, 4B) brain, and 4C) lungs. (N=3/group; Bars=SEM). 4D) Infectious viral load in left lung tissue of inoculated hamsters was assessed by TCID₅₀ assay and expressed as log₁₀ of TCID₅₀/ml. Differences between CDX-005 and wt WA1 treated groups were significant (N=3/group; P<0.001; Bars=SEM). Horizontal lines indicate LOD.

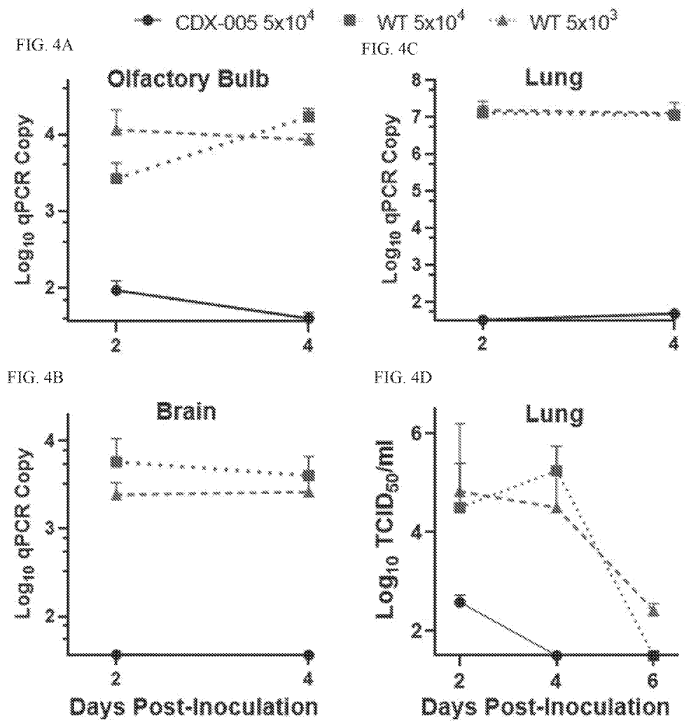

FIGS. 5A-5C depict in vivo attenuation of CDX-005 in hamsters. Hamsters inoculated with 5×10⁴ or 5×10³ PFU/ml of wt WA1 or 5×10⁴ PFU/ml CDX-005. 5A) The weight of hamsters was measured daily for nine days. Weight changes were significantly different between CDX-005 and wt WA1 treated groups (N=10-40/group for CDX-005 and wt WA1 5×10⁴; N=3-12/group wt WA1 5×10³; P<0.001; Bars=SEM). 5B & 5C) Hematoxylin and eosin stained lung sections were examined on Days 2, 4, and 6 PI and scored for cell infiltration. (N=3/group)

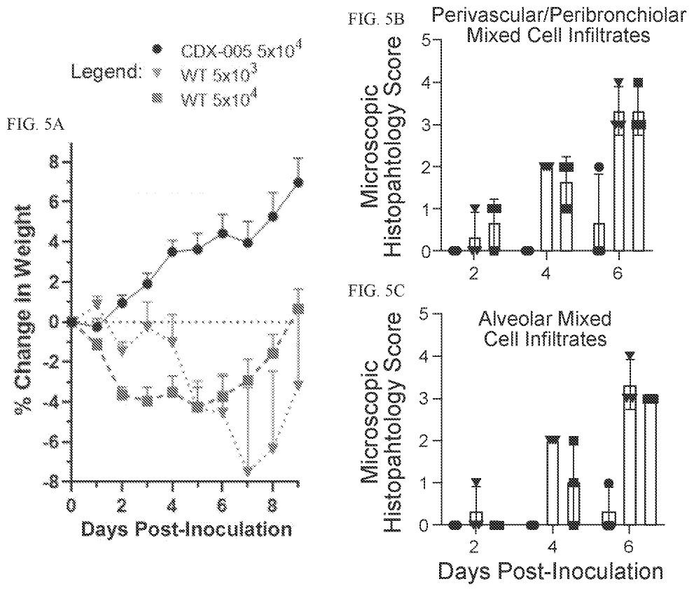

FIGS. 6A-6D depicts efficacy in hamsters. 6A) A Spike-S1 ELISA was performed with naïve hamster control serum or with serum collected from hamsters on Day 16 post-inoculation with wt WA1 or 5×10⁴ PFU CDX-005. Spike S1 IgG in CDX-005 inoculated hamsters was also measured on Day 18 (two days post WA1 challenge). The endpoint IgG titers are shown as the log of the dilution that was 5× above the background. (N=3/group; Bars=SEM) 6B) Plaque Reduction Neutralization Titers (PRNT) against SARS-CoV-2 WA1 were tested in serum of hamsters 16 days after inoculation with 5×10⁴ or 5×10³ PFU of wt WA1 or 5×10⁴ PFU CDX-005. The PRNT is the reciprocal of the last serum dilution that reduced plaque numbers 50, 80, or 90 percent relative to those in wells containing naïve hamster serum. (N=3/group; Bars=SD); 6C) CDX-005 vaccinated hamsters on Day 16 post-vaccination and naïve animals were challenged with 5×10⁴ PFU wt SARS-CoV-2. Lungs were harvested on Day 2 post-challenge and viral loads were measured by qPCR and expressed as log₁₀ of qPCR genomes/ml of tissue. (N=3/group; Bars=SD). 6D) Hamsters vaccinated with vehicle, 5×10⁴ PFU of wt WA1 or 5×10⁴ CDX-005 and challenged with 5×10⁴ PFU/ml wt WA1 intranasally 27 days post-inoculation. Weights were recorded on the day of challenge and daily for 4 days thereafter. (N=5-6 days 0-2, N=3 Days 3-4, Bars=SEM). The results in 6A) and 6B) are from two separate hamster studies.

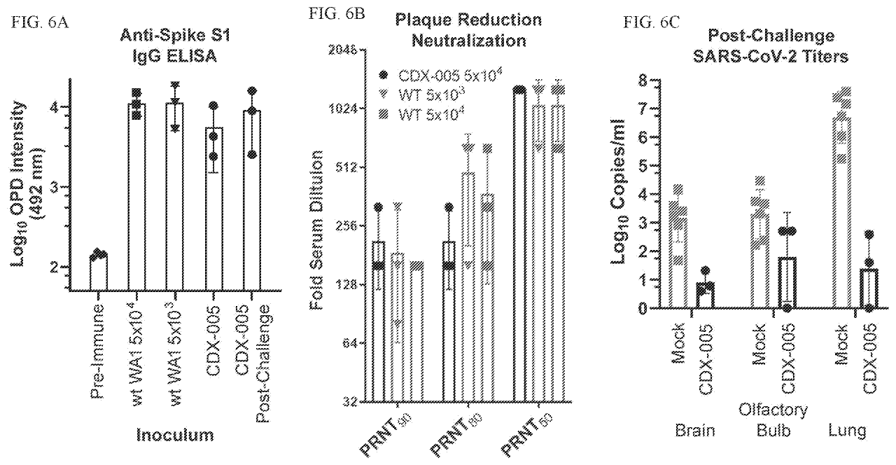

FIG. 7 depicts attenuation in African Green Monkeys. Tracheal lavage fluid was collected from monkeys at Day 4 and Day 6 post-inoculation with 10⁶ PFU wt WA1 or CDX-005. Lavage fluid was subjected to RT-qPCR to detect virus. N=3/group (Day 4) or N=2/group (Day 6).

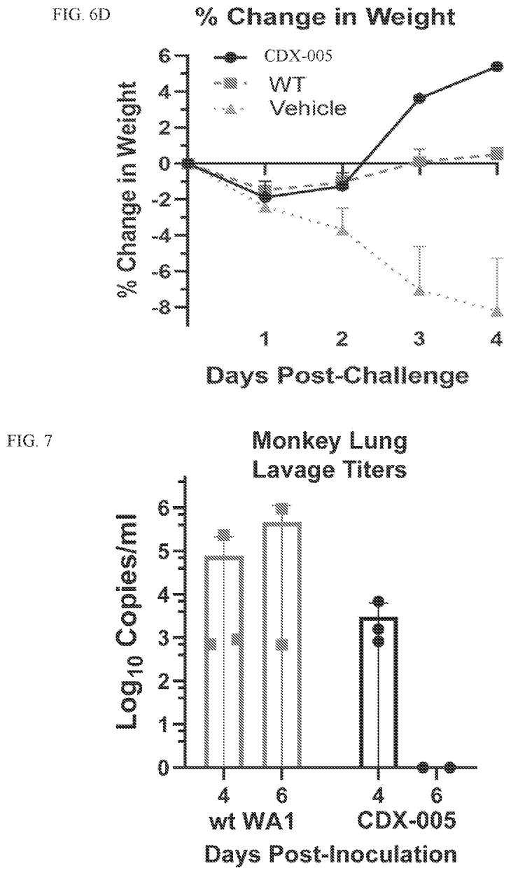

FIG. 8 depicts wt SARS-COV2 v. CDX-005 intranasal dose of 10⁶ in African Green Monkeys.

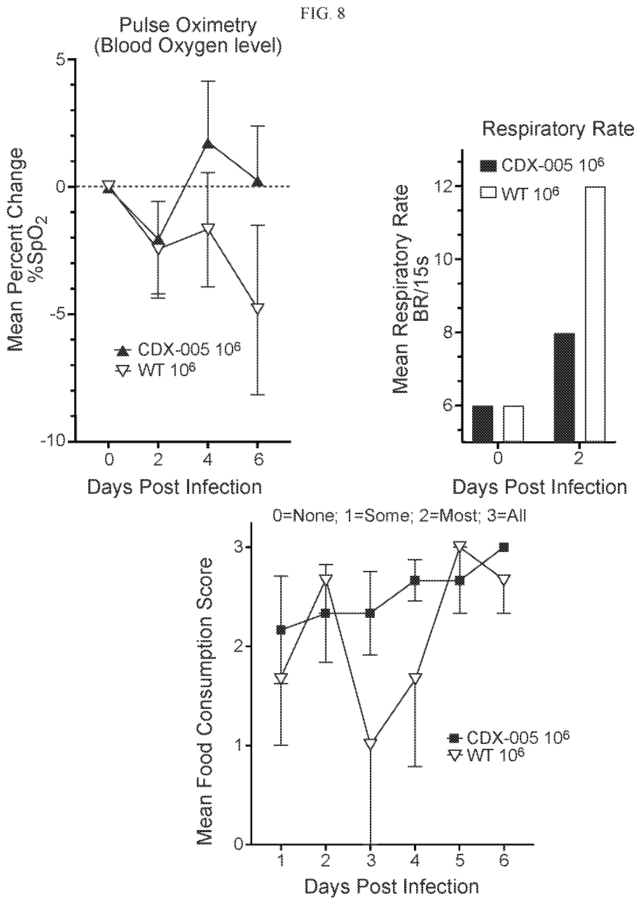

FIG. 9 depicts crude bulk titers of CDX-005 harvested from Vero cells. Vero WHO “10-87” cells were inoculated with 1.8×10⁴ PFU of CDX-005 (˜0.01 MOI) then grown for 48 hr. Virus was harvested using the different schemes shown.

FIG. 10 shows successful rescue of CDX-005.1 virus via Reverse Genetics. On May 7, 2021, 3 days after transfection of Vero WHO 10-87 cells with synthetic genome RNA derived from synthetic genome DNA, cell supernatants were tested for presence of infectious CDX-005.1 virus by plaque assay on VeroE6 cells. Infectious CDX-005.1 virus was detected at a titer of 4.6×10⁵ PFU/mL, and with plaque sizes indistinguishable to those of CDX-005.

FIG. 11 shows successful rescue of CDX-005.2 virus via Reverse Genetics. 4 days after transfection of Vero WHO 10-87 cells with synthetic genome RNA derived from synthetic genome DNA, cell supernatants were tested for presence of infectious CDX-005.2 virus by plaque assay on VeroE6 cells. Infectious CDX-005.2 virus was detected at a titer of 2.5×10⁵ PFU/mL, and with plaque sizes indistinguishable to those of CDX-005.

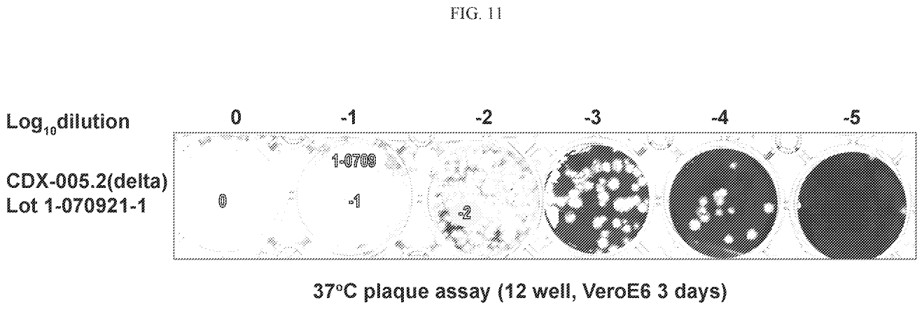

FIG. 12 shows that CDX-005.2 is temperature sensitive at 40° C. Plaque assays were performed in parallel on the same batch of Vero E6 cell monolayers in 12-well clusters and stained with Crystal Violet after 4 days of incubation at 37° C. or 40° C. CDX-005.2 (upper row) and CDX-005 (lower row) samples from the same dilution series were used to perform plaque assays at either 37° C. or 40° C. At 40° C., plaques for both viruses are significantly smaller, irregularly shaped, and much reduced in number (approximately 1,000-fold for CDX-005, and 10,000-fold for CDX-005.2) as compared to the permissive temperature of 37° C. Temperature sensitive phenotypes at elevated temperature (human equivalent of fever) are generally regarded as a positive safety feature of live attenuated virus vaccines.

FIG. 13 shows efficacy of the deoptimized SARS-CoV2 (CDX.005) as a vaccine against S. African variant B.1.351.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, or 0.5% of that referenced numeric indication, if specifically provided for in the claims.

“Parent virus” as used herein refer to a reference virus to which a recoded nucleotide sequence is compared for encoding the same or similar amino acid sequence.

“SARS-CoV-2” and “2019-nCoV” as used herein are interchangeable, and refer to a coronavirus that has a wild-type sequence, natural isolate sequence, or mutant forms of the wild-type sequence or natural isolate sequence that causes COVID-19. Mutant forms arise naturally through the virus' replication cycles, or through genetic engineering.

“SARS-CoV-2 variant” as used herein refers to a mutant form of SARS-CoV-2 that has developed naturally through the virus' replication cycles as it replicates in and/or transmits between hosts such as humans. Examples of SARS-CoV-2 variants include but are not limited to Alpha variant (also known as U.K. variant, 201/501Y.V1, VOC 202012/01, or B.1.1.7), Beta variant (also known as South African variant, 20H/501Y.V2, or B.1.351,), Delta variant (B.1.617.2), Gamma variant (also known as Brazil variant or P.1), Omicron variant (B.1.1.529), Omicron variant lineages (BA.1, BA.1.1, BA.2, BA.3, BA.4 and BA.5).

“Natural isolate” as used herein with reference to SARS-CoV-2 refers to a virus such as SARS-CoV-2 that has been isolated from a host (e.g., human, bat, feline, pig, or any other host) or natural reservoir. The sequence of the natural isolate can be identical or have mutations that arose naturally through the virus' replication cycles as it replicates in and/or transmits between hosts, for example, humans.

“Washington coronavirus isolate” or “Washington isolate” as used herein refers to a wild-type isolate of SARS-CoV-2 that has GenBank accession no. MN985325.1 as of Jul. 5, 2020, which is herein incorporated by reference as though fully set forth in its entirety.

“WW-WWD”, “CDX-005” and “COVI-VAC” are used interchangeably. “COVI-VAC” was a name previously used in the priority application to describe CDX-005.

“Frequently used codons” or “codon usage bias” as used herein refer to differences in the frequency of occurrence of synonymous codons in coding DNA for a particular species, for example, human, coronavirus, or SARS-CoV-2.

“Codon pair bias” as used herein refers to synonymous codon pairs that are used more or less frequently than statistically predicted in a particular species, for example, human, coronavirus, or SARS-CoV-2.

A “subject” as used herein means any animal or artificially modified animal. Animals include, but are not limited to, humans, non-human primates, monkeys, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “viral host” means any animal or artificially modified animal that a virus can infect. Animals include, but are not limited to, humans, non-human primates, monkeys, cows, horses, sheep, pigs, dogs, cats, rabbits, ferrets, rodents such as mice, rats and guinea pigs, and birds. Artificially modified animals include, but are not limited to, SCID mice with human immune systems. In various embodiments, the viral host is a mammal. In various embodiments, the viral host is a primate. In various embodiments, the viral host is human. Embodiments of birds are domesticated poultry species, including, but not limited to, chickens, turkeys, ducks, and geese.

A “prophylactically effective dose” is any amount of a vaccine or virus composition that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold, 25-fold, 50-fold, or 100 fold. For example, if a subject has a 1% chance of becoming infected with a virus, a two-fold reduction in the likelihood of the subject becoming infected with the virus would result in the subject having a 0.5% chance of becoming infected with the virus.

As used herein, a “therapeutically effective dose” is any amount of a vaccine or virus composition that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

“Corresponding sequence” as used herein refers to a comparison sequence by which the modified sequence is encoding the same or similar amino acid sequence of the comparison sequence. In various embodiments, the corresponding sequence is a sequence that encodes a viral protein. In various embodiments, the corresponding sequence is at least 50 codons in length. In various embodiments, the corresponding sequence is at least 100 codons in length. In various embodiments, the corresponding sequence is at least 150 codons in length. In various embodiments, the corresponding sequence is at least 200 codons in length. In various embodiments, the corresponding sequence is at least 250 codons in length. In various embodiments, the corresponding sequence is at least 300 codons in length. In various embodiments, the corresponding sequence is at least 350 codons in length. In various embodiments, the corresponding sequence is at least 400 codons in length. In various embodiments, the corresponding sequence is at least 450 codons in length. In various embodiments, the corresponding sequence is at least 500 codons in length. In various embodiments, the corresponding sequence is a viral protein sequence. In various embodiments, the corresponding sequence is the sequence of the entire virus.

In various embodiments, “similar amino acid sequence” as used herein refers to an amino acid sequence having less than 2% amino acid substitutions, deletions or additions compared to the comparison sequence. In various embodiments, if specifically provided for in the claims, “similar amino acid sequence” refers to an amino acid sequence having less than 1.75% amino acid substitutions, deletions or additions compared to the comparison sequence. In various embodiments, if specifically provided for in the claims, “similar amino acid sequence” refers to an amino acid sequence having less than 1.5% amino acid substitutions, deletions or additions compared to the comparison sequence. In various embodiments, if specifically provided for in the claims, “similar amino acid sequence” refers to an amino acid sequence having less than 1.25% amino acid substitutions, deletions or additions compared to the comparison sequence. In various embodiments, if specifically provided for in the claims, “similar amino acid sequence” refers to an amino acid sequence having less than 1% amino acid substitutions, deletions or additions compared to the comparison sequence. In various embodiments, if specifically provided for in the claims, “similar amino acid sequence” refers to an amino acid sequence having less than 0.75% amino acid substitutions, deletions or additions compared to the comparison sequence. In various embodiments, if specifically provided for in the claims, “similar amino acid sequence” refers to an amino acid sequence having less than 0.5% amino acid substitutions, deletions or additions compared to the comparison sequence. In various embodiments, if specifically provided for in the claims, “similar amino acid sequence” refers to an amino acid sequence having less than 0.25% amino acid substitutions, deletions or additions compared to the comparison sequence.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

Described herein are SARS-CoV-2 variants wherein its genes have been recoded, for example, codon pair bias deoptimized or codon usage deoptimized. In various embodiments, the viral proteins of SARS-CoV-2 variants of the present invention have the same amino acid sequences as its parent SARS-CoV-2 variant; however, the nucleotide sequences have been recoded. Recoding of the nucleotide sequence in accordance with the present invention results in reduced protein expression, attenuation or both. These recoded SARS-CoV-2 variants are useful as vaccines, and particularly, for use as live-attenuated vaccines.

We previously generated a synthetic highly attenuated live vaccine candidate, CDX-005 from wt SARS-CoV-2. While not wishing to be bound by any particular theory, we believe that the most likely mechanism for the attenuation is slowed translation, through errors in translation leading to misfolded proteins, changes in RNA secondary structure, or altered regulatory signals may all contribute to reduced protein production. Whatever the mechanism, the attenuated CDX-005 virus presents every viral antigen in its wt form, providing the potential for a broad immune response and making it likely to retain efficacy even if there is genetic drift in the target strain. CDX-005 is expected to be highly resistant to reversion to pathogenicity since hundreds of silent (synonymous) mutations contribute to the phenotype. Our tests of reversion indicate that the vaccine is stable as assessed by bulk sequencing of late passage virus and evaluation of potential changes in the furin cleavage site.

Our hamster studies demonstrate that CDX-005 is safe in these animals. It is highly attenuated, inducing lower total viral loads in the lungs and olfactory bulb and completely abrogating it in the brain and inducing lower live viral loads in the lung of animals inoculated with CDX-005 than those with wt WA1. Unlike wt virus, CDX-005 did not induce weight loss or significant lung pathology in inoculated hamsters.

The hamster studies also suggest that CDX-005 effectively protect against SARS CoV-2. Assessment of Abs titers demonstrate that it is as effective as wt virus in inducing serum IgG and neutralizing Abs. It is protective against wt challenge; inoculation with CDX-005 leads to lower lung viral titers and complete protection against virus in the brain. Hamsters inoculated with CDX-005 also do not exhibit the weight loss observed in vehicle inoculated animals. Moreover, there is no evidence of disease enhancement.

Together our data indicates that CDX-005 is a part of an important new class of live attenuated vaccines currently being developed for use in animals and humans. It presents all viral antigens similar to their native amino acid sequence, can be administered intranasally, is safe and effective in small animal models with a single dose, is resistant to reversion, and can be grown to high titers at a permissive temperature. Clinical trials are currently underway to test its safety and efficacy in humans.

To construct the deoptimized CDX-005 (e.g., comprising SEQ ID NO:1) live attenuated vaccine candidates, first the genome of the wild-type WA1 donor virus was parsed in silico into 19 overlapping fragments. Each fragment shares approximately 200 bp of sequence overlap with each adjacent fragment. F1-F19 were generated from cDNA of wild-type WA1 virus RNA by RT-PCR. The fragments were sequence confirmed by Sanger sequencing. We then exchanged Fragment 16 of the WT WA1 virus for fragment 16 that had the deoptimized spike gene sequence to generate the cDNA genome of CDX-005.

In various embodiments, the molecular parsing of a target SARS-CoV-2 and its variants into small fragments each with about 50 to 300 bp overlaps via RT-PCR and the exchange of any of these fragments is a process that can be used to construct the cDNA genome or genome fragment of any codon-, or codon-pair-deoptimized virus. This cDNA genome with the deoptimized cassette can then be used to recover a deoptimized virus via reverse genetics.

For CDX-005, we identified one notable difference in the sequence of our WA1 donor virus (Vero cell passage 6) compared to the published WA1 sequence (Vero cell passage 4). During the two additional WA1 virus passages on Vero E6 cells at Codagenix of the WA1 virus received from BEI Resources, a 36 nt deletion occurred in the Spike gene (genome position 23594-23629). The deletion encompasses the 12 amino acids TNSPRRARSVAS (SEQ ID NO: 13) that include the polybasic furin cleavage site. The furin cleavage site in SARS-CoV2 Spike has been proposed as a potential driver of the highly pathogenic phenotype of SARS-CoV2 in the human host. While not wishing to be bound by any particular theory, we believe that absence of the furin cleavage is beneficial to the SARS-CoV-2 virus' and its variants' growth in vitro in Vero cells, and that the deletion evolved during passaging in Vero cell culture. We further believe that the absence of the furin cleavage site may contribute to attenuation in the human host of a SARS-CoV-2 virus or variant carrying such mutation. We therefore decided to incorporate the furin cleavage site deletion that was derived into our vaccine candidate CDX-005. The furin cleavage site deletion is located in assembly fragment F15.

However, since the emergence of SARS-CoV-2 variants, new vaccines are needed to ensure a robust protection against the variant forms. Accordingly, we set out to generate deoptimized SARS-CoV-2 variants to allow for greater protection against SARS-CoV-2 variants.

The present invention is based, at least in part, on the foregoing and on the further information as described herein.

In various embodiments, the viral proteins of SARS-CoV-2 variants of the present invention have the same amino acid sequences as its parent SARS-CoV-2 variants but with up to about 20 amino acid deletion(s), substitution(s), or addition(s). However, the nucleotide sequences have been recoded, which results in reduced protein expression, attenuation or both. In various embodiments, the viral proteins of SARS-CoV-2 variants of the present invention have the same amino acid sequences as its parent SARS-CoV-2 variants but with up to 10 amino acid deletions, substitutions, or additions; however, the nucleotide sequences have been recoded, which results in reduced protein expression, attenuation or both. In various embodiments, the viral proteins of SARS-CoV-2 variants of the present invention have the same amino acid sequences as its parent SARS-CoV-2 variants but between 1-5 amino acid deletion, substitution, or addition. In various embodiments, the viral proteins of SARS-CoV-2 variants of the present invention have the same amino acid sequences as its parent SARS-CoV-2 variants but between 6-10 amino acid deletion, substitution, or addition. In various embodiments, the viral proteins of SARS-CoV-2 variants of the present invention have the same amino acid sequences as its parent SARS-CoV-2 variants but between 11-15 amino acid deletion, substitution, or addition. In various embodiments, the viral proteins of SARS-CoV-2 variants of the present invention have the same amino acid sequences as its parent SARS-CoV-2 variant but between 16-20 amino acid deletion, substitution, or addition. Again, however, the nucleotide sequences have been recoded, which results in reduced protein expression, attenuation or both. In various embodiments, the viral proteins of a SARS-CoV-2 variants of the present invention have the same amino acid sequences as its parent SARS-CoV-2 variant but 12 amino acid deletions, substitutions, or additions; however, the nucleotide sequences have been recoded, which results in reduced protein expression, attenuation or both. In various embodiments, the amino acid deletion, substitution, or addition results from nucleic acid deletion(s), substitution(s) or addition(s) before the polyA tail of the nucleic acid sequence of the parent SARS-CoV-2 variant sequence.

In various embodiments, the viral proteins of SARS-CoV-2 variant of the present invention have the same amino acid sequences as its parent SARS-CoV-2 variant but with a 12 amino acid deletion. In various embodiments, the viral proteins of SARS-CoV-2 variants of the present invention have the same amino acid sequences as its parent SARS-CoV-2 variant but with a 1-5 amino acid deletion, or a 6-10 amino acid deletion, or a 11-15 amino acid deletion, or a 16-20 amino acid deletion. In various embodiments, the amino acid deletion is in the Spike protein that eliminates the furin cleavage site. In various particular embodiments, the viral proteins of SARS-CoV-2 variants of the present invention have the same amino acid sequences as its parent SARS-CoV-2 variant but with a 12 amino acid deletion that results in the elimination of the furin cleavage site on the Spike protein. In various embodiments, the amino acid deletion, substitution, or addition results from nucleic acid deletion(s), substitution(s) or addition(s) before the polyA tail of the nucleic acid sequence of the parent SARS-CoV-2 variant sequence.

In various embodiments, the nucleic acid encoding the spike protein (also known as S gene) of the SARS-CoV-2 variant is recoded. In various embodiments, the recoded spike protein comprises a deletion of nucleotides that eliminates the fin cleavage site; for example, a 36 nucleotide sequence having the following sequence actaattctcctcggcgggcacgtagtgtagctagt (SEQ ID NO:14) or a nucleic acid sequence that encodes TNSPRRARSVAS (SEQ ID NO:13).

The recoding of spike protein encoding sequences of the attenuated viruses of the invention have been made or can be made by one of skill in the art in light of disclosure discussed herein. According to various embodiments of the invention, nucleotide substitutions are engineered in multiple locations in the spike protein coding sequence, wherein the substitutions introduce a plurality of synonymous codons into the genome. In certain embodiments, the synonymous codon substitutions alter codon bias, codon pair bias, the density of infrequent codons or infrequently occurring codon pairs, RNA secondary structure, CG and/or TA (or UA) dinucleotide content, C+G content, translation frameshift sites, translation pause sites, the presence or absence of microRNA recognition sequences or any combination thereof, in the genome. The codon substitutions may be engineered in multiple locations distributed throughout the spike protein coding sequence, or in the multiple locations restricted to a portion of the spike protein coding sequence. Because of the large number of defects (i.e., nucleotide substitutions) involved, the invention allows for production of stably attenuated viruses and live vaccines.

In some embodiments, virus codon pairs are recoded to reduce (i.e., lower the value of) codon-pair bias. In certain embodiments, codon-pair bias is reduced by identifying a codon pair in a spike coding sequence having a codon-pair score that can be reduced and reducing the codon-pair bias by substituting the codon pair with a codon pair that has a lower codon-pair score. In some embodiments, this substitution of codon pairs takes the form of rearranging existing codons of a sequence. In some such embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased CPS at many locations, there may be accompanying CPS increases at other locations, but on average, the codon pair scores, and thus the CPB of the modified sequence, is reduced. In some embodiments, recoding of codons or codon-pairs can take into account altering the G+C content of the spike coding sequence. In some embodiments, recoding of codons or codon-pairs can take into account altering the frequency of CG and/or TA dinucleotides in the spike coding sequence.

In certain embodiments, the recoded spike protein-encoding sequence has a codon pair bias less than −0.1, or less than −0.2, or less than −0.3, or less than −0.4. In some embodiments, the recoded spike protein-encoding sequence has a codon pair bias less than −0.01, less than −0.02, less than −0.03, or less than −0.04. In some embodiments, the recoded spike protein-encoding sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded spike protein encoding sequence is reduced by at least 0.1, or at least 0.2, or at least 0.3, or at least 0.4, compared to the parent spike protein encoding sequence from which it is derived (e.g., the parent sequence spike protein encoding sequence, the variant sequence spike protein encoding sequence). In certain embodiments, rearrangement of synonymous codons of the spike protein-encoding sequence provides a codon-pair bias reduction of at least 0.1, or at least 0.2, or at least 0.3, or at least 0.4, compared to the parent spike protein encoding sequence from which it is derived. In certain embodiments, the codon pair bias of the recoded the spike protein-encoding sequence is reduced by at least 0.01, at least 0.02 at least 0.03, or at least 0.04. In certain embodiments, the codon pair bias of the recoded the spike protein-encoding sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the parent virus. In certain embodiments, it is in comparison corresponding sequence from which the calculation is to be made; for example, the corresponding sequence of a variant virus (e.g., spike protein-encoding sequence on variant virus).

In some embodiments, a virus coding sequence is recoded by substituting one or more codon with synonymous codons used less frequently in the SARS-CoV-2 coronavirus host (e.g., humans, snakes, bats). In some embodiments, a virus coding sequence is recoded by substituting one or more codons with synonymous codons used less frequently in a coronavirus; for example, the SARS-CoV-2 coronavirus. In certain embodiments, the number of codons substituted with synonymous codons is at least 5. In some embodiments, at least 10, 20, 30, 40, 50, 60, 70, 80, 90, 100, 200, 250, 300, 350, 400, 450, or 500 codons are substituted with synonymous codons less frequently used in the host. In certain embodiments, the modified sequence comprises at least 20 codons substituted with synonymous codons less frequently used. In certain embodiments, the modified sequence comprises at least 50 codons substituted with synonymous codons less frequently used. In certain embodiments, the modified sequence comprises at least 100 codons substituted with synonymous codons less frequently used. In certain embodiments, the modified sequence comprises at least 250 codons substituted with synonymous codons less frequently used. In certain embodiments, the modified sequence comprises at least 500 codons substituted with synonymous codons less frequently used.

For example, for the recoded spike protein, the number of codons substituted with synonymous codons less frequently used in the host is at least 5, 10, 20, 30, 40, 50, 60, 70, 80, 90, 100, 150, 200, 250, 300, 350, 400, 450, or 500 codons.

In some embodiments, the substitution of synonymous codons is with those that are less frequent in the viral host; for example, human. Other examples of viral hosts include but are not limited to those noted above. In some embodiments, the substitution of synonymous codons is with those that are less frequent in the virus itself, for example, the SARS-CoV-2 coronavirus.

In embodiments wherein the modified sequence comprises an increased number of CpG or UpA di-nucleotides compared to a corresponding sequence on the parent virus, the increase is of about 15-55 CpG or UpA di-nucleotides compared the corresponding sequence. In various embodiments, increase is of about 15, 20, 25, 30, 35, 40, 45, or 55 CpG or UpA di-nucleotides compared the corresponding sequence. In some embodiments, the increased number of CpG or UpA di-nucleotides compared to a corresponding sequence is about 10-75, 15-25, 25-50, or 50-75 CpG or UpA di-nucleotides compared the corresponding sequence.

Usually, these substitutions and alterations are made and reduce expression of the encoded virus proteins without altering the amino acid sequence of the encoded protein. In certain embodiments, the invention also includes alterations in the spike coding sequence that result in substitution of non-synonymous codons and amino acid substitutions in the encoded protein, which may or may not be conservative. In some embodiments, these substitutions and alterations further include substitutions or alterations that results in amino acid deletions, additions, substitutions. For example, the spike protein can be recoded with a 36 nucleotide deletion that results in the elimination of the furin cleavage site.

In various embodiments, a continuous segment of a viral protein is recoded, wherein the continuous segment is about ¾ the length of the viral protein. In various embodiments, a continuous segment of a viral protein is recoded, wherein the continuous segment is about ½ the length of the viral protein. In various embodiments, a continuous segment of a viral protein is recoded, wherein the continuous segment is about ⅓ the length of the viral protein. In various embodiments, a continuous segment of a viral protein is recoded, wherein the continuous segment is about ¼ the length of the viral protein. In various embodiments, a continuous segment of a viral protein is recoded, wherein the continuous segment is about ⅕ the length of the viral protein.

In various embodiments, a continuous segment of a viral protein is recoded, wherein the continuous segment is about 10-20% of the length of the viral protein. In various embodiments, a continuous segment of a viral protein is recoded, wherein the continuous segment is about 20-30% of the length of the viral protein. In various embodiments, a continuous segment of a viral protein is recoded, wherein the continuous segment is about 25-35% of the length of the viral protein. In various embodiments, a continuous segment of a viral protein is recoded, wherein the continuous segment is about 30-40% of the length of the viral protein. In various embodiments, a continuous segment of a viral protein is recoded, wherein the continuous segment is about 35-45% of the length of the viral protein. In various embodiments, a continuous segment of a viral protein is recoded, wherein the continuous segment is about 40-50% of the length of the viral protein. In various embodiments, a continuous segment of a viral protein is recoded, wherein the continuous segment is about 45-55% of the length of the viral protein. In various embodiments, a continuous segment of a viral protein is recoded, wherein the continuous segment is about 50-60% of the length of the viral protein. In various embodiments, a continuous segment of a viral protein is recoded, wherein the continuous segment is about 55-65% of the length of the viral protein. In various embodiments, a continuous segment of a viral protein is recoded, wherein the continuous segment is about 60-70% of the length of the viral protein. In various embodiments, a continuous segment of a viral protein is recoded, wherein the continuous segment is about 70-80% of the length of the viral protein.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy.

TABLE 1
Genetic Code

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a} The first nucleotide in each codon encoding a particular amino acid is shown in the left-most column; the second nucleotide is shown in the top row; and the third nucleotide is shown in the right-most column.

### Codon Bias

As used herein, a rare codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40 of the time, but use the synonymous CTA only 7G of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source: www.kazusa.or.jp/codon/)

| Amino | | | | | Amino | | | | |
|---|---|---|---|---|---|---|---|---|---|
| Acid | Codon | Number | /1000 | Fraction | Acid | Codon | Number | /1000 | Fraction |
| Gly | GGG | 636457.00 | 16.45 | 0.25 | Trp | TGG | 510256.00 | 13.19 | 1.00 |
| Gly | GGA | 637120.00 | 16.47 | 0.25 | End | TGA | 59528.00 | 1.54 | 0.47 |
| Gly | GGT | 416131.00 | 10.76 | 0.16 | Cys | TGT | 407020.00 | 10.52 | 0.45 |
| Gly | GGC | 862557.00 | 22.29 | 0.34 | Cys | TGC | 487907.00 | 12.61 | 0.55 |
| Glu | GAG | 1532589.00 | 39.61 | 0.58 | End | TAG | 30104.00 | 0.78 | 0.24 |
| Glu | GAA | 1116000.00 | 28.84 | 0.42 | End | TAA | 38222.00 | 0.99 | 0.30 |
| Asp | GAT | 842504.00 | 21.78 | 0.46 | Tyr | TAT | 470083.00 | 12.15 | 0.44 |
| Asp | GAC | 973377.00 | 25.16 | 0.54 | Tyr | TAC | 592163.00 | 15.30 | 0.56 |
| Val | GTG | 1091853.00 | 28.22 | 0.46 | Leu | TTG | 498920.00 | 12.89 | 0.13 |
| Val | GTA | 273515.00 | 7.07 | 0.12 | Leu | TTA | 294684.00 | 7.62 | 0.08 |
| Val | GTT | 426252.00 | 11.02 | 0.18 | Phe | TTT | 676381.00 | 17.48 | 0.46 |
| Val | GTC | 562086.00 | 14.53 | 0.24 | Phe | TTC | 789374.00 | 20.40 | 0.54 |
| Ala | GCG | 286975.00 | 7.42 | 0.11 | Ser | TCG | 171428.00 | 4.43 | 0.05 |
| Ala | GCA | 614754.00 | 15.89 | 0.23 | Ser | TCA | 471469.00 | 12.19 | 0.15 |
| Ala | GCT | 715079.00 | 18.48 | 0.27 | Ser | TCT | 585967.00 | 15.14 | 0.19 |
| Ala | GCC | 1079491.00 | 27.90 | 0.40 | Ser | TCC | 684663.00 | 17.70 | 0.22 |
| Arg | AGG | 461676.00 | 11.93 | 0.21 | Arg | CGG | 443753.00 | 11.47 | 0.20 |
| Arg | AGA | 466435.00 | 12.06 | 0.21 | Arg | CGA | 239573.00 | 6.19 | 0.11 |
| Ser | AGT | 469641.00 | 12.14 | 0.15 | Arg | CGT | 176691.00 | 4.57 | 0.08 |
| Ser | AGC | 753597.00 | 19.48 | 0.24 | Arg | CGC | 405748.00 | 10.49 | 0.18 |
| Lys | AAG | 1236148.00 | 31.95 | 0.57 | Gln | CAG | 1323614.00 | 34.21 | 0.74 |
| Lys | AAA | 940312.00 | 24.30 | 0.43 | Gln | CAA | 473648.00 | 12.24 | 0.26 |
| Asn | AAT | 653566.00 | 16.89 | 0.47 | His | CAT | 419726.00 | 10.85 | 0.42 |
| Asn | AAC | 739007.00 | 19.10 | 0.53 | His | CAC | 583620.00 | 15.08 | 0.58 |
| Met | ATG | 853648.00 | 22.06 | 1.00 | Leu | CTG | 1539118.00 | 39.78 | 0.40 |
| Ile | ATA | 288118.00 | 7.45 | 0.17 | Leu | CTA | 276799.00 | 7.15 | 0.07 |
| Ile | ATT | 615699.00 | 15.91 | 0.36 | Leu | CTT | 508151.00 | 13.13 | 0.13 |
| Ile | ATC | 808306.00 | 20.89 | 0.47 | Leu | CTC | 759527.00 | 19.63 | 0.20 |
| Thr | ACG | 234532.00 | 6.06 | 0.11 | Pro | CCG | 268884.00 | 6.95 | 0.11 |
| Thr | ACA | 580580.00 | 15.01 | 0.28 | Pro | CCA | 653281.00 | 16.88 | 0.28 |
| Thr | ACT | 506277.00 | 13.09 | 0.25 | Pro | CCT | 676401.00 | 17.48 | 0.29 |
| Thr | ACC | 732313.00 | 18.93 | 0.36 | Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both.

### Codon Pair Bias

In addition, a given organism has a preference for the nearest codon neighbor of a given codon A, referred to a bias in codon pair utilization. A change of codon pair bias, without changing the existing codons, can influence the rate of protein synthesis and production of a protein.

Codon pair bias may be illustrated by considering the amino acid pair Ala-Glu, which can be encoded by 8 different codon pairs. If no factors other than the frequency of each individual codon (as shown in Table 2) are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes, the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.097 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Table 3.

TABLE 3
Codon Pair Scores Exemplified by the Amino Pair
Ala-Glu

| Amino acid | Codon | expected | observed | obs/exp |
|---|---|---|---|---|
| pair | pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example, the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$C P B = {\sum }_{i = 1}^{k} \frac{C P S i}{K - 1}$$

Accordingly, similar codon pair bias for a coding sequence can be obtained, for example, by minimized codon pair scores over a subsequence or moderately diminished codon pair scores over the full length of the coding sequence.

### Calculation of Codon Pair Bias

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurrences over the number that would have been expected in this set of genes (in this example the human genome). Determining the actual number of occurrences of a particular codon pair (or in other words the likelihood of a particular amino acid pair being encoded by a particular codon pair) is simply a matter of counting the actual number of occurrences of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair. The detailed equations used to calculate CPB are disclosed in WO 2008/121992 and WO 2011/044561, which are incorporated by reference.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, Pᵢⱼ is a codon pair occurring with a frequency of N_{O}(Pᵢⱼ) in its synonymous group. Cᵢ and Cⱼ are the two codons comprising Pᵢⱼ, occurring with frequencies F(Cᵢ) and F(Cⱼ) in their synonymous groups respectively. More explicitly, F(Cᵢ) is the frequency that corresponding amino acid Xiis coded by codon Cᵢ throughout all coding regions and F(Cᵢ)═N_{O}(Cⱼ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency Nₒ(Pᵢⱼ) over the expected number of occurrences of Nₑ(Pᵢⱼ).

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions.

The “combined” codon pair bias of an individual coding sequence was calculated by averaging all codon pair scores according to the following formula:

$$S ( {P}_{ij} ) = {\sum }_{l = 1}^{k} \frac{S ( {P}_{ij} ) l}{k - 1}$$

The codon pair bias of an entire coding region is thus calculated by adding all of the individual codon pair scores comprising the region and dividing this sum by the length of the coding sequence.

### Calculation of Codon Pair Bias, Implementation of Algorithm to Alter Codon-Pair Bias.

An algorithm was developed to quantify codon pair bias. Every possible individual codon pair was given a “codon pair score”, or “CPS”. CPS is defined as the natural log of the ratio of the observed over the expected number of occurrences of each codon pair over all human coding regions, where humans represent the host species of the instant vaccine virus to be recoded.

$$C P S = ln ( \frac{{F ( AB )}_{O}}{\frac{F ( A ) \times F ( B )}{F ( X ) \times F ( Y )} \times F ( XY )} )$$

Although the calculation of the observed occurrences of a particular codon pair is straightforward (the actual count within the gene set), the expected number of occurrences of a codon pair requires additional calculation. We calculate this expected number to be independent both of amino acid frequency and of codon bias, similar to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

Using these calculated CPSs, any coding region can then be rated as using over- or under-represented codon pairs by taking the average of the codon pair scores, thus giving a Codon Pair Bias (CPB) for the entire gene.

$$C P B = {\sum }_{i = 1}^{k} \frac{C P S i}{k - 1}$$

The CPB has been calculated for all annotated human genes using the equations shown and plotted. Each point in the graph corresponds to the CPB of a single human gene. The peak of the distribution has a positive codon pair bias of 0.07, which is the mean score for all annotated human genes. Also, there are very few genes with a negative codon pair bias. Equations established to define and calculate CPB were then used to manipulate this bias.

### Algorithm for Reducing Codon-Pair Bias.

Recoding of protein-encoding sequences may be performed with or without the aid of a computer, using, for example, a gradient descent, or simulated annealing, or other minimization routine. An example of the procedure that rearranges codons present in a starting sequence can be represented by the following steps:
1) Obtain wild-type viral genome sequence.
2) Select protein coding sequences to target for attenuated design.
3) Lock down known or conjectured DNA segments with non-coding functions.
4) Select desired codon distribution for remaining amino acids in redesigned proteins.
5) Perform random shuffle of at least two synonymous unlocked codon positions and calculate codon-pair score.
6) Further reduce (or increase) codon-pair score optionally employing a simulated annealing procedure.
7) Inspect resulting design for excessive secondary structure and unwanted restriction site:
   if yes->go to step (5) or correct the design by replacing problematic regions with wild-type sequences and go to step (8).
8) Synthesize DNA sequence corresponding to virus design.
9) Create viral construct and assess viral phenotype:
   if too attenuated, prepare subclone construct and go to 9;
   if insufficiently attenuated, go to 2.

Attenuation of viruses by reducing codon pair bias is disclosed in WO 2008/121992 and WO 2011/044561, which are incorporated by reference as though fully set forth.

Methods of obtaining full-length SARS-CoV-2 genome sequence or codon pair deoptimized sequences embedded in a wild-type SARS-CoV-2 genome sequence (or its mutant forms of the wild-type sequence that causes COVID-19) can include for example, constructing an infectious cDNA clone, using BAC vector, using an overlap extension PCR strategy, or long PCR-based fusion strategy.

### Recoded Polynucleotides

Various embodiments of the present invention provide for a polynucleotide encoding a spike protein or a fragment thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide remains the same. In various embodiments, the amino acid sequence of the spike protein or a fragment thereof of the parent SARS-CoV-2 variant encoded by the polynucleotide remains the same before the polyA tail of the nucleic acid sequence of the parent SARS-CoV-2 variant sequence.

Various embodiments of the present invention provide for a polynucleotide encoding spike protein or a fragment thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide comprises up to 20 amino acid substitutions, additions, or deletions. In various embodiments, the amino acid sequence of the spike protein or a fragment thereof of the parent SARS-CoV-2 variant encoded by the polynucleotide comprises up to 20 amino acid substitutions, additions, or deletions is before the polyA tail of the nucleic acid sequence of the parent SARS-CoV-2 variant sequence.

Various embodiments of the present invention provide for a polynucleotide encoding spike protein or a fragment thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide comprises up to 10 amino acid substitutions, additions, or deletions. In various embodiments, the amino acid sequence of the spike protein or a fragment thereof of the parent SARS-CoV-2 variant encoded by the polynucleotide comprises up to 10 amino acid substitutions, additions, or deletions is before the polyA tail of the nucleic acid sequence of the parent SARS-CoV-2 variant sequence.

Various embodiments of the present invention provide for a polynucleotide encoding spike protein or a fragment thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide comprises up to 12 amino acid substitutions, additions, or deletions. In various embodiments, the amino acid sequence of the spike protein or a fragment thereof of the parent SARS-CoV-2 variant encoded by the polynucleotide comprises up to 12 amino acid substitutions, additions, or deletions is before the polyA tail of the nucleic acid sequence of the parent SARS-CoV-2 variant sequence.

In various embodiments, the amino acid sequence comprises up to 2, 3, 4, 5, 6, 7, 8, 9, 10, 11, 12, 13, 14, 15, 16, 17, 18, 19 or 20 amino acid substitutions, additions, or deletions. In various embodiments, the amino acid sequence comprises 1-5, 6-10, 11-15, or 16-20 amino acid substitutions, additions, or deletions. In various embodiments, the amino acid deletion, substitution, or addition results from nucleic acid deletion(s), substitution(s) or addition(s) before the polyA tail of the nucleic acid sequence of the parent SARS-CoV-2 variant sequence.

In various embodiments, the amino acid sequence comprises 12 amino acid deletions. In various embodiments, the amino acid sequence comprises 1-5, 6-10, 11-15, or 16-20 amino acid deletions. In various embodiments, the amino acid substitutions, additions, or deletions can be due to one or more point mutations in the recoded sequence. In various embodiments, the amino acid deletion, substitution, or addition results from nucleic acid deletion(s), substitution(s) or addition(s) before the polyA tail of the nucleic acid sequence of the parent SARS-CoV-2 variant sequence.

Thus, in various embodiments for these recoded polynucleotides (with or without the nucleic acid deletion(s), substitution(s) or addition(s)), the recoded polynucleotide can have a different length for the polyA tail; for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end; or for example, 1-6, 7-12, 13-18, 19-24, 25-30, 31-36, 37-42, 43-48, or 49-54 consecutive adenines on the 3′ end; or for example, 9-37, 12-34, 15-33, 18-30, or 21-27 consecutive adenines on the 3′ end; or for example, 19-25 consecutive adenines on the 3′ end.

In various embodiments, the polynucleotide is recoded by reducing codon-pair bias (CPB) compared to its parent SARS-CoV-2 variant polynucleotide. In various embodiments, the polynucleotide is recoded by reducing codon usage bias compared to its parent SARS-CoV-2 variant polynucleotide. In various embodiments, the polynucleotide is recoded by increasing the number of CpG or UpA di-nucleotides compared to its parent SARS-CoV-2 variant polynucleotide.

In various embodiments, the recoded spike protein or a fragment thereof has a codon pair bias less than, −0.05, less than −0.1, less than −0.2, less than −0.3, or less than −0.4.

In certain embodiments, the recoded spike protein or a fragment thereof has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the recoded spike protein or a fragment thereof is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the parent sequence. In certain embodiments, it is in comparison corresponding sequence on the parent sequence from which the calculation is to be made; for example, the corresponding sequence of a variant virus.

In various embodiments, the parent SARS-CoV-2 coronavirus is a SARS-CoV-2 variant. In various embodiments, SARS-CoV-2 variant is the U.K. variant. In some embodiments, the SARS-CoV-2 variant is the South Africa variant. In some embodiments, the SARS-CoV-2 variant is the Brazil variant. In some embodiments, the SARS-CoV-2 variant is the Delta variant. In some embodiments, the SARS-CoV-2 variant is the Omicron variant. In some embodiments, the SARS-CoV-2 variant is Omicron variant sub-linage BA.1, BA.1.1, BA.2, BA.3, BA.4 or BA.5. In some embodiments, the SARS-CoV-2 variant is Omicron variant sub-linage BA.4. In some embodiments, the SARS-CoV-2 variant is Omicron variant sub-linage BA.5.

Examples of the U.K. variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021, and all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021; and EPI_ISL_581117, EPI_ISL 596982, EPI_ISL_599956, EPI_ISL_600093, EPI_ISL_606375, EPI_ISL 606415, EPI_ISL 606424, EPI_ISL_608363, and EPI_ISL_608430, all as of Jun. 28, 2021; and all incorporated herein by reference as though fully set forth in their entirety.

Examples of the South Africa variant include but are not limited to GISAID ID Nos. EPI_ISL_766709 (hCoV-19/Sweden/20-13194/2020; 2020-12-24), EPI_ISL_768828 (hCoV-19/France/PAC-NRC2933/2020; 2020-12-22), EPI_ISL_770441 (hCoV-19/England/205280030/2020; 2020-12-24), and EPI_ISL_819798 (hCoV-19/England/OXON-F440A7/2020; 2020-12-18), all as of Jan. 20, 2021, and all incorporated herein by reference as though fully set forth in their entirety. Additional examples include but are not limited to and hCoV-19/Sweden/20-13194/2020 (EPI_ISL_766709), hCoV-19/England/205280030/2020 (EPI_ISL_770441), hCoV-19/France/PAC-NRC2933/2020 (EPI_ISL_768828), hCoV-19/South Korea/KDCA0463/2020 (EPI_ISL_762992), hCoV-19/Japan/IC-0433/2020 (EPI_ISL_768642), hCoV-19/Australia/NSW3876/2021 (EPI_ISL_775242), hCoV-19/Australia/NSW3872/2021 (EPI_ISL_775245), hCoV-19/France/PAC-NRC2929/2020 (EPI_ISL_768827), hCoV-19/England/205300109/2020 (EPI_ISL_770467), hCoV-19/England/205320747/2020 (EPI_ISL_770469), hCoV-19/England/205261884/2020 (EPI_ISL_770438), hCoV-19/England/205260233/2020 (EPI_ISL_770437), hCoV-19/England/ALDP-C8FEC7/2020 (EPI_ISL_777292), hCoV-19/England/205221138/2020 (EPI_ISL_766245), hCoV-19/England/205300065/2020 (EPI_ISL_770463), hCoV-19/Botswana/1217-IN1699/2020 (EPI_ISL_770472), hCoV-19/Botswana/1217-IN1660/2020 (EPI_ISL_770471), hCoV-19/England/ALDP-C8E7FA/2020 (EPI_ISL_777266), hCoV-19/England/MILK-C90388/2020 (EPI_ISL_777229), hCoV-19/Botswana/CV1615722/2020 (EPI_ISL_770474), hCoV-19/Botswana/CV1605828/2020 (EPI_ISL_770473), hCoV-19/Scotland/EDB11343/2020 (EPI_ISL_764279), hCoV-19/Scotland/EDB11342/2020 (EPI_ISL_764278), hCoV-19/England/ALDP-C690AF/2020 (EPI_ISL_777190), hCoV-19/Botswana/1223-IN1490/2020 (EPI_ISL_770475), hCoV-19/England/MILK-CA9C09/2020 (EPI_ISL_762362), hCoV-19/England/ALDP-CB4807/2020 (EPI_ISL_761052), hCoV-19/England/205300064/2020 (EPI_ISL_770462), hCoV-19/England/MILK-CA9BB1/2020 (EPI_ISL_762499), hCoV-19/England/MILK-CAE2B7/2020 (EPI_ISL_761059), hCoV-19/England/205390867/2021 (EPI_ISL_768815), hCoV-19/Botswana/1224-IN462/20201 (EPI_ISL_770470), hCoV-19/England/205280028/2020 (EPI_ISL 770439), and hCoV-19/England/205280029/2020 (EPI_ISL_770440), all as of Jun. 28, 2021; and all incorporated herein by reference as though fully set forth in their entirety.

Examples of the Brazil variant include but are not limited to GISAID ID Nos. EPI_ISL_677212 (hCoV-19/USA/VA-DCLS-2187/2020; 2020-11-12), EPI_ISL_723494 (hCoV-19/USA/VA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPI_ISL_848197 (hCoV-19/Canada/LTRI-1258/2020; 2020-12-24), all as of Jan. 20, 2021, and all incorporated herein by reference as though fully set forth in their entirety.

Examples of the Delta (B1.617.2) variant include but are not limited to GISAID ID Nos. EPI_ISL_1653403, EPI_ISL_1697977, EPI_ISL_1718959, EPI_ISL_1719027, EPI_ISL_2121225, EPI_ISL_2121637, EPI_ISL_2121989, EPI_ISL_2122659, EPI_ISL_2125463, EPI_ISL_2126212, EPI_ISL 2126374, EPI_ISL 2127610, EPI_ISL_2127624, EPI_ISL_2127831, and EPI_ISL 2131345, all as of Jun. 28, 2021.

In various embodiments, the parent SARS-CoV-2 variant is a previously modified viral nucleic acid, or a previously attenuated viral nucleic acid.

In various embodiments, the polynucleotide is CPB deoptimized compared to its parent SARS-CoV-2 variant polynucleotide. In various embodiments, the polynucleotide is codon usage deoptimized compared to its parent SARS-CoV-2 variant polynucleotide.

In various embodiments, the CPB deoptimized is based on CPB in humans. In various embodiments, the CPB deoptimized is based on CPB in a coronavirus. In various embodiments, the CPB deoptimized is based on CPB in a SARS-CoV-2 coronavirus. In various embodiments, the CPB deoptimized is based on CPB in a wild-type SARS-CoV-2 coronavirus. The wild-type SARS-CoV-2 coronavirus may be a SARS-CoV-2 variant coronavirus in accordance with various embodiments discuss herein.

In various embodiments, the codon usage deoptimized is based on frequently used codons in humans. In various embodiments, the codon usage deoptimized is based on frequently used codons in a coronavirus. In various embodiments, the codon usage deoptimized is based on frequently used codons or a SARS-CoV-2 coronavirus. In various embodiments, the codon usage deoptimized is based on frequently used codons or CPB in a wild-type SARS-CoV-2 coronavirus. The wild-type SARS-CoV-2 coronavirus may be a SARS-CoV-2 variant coronavirus in accordance with various embodiments discuss herein.

In various embodiments, the polynucleotide comprises a recoded a spike protein, a fragment of spike protein, and combinations thereof. In various embodiments, polynucleotide comprises a deletion of nucleotides that results in a deletion of amino acids in the spike protein that eliminates the furin cleavage site. While not wishing to be bound by any particular theory, the inventors believe that eliminating the furin cleavage site is one of the drivers of safety of the vaccine and/or immune composition.

In various embodiment, the polynucleotide comprises SEQ ID NO:1, wherein the spike protein coding sequence in SEQ ID NO:1 is replaced with a recoded spike protein coding sequence from a SARS-CoV-2 variant.

In various embodiment, the polynucleotide comprises SEQ ID NO:1 wherein nt 9469 is changed from to A to G and nt 26222 changed from T to G, wherein the spike protein coding sequence in SEQ ID NO:1 is replaced with a recoded spike protein coding sequence from a SARS-CoV-2 variant.

In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is one or more mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is two or more mutations in SEQ ID NO:1. In various embodiment, the polynucleotide comprises SEQ ID NO: 1, wherein the spike protein coding sequence in SEQ ID NO:1 is replaced with a recoded spike protein coding sequence from a SARS-CoV-2 variant, and wherein there is up to 5 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 10 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 20 mutations in SEQ ID NO:1. In various embodiment, the polynucleotide comprises SEQ ID NO: 1, wherein the spike protein coding sequence in SEQ ID NO:1 is replaced with a recoded spike protein coding sequence from a SARS-CoV-2 variant, and wherein there is up to 30 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 40 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 50 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 60 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 70 mutations in SEQ ID NO:1. In various embodiments, the mutations in SEQ ID NO:1 is not an Alpha variant, Beta variant, Delta variant, Gamma variant, or Omicron variant.

SEQ ID NO:1 is a deoptimized sequence in comparison to the wild-type WA-1 sequence (GenBank: MN985325.1 herein incorporated by reference as though fully set forth).

In various embodiments, the SARS-CoV-2 variant is the Alpha variant.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8 In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Gamma variant.

In various embodiments, the SARS-CoV-2 variant is the Omicron variant.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

Full Length recoded sequences

| | | SEQ |
|---|---|---|
| | | ID |
| Variant | Recoded Sequence | NO: |
| Beta | ATTAaaggtttataccttcccaggtaacaaaccaaccaactttcgatctcttgta | 8 |
| (P1) w/o | gatctgttctctaaacgaactttaaaatctgtgtggctgtcactcggctgcatgc | |
| polyA tail | ttagtgcactcacgcagtataattaataactaattactgtcgttgacaggacacg | |
| and furin | agtaactcgtctatcttctgcaggctgcttacggtttcgtccgtgttgcagccga | |
| cleavage | tcatcagcacatctaggtttcgtccgggtgtgaccgaaaggtaagatggagagcc | |
| site | ttgtccctggtttcaacgagaaaacacacgtccaactcagtttgcctgttttaca | |
| Spike | ggttcgcgacgtgctcgtacgtggctttggagactccgtggaggaggtcttatca | |
| region | gaggcacgtcaacatcttaaagatggcacttgtggcttagtagaagttgaaaaag | |
| bolded | gcgttttgcctcaacttgaacagccctatgtgttcatcaaacgttcggatgctcg | |
| | aactgcacctcatggtcatgttatggttgagctggtagcagaactcgaaggcatt | |
| | cagtacggtcgtagtggtgagacacttggtgtccttgtccctcatgtgggcgaaa | |
| | taccagtggcttaccgcaaggttcttcttcgtaagaacggtaataaaggagctgg | |
| | tggccatagttacggcgccgatctaaagtcatttgacttaggcgacgagcttggc | |
| | actgatccttatgaagattttcaagaaaactggaacactaaacatagcagtggtg | |
| | ttacccgtgaactcatgcgtgagcttaacggaggggcatacactcgctatgtcga | |
| | taacaacttctgtggccctgatggctaccctcttgagtgcattaaagaccttcta | |
| | gcacgtgctggtaaagcttcatgcactttgtccgaacaactggactttattgaca | |
| | ctaagaggggtgtatactgctgccgtgaacatgagcatgaaattgcttggtacac | |
| | ggaacgttctgaaaagagctatgaattgcagacaccttttgaaattaaattggca | |
| | aagaaatttgacaccttcaatggggaatgtccaaattttgtatttcccttaaatt | |
| | ccataatcaagactattcaaccaagggttgaaaagaaaaagcttgatggctttat | |
| | gggtagaattcgatctgtctatccagttgcgtcaccaaatgaatgcaaccaaatg | |
| | tgcctttcaactctcatgaagtgtgatcattgtggtgaaacttcatggcagacgg | |
| | gcgattttgttaaagccacttgcgaattttgtggcactgagaatttgactaaaga | |
| | aggtgccactacttgtggttacttaccccaaaatgctgttgttaaaatttattgt | |
| | ccagcatgtcacaattcagaagtaggacctgagcatagtcttgccgaataccata | |
| | atgaatctggcttgaaaaccattcttcgtaagggtggtcgcactattgcctttgg | |
| | aggctgtgtgttctcttatgttggttgccataacaagtgtgcctattgggttcca | |
| | cgtgctagcgctaacataggttgtaaccatacaggtgttgttggagaaggttccg | |
| | aaggtcttaatgacaaccttcttgaaatactccaaaaagagaaagtcaacatcaa | |
| | tattgttggtgactttaaacttaatgaagagatcgccattattttggcatctttt | |
| | tctgcttccacaagtgcttttgtggaaactgtgaaaggtttggattataaagcat | |
| | tcaaacaaattgttgaatcctgtggtaattttaaagttacaaaaggaaaagctaa | |
| | aaaaggtgcctggaatattggtgaacagaaatcaatactgagtcctctttatgcG | |
| | tttgcatcagaggctgctcgtgttgtacgatcaattttctcccgcactcttgaaa | |
| | ctgctcaaaattctgtgcgtgttttacagaaggccgctataacaatactagatgg | |
| | aatttcacagtattcactgagactcattgatgctatgatgttcacatctgatttg | |
| | gctactaacaatctagttgtaatggcctacattacaggtggtgttgttcagttga | |
| | cttcgcagtggctaactaacatctttggcactgtttatgaaaaactcaaacccgt | |
| | ccttgattggcttgaagagaagtttaaggaaggtgtagagtttcttagagacggt | |
| | tgggaaattgttaaatttatctcaacctgtgcttgtgaaattgtcggtggacaaa | |
| | ttgtcacctgtgcaaaggaaattaaggagagtgttcagacattctttaagcttgt | |
| | aaataaatttttggctttgtgtgctgactctatcattattggtggagctaaactt | |
| | aaagccttgaatttaggtgaaacatttgtcacgcactcaaagggattgtacagaa | |
| | agtgtgttaaatccagagaagaaactggcctactcatgcctctaaaagccccaaa | |
| | agaaattatcttcttagagggagaaacacttcccacagaagtgttaacagaggaa | |
| | gttgtcttgaaaactggtgatttacaaccattagaacaacctactagtgaagctg | |
| | ttgaagctccattggttggtacaccagtttgtattaacgggcttatgttgctcga | |
| | aatcaaagacacagaaaagtactgtgcccttgcacctaatatgatggtaacaaac | |
| | aataccttcacactcaaaggcggtgcaccaacaaaggttacttttggtgatgaca | |
| | ctgtgatagaagtgcaaggttacaagagtgtgaatatcacttttgaacttgatga | |
| | aaggattgataaagtacttaatgagaagtgctctgcctatacagttgaactcggt | |
| | acagaagtaaatgagttcgcctgtgttgtggcagatgctgtcataaaaactttgc | |
| | aaccagtatctgaattacttacaccactgggcattgatttagatgagtggagtat | |
| | ggctacatactacttatttgatgagtctggtgagtttaTattggcttcacatatg | |
| | tattgttctttctaccctccagatgaggatgaagaagaaggtgattgtgaagaag | |
| | aagagtttgagccatcaactcaatatgagtatggtactgaagatgattaccaagg | |
| | taaacctttggaatttggtgccacttctgctgctcttcaacctgaagaagagcaa | |
| | gaagaagattggttagatgatgatagtcaacaaactgttggtcaacaagacggca | |
| | gtgaggacaatcagacaactactattcaaacaattgttgaggttcaacctcaatt | |
| | agagatggaacttacaccagttgttcagactattgaagtgaatagttttagtggt | |
| | tatttaaaacttactgacaatgtatacattaaaaatgcagacattgtggaagaag | |
| | ctaaaaaggtaaaaccaacagtggttgttaatgcagccaatgtttaccttaaaca | |
| | tggaggaggtgttgcaggagccttaaataaggctactaacaatgccatgcaagtt | |
| | gaatctgatgattacatagctactaatggaccacttaaagtgggtggtagttgtg | |
| | ttttaagcggacacaatcttgctaaacactgtcttcatgttgtcggcccaaatgt | |
| | taacaaaggtgaagacattcaacttcttaagagtgcttatgaaaattttaatcag | |
| | cacgaagttctacttgcaccattattatcagctggtatttttggtgctgacccta | |
| | tacattctttaagagtttgtgtagatactgttcgcacaaatgtctacttagctgt | |
| | ctttgataaaaatctctatgacaaacttgtttcaagctttttggaaatgaagagt | |
| | gaaaagcaagttgaacaaaagatcgctgagattcctaaagaggaagttaagccat | |
| | ttataactgaaagtaaaccttcagttgaacagagaaaacaagatgataagaaaat | |
| | caaagcttgtgttgaagaagttacaacaactctggaagaaactaagttcctcaca | |
| | gaaaacttgttactttatattgacattaatggcaatcttcatccagattctgcca | |
| | ctcttgttagtgacattgacatcactttcttaaagaaagatgctccatatatagt | |
| | gggtgatgttgttcaagagggtgttttaactgctgtggttatacctactaaaaag | |
| | gctggtggcactactgaaatgctagcgaaagctttgagaaaagtgccaacagaca | |
| | attatataaccacttacccgggtcagggtttaaatggttacactgtagaggaggc | |
| | aaagacagtgcttaaaaagtgtaaaagtgccttttacattctaccatctattatc | |
| | tctaatgagaagcaagaaattcttggaactgtttcttggaatttgcgagaaatgc | |
| | ttgcacatgcagaagaaacacgcaaattaatgcctgtctgtgtggaaactaaagc | |
| | catagtttcaactatacagcgtaaatataagggtattaaaatacaagagggtgtg | |
| | gttgattatggtgctagattttacttttacaccagtaaaacaactgtagcgtcac | |
| | ttatcaacacacttaacgatctaaatgaaactcttgttacaatgccacttggcta | |
| | tgtaacacatggcttaaatttggaagaagctgctcggtatatgagatctctcaaa | |
| | gtgccagctacagtttctgtttcttcacctgatgctgttacagcgtataatggtt | |
| | atcttacttcttcttctaaaacacctgaagaacattttattgaaaccatctcact | |
| | tgctggttcctataaagattggtcctattctggacaatctacacaactaggtata | |
| | gaatttcttaagagaggtgataaaagtgtatattacactagtaatcctaccacat | |
| | tccacctagatggtgaagttatcacctttgacaatcttaagacacttctttcttt | |
| | gagagaagtgaggactattaaggtgtttacaacagtagacaacattaacctccac | |
| | acgcaagttgtggacatgtcaatgacatatggacaacagtttggtccaacttatt | |
| | tggatggagctgatgttactaaaataaaacctcataattcacatgaaggtaaaac | |
| | attttatgttttacctaatgatgacactctacgtgttgaggcttttgagtactac | |
| | cacacaactgatcctagttttctgggtaggtacatgtcagcattaaatcacacta | |
| | aaaagtggaaatacccacaagttaatggtttaacttctattaaatgggcagataa | |
| | caactgttatcttgccactgcattgttaacactccaacaaatagagttgaagttt | |
| | aatccacctgctctacaagatgcttattacagagcaagggctggtgaagctgcta | |
| | acttttgtgcacttatcttagcctactgtaataagacagtaggtgagttaggtga | |
| | tgttagagaaacaatgagttacttgtttcaacatgccaatttagattcttgcaaa | |
| | agagtcttgaacgtggtgtgtaaaacttgtggacaacagcagacaacccttaagg | |
| | gtgtagaagctgttatgtacatgggcacactttcttatgaacaatttaagaaagg | |
| | tgttcagataccttgtacgtgtggtaaacaagctacaaaatatctagtacaacag | |
| | gagtcaccttttgttatgatgtcagcaccacctgctcagtatgaacttaagcatg | |
| | gtacatttacttgtgctagtgagtacactggtaattaccagtgtggtcactataa | |
| | acatataacttctaaagaaactttgtattgcatagacggtgctttacttacaaag | |
| | tcctcagaatacaaaggtcctattacggatgttttctacaaagaaaacagttaca | |
| | caacaaccataaaaccagttacttataaattggatggtgttgtttgtacagaaat | |
| | tgaccctaagttggacaattattataagaaagacaattcttatttcacagagcaa | |
| | ccaattgatcttgtaccaaaccaaccatatccaaacgcaagcttcgataatttta | |
| | agtttgtatgtgataatatcaaatttgctgatgatttaaaccagttaactggtta | |
| | taagaaacctgcttcaagagagcttaaagttacatttttccctgacttaaatggt | |
| | gatgtggtggctattgattataaacactacacaccctcttttaagaaaggagcta | |
| | aattgttacataaacctattgtttggcatgttaacaatgcaactaataaagccac | |
| | gtataaaccaaatacctggtgtatacgttgtctttggagcacaaaaccagttgaa | |
| | acatcaaattcgtttgatgtactgaagtcagaggacgcgcagggaatggataatc | |
| | ttgcctgcgaagatctaaaaccagtctctgaagaagtagtggaaaatcctaccat | |
| | acagaaagacgttcttgagtgtaatgtgaaaactaccgaagttgtaggagacatt | |
| | atacttaaaccagcaaataatagtttaaaaattacagaagaggttggccacacag | |
| | atctaatggctgcttatgtagacaattctagtcttactattaagaaacctaatga | |
| | attatctagagtattaggtttgaaaacccttgctactcatggtttagctgctgtt | |
| | aatagtgtcccttgggatactatagctaattatgctaagccttttcttaacaaag | |
| | ttgttagtacaactactaacatagttacacggtgtttaaaccgtgtttgtactaa | |
| | ttatatgccttatttctttactttattgctacaattgtgtacttttactagaagt | |
| | acaaattctagaattaaagcatctatgccgactactatagcaaagaatactgtta | |
| | agagtgtcggtaaattttgtctagaggcttcatttaattatttgaagtcacctaa | |
| | tttttctaaactgataaatattataatttggtttttactattaagtgtttgccta | |
| | ggttctttaatctactcaaccgctgctttaggtgttttaatgtctaatttaggca | |
| | tgccttcttactgtactggttacagagaaggctatttgaactctactaatgtcac | |
| | tattgcaacctactgtactggttctataccttgtagtgtttgtcttagtggttta | |
| | gattctttagacacctatccttctttagaaactatacaaattaccatttcatctt | |
| | ttaaatgggatttaactgcttttggcttagttgcagagtggtttttggcatatat | |
| | tcttttcactaggtttttctatgtacttggattggctgcaatcatgcaattgttt | |
| | ttcagctattttgcagtacattttattagtaattcttggcttatgtggttaataa | |
| | ttaatcttgtacaaatggccccgatttcagctatggttagaatgtacatcttctt | |
| | tgcatcattttattatgtatggaaaagttatgtgcatgttgtagacggttgtaat | |
| | tcatcaacttgtatgatgtgttacaaacgtaatagagcaacaagagtcgaatgta | |
| | caactattgttaatggtgttagaaggtccttttatgtctatgctaatggaggtaa | |
| | aggcttttgcaaactacacaattggaattgtgttaattgtgatacattctgtgct | |
| | ggtagtacatttattagtgatgaagttgcgagagacttgtcactacagtttaaaa | |
| | gaccaataaatcctactgaccagtcttcttacatcgttgatagtgttacagtgaa | |
| | gaatggttccatccatctttactttgataaagctggtcaaaagacttatgaaaga | |
| | cattctctctctcattttgttaacttagacaacctgagagctaataacactaaag | |
| | gttcattgcctattaatgttatagtttttgatggtaaatcaaaatgtgaagTatc | |
| | atctgcaaaatcagcgtctgtttactacagtcagcttatgtgtcaacctatactg | |
| | ttactagatcaggcattagtgtctgatgttggtgatagtgcggaagttgcagtta | |
| | aaatgtttgatgcttacgttaatacgttttcatcaacttttaacgtaccaatgga | |
| | aaaactcaaaacactagttgcaactgcagaagctgaacttgcaaagaatgtgtcc | |
| | ttagacaatgtcttatctacttttatttcagcagctcggcaagggtttgttgatt | |
| | cagatgtagaaactaaagatgttgttgaatgtcttaaattgtcacatcaatctga | |
| | catagaagttactggcgatagttgtaataactatatgctcacctataacaaagtt | |
| | gaaaacatgacaccccgtgaccttggtgcttgtattgactgtagtgcgcgtcata | |
| | ttaatgcgcaggtagcaaaaagtcacaacattgctttgatatggaacgttaaaga | |
| | tttcatgtcattgtctgaacaactacgaaaacaaatacgtagtgctgctaaaaag | |
| | aataacttaccttttaagttgacatgtgcaactactagacaagttgttaatgttg | |
| | taacaacaaagatagcacttaagggtggtaaaattgttaataattggttgaagca | |
| | gttaattaaagttacacttgtgttcctttttgttgctgctattttctatttaata | |
| | acacctgttcatgtcatgtctaaacatactgacttttcaagtgaaatcataggat | |
| | acaaggctattgatggtggtgtcactcgtgacatagcatctacagatacttgttt | |
| | tgctaacaaacatgctgattttgacacatggtttagtcagcgtggtggtagttat | |
| | actaatgacaaagcttgcccattgattgctgcagtcataacaagagaagtgggtt | |
| | ttgtcgtgcctggtttgcctggcacgatattacgcacaactaatggtgacttttt | |
| | gcatttcttacctagagtttttagtgcagttggtaacatctgttacacaccatca | |
| | aaacttatagagtacactgactttgcaacatcagcttgtgttttggctgctgaat | |
| | gtacaatttttaaagatgcttctggtaagccagtaccatattgttatgataccaa | |
| | tgtactagaaggttctgttgcttatgaaagtttacgccctgacacacgttatgtg | |
| | ctcatggatggctctattattcaatttcctaacacctaccttgaaggttctgtta | |
| | gagtggtaacaacCtttgattctgagtactgtaggcacggcacttgtgaaagatc | |
| | agaagctggtgtttgtgtatctactagtggtagatgggtacttaacaatgattat | |
| | tacagatctttaccaggagttttctgtggtgtagatgctgtaaatttacttacta | |
| | atatgtttacaccactaattcaacctattggtgctttggacatatcagcatctat | |
| | agtagctggtggtattgtagctatcgtagtaacatgccttgcctactattttatg | |
| | aggtttagaagagcttttggtgaatacagtcatgtagttgcctttaatactttac | |
| | tattccttatgtcattcactgtactctgtttaacaccagtttactcattcttacc | |
| | tggtgtttattctgttatttacttgtacttgacattttatcttactaatgatgtt | |
| | tcttttttagcacatattcagtggatggttatgttcacacctttagtacctttct | |
| | ggataacaattgcttatatcatttgtatttccacaaagcatttctattggttctt | |
| | tagtaattacctaaagagacgtgtagtctttaatggtgtttcctttagtactttt | |
| | gaagaagctgcgctgtgcacctttttgttaaataaagaaatgtatctaaagttgc | |
| | gtagtgatgtgctattacctcttacgcaatataatagatacttagctctttataa | |
| | taagtacaagtattttagtggagcaatggatacaactagctacagagaagctgct | |
| | tgttgtcatctcgcaaaggctctcaatgacttcagtaactcaggttctgatgttc | |
| | tttaccaaccaccacaaacctctatcacctcagctgttttgcagagtggttttag | |
| | aaaaatggcattcccatctggtaaagttgagggttgtatggtacaagtaacttgt | |
| | ggtacaactacacttaacggtctttggcttgatgacgtagtttactgtccaagac | |
| | atgtgatctgcacctctgaagacatgcttaaccctaattatgaagatttactcat | |
| | tcgtaagtctaatcataatttcttggtacaggctggtaatgttcaactcagggtt | |
| | attggacattctatgcaaaattgtgtacttaagcttaaggttgatacagccaatc | |
| | ctaagacacctaagtataagtttgttcgcattcaaccaggacagactttttcagt | |
| | gttagcttgttacaatggttcaccatctggtgtttaccaatgtgctatgaggccc | |
| | aatttcactattaagggttcattccttaatggttcatgtggtagtgttggtttta | |
| | acatagattatgactgtgtctctttttgttacatgcaccatatggaattaccaac | |
| | tggagttcatgctggcacagacttagaaggtaacttttatggaccttttgttgac | |
| | aggcaaacagcacaagcagctggtacggacacaactattacagttaatgttttag | |
| | cttggttgtacgctgctgttataaatggagacaggtggtttctcaatcgatttac | |
| | cacaactcttaatgactttaaccttgtggctatgaagtacaattatgaacctcta | |
| | acacaagaccatgttgacatactaggacctctttctgctcaaactggaattgccg | |
| | ttttagatatgtgtgcttcattaaaagaattactgcaaaatggtatgaatggacg | |
| | taccatattgggtagtgctttattagaagatgaatttacaccttttgatgttgtt | |
| | agacaatgctcaggtgttactttccaaagtgcagtgaaaagaacaatcaagggta | |
| | cacaccactggttgttactcacaattttgacttcacttttagttttagtccagag | |
| | tactcaatggtctttgttcttttttttgtatgaaaatgcctttttaccttttgct | |
| | atgggtattattgctatgtctgcttttgcaatgatgtttgtcaaacataagcatg | |
| | catttctctgtttgtttttgttaccttctcttgccactgtagcttattttaatat | |
| | ggtctatatgcctgctagttgggtgatgcgtattatgacatggttggatatggtt | |
| | gatactagtttgtctggttttaagctaaaagactgtgttatgtatgcatcagctg | |
| | tagtgttactaatccttatgacagcaagaactgtgtatgatgatggtgctaggag | |
| | agtgtggacacttatgaatgtcttgacactcgtttataaagtttattatggtaat | |
| | gctttagatcaagccatttccatgtgggctcttataatctctgttacttctaact | |
| | actcaggtgtagttacaactgtcatgttCttggccagaggtattgtttttatgtg | |
| | tgttgagtattgccctattttcttcataactggtaatacacttcagtgtataatg | |
| | ctagtttattgtttcttaggctatttttgtacttgttactttggcctcttttgtt | |
| | tactcaaccgctactttagactgactcttggtgtttatgattacttagtttctac | |
| | acaggagtttagatatatgaattcacagggactactcccacccaagaatagcata | |
| | gatgccttcaaactcaacattaaattgttgggtgttggtggcaaaccttgtatca | |
| | aagtagccactgtacagtctaaaatgtcagatgtaaagtgcacatcagtagtctt | |
| | actctcagttttgcaacaactcagagtagaatcatcatctaaattgtgggctcaa | |
| | tgtgtccagttacacaatgacattctcttagctaaagatactactgaagcctttg | |
| | aaaaaatggtttcactactttctgttttgctttccatgcagggtgctgtagacat | |
| | aaacaagctttgtgaagaaatgctggacaacagggcaaccttacaagctatagcc | |
| | tcagagtttagttcccttccatcatatgcagcttttgctactgctcaagaagctt | |
| | atgagcaggctgttgctaatggtgattctgaagttgttcttaaaaagttgaagaa | |
| | gtctttgaatgtggctaaatctgaatttgaccgtgatgcagccatgcaacgtaag | |
| | ttggaaaagatggctgatcaagctatgacccaaatgtataaacaggctagatctg | |
| | aggacaagagggcaaaagttactagtgctatgcagacaatgcttttcactatgct | |
| | tagaaagttggataatgatgcactcaacaacattatcaacaatgcaagagatggt | |
| | tgtgttcccttgaacataatacctcttacaacagcagccaaactaatggttgtca | |
| | taccagactataacacatataaaaatacgtgtgatggtacaacatttacttatgc | |
| | atcagcattgtgggaaatccaacaggttgtagatgcagatagtaaaattgttcaa | |
| | cttagtgaaattagtatggacaattcacctaatttagcatggcctcttattgtaa | |
| | cagctttaagggccaattctgctgtcaaattacagaataatgagcttagtcctgt | |
| | tgcactacgacagatgtcttgtgctgccggtactacacaaactgcttgcactgat | |
| | gacaatgcgttagcttactacaacacaacaaagggaggtaggtttgtacttgcac | |
| | tgttatccgatttacaggatttgaaatgggctagattccctaagagtgatggaac | |
| | tggtactatctatacagaactggaaccaccttgtaggtttgttacagacacacct | |
| | aaaggtcctaaagtgaagtatttatactttattaaaggattaaacaacctaaata | |
| | gaggtatggtacttggtagtttagctgccacagtacgtctacaagctggtaatgc | |
| | aacagaagtgcctgccaattcaactgtattatctttctgtgcttttgctgtagat | |
| | gctgctaaagcttacaaagattatctagctagtgggggacaaccaatcactaatt | |
| | gtgttaagatgttgtgtacacacactggtactggtcaggcaataacagttacacc | |
| | ggaagccaatatggatcaagaatcctttggtggtgcatcgtgttgtctgtactgc | |
| | cgttgccacatagatcatccaaatcctaaaggattttgtgacttaaaaggtaagt | |
| | atgtacaaatacctacaacttgtgctaatgaccctgtgggttttacacttaaaaa | |
| | cacagtctgtaccgtctgcggtatgtggaaaggttatggctgtagttgtgatcaa | |
| | ctccgcgaacccatgcttcagtcagctgatgcacaatcgtttttaaacgggtttg | |
| | cggtgtaagtgcagcccgtcttacaccgtgcggcacaggcactagtactgatgtc | |
| | gtatacagggcttttgacatctacaatgataaagtagctggttttgctaaattcc | |
| | taaaaactaattgttgtcgcttccaagaaaaggacgaagatgacaatttaattga | |
| | ttcttactttgtagttaagagacacactttctctaactaccaacatgaagaaaca | |
| | atttataatttacttaaggattgtccagctgttgctaaacatgacttctttaagt | |
| | ttagaatagacggtgacatggtaccacatatatcacgtcaacgtcttactaaata | |
| | cacaatggcagacctcgtctatgctttaaggcattttgatgaaggtaattgtgac | |
| | acattaaaagaaatacttgtcacatacaattgttgtgatgatgattatttcaata | |
| | aaaaggactggtatgattttgtagaaaacccagatatattacgcgtatacgccaa | |
| | cttaggtgaacgtgtacgccaagctttgttaaaaacagtacaattctgtgatgcc | |
| | atgcgaaatgctggtattgttggtgtactgacattagataatcaagatctcaatg | |
| | gtaactggtatgatttcggtgatttcatacaaaccacgccaggtagtggagttcc | |
| | tgttgtagattcttattattcattgttaatgcctatattaaccttgaccagggct | |
| | ttaactgcagagtcacatgttgacactgacttaacaaagccttacattaagtggg | |
| | atttgttaaaatatgacttcacggaagagaggttaaaactctttgaccgttattt | |
| | taaatattgggatcagacataccacccaaattgtgttaactgtttggatgacaga | |
| | tgcattctgcattgtgcaaactttaatgttttattctctacagtgttcccaccta | |
| | caagttttggaccactagtgagaaaaatatttgttgatggtgttccatttgtagt | |
| | ttcaactggataccacttcagagagctaggtgttgtacataatcaggatgtaaac | |
| | ttacatagctctagacttaTttttaaggaattacttgtgtatgctgctgaccctg | |
| | ctatgcacgctgcttctggtaatctattactagataaacgcactacgtgcttttc | |
| | agtagctgcacttactaacaatgttgcttttcaaactgtcaaacccggtaatttt | |
| | aacaaagacttctatgactttgctgtgtctaagggtttctttaaggaaggaagtt | |
| | ctgttgaattaaaacacttcttctttgctcaggatggtaatgctgctatcagcga | |
| | ttatgactactatcgttataatctaccaacaatgtgtgatatcagacaactacta | |
| | tttgtagttgaagttgttgataagtactttgattgttacgatggtggctgtatta | |
| | atgctaaccaagtcatcgtcaacaacctagacaaatcagctggttttccatttaa | |
| | taaatggggtaaggctagactttattatgattcaatgagttatgaggatcaagat | |
| | gcacttttcgcatatacaaaacgtaatgtcatccctactataactcaaatgaatc | |
| | ttaagtatgccattagtgcaaagaatagagctcgcaccgtagctggtgtctctat | |
| | ctgtagtactatgaccaatagacagtttcatcaaaaattattgaaatcaatagcc | |
| | gccactagaggagctactgtagtaattggaacaagcaaattctatggtggttggc | |
| | acaacatgttaaaaactgtttatagtgatgtagaaaaccctcaccttatgggttg | |
| | ggattatcctaaatgtgatagagccatgcctaacatgcttagaattatggcctca | |
| | cttgttcttgctcgcaaacatacaacgtgttgtagcttgtcacaccgtttctata | |
| | gattagctaatgagtgtgctcaagtattgagtgaaatggtcatgtgtggcggttc | |
| | actatatgttaaaccaggtggaacctcatcaggagatgccacaactgcttatgct | |
| | aatagtgtttttaacatttgtcaagctgtcacggccaatgttaatgcacttttat | |
| | ctactgatggtaacaaaattgccgataagtatgtccgcaatttacaacacagact | |
| | ttatgagtgtctctatagaaatagagatgttgacacagactttgtgaatgagttt | |
| | tacgcatatttgcgtaaacatttctcaatgatgatactctctgacgatgctgttg | |
| | tgtgtttcaatagcacttatgcatctcaaggtctagtggctagcataaagaactt | |
| | taagtcagttctttattatcaaaacaatgtttttatgtctgaagcaaaatgttgg | |
| | actgagactgaccttactaaaggacctcatgaattttgctctcaacatacaatgc | |
| | tagttaaacagggtgatgattatgtgtaccttccttacccagatccatcaagaat | |
| | cctaggggccggctgttttgtagatgatatcgtaaaaacagatggtacacttatg | |
| | attgaacggttcgtgtctttagctatagatgcttacccacttactaaacatccta | |
| | atcaggagtatgctgatgtctttcatttgtacttacaatacataagaaagctaca | |
| | tgatgagttaacaggacacatgttagacatgtattctgttatgcttactaatgat | |
| | aacacttcaaggtattgggaacctgagttttatgaggctatgtacacaccgcata | |
| | cagtcttacaggctgttggggcttgtgttctttgcaattcacagacttcattaag | |
| | atgtggtgcttgcatacgtagaccattcttatgttgtaaatgctgttacgaccat | |
| | gtcatatcaacatcacataaattagtcttgtctgttaatccgtatgtttgcaGtg | |
| | ctccaggttgtgatgtcacagatgtgactcaactttacttaggaggtatgagcta | |
| | ttattgtaaatcacataaaccacccattagttttccattgtgtgctaatggacaa | |
| | gtttttggtttatataaaaatacatgtgttggtagcgataatgttactgacttta | |
| | atgcaattgcaacatgtgactggacaaatgctggtgattacattttagctaacac | |
| | ctgtactgaaagactcaagctttttgcagcagaaacgctcaaagctactgaggag | |
| | acatttaaactgtcttatggtattgctactgtacgtgaagtgctgtctgacagag | |
| | aattacatctttcatgggaagttggtaaacctagaccaccacttaaccgaaatta | |
| | tgtctttactggttatcgtgtaactaaaaacagtaaagtacaaataggagagtac | |
| | acctttgaaaaaggtgactatggtgatgctgttgtttaccgaggtacaacaactt | |
| | acaaattaaatgttggtgattattttgtgctgacatcacatacagtaatgccatt | |
| | aagtgcacctacactagtgccacaagagcactatgttagaattactggcttatac | |
| | ccaacactcaatatctcagatgagttttctagcaatgttgcaaattatcaaaagg | |
| | ttggtatgcaaaagtattctacactccagggaccacctggtactggtaagagtca | |
| | ttttgctattggcctagctctctactacccttctgctcgcatagtgtatacagct | |
| | tgctctcatgccgctgttgatgcactatgtgagaaggcattaaaatatttgccta | |
| | tagataaatgtagtagaattatacctgcacgtgctcgtgtagagtgttttgataa | |
| | attcaaagtgaattcaacattagaacagtatgtcttttgtactgtaaatgcattg | |
| | cctgagacgacagcagatatagttgtctttgatgaaatttcaatggccacaaatt | |
| | atgatttgagtgttgtcaatgccagattacgtgctaagcactatgtgtacattgg | |
| | cgaccctgctcaattacctgcaccacgcacattgctaactaagggcacactagaa | |
| | ccagaatatttcaattcagtgtgtagacttatgaaaactataggtccagacatgt | |
| | tcctcggaacttgtcggcgttgtcctgctgaaattgttgacactgtgagtgcttt | |
| | ggtttatgataataagcttaaagcacataaagacaaatcagctcaatgctttaaa | |
| | atgttttataagggtgttatcacgcatgatgtttcatctgcaattaacaggccac | |
| | aaataggcgtggtaagagaattccttacacgtaaccctgcttggagaaaagctgt | |
| | ctttatttcaccttataattcacagaatgctgtagcctcaaagattttgggacta | |
| | ccaactcaaactgttgattcatcacagggctcagaatatgactatgtcatattca | |
| | ctcaaaccactgaaacagctcactcttgtaatgtaaacagatttaatgttgctat | |
| | taccagagcaaaagtaggcatactttgcataatgtctgatagagacctttatgac | |
| | aagttgcaatttacaagtcttgaaattccacgtaggaatgtggcaactttacaag | |
| | ctgaaaatgtaacaggactttttaaagattgtagtaaggtaatcactgggttaca | |
| | tcctacacaggcacctacacacctcagtgttgacactaaattcaaaactgaaggt | |
| | ttatgtgttgacatacctggcatacctaaggacatgacctatagaagactcatct | |
| | ctatgatgggttttaaaatgaattatcaagttaatggttaccctaacatgtttat | |
| | cacccgcgaagaagctataagacatgtacgtgcatggattggcttcgatgtcgag | |
| | gggtgtcatgctactagagaagctgttggtaccaatttacctttacagctaggtt | |
| | tttctacaggtgttaacctagttgctgtacctacaggttatgttgatacacctaa | |
| | taatacagatttttccagagttagtgctaaaccaccgcctggagatcaatttaaa | |
| | cacctcataccacttatgtacaaaggacttccttggaatgtagtgcgtataaaga | |
| | ttgtacaaatgttaagtgacacacttaaaaatctctctgacagagtcgtatttgt | |
| | cttatgggcacatggctttgagttgacatctatgaagtattttgtgaaaatagga | |
| | cctgagcgcacctgttgtctatgtgatagacgtgccacatgcttttccactgctt | |
| | cagacacttatgcctgttggcatcattctattggatttgattacgtctataatcc | |
| | gtttatgattgatgttcaacaatggggttttacaggtaacctacaaagcaaccat | |
| | gatctgtattgtcaagtccatggtaatgcacatgtagctagttgtgatgcaatca | |
| | tgactaggtgtctagctgtccacgagtgctttgttaagcgtgttgactggactat | |
| | tgaatatcctataattggtgatgaactgaagattaatgcggcttgtagaaaggtt | |
| | caacacatggttgttaaagctgcattattagcagacaaattcccagttcttcacg | |
| | acattggtaaccctaaagctattaagtgtgtacctcaagctgatgtagaatggaa | |
| | gttctatgatgcacagccttgtagtgacaaagcttataaaatagaagaattattc | |
| | tattcttatgccacacattctgacaaattcacagatggtgtatgcctattttgga | |
| | attgcaatgtcgatagatatcctgctaattccattgtttgtagatttgacactag | |
| | agtgctatctaaccttaacttgcctggttgtgatggtggcagtttgtatgtaaat | |
| | aaacatgcattccacacaccagcttttgataaaagtgcttttgttaatttaaaac | |
| | aattaccatttttctattactctgacagtccatgtgagtctcatggaaaacaagt | |
| | agtgtcagatatagattatgtaccactaaagtctgctacgtgtataacacgttgc | |
| | aatttaggtggtgctgtctgtagacatcatgctaatgagtacagattgtatctcg | |
| | atgcttataacatgatgatctcagctggctttagcttgtgggtttacaaacaatt | |
| | tgatacttataacctctggaacacttttacaagacttcagagtttagaaaatgtg | |
| | gcttttaatgttgtaaataagggacactttgatggacaacagggtgaagtaccag | |
| | tttctatcattaataacactgtttacacaaaagttgatggtgttgatgtagaatt | |
| | gtttgaaaataaaacaacattacctgttaatgtagcatttgagctttgggctaag | |
| | cgcaacattaaaccagtaccagaggtgaaaatactcaataatttgggtgtggaca | |
| | ttgctgctaatactgtgatctgggactacaaaagagatgctccagcacatatatc | |
| | tactattggtgtttgttctatgactgacatagccaagaaaccaactgaaacgatt | |
| | tgtgcaccactcactgtcttttttgatggtagagttgatggtcaagtagacttat | |
| | ttagaaatgcccgtaatggtgttcttattacagaaggtagtgttaaaggtttaca | |
| | accatctgtaggtcccaaacaagctagtcttaatggagtcacattaattggagaa | |
| | gccgtaaaaacacagttcaattattataagaaagttgatggtgttgtccaacaat | |
| | tacctgaaacttactttactcagagtagaaatttacaagaatttaaacccaggag | |
| | tcaaatggaaattgatttcttagaattagctatggatgaattcattgaacggtat | |
| | aaattagaaggctatgccttcgaacatatcgtttatggagattttagtcatagtc | |
| | agttaggtggtttacatctactgattggactagctaaacgttttaaggaatcacc | |
| | ttttgaattagaagattttattcctatggacagtacagttaaaaactatttcata | |
| | acagatgcgcaaacaggttcatctaagtgtgtgtgttctgttattgatttattac | |
| | ttgatgattttgttgaaataataaaatcccaagatttatctgtagtttctaaggt | |
| | tgtcaaagtgactattgactatacagaaatttcatttatgctttggtgtaaagat | |
| | ggccatgtagaaacattttacccaaaattacaatctagtcaagcgtggcaaccgg | |
| | gtgttgctatgcctaatctttacaaaatGCAAAGAATGCTATTAGAAAAGTGTGA | |
| | CCTTCAAAATTATGGTGATAGTGCAACATTACCTAAAGGCATAATGATGAATGTC | |
| | GCAAAATATACTCAACTGTGTCAATATTTAAACACATTAACATTAGCTGTACCCT | |
| | ATAATATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATTCAGAT | |
| | CTTAATGACTTTGTCTCTGATGCAGATTCAACTTTGATTGGTGATTGTGCAACTG | |
| | TACATACAGCTAATAAATGGGATCTCATTATTAGTGATATGTACGACCCTAAGAC | |
| | TAAAAATGTTACAAAAGAAAATGACTCTAAAGAGGGTTTTTTCACTTACATTTGT | |
| | GGGTTTATACAACAAAAGCTAGCTCTTGGAGGTTCCGTGGCTATAAAGATAACAG | |
| | AACATTCTTGGAATGCTGATCTTTATAAGCTCATGGGACACTTCGCATGGTGGAC | |
| | AGCCTTTGTTACTAATGTGAATGCGTCATCATCTGAAGCATTTTTAATTGGATGT | |
| | AATTATCTTGGCAAACCACGCGAACAAATAGATGGTTATGTCATGCATGCAAATT | |
| | ACATATTTTGGAGGAATACAAATCCAATTCAGTTGTCTTCCTATTCTTTATTTGA | |
| | CATGAGTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTATAATTA | |
| | GAGAAAACAACAGAGTTGTTATTTCTAGTGATGTTCTTGTTAACAACTAAACGAA | |
| | CAATGTTTGTTTTTCTTGTTTTATTGCCACTAGTCTCTAGTCAGTGTGTTAATtT | |
| | TACAACCAGAACTCAATTACCCCCTGCATACACTAATTCTTTCACACGTGGTGTT | |
| | TATTACCCTGACAAAGTTTTCAGATCCTCAGTTTTACATTCAACTCAGGACTTGT | |
| | TCTTACCTTTCTTTTCCAATGTTACTTGGTTCCATGCTATACATGTCTCTGGGAC | |
| | CAATGGTACTAAGAGGTTTGCTAACCCTGTCCTACCATTTAATGATGGTGTTTAT | |
| | TTTGCTTCCACTGAGAAGTCTAACATAATAAGAGGCTGGATTTTTGGTACTACTT | |
| | TAGATTCGAAGACCCAGTCCCTACTTATTGTTAATAACGCTACTAATGTTGTTAT | |
| | TAAAGTCTGTGAATTTCAATTTTGTAATGATCCATTTTTGGGTGTTTATTACCAC | |
| | AAAAACAACAAAAGTTGGATGGAAAGTGAGTTCAGAGTTTATTCTAGTGCGAATA | |
| | ATTGCACTTTTGAATATGTCTCTCAGCCTTTTCTTATGGACCTTGAAGGAAAACA | |
| | GGGTAATTTCAAAAATCTTAGGGAATTTGTGTTTAAGAATATTGATGGTTATTTT | |
| | AAAATATATTCTAAGCACACGCCTATTAATTTAGTGCGTGgTCTCCCTCAGGGTT | |
| | TTTCGGCTTTAGAACCATTGGTAGATTTGCCAATAGGTATTAACATCACTAGGTT | |
| | TCAAACTTTACATAGAAGTTATTTGACTCCTGGTGATTCTTCTTCAGGTTGGACA | |
| | GCTGGTGCTGCAGCTTATTATGTGGGTTATCTTCAACCTAGGACTTTTCTATTAA | |
| | AATATAATGAAAATGGAACCATTACAGATGCTGTAGACTGTGCACTTGACCCTCT | |
| | CTCAGAAACAAAGTGTACGTTGAAATCCTTCACTGTAGAAAAAGGAATCTATCAA | |
| | ACTTCTAACTTTAGAGTCCAACCAACAGAATCTATTGTTAGATTTCCTAATATTA | |
| | CAAACTTGTGCCCTTTTGGTGAAGTTTTTAACGCCACCAGATTTGCATCTGTTTA | |
| | TGCTTGGAACAGGAAGAGAATCAGCAACTGTGTTGCTGATTATTCTGTCCTATAT | |
| | AATTCCGCATCATTTTCCACTTTTAAGTGTTATGGAGTGTCTCCTACTAAATTAA | |
| | ATGATCTCTGCTTTACTAATGTCTATGCAGATTCATTTGTAATTAGAGGTGATGA | |
| | AGTCAGACAAATCGCTCCAGGGCAAACTGGAAAtATTGCTGATTATAATTATAAA | |
| | TTACCAGATGATTTTACAGGCTGCGTTATAGCTTGGAATTCTAACAATCTTGATT | |
| | CTAAGGTTGGTGGTAATTATAATTACCTGTATAGATTGTTTAGGAAGTCTAATCT | |
| | CAAACCTTTTGAGAGAGATATTTCAACTGAAATCTATCAGGCCGGTAGCACACCT | |
| | TGTAATGGTGTTaAAGGTTTTAATTGTTACTTTCCTTTACAATCATATGGTTTCC | |
| | AACCCACTtATGGTGTTGGTTACCAACCATACAGAGTAGTAGTACTTTCTTTTGA | |
| | ACTTCTACATGCACCAGCAACTGTTTGTGGACCTAAAAAGTCTACTAATTTGGTT | |
| | AAAAACAAATGTGTCAATTTCAACTTCAATGGTTTAACAGGCACAGGTGTTCTTA | |
| | CTGAGTCTAACAAAAAGTTTCTGCCTTTCCAACAATTTGGCAGAGACATTGCTGA | |
| | CACTACTGATGCTGTCCGTGATCCACAGACACTTGAGATTCTTGACATTACACCA | |
| | TGTTCTTTTGGTGGTGTCAGTGTTATAACACCAGGAACAAATACTTCTAACCAGG | |
| | TTGCTGTTCTTTATCAGGgTGTTAACTGCACAGAAGTCCCTGTTGCTATTCATGC | |
| | AGATCAACTTACTCCTACTTGGCGTGTTTATTCTACAGGTTCTAATGTTTTTCAA | |
| | ACACGTGCAGGCTGTTTAATAGGGGCTGAACATGTCAACAACTCATATGAGTGTG | |
| | ACATACCCATTGGTGCAGGTATATGCGCTAGTTATCAGACTCAGCAATCCATCAT | |
| | TGCCTACACTATGTCACTTGGTGtAGAAAATTCAGTTGCTTACTCTAATAACTCT | |
| | ATTGCCATACCCACAAATTTTACTATTAGTGTTACCACAGAAATTCTACCAGTGT | |
| | CTATGACCAAGACATCAGTAGATTGTACAATGTACATTTGTGGTGATTCAACTGA | |
| | ATGCAGCAATCTTTTGTTGCAATATGGCAGTTTTTGTACACAATTAAACCGTGCT | |
| | TTAACTGGAATAGCTGTTGAACAAGACAAAAACACCCAAGAAGTTTTTGCACAAG | |
| | TCAAACAAATTTACAAAACACCACCAATTAAAGATTTTGGTGGTTTTAATTTTTC | |
| | ACAAATATTACCAGATCCATCAAAACCAAGCAAGAGGTCATTTATTGAAGATCTA | |
| | CTTTTCAACAAAGTGACACTTGCAGATGCTGGCTTCATCAAACAATATGGTGATT | |
| | GCCTTGGTGATATTGCTGCTAGAGAtctcatttgcgctcaaaaatttaacggact | |
| | tacagttttaccacctttacttactgacgaaatgattgcgcaatatacatccgca | |
| | ttgttagccggaactattacatccggatggacttttggcgcaggcgTagcattac | |
| | agattccattcgctatgcaaatggcttataggtttaacggtataggcgttacgca | |
| | aaacgtactttatgagaatcaaaaacttatcgctaaccaatttaattccgctatc | |
| | ggtaagattcaggattcattgtctagtactgctagtgcactcggtaagttgcaag | |
| | acgtagtgaatcaaaacgctcaagcacttaatacactcgttaaacagcttagttc | |
| | taattttggcgcaatttctagtgtgcttaacgatatactatctagactcgataaa | |
| | gtcgaagccgaagtgcaaatcgatagattgattaccggtaggttgcaatcattgc | |
| | aaacatacgttacacagcaattgattagggccgcagagatacgcgctagcgctaa | |
| | tctcgcagctactaaaatgtctgaatgcgtactcggacaatctaaacgtgtcgat | |
| | ttttgcggtaagggatatcatcttatgtcttttccacaatctgcacctcacggag | |
| | tcgtgtttttacacgttacttatgtgccagctcaagagaaaaattttacaaccgc | |
| | tcctgctatttgtcatgacggtaaggcacattttcctagagagggcgtattcgtt | |
| | tctaacggtacacattggttcgttacacaacgtaatttttacgaacctcaaatta | |
| | ttactactgataatacattcgtatcaggtaattgtgacgtagtgataggtatcgt | |
| | taataatacagtttacgatccacttcaacctgaactcgatagttttaaagaggaa | |
| | ctcgataagtattttaaaaatcatacatcacctgacgtcgacttaggcgatattt | |
| | caggtattaacgctagtgtcgttaacattcaaaaagagattgatagacttaacga | |
| | agtcgctaaaaatcttaacgaatcacttatcgatctgcaagagttaggtaagtat | |
| | gagcaatatattaaatggccttggtatatttggttaggctttatagccggattga | |
| | tcgcaatcgttatggttacaattatgttatgttgtatgacatcatgttgttcatg | |
| | tcttaagggatgttgttcatgcggatcatgttgtaaatttgacgaagacgattcc | |
| | gaaccagtgcttaaaggcgttaagttacattatacataaacgaacttatggattt | |
| | gtttatgagaatcttcacaattggaactgtaactttgaagcaaggtgaaatcaag | |
| | gatgctactccttcagattttgttcgcgctactgcaacgataccgatacaagcct | |
| | cactccctttcggatggcttattgttggcgttgcacttcttgctgtttttcagag | |
| | cgcttccaaaatcataaccctcaaaaagagatggcaactagcactctccaagggt | |
| | gttcactttgtttgcaacttgctgttgttgtttgtaacagtttactcacaccttt | |
| | tgctcgttgctgctggccttgaagccccttttctctatctttatgctttagtcta | |
| | cttcttgcagagtataaactttgtaagaataataatgaggctttggctttgctgg | |
| | aaatgccgttccaaaaacccattactttatgatgccaactattttctttgctggc | |
| | atactaattgttacgactattgtataccttacaatagtgtaacttcttcaattgt | |
| | cattacttcaggtgatggcacaacaagtcctatttctgaacatgactaccagatt | |
| | ggtggttatactgaaaaatgggaatctggagtaaaagactgtgttgtattacaca | |
| | gttacttcacttcagactattaccagctgtactcaactcaattgagtacagacac | |
| | tggtgttgaacatgttaccttcttcatctacaataaaattgttgatgagcctgaa | |
| | gaacatgtccaaattcacacaatcgacggttcatccggagttgttaatccagtaa | |
| | tggaaccaatttatgatgaaccgacgacgactactagcgtgcctttgtaagcaca | |
| | agctgatgagtacgaacttatgtactcattcgtttcggaagagacaggtacgtta | |
| | atagttaatagcgtacttctttttcttgctttcgtggtattcttgctagttacac | |
| | tagccatccttactgcgcttcgattgtgtgcgtactgctgcaatattgttaacgt | |
| | gagtcttgtaaaaccttctttttacgtttactctcgtgttaaaaatctgaattct | |
| | tctagagttcctgatcttctggtctaaacgaactaaatattatattagtttttct | |
| | gtttggaactttaattttagccatggcagattccaacggtactattaccgttgaa | |
| | gagcttaaaaagctccttgaacaatggaacctagtaataggtttcctattcctta | |
| | catggatttgtcttctacaatttgcctatgccaacaggaataggtttttgtatat | |
| | aattaagttaattttcctctggctgttatggccagtaactttagcttgttttgtg | |
| | cttgctgctgtttacagaataaattggatcaccggtggaattgctatcgcaatgg | |
| | cttgtcttgtaggcttgatgtggctcagctacttcattgcttctttcagactgtt | |
| | tgcgcgtacgcgttccatgtggtcattcaatccagaaactaacattcttctcaac | |
| | gtgccactccatggcactattctgaccagaccgcttctagaaagtgaactcgtaa | |
| | tcggagctgtgatccttcgtggacatcttcgtattgctggacaccatctaggacg | |
| | ctgtgacatcaaggacctgcctaaagaaatcactgttgctacatcacgaacgctt | |
| | tcttattacaaattgggagcttcgcagcgtgtagcaggtgactcaggttttgctg | |
| | catacagtcgctacaggattggcaactataaattaaacacagaccattccagtag | |
| | cagtgacaatattgctttgcttgtacagtaagtgacaacagatgtttcatctcgt | |
| | tgactttcaggttactatagcagagatattactaattattatgaggacttttaaa | |
| | gtttccatttggaatcttgattacatcataaacctcataattaaaaatttatcta | |
| | agtcactaactgagaataaatattctcaattagatgaagagcaaccaatggagat | |
| | tgattaaacgaacatgaaaattattcttttcttggcactgataacactcgctact | |
| | tgtgagctttatcactaccaagagtgtgttagaggtacaacagtacttttaaaag | |
| | aaccttgctcttctggaacatacgagggcaattcaccatttcatcctctagctga | |
| | taacaaatttgcactgacttgctttagcactcaatttgcttttgcttgtcctgac | |
| | ggcgtaaaacacgtctatcagttacgtgccagatcagtttcacctaaactgttca | |
| | tcagacaagaggaagttcaagaactttactctccaatttttcttattgttgcggc | |
| | aatagtgtttataacactttgcttcacactcaaaagaaagacagaatgattgaac | |
| | tttcattaattgacttctatttgtgctttttagcctttctgctattccttgtttt | |
| | aattatgcttattatcttttggttctcacttgaactgcaagatcataatgaaact | |
| | tgtcacgcctaaacgaacatgaaatttcttgttttcttaggaatcatcacaactg | |
| | tagctgcatttcaccaagaatgtagtttacagtcatgtactcaacatcaaccata | |
| | tgtagttgatgacccgtgtcctattcacttctattctaaatggtatattagagta | |
| | ggagctagaaaatcagcacctttaattgaattgtgcgtggatgaggctggttcta | |
| | aatcacccattcagtacatcgatatcggtaattatacagtttcctgttcaccttt | |
| | tacaattaattgccaggaacctaaattgggtagtcttgtagtgcgttgttcgttc | |
| | tatgaagactttttagagtatcatgacgttcgtgttgttttagatttcatctaaa | |
| | cgaacaaactaaaatgtctgataatggaccccaaaatcagcgaaatgcaccccgc | |
| | attacgtttggtggaccctcagattcaactggcagtaaccagaatggagaacgca | |
| | gtggggcgcgatcaaaacaacgtcggccccaaggtttacccaataatactgcgtc | |
| | ttggttcaccgctctcactcaacatggcaaggaagaccttaaattccctcgagga | |
| | caaggcgttccaattaacaccaatagcagtccagatgaccaaattggctactacc | |
| | gaagagctaccagacgaattcgtggtggtgacggtaaaatgaaagatctcagtcc | |
| | aagatggtatttctactacctaggaactgggccagaagctggacttccctatggt | |
| | gctaacaaagacggcatcatatgggttgcaactgagggagccttgaatacaccaa | |
| | aagatcacattggcacccgcaatcctgctaacaatgctgcaatcgtgctacaact | |
| | tcctcaaggaacaacattgccaaaaggcttctacgcagaagggagcagaggcggc | |
| | agtcaagcctcttctcgttcctcatcacgtagtcgcaacagttcaagaaattcaa | |
| | ctccaggcagcagtaggggaacttctcctgctagaatggctggcaatggcggtga | |
| | tgctgctcttgctttgctgctgcttgacagattgaaccagcttgagagcaaaatg | |
| | tctggtaaaggccaacaacaacaaggccaaactgtcactaagaaatctgctgctg | |
| | aggcttctaagaagcctcggcaaaaacgtactgccactaaagcatacaatgtaac | |
| | acaagctttcggcagacgtggtccagaacaaacccaaggaaattttggggaccag | |
| | gaactaatcagacaaggaactgattacaaacattggccgcaaattgcacaatttg | |
| | cccccagcgcttcagcgttcttcggaatgtcgcgcattggcatggaagtcacacc | |
| | ttcgggaacgtggttgacctacacaggtgccatcaaattggatgacaaagatcca | |
| | aatttcaaagatcaagtcattttgctgaataagcatattgacgcatacaaaacat | |
| | tcccaccaacagagcctaaaaaggacaaaaagaagaaggctgatgaaactcaagc | |
| | cttaccgcagagacagaagaaacagcaaactgtgactcttcttcctgctgcagat | |
| | ttggatgatttctccaaacaattgcaacaatccatgagcagtgctgactcaactc | |
| | aggcctaaactcatgcagaccacacaaggcagatgggctatataaacgttttcgc | |
| | ttttccgtttacgatatatagtctactcttgtgcagaatgaattctcgtaactac | |
| | atagcacaagtagatgtagttaactttaatctcacatagcaatctttaatcagtg | |
| | tgtaacattagggaggacttgaaagagccaccacattttcaccgaggccacgcgg | |
| | agtacgatcgagtgtacagtgaacaatgctagggagagctgcctatatggaagag | |
| | ccctaatgtgtaaaattaattttagtagtgctatccccatgtgattttaatagct | |
| | tcttaggagaatgac | |
| Delta (P2) | ATTAaaggtttataccttcccaggtaacaaaccaaccaactttcgatctcttgta | 9 |
| w/o polyA | gatctgttctctaaacgaactttaaaatctgtgtggctgtcactcggctgcatgc | |
| tail | ttagtgcactcacgcagtataattaataactaattactgtcgttgacaggacacg | |
| | agtaactcgtctatcttctgcaggctgcttacggtttcgtccgtgttgcagccga | |
| | tcatcagcacatctaggtttcgtccgggtgtgaccgaaaggtaagatggagagcc | |
| | ttgtccctggtttcaacgagaaaacacacgtccaactcagtttgcctgttttaca | |
| | ggttcgcgacgtgctcgtacgtggctttggagactccgtggaggaggtcttatca | |
| | gaggcacgtcaacatcttaaagatggcacttgtggcttagtagaagttgaaaaag | |
| | gcgttttgcctcaacttgaacagccctatgtgttcatcaaacgttcggatgctcg | |
| | aactgcacctcatggtcatgttatggttgagctggtagcagaactcgaaggcatt | |
| | cagtacggtcgtagtggtgagacacttggtgtccttgtccctcatgtgggcgaaa | |
| | taccagtggcttaccgcaaggttcttcttcgtaagaacggtaataaaggagctgg | |
| | tggccatagttacggcgccgatctaaagtcatttgacttaggcgacgagcttggc | |
| | actgatccttatgaagattttcaagaaaactggaacactaaacatagcagtggtg | |
| | ttacccgtgaactcatgcgtgagcttaacggaggggcatacactcgctatgtcga | |
| | taacaacttctgtggccctgatggctaccctcttgagtgcattaaagaccttcta | |
| | gcacgtgctggtaaagcttcatgcactttgtccgaacaactggactttattgaca | |
| | ctaagaggggtgtatactgctgccgtgaacatgagcatgaaattgcttggtacac | |
| | ggaacgttctgaaaagagctatAaattgcagacaccttttgaaattaaattggca | |
| | aagaaatttgacaccttcaatggggaatgtccaaattttgtatttcccttaaatt | |
| | ccataatcaagactattcaaccaagggttgaaaagaaaaagcttgatggctttat | |
| | gggtagaattcgatctgtctatccagttgcgtcaccaaatgaatgcaaccaaatg | |
| | tgcctttcaactctcatgaagtgtgatcattgtggtgaaacttcatggcagacgg | |
| | gcgattttgttaaagccacttgcgaattttgtggcactgagaatttgactaaaga | |
| | aggtgccactacttgtggttacttaccccaaaatgctgttgttaaaatttattgt | |
| | ccagcatgtcacaattcagaagtaggacctgagcatagtcttgccgaataccata | |
| | atgaatctggcttgaaaaccattcttcgtaagggtggtcgcactattgcctttgg | |
| | aggctgtgtgttctcttatgttggttgccataacaagtgtgcctattgggttcca | |
| | cgtgctagcgctaacataggttgtaaccatacaggtgttgttggagaaggttccg | |
| | aaggtcttaatgacaaccttcttgaaatactccaaaaagagaaagtcaacatcaa | |
| | tattgttggtgactttaaacttaatgaagagatcgccattattttggcatctttt | |
| | tctgcttccacaagtgcttttgtggaaactgtgaaaggtttggattataaagcat | |
| | tcaaacaaattgttgaatcctgtggtaattttaaagttacaaaaggaaaagctaa | |
| | aaaaggtgcctggaatattggtgaacagaaatcaatactgagtcctctttatgca | |
| | tttgcatcagaggctgctcgtgttgtacgatcaattttctcccgcactcttgaaa | |
| | ctgctcaaaattctgtgcgtgttttacagaaggccgctataacaatactagatgg | |
| | aatttcacagtattcactgagactcattgatgctatgatgttcacatctgatttg | |
| | gctactaacaatctagttgtaatggcctacattacaggtggtgttgttcagttga | |
| | cttcgcagtggctaactaacatctttggcactgtttatgaaaaactcaaacccgt | |
| | ccttgattggcttgaagagaagtttaaggaaggtgtagagtttcttagagacggt | |
| | tgggaaattgttaaatttatctcaacctgtgcttgtgaaattgtcggtggacaaa | |
| | ttgtcacctgtgcaaaggaaattaaggagagtgttcagacattctttaagcttgt | |
| | aaataaatttttggctttgtgtgctgactctatcattattggtggagctaaactt | |
| | aaagccttgaatttaggtgaaacatttgtcacgcactcaaagggattgtacagaa | |
| | agtgtgttaaatccagagaagaaactggcctactcatgcctctaaaagccccaaa | |
| | agaaattatcttcttagagggagaaacacttcccacagaagtgttaacagaggaa | |
| | gttgtcttgaaaactggtgatttacaaccattagaacaacctactagtgaagctg | |
| | ttgaagctccattggttggtacaccagtttgtattaacgggcttatgttgctcga | |
| | aatcaaagacacagaaaagtactgtgcccttgcacctaatatgatggtaacaaac | |
| | aataccttcacactcaaaggcggtgcaccaacaaaggttacttttggtgatgaca | |
| | ctgtgatagaagtgcaaggttacaagagtgtgaatatcacttttgaacttgatga | |
| | aaggattgataaagtacttaatgagaagtgctctgcctatacagttgaactcggt | |
| | acagaagtaaatgagttcgcctgtgttgtggcagatgctgtcataaaaactttgc | |
| | aaccagtatctgaattacttacaccactgggcattgatttagatgagtggagtat | |
| | ggctacatactacttatttgatgagtctggtgagtttaTattggcttcacatatg | |
| | tattgttctttctaccctccagatgaggatgaagaagaaggtgattgtgaagaag | |
| | aagagtttgagccatcaactcaatatgagtatggtactgaagatgattaccaagg | |
| | taaacctttggaatttggtgccacttctgctgctcttcaacctgaagaagagcaa | |
| | gaagaagattggttagatgatgatagtcaacaaactgttggtcaacaagacggca | |
| | gtgaggacaatcagacaactactattcaaacaattgttgaggttcaacctcaatt | |
| | agagatggaacttacaccagttgttcagactattgaagtgaatagttttagtggt | |
| | tatttaaaacttactgacaatgtatacattaaaaatgcagacattgtggaagaag | |
| | ctaaaaaggtaaaaccaacagtggttgttaatgcagccaatgtttaccttaaaca | |
| | tggaggaggtgttgcaggagccttaaataaggctactaacaatgccatgcaagtt | |
| | gaatctgatgattacatagctactaatggaccacttaaagtgggtggtagttgtg | |
| | ttttaagcggacacaatcttgctaaacactgtcttcatgttgtcggcccaaatgt | |
| | taacaaaggtgaagacattcaacttcttaagagtgcttatgaaaattttaatcag | |
| | cacgaagttctacttgcaccattattatcagctggtatttttggtgctgacccta | |
| | tacattctttaagagtttgtgtagatactgttcgcacaaatgtctacttagctgt | |
| | ctttgataaaaatctctatgacaaacttgtttcaagctttttggaaatgaagagt | |
| | gaaaagcaagttgaacaaaagatcgctgagattcctaaagaggaagttaagccat | |
| | ttataactgaaagtaaaccttcagttgaacagagaaaacaagatgataagaaaat | |
| | caaagcttgtgttgaagaagttacaacaactctggaagaaactaagttcctcaca | |
| | gaaaacttgttactttatattgacattaatggcaatcttcatccagattctgcca | |
| | ctcttgttagtgacattgacatcactttcttaaagaaagatgctccatatatagt | |
| | gggtgatgttgttcaagagggtgttttaactgctgtggttatacctactaaaaag | |
| | gctggtggcactactgaaatgctagcgaaagctttgagaaaagtgccaacagaca | |
| | attatataaccacttacccgggtcagggtttaaatggttacactgtagaggaggc | |
| | aaagacagtgcttaaaaagtgtaaaagtgccttttacattctaccatctattatc | |
| | tctaatgagaagcaagaaattcttggaactgtttcttggaatttgcgagaaatgc | |
| | ttgcacatgcagaagaaacacgcaaattaatgcctgtctgtgtggaaactaaagc | |
| | catagtttcaactatacagcgtaaatataagggtattaaaatacaagagggtgtg | |
| | gttgattatggtgctagattttacttttacaccagtaaaacaactgtagcgtcac | |
| | ttatcaacacacttaacgatctaaatgaaactcttgttacaatgccacttggcta | |
| | tgtaacacatggcttaaatttggaagaagctgctcggtatatgagatctctcaaa | |
| | gtgccagctacagtttctgtttcttcacctgatgctgttacagcgtataatggtt | |
| | atcttacttcttcttctaaaacacctgaagaacattttattgaaaccatctcact | |
| | tgctggttcctataaagattggtcctattctggacaatctacacaactaggtata | |
| | gaatttcttaagagaggtgataaaagtgtatattacactagtaatcctaccacat | |
| | tccacctagatggtgaagttatcacctttgacaatcttaagacacttctttcttt | |
| | gagagaagtgaggactattaaggtgtttacaacagtagacaacattaacctccac | |
| | acgcaagttgtggacatgtcaatgacatatggacaacagtttggtccaacttatt | |
| | tggatggagctgatgttactaaaataaaacctcataattcacatgaaggtaaaac | |
| | attttatgttttacctaatgatgacactctacgtgttgaggcttttgagtactac | |
| | cacacaactgatcctagttttctgggtaggtacatgtcagcattaaatcacacta | |
| | aaaagtggaaatacccacaagttaatggtttaacttctattaaatgggcagataa | |
| | caactgttatcttgccactgcattgttaacactccaacaaatagagttgaagttt | |
| | aatccacctgctctacaagatgcttattacagagcaagggctggtgaagctgcta | |
| | acttttgtgcacttatcttagcctactgtaataagacagtaggtgagttaggtga | |
| | tgttagagaaacaatgagttacttgtttcaacatgccaatttagattcttgcaaa | |
| | agagtcttgaacgtggtgtgtaaaacttgtggacaacagcagacaacccttaagg | |
| | gtgtagaagctgttatgtacatgggcacactttcttatgaacaatttaagaaagg | |
| | tgttcagataccttgtacgtgtggtaaacaagctacaaaatatctagtacaacag | |
| | gagtcaccttttgttatgatgtcagcaccacctgctcagtatgaacttaagcatg | |
| | gtacatttacttgtgctagtgagtacactggtaattaccagtgtggtcactataa | |
| | acatataacttctaaagaaactttgtattgcatagacggtgctttacttacaaag | |
| | tcctcagaatacaaaggtcctattacggatgttttctacaaagaaaacagttaca | |
| | caacaaccataaaaccagttacttataaattggatggtgttgtttgtacagaaat | |
| | tgaccctaagttggacaattattataagaaagacaattcttatttcacagagcaa | |
| | ccaattgatcttgtaccaaaccaaccatatccaaacgcaagcttcgataatttta | |
| | agtttgtatgtgataatatcaaatttgctgatgatttaaaccagttaactggtta | |
| | taagaaacctgcttcaagagagcttaaagttacatttttccctgacttaaatggt | |
| | gatgtggtggctattgattataaacactacacaccctcttttaagaaaggagcta | |
| | aattgttacataaacctattgtttggcatgttaacaatgcaactaataaagccac | |
| | gtataaaccaaatacctggtgtatacgttgtctttggagcacaaaaccagttgaa | |
| | acatcaaattcgtttgatgtactgaagtcagaggacgcgcagggaatggataatc | |
| | ttgcctgcgaagatctaaaaccagtctctgaagaagtagtggaaaatcctaccat | |
| | acagaaagacgttcttgagtgtaatgtgaaaactaccgaagttgtaggagacatt | |
| | atacttaaaccagcaaataatagtttaaaaattacagaagaggttggccacacag | |
| | atctaatggctgcttatgtagacaattctagtcttactattaagaaacctaatga | |
| | attatctagagtattaggtttgaaaacccttgctactcatggtttagctgctgtt | |
| | aatagtgtcccttgggatactatagctaattatgctaagccttttcttaacaaag | |
| | ttgttagtacaactactaacatagttacacggtgtttaaaccgtgtttgtactaa | |
| | ttatatgccttatttctttactttattgctacaattgtgtacttttactagaagt | |
| | acaaattctagaattaaagcatctatgccgactactatagcaaagaatactgtta | |
| | agagtgtcggtaaattttgtctagaggcttcatttaattatttgaagtcacctaa | |
| | tttttctaaactgataaatattataatttggtttttactattaagtgtttgccta | |
| | ggttctttaatctactcaaccgctgctttaggtgttttaatgtctaatttaggca | |
| | tgccttcttactgtactggttacagagaaggctatttgaactctactaatgtcac | |
| | tattgcaacctactgtactggttctataccttgtagtgtttgtcttagtggttta | |
| | gattctttagacacctatccttctttagaaactatacaaattaccatttcatctt | |
| | ttaaatgggatttaactgcttttggcttagttgcagagtggtttttggcatatat | |
| | tcttttcactaggtttttctatgtacttggattggctgcaatcatgcaattgttt | |
| | ttcagctattttgcagtacattttattagtaattcttggcttatgtggttaataa | |
| | ttaatcttgtacaaatggccccgatttcagctatggttagaatgtacatcttctt | |
| | tgcatcattttattatgtatggaaaagttatgtgcatgttgtagacggttgtaat | |
| | tcatcaacttgtatgatgtgttacaaacgtaatagagcaacaagagtcgaatgta | |
| | caactattgttaatggtgttagaaggtccttttatgtctatgctaatggaggtaa | |
| | aggcttttgcaaactacacaattggaattgtgttaattgtgatacattctgtgct | |
| | ggtagtacatttattagtgatgaagttgcgagagacttgtcactacagtttaaaa | |
| | gaccaataaatcctactgaccagtcttcttacatcgttgatagtgttacagtgaa | |
| | gaatggttccatccatctttactttgataaagctggtcaaaagacttatgaaaga | |
| | cattctctctctcattttgttaacttagacaacctgagagctaataacactaaag | |
| | gttcattgcctattaatgttatagtttttgatggtaaatcaaaatgtgaagaatc | |
| | atctgcaaaatcagcgtctgtttactacagtcagcttatgtgtcaacctatactg | |
| | ttactagatcaggcattagtgtctgatgttggtgatagtgcggaagttgcagtta | |
| | aaatgtttgatgcttacgttaatacgttttcatcaacttttaacgtaccaatgga | |
| | aaaactcaaaacactagttgcaactgcagaagctgaacttgcaaagaatgtgtcc | |
| | ttagacaatgtcttatctacttttatttcagcagctcggcaagggtttgttgatt | |
| | cagatgtagaaactaaagatgttgttgaatgtcttaaattgtcacatcaatctga | |
| | catagaagttactggcgatagttgtaataactatatgctcacctataacaaagtt | |
| | gaaaacatgacaccccgtgaccttggtgcttgtattgactgtagtgcgcgtcata | |
| | ttaatgcgcaggtagcaaaaagtcacaacattgctttgatatggaacgttaaaga | |
| | tttcatgtcattgtctgaacaactacgaaaacaaatacgtagtgctgctaaaaag | |
| | aataacttaccttttaagttgacatgtgcaactactagacaagttgttaatgttg | |
| | taacaacaaagatagcacttaagggtggtaaaattgttaataattggttgaagca | |
| | gttaattaaagttacacttgtgttcctttttgttgctgctattttctatttaata | |
| | acacctgttcatgtcatgtctaaacatactgacttttcaagtgaaatcataggat | |
| | acaaggctattgatggtggtgtcactcgtgacatagcatctacagatacttgttt | |
| | tgctaacaaacatgctgattttgacacatggtttagtcagcgtggtggtagttat | |
| | actaatgacaaagcttgcccattgattgctgcagtcataacaagagaagtgggtt | |
| | ttgtcgtgcctggtttgcctggcacgatattacgcacaactaatggtgacttttt | |
| | gcatttcttacctagagtttttagtgcagttggtaacatctgttacacaccatca | |
| | aaacttatagagtacactgactttgcaacatcagcttgtgttttggctgctgaat | |
| | gtacaatttttaaagatgcttctggtaagccagtaccatattgttatgataccaa | |
| | tgtactagaaggttctgttgcttatgaaagtttacgccctgacacacgttatgtg | |
| | ctcatggatggctctattattcaatttcctaacacctaccttgaaggttctgtta | |
| | gagtggtaacaacCtttgattctgagtactgtaggcacggcacttgtgaaagatc | |
| | agaagctggtgtttgtgtatctactagtggtagatgggtacttaacaatgattat | |
| | tacagatctttaccaggagttttctgtggtgtagatgctgtaaatttacttacta | |
| | atatgtttacaccactaattcaacctattggtgctttggacatatcagcatctat | |
| | agtagctggtggtattgtagctatcgtagtaacatgccttgcctactattttatg | |
| | aggtttagaagagcttttggtgaatacagtcatgtagttgcctttaatactttac | |
| | tattccttatgtcattcactgtactctgtttaacaccagtttactcattcttacc | |
| | tggtgtttattctgttatttacttgtacttgacattttatcttactaatgatgtt | |
| | tcttttttagcacatattcagtggatggttatgttcacacctttagtacctttct | |
| | ggataacaattgcttatatcatttgtatttccacaaagcatttctattggttctt | |
| | tagtaattacctaaagagacgtgtagtctttaatggtgtttcctttagtactttt | |
| | gaagaagctgcgctgtgcacctttttgttaaataaagaaatgtatctaaagttgc | |
| | gtagtgatgtgctattacctcttacgcaatataatagatacttagctctttataa | |
| | taagtacaagtattttagtggagcaatggatacaactagctacagagaagctgct | |
| | tgttgtcatctcgcaaaggctctcaatgacttcagtaactcaggttctgatgttc | |
| | tttaccaaccaccacaaacctctatcacctcagctgttttgcagagtggttttag | |
| | aaaaatggcattcccatctggtaaagttgagggttgtatggtacaagtaacttgt | |
| | ggtacaactacacttaacggtctttggcttgatgacgtagtttactgtccaagac | |
| | atgtgatctgcacctctgaagacatgcttaaccctaattatgaagatttactcat | |
| | tcgtaagtctaatcataatttcttggtacaggctggtaatgttcaactcagggtt | |
| | attggacattctatgcaaaattgtgtacttaagcttaaggttgatacagccaatc | |
| | ctaagacacctaagtataagtttgttcgcattcaaccaggacagactttttcagt | |
| | gttagcttgttacaatggttcaccatctggtgtttaccaatgtgctatgaggccc | |
| | aatttcactattaagggttcattccttaatggttcatgtggtagtgttggtttta | |
| | acatagattatgactgtgtctctttttgttacatgcaccatatggaattaccaac | |
| | tggagttcatgctggcacagacttagaaggtaacttttatggaccttttgttgac | |
| | aggcaaacagcacaagcagctggtacggacacaactattacagttaatgttttag | |
| | cttggttgtacgctgctgttataaatggagacaggtggtttctcaatcgatttac | |
| | cacaactcttaatgactttaaccttgtggctatgaagtacaattatgaacctcta | |
| | acacaagaccatgttgacatactaggacctctttctgctcaaactggaattgAcg | |
| | ttttagatatgtgtgcttcattaaaagaattactgcaaaatggtatgaatggacg | |
| | taccatattgggtagtgctttattagaagatgaatttacaccttttgatgttgtt | |
| | agacaatgctcaggtgttactttccaaagtgcagtgaaaagaacaatcaagggta | |
| | cacaccactggttgttactcacaattttgacttcacttttagttttagtccagag | |
| | tactcaatggtctttgttcttttttttgtatgaGaatgcctttttaccttttgct | |
| | atgggtattattgctatgtctgcttttgcaatgatgtttgtcaaacataagcatg | |
| | catttctctgtttgtttttgttaccttctcttgccactgtagcttattttaatat | |
| | ggtctatatgcctgctagttgggtgatgcgtattatgacatggttggatatggtt | |
| | gatactagtttgtctggttttaagctaaaagactgtgttatgtatgcatcagctg | |
| | tagtgttactaatccttatgacagcaagaactgtgtatgatgatggtgctaggag | |
| | agtgtggacacttatgaatgtcttgacactcgtttataaagtttattatggtaat | |
| | gctttagatcaagccatttccatgtgggctcttataatctctgttacttctaact | |
| | actcaggtgtagttacaactgtcatgttCttggccagaggtattgtttttatgtg | |
| | tgttgagtattgccctattttcttcataactggtaatacacttcagtgtataatg | |
| | ctagtttattgtttcttaggctatttttgtacttgttactttggcctcttttgtt | |
| | tactcaaccgctactttagactgactcttggtgtttatgattacttagtttctac | |
| | acaggagtttagatatatgaattcacagggactactcccacccaagaatagcata | |
| | gatgccttcaaactcaacattaaattgttgggtgttggtggcaaaccttgtatca | |
| | aagtagccactgtacagtctaaaatgtcagatgtaaagtgcacatcagtagtctt | |
| | actctcagttttgcaacaactcagagtagaatcatcatctaaattgtgggctcaa | |
| | tgtgtccagttacacaatgacattctcttagctaaagatactactgaagcctttg | |
| | aaaaaatggtttcactactttctgttttgctttccatgcagggtgctgtagacat | |
| | aaacaagctttgtgaagaaatgctggacaacagggcaaccttacaagctatagcc | |
| | tcagagtttagttcccttccatcatatgcagcttttgctactgctcaagaagctt | |
| | atgagcaggctgttgctaatggtgattctgaagttgttcttaaaaagttgaagaa | |
| | gtctttgaatgtggctaaatctgaatttgaccgtgatgcagccatgcaacgtaag | |
| | ttggaaaagatggctgatcaagctatgacccaaatgtataaacaggctagatctg | |
| | aggacaagagggcaaaagttactagtgctatgcagacaatgcttttcactatgct | |
| | tagaaagttggataatgatgcactcaacaacattatcaacaatgcaagagatggt | |
| | tgtgttcccttgaacataatacctcttacaacagcagccaaactaatggttgtca | |
| | taccagactataacacatataaaaatacgtgtgatggtacaacatttacttatgc | |
| | atcagcattgtgggaaTtccaacaggttgtagatgcagatagtaaaattgttcaa | |
| | cttagtgaaattagtatggacaattcacctaatttagcatggcctcttattgtaa | |
| | cagctttaagggccaattctgctgtcaaattacagaataatgagcttagtcctgt | |
| | tgcactacgacagatgtcttgtgctgccggtactacacaaactgcttgcactgat | |
| | gacaatgcgttagcttactacaacacaacaaagggaggtaggtttgtacttgcac | |
| | tgttatccgatttacaggatttgaaatgggctagattccctaagagtgatggaac | |
| | tggtactatctatacagaactggaaccaccttgtaggtttgttacagacacacct | |
| | aaaggtcctaaagtgaagtatttatactttattaaaggattaaacaacctaaata | |
| | gaggtatggtacttggtagtttagctgccacagtacgtctacaagctggtaatgc | |
| | aacagaagtgcctgccaattcaactgtattatctttctgtgcttttgctgtagat | |
| | gctgctaaagcttacaaagattatctagctagtgggggacaaccaatcactaatt | |
| | gtgttaagatgttgtgtacacacactggtactggtcaggcaataacagttacacc | |
| | ggaagccaatatggatcaagaatcctttggtggtgcatcgtgttgtctgtactgc | |
| | cgttgccacatagatcatccaaatcctaaaggattttgtgacttaaaaggtaagt | |
| | atgtacaaatacctacaacttgtgctaatgaccctgtgggttttacacttaaaaa | |
| | cacagtctgtaccgtctgcggtatgtggaaaggttatggctgtagttgtgatcaa | |
| | ctccgcgaacccatgcttcagtcagctgatgcacaatcgtttttaaacgggtttg | |
| | cggtgtaagtgcagcccgtcttacaccgtgcggcacaggcactagtactgatgtc | |
| | gtatacagggcttttgacatctacaatgataaagtagctggttttgctaaattcc | |
| | taaaaactaattgttgtcgcttccaagaaaaggacgaagatgacaatttaattga | |
| | ttcttactttgtagttaagagacacactttctctaactaccaacatgaagaaaca | |
| | atttataatttacttaaggattgtccagctgttgctaaacatgacttctttaagt | |
| | ttagaatagacggtgacatggtaccacatatatcacgtcaacgtcttactaaata | |
| | cacaatggcagacctcgtctatgctttaaggcattttgatgaaggtaattgtgac | |
| | acattaaaagaaatacttgtcacatacaattgttgtgatgatgattatttcaata | |
| | aaaaggactggtatgattttgtagaaaacccagatatattacgcgtatacgccaa | |
| | cttaggtgaacgtgtacgccaagctttgttaaaaacagtacaattctgtgatgcc | |
| | atgcgaaatgctggtattgttggtgtactgacattagataatcaagatctcaatg | |
| | gtaactggtatgatttcggtgatttcatacaaaccacgccaggtagtggagttcc | |
| | tgttgtagattcttattattcattgttaatgcctatattaaccttgaccagggct | |
| | ttaactgcagagtcacatgttgacactgacttaacaaagccttacattaagtggg | |
| | atttgttaaaatatgacttcacggaagagaggttaaaactctttgaccgttattt | |
| | taaatattgggatcagacataccacccaaattgtgttaactgtttggatgacaga | |
| | tgcattctgcattgtgcaaactttaatgttttattctctacagtgttcccaccta | |
| | caagttttggaccactagtgagaaaaatatttgttgatggtgttccatttgtagt | |
| | ttcaactggataccacttcagagagctaggtgttgtacataatcaggatgtaaac | |
| | ttacatagctctagacttagttttaaggaattacttgtgtatgctgctgaccctg | |
| | ctatgcacgctgcttctggtaatctattactagataaacgcactacgtgcttttc | |
| | agtagctgcacttactaacaatgttgcttttcaaactgtcaaacccggtaatttt | |
| | aacaaagacttctatgactttgctgtgtctaagggtttctttaaggaaggaagtt | |
| | ctgttgaattaaaacacttcttctttgctcaggatggtaatgctgctatcagcga | |
| | ttatgactactatcgttataatctaccaacaatgtgtgatatcagacaactacta | |
| | tttgtagttgaagttgttgataagtactttgattgttacgatggtggctgtatta | |
| | atgctaaccaagtcatcgtcaacaacctagacaaatcagctggttttccatttaa | |
| | taaatggggtaaggctagactttattatgattcaatgagttatgaggatcaagat | |
| | gcacttttcgcatatacaaaacgtaatgtcatccctactataactcaaatgaatc | |
| | ttaagtatgccattagtgcaaagaatagagctcgcaccgtagctggtgtctctat | |
| | ctgtagtactatgaccaatagacagtttcatcaaaaattattgaaatcaatagcc | |
| | gccactagaggagctactgtagtaattggaacaagcaaattctatggtggttggc | |
| | acaacatgttaaaaactgtttatagtgatgtagaaaaccctcaccttatgggttg | |
| | ggattatcctaaatgtgatagagccatgcctaacatgcttagaattatggcctca | |
| | cttgttcttgctcgcaaacatacaacgtgttgtagcttgtcacaccgtttctata | |
| | gattagctaatgagtgtgctcaagtattgagtgaaatggtcatgtgtggcggttc | |
| | actatatgttaaaccaggtggaacctcatcaggagatgccacaactgcttatgct | |
| | aatagtgtttttaacatttgtcaagctgtcacggccaatgttaatgcacttttat | |
| | ctactgatggtaacaaaattgccgataagtatgtccgcaatttacaacacagact | |
| | ttatgagtgtctctatagaaatagagatgttgacacagactttgtgaatgagttt | |
| | tacgcatatttgcgtaaacatttctcaatgatgatactctctgacgatgctgttg | |
| | tgtgtttcaatagcacttatgcatctcaaggtctagtggctagcataaagaactt | |
| | taagtcagttctttattatcaaaacaatgtttttatgtctgaagcaaaatgttgg | |
| | actgagactgaccttactaaaggacctcatgaattttgctctcaacatacaatgc | |
| | tagttaaacagggtgatgattatgtgtaccttccttacccagatccatcaagaat | |
| | cctaggggccggctgttttgtagatgatatcgtaaaaacagatggtacacttatg | |
| | attgaacggttcgtgtctttagctatagatgcttacccacttactaaacatccta | |
| | atcaggagtatgctgatgtctttcatttgtacttacaatacataagaaagctaca | |
| | tgatgagttaacaggacacatgttagacatgtattctgttatgcttactaatgat | |
| | aacacttcaaggtattgggaacctgagttttatgaggctatgtacacaccgcata | |
| | cagtcttacaggctgttggggcttgtgttctttgcaattcacagacttcattaag | |
| | atgtggtgcttgcatacgtagaccattcttatgttgtaaatgctgttacgaccat | |
| | gtcatatcaacatcacataaattagtcttgtctgttaatccgtatgtttgcaGtg | |
| | ctccaggttgtgatgtcacagatgtgactcaactttacttaggaggtatgagcta | |
| | ttattgtaaatcacataaaccacccattagttttccattgtgtgctaatggacaa | |
| | gtttttggtttatataaaaatacatgtgttggtagcgataatgttactgacttta | |
| | atgcaattgcaacatgtgactggacaaatgctggtgattacattttagctaacac | |
| | ctgtactgaaagactcaagctttttgcagcagaaacgctcaaagctactgaggag | |
| | acatttaaactgtcttatggtattgctactgtacgtgaagtgctgtctgacagag | |
| | aattacatctttcatgggaagttggtaaacctagaccaccacttaaccgaaatta | |
| | tgtctttactggttatcgtgtaactaaaaacagtaaagtacaaataggagagtac | |
| | acctttgaaaaaggtgactatggtgatgctgttgtttaccgaggtacaacaactt | |
| | acaaattaaatgttggtgattattttgtgctgacatcacatacagtaatgccatt | |
| | aagtgcacctacactagtgccacaagagcactatgttagaattactggcttatac | |
| | ccaacactcaatatctcagatgagttttctagcaatgttgcaaattatcaaaagg | |
| | ttggtatgcaaaagtattctacactccagggaccacctggtactggtaagagtca | |
| | ttttgctattggcctagctctctactacccttctgctcgcatagtgtatacagct | |
| | tgctctcatgccgctgttgatgcactatgtgagaaggcattaaaatatttgccta | |
| | tagataaatgtagtagaattatacctgcacgtgctcgtgtagagtgttttgataa | |
| | attcaaagtgaattcaacattagaacagtatgtcttttgtactgtaaatgcattg | |
| | cctgagacgacagcagatatagttgtctttgatgaaatttcaatggccacaaatt | |
| | atgatttgagtgttgtcaatgccagattacgtgctaagcactatgtgtacattgg | |
| | cgaccctgctcaattacctgcaccacgcacattgctaactaagggcacactagaa | |
| | ccagaatatttcaattcagtgtgtagacttatgaaaactataggtccagacatgt | |
| | tcctcggaacttgtcggcgttgtcctgctgaaattgttgacactgtgagtgcttt | |
| | ggtttatgataataagcttaaagcacataaagacaaatcagctcaatgctttaaa | |
| | atgttttataagggtgttatcacgcatgatgtttcatctgcaattaacaggccac | |
| | aaataggcgtggtaagagaattccttacacgtaaccctgcttggagaaaagctgt | |
| | ctttatttcaccttataattcacagaatgctgtagcctcaaagattttgggacta | |
| | ccaactcaaactgttgattcatcacagggctcagaatatgactatgtcatattca | |
| | ctcaaaccactgaaacagctcactcttgtaatgtaaacagatttaatgttgctat | |
| | taccagagcaaaagtaggcatactttgcataatgtctgatagagacctttatgac | |
| | aagttgcaatttacaagtcttgaaattccacgtaggaatgtggcaactttacaag | |
| | ctgaaaatgtaacaggactttttaaagattgtagtaaggtaatcactgggttaca | |
| | tcctacacaggcacctacacacctcagtgttgacactaaattcaaaactgaaggt | |
| | ttatgtgttgacatacctggcatacctaaggacatgacctatagaagactcatct | |
| | ctatgatgggttttaaaatgaattatcaagttaatggttaccctaacatgtttat | |
| | cacccgcgaagaagctataagacatgtacgtgcatggattggcttcgatgtcgag | |
| | gggtgtcatgctactagagaagctgttggtaccaatttacctttacagctaggtt | |
| | tttctacaggtgttaacctagttgctgtacctacaggttatgttgatacacctaa | |
| | taatacagatttttccagagttagtgctaaaccaccgcctggagatcaatttaaa | |
| | cacctcataccacttatgtacaaaggacttccttggaatgtagtgcgtataaaga | |
| | ttgtacaaatgttaagtgacacacttaaaaatctctctgacagagtcgtatttgt | |
| | cttatgggcacatggctttgagttgacatctatgaagtattttgtgaaaatagga | |
| | cctgagcgcacctgttgtctatgtgatagacgtgccacatgcttttccactgctt | |
| | cagacacttatgcctgttggcatcattctattggatttgattacgtctataatcc | |
| | gtttatgattgatgttcaacaatggggttttacaggtaacctacaaagcaaccat | |
| | gatctgtattgtcaagtccatggtaatgcacatgtagctagttgtgatgcaatca | |
| | tgactaggtgtctagctgtccacgagtgctttgttaagcgtgttgactggactat | |
| | tgaatatcctataattggtgatgaactgaagattaatgcggcttgtagaaaggtt | |
| | caacacatggttgttaaagctgcattattagcagacaaattcccagttcttcacg | |
| | acattggtaaccctaaagctattaagtgtgtacctcaagctgatgtagaatggaa | |
| | gttctatgatgcacagccttgtagtgacaaagcttataaaatagaagaattattc | |
| | tattcttatgccacacattctgacaaattcacagatggtgtatgcctattttgga | |
| | attgcaatgtcgatagatatcctgctaattccattgtttgtagatttgacactag | |
| | agtgctatctaaccttaacttgcctggttgtgatggtggcagtttgtatgtaaat | |
| | aaacatgcattccacacaccagcttttgataaaagtgcttttgttaatttaaaac | |
| | aattaccatttttctattactctgacagtccatgtgagtctcatggaaaacaagt | |
| | agtgtcagatatagattatgtaccactaaagtctgctacgtgtataacacgttgc | |
| | aatttaggtggtgctgtctgtagacatcatgctaatgagtacagattgtatctcg | |
| | atgcttataacatgatgatctcagctggctttagcttgtgggtttacaaacaatt | |
| | tgatacttataacctctggaacacttttacaagacttcagagtttagaaaatgtg | |
| | gcttttaatgttgtaaataagggacactttgatggacaacagggtgaagtaccag | |
| | tttctatcattaataacactgtttacacaaaagttgatggtgttgatgtagaatt | |
| | gtttgaaaataaaacaacattacctgttaatgtagcatttgagctttgggctaag | |
| | cgcaacattaaaccagtaccagaggtgaaaatactcaataatttgggtgtggaca | |
| | ttgctgctaatactgtgatctgggactacaaaagagatgctccagcacatatatc | |
| | tactattggtgtttgttctatgactgacatagccaagaaaccaactgaaacgatt | |
| | tgtgcaccactcactgtcttttttgatggtagagttgatggtcaagtagacttat | |
| | ttagaaatgcccgtaatggtgttcttattacagaaggtagtgttaaaggtttaca | |
| | accatctgtaggtcccaaacaagctagtcttaatggagtcacattaattggagaa | |
| | gccgtaaaaacacagttcaattattataagaaagttgatggtgttgtccaacaat | |
| | tacctgaaacttactttactcagagtagaaatttacaagaatttaaacccaggag | |
| | tcaaatggaaattgatttcttagaattagctatggatgaattcattgaacggtat | |
| | aaattagaaggctatgccttcgaacatatcgtttatggagattttagtcatagtc | |
| | agttaggtggtttacatctactgattggactagctaaacgttttaaggaatcacc | |
| | ttttgaattagaagattttattcctatggacagtacagttaaaaactatttcata | |
| | acagatgcgcaaacaggttcatctaagtgtgtgtgttctgttattgatttattac | |
| | ttgatgattttgttgaaataataaaatcccaagatttatctgtagtttctaaggt | |
| | tgtcaaagtgactattgactatacagaaatttcatttatgctttggtgtaaagat | |
| | ggccatgtagaaacattttacccaaaattacaatctagtcaagcgtggcaaccgg | |
| | gtgttgctatgcctaatctttacaaaatGCAAAGAATGCTATTAGAAAAGTGTGA | |
| | CCTTCAAAATTATGGTGATAGTGCAACATTACCTAAAGGCATAATGATGAATGTC | |
| | GCAAAATATACTCAACTGTGTCAATATTTAAACACATTAACATTAGCTGTACCCT | |
| | ATAATATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATTCAGAT | |
| | CTTAATGACTTTGTCTCTGATGCAGATTCAACTTTGATTGGTGATTGTGCAACTG | |
| | TACATACAGCTAATAAATGGGATCTCATTATTAGTGATATGTACGACCCTAAGAC | |
| | TAAAAATGTTACAAAAGAAAATGACTCTAAAGAGGGTTTTTTCACTTACATTTGT | |
| | GGGTTTATACAACAAAAGCTAGCTCTTGGAGGTTCCGTGGCTATAAAGATAACAG | |
| | AACATTCTTGGAATGCTGATCTTTATAAGCTCATGGGACACTTCGCATGGTGGAC | |
| | AGCCTTTGTTACTAATGTGAATGCGTCATCATCTGAAGCATTTTTAATTGGATGT | |
| | AATTATCTTGGCAAACCACGCGAACAAATAGATGGTTATGTCATGCATGCAAATT | |
| | ACATATTTTGGAGGAATACAAATCCAATTCAGTTGTCTTCCTATTCTTTATTTGA | |
| | CATGAGTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTATAATTA | |
| | GAGAAAACAACAGAGTTGTTATTTCTAGTGATGTTCTTGTTAACAACTAAACGAA | |
| | CAATGTTTGTTTTTCTTGTTTTATTGCCACTAGTCTCTAGTCAGTGTGTTAATCT | |
| | TAgAACCAGAACTCAATTACCCCCTGCATACACTAATTCTTTCACACGTGGTATT | |
| | TATTACCCTGACAAAGTTTTCAGATCCTCAGTTTTACATTCAACTCAGGACTTGT | |
| | TCTTACCTTTCTTTTCCAATGTTACTTGGTTCCATGCTATACATGTCTCTGGGAC | |
| | CAATGGTACTAAGAGGTTTGATAACCCTGTCCTACCATTTAATGATGGTGTTTAT | |
| | TTTGCTTCCACTGAGAAGTCTAACATAATAAGAGGCTGGATTTTTGGTACTACTT | |
| | TAGATTCGAAGACCCAGTCCCTACTTATTGTTAATAACGCTACTAATGTTGTTAT | |
| | TAAAGTCTGTGAATTTCAATTTTGTAATGATCCATTTTTGGaTGTTTATTACCAC | |
| | AAAAACAACAAAAGTTGGATGGAAAGTGGAGTTTATTCTAGTGCGAATAATTGCA | |
| | CTTTTGAATATGTCTCTCAGCCTTTTCTTATGGACCTTGAAGGAAAACAGGGTAA | |
| | TTTCAAAAATCTTAGGGAATTTGTGTTTAAGAATATTGATGGTTATTTTAAAATA | |
| | TATTCTAAGCACACGCCTATTAATTTAGTGCGTGATCTCCCTCAGGGTTTTTCGG | |
| | CTTTAGAACCATTGGTAGATTTGCCAATAGGTATTAACATCACTAGGTTTCAAAC | |
| | TTTACTTGCTTTACATAGAAGTTATTTGACTCCTGGTGATTCTTCTTCAGGTTGG | |
| | ACAGCTGGTGCTGCAGCTTATTATGTGGGTTATCTTCAACCTAGGACTTTTCTAT | |
| | TAAAATATAATGAAAATGGAACCATTACAGATGCTGTAGACTGTGCACTTGACCC | |
| | TCTCTCAGAAACAAAGTGTACGTTGAAATCCTTCACTGTAGAAAAAGGAATCTAT | |
| | CAAACTTCTAACTTTAGAGTCCAACCAACAGAATCTATTGTTAGATTTCCTAATA | |
| | TTACAAACTTGTGCCCTTTTGGTGAAGTTTTTAACGCCACCAGATTTGCATCTGT | |
| | TTATGCTTGGAACAGGAAGAGAATCAGCAACTGTGTTGCTGATTATTCTGTCCTA | |
| | TATAATTCCGCATCATTTTCCACTTTTAAGTGTTATGGAGTGTCTCCTACTAAAT | |
| | TAAATGATCTCTGCTTTACTAATGTCTATGCAGATTCATTTGTAATTAGAGGTGA | |
| | TGAAGTCAGACAAATCGCTCCAGGGCAAACTGGAAAGATTGCTGATTATAATTAT | |
| | AAATTACCAGATGATTTTACAGGCTGCGTTATAGCTTGGAATTCTAACAATCTTG | |
| | ATTCTAAGGTTGGTGGTAATTATAATTACCgGTATAGATTGTTTAGGAAGTCTAA | |
| | TCTCAAACCTTTTGAGAGAGATATTTCAACTGAAATCTATCAGGCCGGTAGCAaA | |
| | CCTTGTAATGGTGTTGAAGGTTTTAATTGTTACTTTCCTTTACAATCATATGGTT | |
| | TCCAACCCACTAATGGTGTTGGTTACCAACCATACAGAGTAGTAGTACTTTCTTT | |
| | TGAACTTCTACATGCACCAGCAACTGTTTGTGGACCTAAAAAGTCTACTAATTTG | |
| | GTTAAAAACAAATGTGTCAATTTCAACTTCAATGGTTTAACAGGCACAGGTGTTC | |
| | TTACTGAGTCTAACAAAAAGTTTCTGCCTTTCCAACAATTTGGCAGAGACATTGC | |
| | TGACACTACTGATGCTGTCCGTGATCCACAGACACTTGAGATTCTTGACATTACA | |
| | CCATGTTCTTTTGGTGGTGTCAGTGTTATAACACCAGGAACAAATACTTCTAACC | |
| | AGGTTGCTGTTCTTTATCAGGgTGTTAACTGCACAGAAGTCCCTGTTGCTATTCA | |
| | TGCAGATCAACTTACTCCTACTTGGCGTGTTTATTCTACAGGTTCTAATGTTTTT | |
| | CAAACACGTGCAGGCTGTTTAATAGGGGCTGAACATGTCAACAACTCATATGAGT | |
| | GTGACATACCCATTGGTGCAGGTATATGCGCTAGTTATCAGACTCAGCAATCCAT | |
| | CATTGCCTACACTATGTCACTTGGTGCAGAAAATTCAGTTGCTTACTCTAATAAC | |
| | TCTATTGCCATACCCACAAATTTTACTATTAGTGTTACCACAGAAATTCTACCAG | |
| | TGTCTATGACCAAGACATCAGTAGATTGTACAATGTACATTTGTGGTGATTCAAC | |
| | TGAATGCAGCAATCTTTTGTTGCAATATGGCAGTTTTTGTACACAATTAAACCGT | |
| | GCTTTAACTGGAATAGCTGTTGAACAAGACAAAAACACCCAAGAAGTTTTTGCAC | |
| | AAGTCAAACAAATTTACAAAACACCACCAATTAAAGATTTTGGTGGTTTTAATTT | |
| | TTCACAAATATTACCAGATCCATCAAAACCAAGCAAGAGGTCATTTATTGAAGAT | |
| | CTACTTTTCAACAAAGTGACACTTGCAGATGCTGGCTTCATCAAACAATATGGTG | |
| | ATTGCCTTGGTGATATTGCTGCTAGAGATCTCATTTGCGCTCAAAAATTTAACGG | |
| | ACTTACAGTTTTACCACCTTTACTTACTGACGAAATGATTGCGCAATATACATCC | |
| | GCATTGTTAGCCGGAACTATTACATCCGGATGGACTTTTGGCGCAGGCGCAGCAT | |
| | TACAGATTCCATTCGCTATGCAAATGGCTTATAGGTTTAACGGTATAGGCGTTAC | |
| | GCAAAACGTACTTTATGAGAATCAAAAACTTATCGCTAACCAATTTAATTCCGCT | |
| | ATCGGTAAGATTCAGGATTCATTGTCTAGTACTGCTAGTGCACTCGGTAAGTTGC | |
| | AAaAtGTAGTGAATCAAAACGCTCAAGCACTTAATACACTCGTTAAACAGCTTAG | |
| | TTCTAATTTTGGCGCAATTTCTAGTGTGCTTAACGATATACTATCTAGACTCGAT | |
| | AAAGTCGAAGCCGAAGTGCAAATCGATAGATTGATTACCGGTAGGTTGCAATCAT | |
| | TGCAAACATACGTTACACAGCAATTGATTAGGGCCGCAGAGATACGCGCTAGCGC | |
| | TAATCTCGCAGCTACTAAAATGTCTGAATGCGTACTCGGACAATCTAAACGTGTC | |
| | GATTTTTGCGGTAAGGGATATCATCTTATGTCTTTTCCACAATCTGCACCTCACG | |
| | GAGTCGTGTTTTTACACGTTACTTATGTGCCAGCTCAAGAGAAAAATTTTACAAC | |
| | CGCTCCTGCTATTTGTCATGACGGTAAGGCACATTTTCCTAGAGAGGGCGTATTC | |
| | GTTTCTAACGGTACACATTGGTTCGTTACACAACGTAATTTTTACGAACCTCAAA | |
| | TTATTACTACTGATAATACATTCGTATCAGGTAATTGTGACGTAGTGATAGGTAT | |
| | CGTTAATAATACAGTTTACGATCCACTTCAACCTGAACTCGATAGTTTTAAAGAG | |
| | GAACTCGATAAGTATTTTAAAAATCATACATCACCTGACGTCGACTTAGGCGATA | |
| | TTTCAGGTATTAACGCTAGTGTCGTTAACATTCAAAAAGAGATTGATAGACTTAA | |
| | CGAAGTCGCTAAAAATCTTAACGAATCACTTATCGATCTGCAAGAGTTAGGTAAG | |
| | TATGAGCAATATATTAAATGGCCTTGGTATATTTGGTTAGGCTTTATAGCCGGAT | |
| | TGATCGCAATCGTTATGGTTACAATTATGTTATGTTGTATGACATCATGTTGTTC | |
| | ATGTCTTAAGGGATGTTGTTCATGCGGATCATGTTGTAAATTTGACGAAGACGAT | |
| | TCCGAACCAGTGCTTAAAGGCGTTAAGTTACATTATACATAAACGAACTTATGGA | |
| | TTTGTTTATGAGAATCTTCACAATTGGAACTGTAACTTTGAAGCAAGGTGAAATC | |
| | AAGGATGCTACTCCTTCAGATTTTGTTCGCGCTACTGCAACGATACCGATACAAG | |
| | CCTCACTCCCTTTCGGATGGCTTATTGTTGGCGTTGCACTTCTTGCTGTTTTTCA | |
| | GAGCGCTTCCAAAATCATAACCCTCAAAAAGAGATGGCAACTAGCACTCTCCAAG | |
| | GGTGTTCACTTTGTTTGCAACTTGCTGTTGTTGTTTGTAACAGTTTACTCACACC | |
| | TTTTGCTCGTTGCTGCTGGCCTTGAAGCcccttttctctatctttatgctttagt | |
| | ctacttcttgcagagtataaactttgtaagaataataatgaggctttggctttgc | |
| | tggaaatgccgttccaaaaacccattactttatgatgccaactattttctttgct | |
| | ggcatactaattgttacgactattgtataccttacaatagtgtaacttcttcaat | |
| | tgtcattacttcaggtgatggcacaacaagtcctatttctgaacatgactaccag | |
| | attggtggttatactgaaaaatgggaatctggagtaaaagactgtgttgtattac | |
| | acagttacttcacttcagactattaccagctgtactcaactcaattgagtacaga | |
| | cactggtgttgaacatgttaccttcttcatctacaataaaattgttgatgagcct | |
| | gaagaacatgtccaaattcacacaatcgacggttcatccggagttgttaatccag | |
| | taatggaaccaatttatgatgaaccgacgacgactactagcgtgcctttgtaagc | |
| | acaagctgatgagtacgaacttatgtactcattcgtttcggaagagacaggtacg | |
| | ttaatagttaatagcgtacttctttttcttgctttcgtggtattcttgctagtta | |
| | cactagccatccttactgcgcttcgattgtgtgcgtactgctgcaatattgttaa | |
| | cgtgagtcttgtaaaaccttctttttacgtttactctcgtgttaaaaatctgaat | |
| | tcttctagagttcctgatcttctggtctaaacgaactaaatattatattagtttt | |
| | tctgtttggaactttaattttagccatggcagattccaacggtactattaccgtt | |
| | gaagagcttaaaaagctccttgaacaatggaacctagtaataggtttcctattcc | |
| | ttacatggatttgtcttctacaatttgcctatgccaacaggaataggtttttgta | |
| | tataattaagttaattttcctctggctgttatggccagtaactttagcttgtttt | |
| | gtgcttgctgctgtttacagaataaattggatcaccggtggaattgctatcgcaa | |
| | tggcttgtcttgtaggcttgatgtggctcagctacttcattgcttctttcagact | |
| | gtttgcgcgtacgcgttccatgtggtcattcaatccagaaactaacattcttctc | |
| | aacgtgccactccatggcactattctgaccagaccgcttctagaaagtgaactcg | |
| | taatcggagctgtgatccttcgtggacatcttcgtattgctggacaccatctagg | |
| | acgctgtgacatcaaggacctgcctaaagaaatcactgttgctacatcacgaacg | |
| | ctttcttattacaaattgggagcttcgcagcgtgtagcaggtgactcaggttttg | |
| | ctgcatacagtcgctacaggattggcaactataaattaaacacagaccattccag | |
| | tagcagtgacaatattgctttgcttgtacagtaagtgacaacagatgtttcatct | |
| | cgttgactttcaggttactatagcagagatattactaattattatgaggactttt | |
| | aaagtttccatttggaatcttgattacatcataaacctcataattaaaaatttat | |
| | ctaagtcactaactgagaataaatattctcaattagatgaagagcaaccaatgga | |
| | gattgattaaacgaacatgaaaattattcttttcttggcactgataacactcgct | |
| | acttgtgagctttatcactaccaagagtgtgttagaggtacaacagtacttttaa | |
| | aagaaccttgctcttctggaacatacgagggcaattcaccatttcatcctctagc | |
| | tgataacaaatttgcactgacttgctttagcactcaatttgcttttgcttgtcct | |
| | gacggcgtaaaacacgtctatcagttacgtgccagatcagtttcacctaaactgt | |
| | tcatcagacaagaggaagttcaagaactttactctccaatttttcttattgttgc | |
| | ggcaatagtgtttataacactttgcttcacactcaaaagaaagacagaatgattg | |
| | aactttcattaattgacttctatttgtgctttttagcctttctgctattccttgt | |
| | tttaattatgcttattatcttttggttctcacttgaactgcaagatcataatgaa | |
| | acttgtcacgcctaaacgaacatgaaatttcttgttttcttaggaatcatcacaa | |
| | ctgtagctgcatttcaccaagaatgtagtttacagtcatgtactcaacatcaacc | |
| | atatgtagttgatgacccgtgtcctattcacttctattctaaatggtatattaga | |
| | gtaggagctagaaaatcagcacctttaattgaattgtgcgtggatgaggctggtt | |
| | ctaaatcacccattcagtacatcgatatcggtaattatacagtttcctgttcacc | |
| | ttttacaattaattgccaggaacctaaattgggtagtcttgtagtgcgttgttcg | |
| | ttctatgaagactttttagagtatcatgacgttcgtgttgttttagatttcatct | |
| | aaacgaacaaactaaaatgtctgataatggaccccaaaatcagcgaaatgcaccc | |
| | cgcattacgtttggtggaccctcagattcaactggcagtaaccagaatggagaac | |
| | gcagtggggcgcgatcaaaacaacgtcggccccaaggtttacccaataatactgc | |
| | gtcttggttcaccgctctcactcaacatggcaaggaagaccttaaattccctcga | |
| | ggacaaggcgttccaattaacaccaatagcagtccagatgaccaaattggctact | |
| | accgaagagctaccagacgaattcgtggtggtgacggtaaaatgaaagatctcag | |
| | tccaagatggtatttctactacctaggaactgggccagaagctggacttccctat | |
| | ggtgctaacaaagacggcatcatatgggttgcaactgagggagccttgaatacac | |
| | caaaagatcacattggcacccgcaatcctgctaacaatgctgcaatcgtgctaca | |
| | acttcctcaaggaacaacattgccaaaaggcttctacgcagaagggagcagaggc | |
| | ggcagtcaagcctcttctcgttcctcatcacgtagtcgcaacagttTaagaaatt | |
| | caactccaggcagcagtaggggaacttctcctgctagaatggctggcaatggcgg | |
| | tgatgctgctcttgctttgctgctgcttgacagattgaaccagcttgagagcaaa | |
| | atgtctggtaaaggccaacaacaacaaggccaaactgtcactaagaaatctgctg | |
| | ctgaggcttctaagaagcctcggcaaaaacgtactgccactaaagcatacaatgt | |
| | aacacaagctttcggcagacgtggtccagaacaaacccaaggaaattttggggac | |
| | caggaactaatcagacaaggaactgattacaaacattggccgcaaattgcacaat | |
| | ttgcccccagcgcttcagcgttcttcggaatgtcgcgcattggcatggaagtcac | |
| | accttcgggaacgtggttgacctacacaggtgccatcaaattggatgacaaagat | |
| | ccaaatttcaaagatcaagtcattttgctgaataagcatattgacgcatacaaaa | |
| | cattcccaccaacagagcctaaaaaggacaaaaagaagaaggctgatgaaactca | |
| | agccttaccgcagagacagaagaaacagcaaactgtgactcttcttcctgctgca | |
| | gatttggatgatttctccaaacaattgcaacaatccatgagcagtgctgactcaa | |
| | ctcaggcctaaactcatgcagaccacacaaggcagatgggctatataaacgtttt | |
| | cgcttttccgtttacgatatatagtctactcttgtgcagaatgaattctcgtaac | |
| | tacatagcacaagtagatgtagttaactttaatctcacatagcaatctttaatca | |
| | gtgtgtaacattagggaggacttgaaagagccaccacattttcaccgaggccacg | |
| | cggagtacgatcgagtgtacagtgaacaatgctagggagagctgcctatatggaa | |
| | gagccctaatgtgtaaaattaattttagtagtgctatccccatgtgattttaata | |
| | gcttcttaggagaatgac | |
| CoV 2- | ATTAAAGGTTTATACCTTCCCAGGTAACAAACCAACCAACTTTCGATCTCTTGTA | 10 |
| Omicron | GATCTGTTCTCTAAACGAACTTTAAAATCTGTGTGGCTGTCACTCGGCTGCATGC | |
| BA1- | TTAGTGCACTCACGCAGTATAATTAATAACTAATTACTGTCGTTGACAGGACACG | |
| WWD FL | AGTAACTCGTCTATCTTCTGCAGGCTGCTTACGGTTTCGTCCGTGTTGCAGCCGA | |
| genome | TCATCAGCACATCTAGGTTTCGTCCGGGTGTGACCGAAAGGTAAGATGGAGAGCC | |
| (w/o furin | TTGTCCCTGGTTTCAACGAGAAAACACACGTCCAACTCAGTTTGCCTGTTTTACA | |
| cleavage | GGTTCGCGACGTGCTCGTACGTGGCTTTGGAGACTCCGTGGAGGAGGTCTTATCA | |
| site and | GAGGCACGTCAACATCTTAAAGATGGCACTTGTGGCTTAGTAGAAGTTGAAAAAG | |
| poly(A)) | GCGTTTTGCCTCAACTTGAACAGCCCTATGTGTTCATCAAACGTTCGGATGCTCG | |
| Spike | AACTGCACCTCATGGTCATGTTATGGTTGAGCTGGTAGCAGAACTCGAAGGCATT | |
| sequence | CAGTACGGTCGTAGTGGTGAGACACTTGGTGTCCTTGTCCCTCATGTGGGCGAAA | |
| bolded. | TACCAGTGGCTTACCGCAAGGTTCTTCTTCGTAAGAACGGTAATAAAGGAGCTGG | |
| | TGGCCATAGTTACGGCGCCGATCTAAAGTCATTTGACTTAGGCGACGAGCTTGGC | |
| | ACTGATCCTTATGAAGATTTTCAAGAAAACTGGAACACTAAACATAGCAGTGGTG | |
| | TTACCCGTGAACTCATGCGTGAGCTTAACGGAGGGGCATACACTCGCTATGTCGA | |
| | TAACAACTTCTGTGGCCCTGATGGCTACCCTCTTGAGTGCATTAAAGACCTTCTA | |
| | GCACGTGCTGGTAAAGCTTCATGCACTTTGTCCGAACAACTGGACTTTATTGACA | |
| | CTAAGAGGGGTGTATACTGCTGCCGTGAACATGAGCATGAAATTGCTTGGTACAC | |
| | GGAACGTTCTGAAAAGAGCTATGAATTGCAGACACCTTTTGAAATTAAATTGGCA | |
| | AAGAAATTTGACACCTTCAATGGGGAATGTCCAAATTTTGTATTTCCCTTAAATT | |
| | CCATAATCAAGACTATTCAACCAAGGGTTGAAAAGAAAAAGCTTGATGGCTTTAT | |
| | GGGTAGAATTCGATCTGTCTATCCAGTTGCGTCACCAAATGAATGCAACCAAATG | |
| | TGCCTTTCAACTCTCATGAAGTGTGATCATTGTGGTGAAACTTCATGGCAGACGG | |
| | GCGATTTTGTTAAAGCCACTTGCGAATTTTGTGGCACTGAGAATTTGACTAAAGA | |
| | AGGTGCCACTACTTGTGGTTACTTACCCCAAAATGCTGTTGTTAAAATTTATTGT | |
| | CCAGCATGTCACAATTCAGAAGTAGGACCTGAGCATAGTCTTGCCGAATACCATA | |
| | ATGAATCTGGCTTGAAAACCATTCTTCGTAAGGGTGGTCGCACTATTGCCTTTGG | |
| | AGGCTGTGTGTTCTCTTATGTTGGTTGCCATAACAAGTGTGCCTATTGGGTTCCA | |
| | CGTGCTAGCGCTAACATAGGTTGTAACCATACAGGTGTTGTTGGAGAAGGTTCCG | |
| | AAGGTCTTAATGACAACCTTCTTGAAATACTCCAAAAAGAGAAAGTCAACATCAA | |
| | TATTGTTGGTGACTTTAAACTTAATGAAGAGATCGCCATTATTTTGGCATCTTTT | |
| | TCTGCTTCCACAAGTGCTTTTGTGGAAACTGTGAAAGGTTTGGATTATAAAGCAT | |
| | TCAAACAAATTGTTGAATCCTGTGGTAATTTTAAAGTTACAAAAGGAAAAGCTAA | |
| | AAAAGGTGCCTGGAATATTGGTGAACAGAAATCAATACTGAGTCCTCTTTATGCA | |
| | TTTGCATCAGAGGCTGCTCGTGTTGTACGATCAATTTTCTCCCGCACTCTTGAAA | |
| | CTGCTCAAAATTCTGTGCGTGTTTTACAGAAGGCCGCTATAACAATACTAGATGG | |
| | AATTTCACAGTATTCACTGAGACTCATTGATGCTATGATGTTCACATCTGATTTG | |
| | GCTACTAACAATCTAGTTGTAATGGCCTACATTACAGGTGGTGTTGTTCAGTTGA | |
| | CTTCGCAGTGGCTAACTAACATCTTTGGCACTGTTTATGAAAAACTCAAACCCGT | |
| | CCTTGATTGGCTTGAAGAGAAGTTTAAGGAAGGTGTAGAGTTTCTTAGAGACGGT | |
| | TGGGAAATTGTTAAATTTATCTCAACCTGTGCTTGTGAAATTGTCGGTGGACAAA | |
| | TTGTCACCTGTGCAAAGGAAATTAAGGAGAGTGTTCAGACATTCTTTAAGCTTGT | |
| | AAATAAATTTTTGGCTTTGTGTGCTGACTCTATCATTATTGGTGGAGCTAAACTT | |
| | AAAGCCTTGAATTTAGGTGAAACATTTGTCACGCACTCAAAGGGATTGTACAGAA | |
| | AGTGTGTTAAATCCAGAGAAGAAACTGGCCTACTCATGCCTCTAAAAGCCCCAAA | |
| | AGAAATTATCTTCTTAGAGGGAGAAACACTTCCCACAGAAGTGTTAACAGAGGAA | |
| | GTTGTCTTGAAAACTGGTGATTTACAACCATTAGAACAACCTACTAGTGAAGCTG | |
| | TTGAAGCTCCATTGGTTGGTACACCAGTTTGTATTAACGGGCTTATGTTGCTCGA | |
| | AATCAAAGACACAGAAAAGTACTGTGCCCTTGCACCTAATATGATGGTAACAAAC | |
| | AATACCTTCACACTCAAAGGCGGTGCACCAACAAAGGTTACTTTTGGTGATGACA | |
| | CTGTGATAGAAGTGCAAGGTTACAAGAGTGTGAATATCACTTTTGAACTTGATGA | |
| | AAGGATTGATAAAGTACTTAATGAGAAGTGCTCTGCCTATACAGTTGAACTCGGT | |
| | ACAGAAGTAAATGAGTTCGCCTGTGTTGTGGCAGATGCTGTCATAAAAACTTTGC | |
| | AACCAGTATCTGAATTACTTACACCACTGGGCATTGATTTAGATGAGTGGAGTAT | |
| | GGCTACATACTACTTATTTGATGAGTCTGGTGAGTTTATATTGGCTTCACATATG | |
| | TATTGTTCTTTCTACCCTCCAGATGAGGATGAAGAAGAAGGTGATTGTGAAGAAG | |
| | AAGAGTTTGAGCCATCAACTCAATATGAGTATGGTACTGAAGATGATTACCAAGG | |
| | TAAACCTTTGGAATTTGGTGCCACTTCTGCTGCTCTTCAACCTGAAGAAGAGCAA | |
| | GAAGAAGATTGGTTAGATGATGATAGTCAACAAACTGTTGGTCAACAAGACGGCA | |
| | GTGAGGACAATCAGACAACTACTATTCAAACAATTGTTGAGGTTCAACCTCAATT | |
| | AGAGATGGAACTTACACCAGTTGTTCAGACTATTGAAGTGAATAGTTTTAGTGGT | |
| | TATTTAAAACTTACTGACAATGTATACATTAAAAATGCAGACATTGTGGAAGAAG | |
| | CTAAAAAGGTAAAACCAACAGTGGTTGTTAATGCAGCCAATGTTTACCTTAAACA | |
| | TGGAGGAGGTGTTGCAGGAGCCTTAAATAAGGCTACTAACAATGCCATGCAAGTT | |
| | GAATCTGATGATTACATAGCTACTAATGGACCACTTAAAGTGGGTGGTAGTTGTG | |
| | TTTTAAGCGGACACAATCTTGCTAAACACTGTCTTCATGTTGTCGGCCCAAATGT | |
| | TAACAAAGGTGAAGACATTCAACTTCTTAAGAGTGCTTATGAAAATTTTAATCAG | |
| | CACGAAGTTCTACTTGCACCATTATTATCAGCTGGTATTTTTGGTGCTGACCCTA | |
| | TACATTCTTTAAGAGTTTGTGTAGATACTGTTCGCACAAATGTCTACTTAGCTGT | |
| | CTTTGATAAAAATCTCTATGACAAACTTGTTTCAAGCTTTTTGGAAATGAAGAGT | |
| | GAAAAGCAAGTTGAACAAAAGATCGCTGAGATTCCTAAAGAGGAAGTTAAGCCAT | |
| | TTATAACTGAAAGTAAACCTTCAGTTGAACAGAGAAAACAAGATGATAAGAAAAT | |
| | CAAAGCTTGTGTTGAAGAAGTTACAACAACTCTGGAAGAAACTAAGTTCCTCACA | |
| | GAAAACTTGTTACTTTATATTGACATTAATGGCAATCTTCATCCAGATTCTGCCA | |
| | CTCTTGTTAGTGACATTGACATCACTTTCTTAAAGAAAGATGCTCCATATATAGT | |
| | GGGTGATGTTGTTCAAGAGGGTGTTTTAACTGCTGTGGTTATACCTACTAAAAAG | |
| | GCTGGTGGCACTACTGAAATGCTAGCGAAAGCTTTGAGAAAAGTGCCAACAGACA | |
| | ATTATATAACCACTTACCCGGGTCAGGGTTTAAATGGTTACACTGTAGAGGAGGC | |
| | AAAGACAGTGCTTAAAAAGTGTAAAAGTGCCTTTTACATTCTACCATCTATTATC | |
| | TCTAATGAGAAGCAAGAAATTCTTGGAACTGTTTCTTGGAATTTGCGAGAAATGC | |
| | TTGCACATGCAGAAGAAACACGCAAATTAATGCCTGTCTGTGTGGAAACTAAAGC | |
| | CATAGTTTCAACTATACAGCGTAAATATAAGGGTATTAAAATACAAGAGGGTGTG | |
| | GTTGATTATGGTGCTAGATTTTACTTTTACACCAGTAAAACAACTGTAGCGTCAC | |
| | TTATCAACACACTTAACGATCTAAATGAAACTCTTGTTACAATGCCACTTGGCTA | |
| | TGTAACACATGGCTTAAATTTGGAAGAAGCTGCTCGGTATATGAGATCTCTCAAA | |
| | GTGCCAGCTACAGTTTCTGTTTCTTCACCTGATGCTGTTACAGCGTATAATGGTT | |
| | ATCTTACTTCTTCTTCTAAAACACCTGAAGAACATTTTATTGAAACCATCTCACT | |
| | TGCTGGTTCCTATAAAGATTGGTCCTATTCTGGACAATCTACACAACTAGGTATA | |
| | GAATTTCTTAAGAGAGGTGATAAAAGTGTATATTACACTAGTAATCCTACCACAT | |
| | TCCACCTAGATGGTGAAGTTATCACCTTTGACAATCTTAAGACACTTCTTTCTTT | |
| | GAGAGAAGTGAGGACTATTAAGGTGTTTACAACAGTAGACAACATTAACCTCCAC | |
| | ACGCAAGTTGTGGACATGTCAATGACATATGGACAACAGTTTGGTCCAACTTATT | |
| | TGGATGGAGCTGATGTTACTAAAATAAAACCTCATAATTCACATGAAGGTAAAAC | |
| | ATTTTATGTTTTACCTAATGATGACACTCTACGTGTTGAGGCTTTTGAGTACTAC | |
| | CACACAACTGATCCTAGTTTTCTGGGTAGGTACATGTCAGCATTAAATCACACTA | |
| | AAAAGTGGAAATACCCACAAGTTAATGGTTTAACTTCTATTAAATGGGCAGATAA | |
| | CAACTGTTATCTTGCCACTGCATTGTTAACACTCCAACAAATAGAGTTGAAGTTT | |
| | AATCCACCTGCTCTACAAGATGCTTATTACAGAGCAAGGGCTGGTGAAGCTGCTA | |
| | ACTTTTGTGCACTTATCTTAGCCTACTGTAATAAGACAGTAGGTGAGTTAGGTGA | |
| | TGTTAGAGAAACAATGAGTTACTTGTTTCAACATGCCAATTTAGATTCTTGCAAA | |
| | AGAGTCTTGAACGTGGTGTGTAAAACTTGTGGACAACAGCAGACAACCCTTAAGG | |
| | GTGTAGAAGCTGTTATGTACATGGGCACACTTTCTTATGAACAATTTAAGAAAGG | |
| | TGTTCAGATACCTTGTACGTGTGGTAAACAAGCTACAAAATATCTAGTACAACAG | |
| | GAGTCACCTTTTGTTATGATGTCAGCACCACCTGCTCAGTATGAACTTAAGCATG | |
| | GTACATTTACTTGTGCTAGTGAGTACACTGGTAATTACCAGTGTGGTCACTATAA | |
| | ACATATAACTTCTAAAGAAACTTTGTATTGCATAGACGGTGCTTTACTTACAAAG | |
| | TCCTCAGAATACAAAGGTCCTATTACGGATGTTTTCTACAAAGAAAACAGTTACA | |
| | CAACAACCATAAAACCAGTTACTTATAAATTGGATGGTGTTGTTTGTACAGAAAT | |
| | TGACCCTAAGTTGGACAATTATTATAAGAAAGACAATTCTTATTTCACAGAGCAA | |
| | CCAATTGATCTTGTACCAAACCAACCATATCCAAACGCAAGCTTCGATAATTTTA | |
| | AGTTTGTATGTGATAATATCAAATTTGCTGATGATTTAAACCAGTTAACTGGTTA | |
| | TAAGAAACCTGCTTCAAGAGAGCTTAAAGTTACATTTTTCCCTGACTTAAATGGT | |
| | GATGTGGTGGCTATTGATTATAAACACTACACACCCTCTTTTAAGAAAGGAGCTA | |
| | AATTGTTACATAAACCTATTGTTTGGCATGTTAACAATGCAACTAATAAAGCCAC | |
| | GTATAAACCAAATACCTGGTGTATACGTTGTCTTTGGAGCACAAAACCAGTTGAA | |
| | ACATCAAATTCGTTTGATGTACTGAAGTCAGAGGACGCGCAGGGAATGGATAATC | |
| | TTGCCTGCGAAGATCTAAAACCAGTCTCTGAAGAAGTAGTGGAAAATCCTACCAT | |
| | ACAGAAAGACGTTCTTGAGTGTAATGTGAAAACTACCGAAGTTGTAGGAGACATT | |
| | ATACTTAAACCAGCAAATAATAGTTTAAAAATTACAGAAGAGGTTGGCCACACAG | |
| | ATCTAATGGCTGCTTATGTAGACAATTCTAGTCTTACTATTAAGAAACCTAATGA | |
| | ATTATCTAGAGTATTAGGTTTGAAAACCCTTGCTACTCATGGTTTAGCTGCTGTT | |
| | AATAGTGTCCCTTGGGATACTATAGCTAATTATGCTAAGCCTTTTCTTAACAAAG | |
| | TTGTTAGTACAACTACTAACATAGTTACACGGTGTTTAAACCGTGTTTGTACTAA | |
| | TTATATGCCTTATTTCTTTACTTTATTGCTACAATTGTGTACTTTTACTAGAAGT | |
| | ACAAATTCTAGAATTAAAGCATCTATGCCGACTACTATAGCAAAGAATACTGTTA | |
| | AGAGTGTCGGTAAATTTTGTCTAGAGGCTTCATTTAATTATTTGAAGTCACCTAA | |
| | TTTTTCTAAACTGATAAATATTATAATTTGGTTTTTACTATTAAGTGTTTGCCTA | |
| | GGTTCTTTAATCTACTCAACCGCTGCTTTAGGTGTTTTAATGTCTAATTTAGGCA | |
| | TGCCTTCTTACTGTACTGGTTACAGAGAAGGCTATTTGAACTCTACTAATGTCAC | |
| | TATTGCAACCTACTGTACTGGTTCTATACCTTGTAGTGTTTGTCTTAGTGGTTTA | |
| | GATTCTTTAGACACCTATCCTTCTTTAGAAACTATACAAATTACCATTTCATCTT | |
| | TTAAATGGGATTTAACTGCTTTTGGCTTAGTTGCAGAGTGGTTTTTGGCATATAT | |
| | TCTTTTCACTAGGTTTTTCTATGTACTTGGATTGGCTGCAATCATGCAATTGTTT | |
| | TTCAGCTATTTTGCAGTACATTTTATTAGTAATTCTTGGCTTATGTGGTTAATAA | |
| | TTAATCTTGTACAAATGGCCCCGATTTCAGCTATGGTTAGAATGTACATCTTCTT | |
| | TGCATCATTTTATTATGTATGGAAAAGTTATGTGCATGTTGTAGACGGTTGTAAT | |
| | TCATCAACTTGTATGATGTGTTACAAACGTAATAGAGCAACAAGAGTCGAATGTA | |
| | CAACTATTGTTAATGGTGTTAGAAGGTCCTTTTATGTCTATGCTAATGGAGGTAA | |
| | AGGCTTTTGCAAACTACACAATTGGAATTGTGTTAATTGTGATACATTCTGTGCT | |
| | GGTAGTACATTTATTAGTGATGAAGTTGCGAGAGACTTGTCACTACAGTTTAAAA | |
| | GACCAATAAATCCTACTGACCAGTCTTCTTACATCGTTGATAGTGTTACAGTGAA | |
| | GAATGGTTCCATCCATCTTTACTTTGATAAAGCTGGTCAAAAGACTTATGAAAGA | |
| | CATTCTCTCTCTCATTTTGTTAACTTAGACAACCTGAGAGCTAATAACACTAAAG | |
| | GTTCATTGCCTATTAATGTTATAGTTTTTGATGGTAAATCAAAATGTGAAGAATC | |
| | ATCTGCAAAATCAGCGTCTGTTTACTACAGTCAGCTTATGTGTCAACCTATACTG | |
| | TTACTAGATCAGGCATTAGTGTCTGATGTTGGTGATAGTGCGGAAGTTGCAGTTA | |
| | AAATGTTTGATGCTTACGTTAATACGTTTTCATCAACTTTTAACGTACCAATGGA | |
| | AAAACTCAAAACACTAGTTGCAACTGCAGAAGCTGAACTTGCAAAGAATGTGTCC | |
| | TTAGACAATGTCTTATCTACTTTTATTTCAGCAGCTCGGCAAGGGTTTGTTGATT | |
| | CAGATGTAGAAACTAAAGATGTTGTTGAATGTCTTAAATTGTCACATCAATCTGA | |
| | CATAGAAGTTACTGGCGATAGTTGTAATAACTATATGCTCACCTATAACAAAGTT | |
| | GAAAACATGACACCCCGTGACCTTGGTGCTTGTATTGACTGTAGTGCGCGTCATA | |
| | TTAATGCGCAGGTAGCAAAAAGTCACAACATTGCTTTGATATGGAACGTTAAAGA | |
| | TTTCATGTCATTGTCTGAACAACTACGAAAACAAATACGTAGTGCTGCTAAAAAG | |
| | AATAACTTACCTTTTAAGTTGACATGTGCAACTACTAGACAAGTTGTTAATGTTG | |
| | TAACAACAAAGATAGCACTTAAGGGTGGTAAAATTGTTAATAATTGGTTGAAGCA | |
| | GTTAATTAAAGTTACACTTGTGTTCCTTTTTGTTGCTGCTATTTTCTATTTAATA | |
| | ACACCTGTTCATGTCATGTCTAAACATACTGACTTTTCAAGTGAAATCATAGGAT | |
| | ACAAGGCTATTGATGGTGGTGTCACTCGTGACATAGCATCTACAGATACTTGTTT | |
| | TGCTAACAAACATGCTGATTTTGACACATGGTTTAGTCAGCGTGGTGGTAGTTAT | |
| | ACTAATGACAAAGCTTGCCCATTGATTGCTGCAGTCATAACAAGAGAAGTGGGTT | |
| | TTGTCGTGCCTGGTTTGCCTGGCACGATATTACGCACAACTAATGGTGACTTTTT | |
| | GCATTTCTTACCTAGAGTTTTTAGTGCAGTTGGTAACATCTGTTACACACCATCA | |
| | AAACTTATAGAGTACACTGACTTTGCAACATCAGCTTGTGTTTTGGCTGCTGAAT | |
| | GTACAATTTTTAAAGATGCTTCTGGTAAGCCAGTACCATATTGTTATGATACCAA | |
| | TGTACTAGAAGGTTCTGTTGCTTATGAAAGTTTACGCCCTGACACACGTTATGTG | |
| | CTCATGGATGGCTCTATTATTCAATTTCCTAACACCTACCTTGAAGGTTCTGTTA | |
| | GAGTGGTAACAACCTTTGATTCTGAGTACTGTAGGCACGGCACTTGTGAAAGATC | |
| | AGAAGCTGGTGTTTGTGTATCTACTAGTGGTAGATGGGTACTTAACAATGATTAT | |
| | TACAGATCTTTACCAGGAGTTTTCTGTGGTGTAGATGCTGTAAATTTACTTACTA | |
| | ATATGTTTACACCACTAATTCAACCTATTGGTGCTTTGGACATATCAGCATCTAT | |
| | AGTAGCTGGTGGTATTGTAGCTATCGTAGTAACATGCCTTGCCTACTATTTTATG | |
| | AGGTTTAGgAGAGCTTTTGGTGAATACAGTCATGTAGTTGCCTTTAATACTTTAC | |
| | TATTCCTTATGTCATTCACTGTACTCTGTTTAACACCAGTTTACTCATTCTTACC | |
| | TGGTGTTTATTCTGTTATTTACTTGTACTTGACATTTTATCTTACTAATGATGTT | |
| | TCTTTTTTAGCACATATTCAGTGGATGGTTATGTTCACACCTTTAGTACCTTTCT | |
| | GGATAACAATTGCTTATATCATTTGTATTTCCACAAAGCATTTCTATTGGTTCTT | |
| | TAGTAATTACCTAAAGAGACGTGTAGTCTTTAATGGTGTTTCCTTTAGTACTTTT | |
| | GAAGAAGCTGCGCTGTGCACCTTTTTGTTAAATAAAGAAATGTATCTAAAGTTGC | |
| | GTAGTGATGTGCTATTACCTCTTACGCAATATAATAGATACTTAGCTCTTTATAA | |
| | TAAGTACAAGTATTTTAGTGGAGCAATGGATACAACTAGCTACAGAGAAGCTGCT | |
| | TGTTGTCATCTCGCAAAGGCTCTCAATGACTTCAGTAACTCAGGTTCTGATGTTC | |
| | TTTACCAACCACCACAAACCTCTATCACCTCAGCTGTTTTGCAGAGTGGTTTTAG | |
| | AAAAATGGCATTCCCATCTGGTAAAGTTGAGGGTTGTATGGTACAAGTAACTTGT | |
| | GGTACAACTACACTTAACGGTCTTTGGCTTGATGACGTAGTTTACTGTCCAAGAC | |
| | ATGTGATCTGCACCTCTGAAGACATGCTTAACCCTAATTATGAAGATTTACTCAT | |
| | TCGTAAGTCTAATCATAATTTCTTGGTACAGGCTGGTAATGTTCAACTCAGGGTT | |
| | ATTGGACATTCTATGCAAAATTGTGTACTTAAGCTTAAGGTTGATACAGCCAATC | |
| | CTAAGACACCTAAGTATAAGTTTGTTCGCATTCAACCAGGACAGACTTTTTCAGT | |
| | GTTAGCTTGTTACAATGGTTCACCATCTGGTGTTTACCAATGTGCTATGAGGCCC | |
| | AATTTCACTATTAAGGGTTCATTCCTTAATGGTTCATGTGGTAGTGTTGGTTTTA | |
| | ACATAGATTATGACTGTGTCTCTTTTTGTTACATGCACCATATGGAATTACCAAC | |
| | TGGAGTTCATGCTGGCACAGACTTAGAAGGTAACTTTTATGGACCTTTTGTTGAC | |
| | AGGCAAACAGCACAAGCAGCTGGTACGGACACAACTATTACAGTTAATGTTTTAG | |
| | CTTGGTTGTACGCTGCTGTTATAAATGGAGACAGGTGGTTTCTCAATCGATTTAC | |
| | CACAACTCTTAATGACTTTAACCTTGTGGCTATGAAGTACAATTATGAACCTCTA | |
| | ACACAAGACCATGTTGACATACTAGGACCTCTTTCTGCTCAAACTGGAATTGCCG | |
| | TTTTAGATATGTGTGCTTCATTAAAAGAATTACTGCAAAATGGTATGAATGGACG | |
| | TACCATATTGGGTAGTGCTTTATTAGAAGATGAATTTACACCTTTTGATGTTGTT | |
| | AGACAATGCTCAGGTGTTACTTTCCAAAGTGCAGTGAAAAGAACAATCAAGGGTA | |
| | CACACCACTGGTTGTTACTCACAATTTTGACTTCACTTTTAGTTTTAGTCCAGAG | |
| | TACTCAATGGTCTTTGTTCTTTTTTTTGTATGAAAATGCCTTTTTACCTTTTGCT | |
| | ATGGGTATTATTGCTATGTCTGCTTTTGCAATGATGTTTGTCAAACATAAGCATG | |
| | CATTTCTCTGTTTGTTTTTGTTACCTTCTCTTGCCACTGTAGCTTATTTTAATAT | |
| | GGTCTATATGCCTGCTAGTTGGGTGATGCGTATTATGACATGGTTGGATATGGTT | |
| | GATACTAGTTTGTCTGGTTTTAAGCTAAAAGACTGTGTTATGTATGCATCAGCTG | |
| | TAGTGTTACTAATCCTTATGACAGCAAGAACTGTGTATGATGATGGTGCTAGGAG | |
| | AGTGTGGACACTTATGAATGTCTTGACACTCGTTTATAAAGTTTATTATGGTAAT | |
| | GCTTTAGATCAAGCCATTTCCATGTGGGCTCTTATAATCTCTGTTACTTCTAACT | |
| | ACTCAGGTGTAGTTACAACTGTCATGTTCTTGGCCAGAGGTATTGTTTTTATGTG | |
| | TGTTGAGTATTGCCCTATTTTCTTCATAACTGGTAATACACTTCAGTGTATAATG | |
| | CTAGTTTATTGTTTCTTAGGCTATTTTTGTACTTGTTACTTTGGCCTCTTTTGTT | |
| | TACTCAACCGCTACTTTAGACTGACTCTTGGTGTTTATGATTACTTAGTTTCTAC | |
| | ACAGGAGTTTAGATATATGAATTCACAGGGACTACTCCCACCCAAGAATAGCATA | |
| | GATGCCTTCAAACTCAACATTAAATTGTTGGGTGTTGGTGGCAAACCTTGTATCA | |
| | AAGTAGCCACTGTACAGTCTAAAATGTCAGATGTAAAGTGCACATCAGTAGTCTT | |
| | ACTCTCAGTTTTGCAACAACTCAGAGTAGAATCATCATCTAAATTGTGGGCTCAA | |
| | TGTGTCCAGTTACACAATGACATTCTCTTAGCTAAAGATACTACTGAAGCCTTTG | |
| | AAAAAATGGTTTCACTACTTTCTGTTTTGCTTTCCATGCAGGGTGCTGTAGACAT | |
| | AAACAAGCTTTGTGAAGAAATGCTGGACAACAGGGCAACCTTACAAGCTATAGCC | |
| | TCAGAGTTTAGTTCCCTTCCATCATATGCAGCTTTTGCTACTGCTCAAGAAGCTT | |
| | ATGAGCAGGCTGTTGCTAATGGTGATTCTGAAGTTGTTCTTAAAAAGTTGAAGAA | |
| | GTCTTTGAATGTGGCTAAATCTGAATTTGACCGTGATGCAGCCATGCAACGTAAG | |
| | TTGGAAAAGATGGCTGATCAAGCTATGACCCAAATGTATAAACAGGCTAGATCTG | |
| | AGGACAAGAGGGCAAAAGTTACTAGTGCTATGCAGACAATGCTTTTCACTATGCT | |
| | TAGAAAGTTGGATAATGATGCACTCAACAACATTATCAACAATGCAAGAGATGGT | |
| | TGTGTTCCCTTGAACATAATACCTCTTACAACAGCAGCCAAACTAATGGTTGTCA | |
| | TACCAGACTATAACACATATAAAAATACGTGTGATGGTACAACATTTACTTATGC | |
| | ATCAGCATTGTGGGAAATCCAACAGGTTGTAGATGCAGATAGTAAAATTGTTCAA | |
| | CTTAGTGAAATTAGTATGGACAATTCACCTAATTTAGCATGGCCTCTTATTGTAA | |
| | CAGCTTTAAGGGCCAATTCTGCTGTCAAATTACAGAATAATGAGCTTAGTCCTGT | |
| | TGCACTACGACAGATGTCTTGTGCTGCCGGTACTACACAAACTGCTTGCACTGAT | |
| | GACAATGCGTTAGCTTACTACAACACAACAAAGGGAGGTAGGTTTGTACTTGCAC | |
| | TGTTATCCGATTTACAGGATTTGAAATGGGCTAGATTCCCTAAGAGTGATGGAAC | |
| | TGGTACTATCTATACAGAACTGGAACCACCTTGTAGGTTTGTTACAGACACACCT | |
| | AAAGGTCCTAAAGTGAAGTATTTATACTTTATTAAAGGATTAAACAACCTAAATA | |
| | GAGGTATGGTACTTGGTAGTTTAGCTGCCACAGTACGTCTACAAGCTGGTAATGC | |
| | AACAGAAGTGCCTGCCAATTCAACTGTATTATCTTTCTGTGCTTTTGCTGTAGAT | |
| | GCTGCTAAAGCTTACAAAGATTATCTAGCTAGTGGGGGACAACCAATCACTAATT | |
| | GTGTTAAGATGTTGTGTACACACACTGGTACTGGTCAGGCAATAACAGTTACACC | |
| | GGAAGCCAATATGGATCAAGAATCCTTTGGTGGTGCATCGTGTTGTCTGTACTGC | |
| | CGTTGCCACATAGATCATCCAAATCCTAAAGGATTTTGTGACTTAAAAGGTAAGT | |
| | ATGTACAAATACCTACAACTTGTGCTAATGACCCTGTGGGTTTTACACTTAAAAA | |
| | CACAGTCTGTACCGTCTGCGGTATGTGGAAAGGTTATGGCTGTAGTTGTGATCAA | |
| | CTCCGCGAACCCATGCTTCAGTCAGCTGATGCACAATCGTTTTTAAACGGGTTTG | |
| | CGGTGTAAGTGCAGCCCGTCTTACACCGTGCGGCACAGGCACTAGTACTGATGTC | |
| | GTATACAGGGCTTTTGACATCTACAATGATAAAGTAGCTGGTTTTGCTAAATTCC | |
| | TAAAAACTAATTGTTGTCGCTTCCAAGAAAAGGACGAAGATGACAATTTAATTGA | |
| | TTCTTACTTTGTAGTTAAGAGACACACTTTCTCTAACTACCAACATGAAGAAACA | |
| | ATTTATAATTTACTTAAGGATTGTCCAGCTGTTGCTAAACATGACTTCTTTAAGT | |
| | TTAGAATAGACGGTGACATGGTACCACATATATCACGTCAACGTCTTACTAAATA | |
| | CACAATGGCAGACCTCGTCTATGCTTTAAGGCATTTTGATGAAGGTAATTGTGAC | |
| | ACATTAAAAGAAATACTTGTCACATACAATTGTTGTGATGATGATTATTTCAATA | |
| | AAAAGGACTGGTATGATTTTGTAGAAAACCCAGATATATTACGCGTATACGCCAA | |
| | CTTAGGTGAACGTGTACGCCAAGCTTTGTTAAAAACAGTACAATTCTGTGATGCC | |
| | ATGCGAAATGCTGGTATTGTTGGTGTACTGACATTAGATAATCAAGATCTCAATG | |
| | GTAACTGGTATGATTTCGGTGATTTCATACAAACCACGCCAGGTAGTGGAGTTCC | |
| | TGTTGTAGATTCTTATTATTCATTGTTAATGCCTATATTAACCTTGACCAGGGCT | |
| | TTAACTGCAGAGTCACATGTTGACACTGACTTAACAAAGCCTTACATTAAGTGGG | |
| | ATTTGTTAAAATATGACTTCACGGAAGAGAGGTTAAAACTCTTTGACCGTTATTT | |
| | TAAATATTGGGATCAGACATACCACCCAAATTGTGTTAACTGTTTGGATGACAGA | |
| | TGCATTCTGCATTGTGCAAACTTTAATGTTTTATTCTCTACAGTGTTCCCACCTA | |
| | CAAGTTTTGGACCACTAGTGAGAAAAATATTTGTTGATGGTGTTCCATTTGTAGT | |
| | TTCAACTGGATACCACTTCAGAGAGCTAGGTGTTGTACATAATCAGGATGTAAAC | |
| | TTACATAGCTCTAGACTTAGTTTTAAGGAATTACTTGTGTATGCTGCTGACCCTG | |
| | CTATGCACGCTGCTTCTGGTAATCTATTACTAGATAAACGCACTACGTGCTTTTC | |
| | AGTAGCTGCACTTACTAACAATGTTGCTTTTCAAACTGTCAAACCCGGTAATTTT | |
| | AACAAAGACTTCTATGACTTTGCTGTGTCTAAGGGTTTCTTTAAGGAAGGAAGTT | |
| | CTGTTGAATTAAAACACTTCTTCTTTGCTCAGGATGGTAATGCTGCTATCAGCGA | |
| | TTATGACTACTATCGTTATAATCTACCAACAATGTGTGATATCAGACAACTACTA | |
| | TTTGTAGTTGAAGTTGTTGATAAGTACTTTGATTGTTACGATGGTGGCTGTATTA | |
| | ATGCTAACCAAGTCATCGTCAACAACCTAGACAAATCAGCTGGTTTTCCATTTAA | |
| | TAAATGGGGTAAGGCTAGACTTTATTATGATTCAATGAGTTATGAGGATCAAGAT | |
| | GCACTTTTCGCATATACAAAACGTAATGTCATCCCTACTATAACTCAAATGAATC | |
| | TTAAGTATGCCATTAGTGCAAAGAATAGAGCTCGCACCGTAGCTGGTGTCTCTAT | |
| | CTGTAGTACTATGACCAATAGACAGTTTCATCAAAAATTATTGAAATCAATAGCC | |
| | GCCACTAGAGGAGCTACTGTAGTAATTGGAACAAGCAAATTCTATGGTGGTTGGC | |
| | ACAACATGTTAAAAACTGTTTATAGTGATGTAGAAAACCCTCACCTTATGGGTTG | |
| | GGATTATCCTAAATGTGATAGAGCCATGCCTAACATGCTTAGAATTATGGCCTCA | |
| | CTTGTTCTTGCTCGCAAACATACAACGTGTTGTAGCTTGTCACACCGTTTCTATA | |
| | GATTAGCTAATGAGTGTGCTCAAGTATTGAGTGAAATGGTCATGTGTGGCGGTTC | |
| | ACTATATGTTAAACCAGGTGGAACCTCATCAGGAGATGCCACAACTGCTTATGCT | |
| | AATAGTGTTTTTAACATTTGTCAAGCTGTCACGGCCAATGTTAATGCACTTTTAT | |
| | CTACTGATGGTAACAAAATTGCCGATAAGTATGTCCGCAATTTACAACACAGACT | |
| | TTATGAGTGTCTCTATAGAAATAGAGATGTTGACACAGACTTTGTGAATGAGTTT | |
| | TACGCATATTTGCGTAAACATTTCTCAATGATGATACTCTCTGACGATGCTGTTG | |
| | TGTGTTTCAATAGCACTTATGCATCTCAAGGTCTAGTGGCTAGCATAAAGAACTT | |
| | TAAGTCAGTTCTTTATTATCAAAACAATGTTTTTATGTCTGAAGCAAAATGTTGG | |
| | ACTGAGACTGACCTTACTAAAGGACCTCATGAATTTTGCTCTCAACATACAATGC | |
| | TAGTTAAACAGGGTGATGATTATGTGTACCTTCCTTACCCAGATCCATCAAGAAT | |
| | CCTAGGGGCCGGCTGTTTTGTAGATGATATCGTAAAAACAGATGGTACACTTATG | |
| | ATTGAACGGTTCGTGTCTTTAGCTATAGATGCTTACCCACTTACTAAACATCCTA | |
| | ATCAGGAGTATGCTGATGTCTTTCATTTGTACTTACAATACATAAGAAAGCTACA | |
| | TGATGAGTTAACAGGACACATGTTAGACATGTATTCTGTTATGCTTACTAATGAT | |
| | AACACTTCAAGGTATTGGGAACCTGAGTTTTATGAGGCTATGTACACACCGCATA | |
| | CAGTCTTACAGGCTGTTGGGGCTTGTGTTCTTTGCAATTCACAGACTTCATTAAG | |
| | ATGTGGTGCTTGCATACGTAGACCATTCTTATGTTGTAAATGCTGTTACGACCAT | |
| | GTCATATCAACATCACATAAATTAGTCTTGTCTGTTAATCCGTATGTTTGCAGTG | |
| | CTCCAGGTTGTGATGTCACAGATGTGACTCAACTTTACTTAGGAGGTATGAGCTA | |
| | TTATTGTAAATCACATAAACCACCCATTAGTTTTCCATTGTGTGCTAATGGACAA | |
| | GTTTTTGGTTTATATAAAAATACATGTGTTGGTAGCGATAATGTTACTGACTTTA | |
| | ATGCAATTGCAACATGTGACTGGACAAATGCTGGTGATTACATTTTAGCTAACAC | |
| | CTGTACTGAAAGACTCAAGCTTTTTGCAGCAGAAACGCTCAAAGCTACTGAGGAG | |
| | ACATTTAAACTGTCTTATGGTATTGCTACTGTACGTGAAGTGCTGTCTGACAGAG | |
| | AATTACATCTTTCATGGGAAGTTGGTAAACCTAGACCACCACTTAACCGAAATTA | |
| | TGTCTTTACTGGTTATCGTGTAACTAAAAACAGTAAAGTACAAATAGGAGAGTAC | |
| | ACCTTTGAAAAAGGTGACTATGGTGATGCTGTTGTTTACCGAGGTACAACAACTT | |
| | ACAAATTAAATGTTGGTGATTATTTTGTGCTGACATCACATACAGTAATGCCATT | |
| | AAGTGCACCTACACTAGTGCCACAAGAGCACTATGTTAGAATTACTGGCTTATAC | |
| | CCAACACTCAATATCTCAGATGAGTTTTCTAGCAATGTTGCAAATTATCAAAAGG | |
| | TTGGTATGCAAAAGTATTCTACACTCCAGGGACCACCTGGTACTGGTAAGAGTCA | |
| | TTTTGCTATTGGCCTAGCTCTCTACTACCCTTCTGCTCGCATAGTGTATACAGCT | |
| | TGCTCTCATGCCGCTGTTGATGCACTATGTGAGAAGGCATTAAAATATTTGCCTA | |
| | TAGATAAATGTAGTAGAATTATACCTGCACGTGCTCGTGTAGAGTGTTTTGATAA | |
| | ATTCAAAGTGAATTCAACATTAGAACAGTATGTCTTTTGTACTGTAAATGCATTG | |
| | CCTGAGACGACAGCAGATATAGTTGTCTTTGATGAAATTTCAATGGCCACAAATT | |
| | ATGATTTGAGTGTTGTCAATGCCAGATTACGTGCTAAGCACTATGTGTACATTGG | |
| | CGACCCTGCTCAATTACCTGCACCACGCACATTGCTAACTAAGGGCACACTAGAA | |
| | CCAGAATATTTCAATTCAGTGTGTAGACTTATGAAAACTATAGGTCCAGACATGT | |
| | TCCTCGGAACTTGTCGGCGTTGTCCTGCTGAAATTGTTGACACTGTGAGTGCTTT | |
| | GGTTTATGATAATAAGCTTAAAGCACATAAAGACAAATCAGCTCAATGCTTTAAA | |
| | ATGTTTTATAAGGGTGTTATCACGCATGATGTTTCATCTGCAATTAACAGGCCAC | |
| | AAATAGGCGTGGTAAGAGAATTCCTTACACGTAACCCTGCTTGGAGAAAAGCTGT | |
| | CTTTATTTCACCTTATAATTCACAGAATGCTGTAGCCTCAAAGATTTTGGGACTA | |
| | CCAACTCAAACTGTTGATTCATCACAGGGCTCAGAATATGACTATGTCATATTCA | |
| | CTCAAACCACTGAAACAGCTCACTCTTGTAATGTAAACAGATTTAATGTTGCTAT | |
| | TACCAGAGCAAAAGTAGGCATACTTTGCATAATGTCTGATAGAGACCTTTATGAC | |
| | AAGTTGCAATTTACAAGTCTTGAAATTCCACGTAGGAATGTGGCAACTTTACAAG | |
| | CTGAAAATGTAACAGGACTTTTTAAAGATTGTAGTAAGGTAATCACTGGGTTACA | |
| | TCCTACACAGGCACCTACACACCTCAGTGTTGACACTAAATTCAAAACTGAAGGT | |
| | TTATGTGTTGACATACCTGGCATACCTAAGGACATGACCTATAGAAGACTCATCT | |
| | CTATGATGGGTTTTAAAATGAATTATCAAGTTAATGGTTACCCTAACATGTTTAT | |
| | CACCCGCGAAGAAGCTATAAGACATGTACGTGCATGGATTGGCTTCGATGTCGAG | |
| | GGGTGTCATGCTACTAGAGAAGCTGTTGGTACCAATTTACCTTTACAGCTAGGTT | |
| | TTTCTACAGGTGTTAACCTAGTTGCTGTACCTACAGGTTATGTTGATACACCTAA | |
| | TAATACAGATTTTTCCAGAGTTAGTGCTAAACCACCGCCTGGAGATCAATTTAAA | |
| | CACCTCATACCACTTATGTACAAAGGACTTCCTTGGAATGTAGTGCGTATAAAGA | |
| | TTGTACAAATGTTAAGTGACACACTTAAAAATCTCTCTGACAGAGTCGTATTTGT | |
| | CTTATGGGCACATGGCTTTGAGTTGACATCTATGAAGTATTTTGTGAAAATAGGA | |
| | CCTGAGCGCACCTGTTGTCTATGTGATAGACGTGCCACATGCTTTTCCACTGCTT | |
| | CAGACACTTATGCCTGTTGGCATCATTCTATTGGATTTGATTACGTCTATAATCC | |
| | GTTTATGATTGATGTTCAACAATGGGGTTTTACAGGTAACCTACAAAGCAACCAT | |
| | GATCTGTATTGTCAAGTCCATGGTAATGCACATGTAGCTAGTTGTGATGCAATCA | |
| | TGACTAGGTGTCTAGCTGTCCACGAGTGCTTTGTTAAGCGTGTTGACTGGACTAT | |
| | TGAATATCCTATAATTGGTGATGAACTGAAGATTAATGCGGCTTGTAGAAAGGTT | |
| | CAACACATGGTTGTTAAAGCTGCATTATTAGCAGACAAATTCCCAGTTCTTCACG | |
| | ACATTGGTAACCCTAAAGCTATTAAGTGTGTACCTCAAGCTGATGTAGAATGGAA | |
| | GTTCTATGATGCACAGCCTTGTAGTGACAAAGCTTATAAAATAGAAGAATTATTC | |
| | TATTCTTATGCCACACATTCTGACAAATTCACAGATGGTGTATGCCTATTTTGGA | |
| | ATTGCAATGTCGATAGATATCCTGCTAATTCCATTGTTTGTAGATTTGACACTAG | |
| | AGTGCTATCTAACCTTAACTTGCCTGGTTGTGATGGTGGCAGTTTGTATGTAAAT | |
| | AAACATGCATTCCACACACCAGCTTTTGATAAAAGTGCTTTTGTTAATTTAAAAC | |
| | AATTACCATTTTTCTATTACTCTGACAGTCCATGTGAGTCTCATGGAAAACAAGT | |
| | AGTGTCAGATATAGATTATGTACCACTAAAGTCTGCTACGTGTATAACACGTTGC | |
| | AATTTAGGTGGTGCTGTCTGTAGACATCATGCTAATGAGTACAGATTGTATCTCG | |
| | ATGCTTATAACATGATGATCTCAGCTGGCTTTAGCTTGTGGGTTTACAAACAATT | |
| | TGATACTTATAACCTCTGGAACACTTTTACAAGACTTCAGAGTTTAGAAAATGTG | |
| | GCTTTTAATGTTGTAAATAAGGGACACTTTGATGGACAACAGGGTGAAGTACCAG | |
| | TTTCTATCATTAATAACACTGTTTACACAAAAGTTGATGGTGTTGATGTAGAATT | |
| | GTTTGAAAATAAAACAACATTACCTGTTAATGTAGCATTTGAGCTTTGGGCTAAG | |
| | CGCAACATTAAACCAGTACCAGAGGTGAAAATACTCAATAATTTGGGTGTGGACA | |
| | TTGCTGCTAATACTGTGATCTGGGACTACAAAAGAGATGCTCCAGCACATATATC | |
| | TACTATTGGTGTTTGTTCTATGACTGACATAGCCAAGAAACCAACTGAAACGATT | |
| | TGTGCACCACTCACTGTCTTTTTTGATGGTAGAGTTGATGGTCAAGTAGACTTAT | |
| | TTAGAAATGCCCGTAATGGTGTTCTTATTACAGAAGGTAGTGTTAAAGGTTTACA | |
| | ACCATCTGTAGGTCCCAAACAAGCTAGTCTTAATGGAGTCACATTAATTGGAGAA | |
| | GCCGTAAAAACACAGTTCAATTATTATAAGAAAGTTGATGGTGTTGTCCAACAAT | |
| | TACCTGAAACTTACTTTACTCAGAGTAGAAATTTACAAGAATTTAAACCCAGGAG | |
| | TCAAATGGAAATTGATTTCTTAGAATTAGCTATGGATGAATTCATTGAACGGTAT | |
| | AAATTAGAAGGCTATGCCTTCGAACATATCGTTTATGGAGATTTTAGTCATAGTC | |
| | AGTTAGGTGGTTTACATCTACTGATTGGACTAGCTAAACGTTTTAAGGAATCACC | |
| | TTTTGAATTAGAAGATTTTATTCCTATGGACAGTACAGTTAAAAACTATTTCATA | |
| | ACAGATGCGCAAACAGGTTCATCTAAGTGTGTGTGTTCTGTTATTGATTTATTAC | |
| | TTGATGATTTTGTTGAAATAATAAAATCCCAAGATTTATCTGTAGTTTCTAAGGT | |
| | TGTCAAAGTGACTATTGACTATACAGAAATTTCATTTATGCTTTGGTGTAAAGAT | |
| | GGCCATGTAGAAACATTTTACCCAAAATTACAATCTAGTCAAGCGTGGCAACCGG | |
| | GTGTTGCTATGCCTAATCTTTACAAAATGCAAAGAATGCTATTAGAAAAGTGTGA | |
| | CCTTCAAAATTATGGTGATAGTGCAACATTACCTAAAGGCATAATGATGAATGTC | |
| | GCAAAATATACTCAACTGTGTCAATATTTAAACACATTAACATTAGCTGTACCCT | |
| | ATAATATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATTCAGAT | |
| | CTTAATGACTTTGTCTCTGATGCAGATTCAACTTTGATTGGTGATTGTGCAACTG | |
| | TACATACAGCTAATAAATGGGATCTCATTATTAGTGATATGTACGACCCTAAGAC | |
| | TAAAAATGTTACAAAAGAAAATGACTCTAAAGAGGGTTTTTTCACTTACATTTGT | |
| | GGGTTTATACAACAAAAGCTAGCTCTTGGAGGTTCCGTGGCTATAAAGATAACAG | |
| | AACATTCTTGGAATGCTGATCTTTATAAGCTCATGGGACACTTCGCATGGTGGAC | |
| | AGCCTTTGTTACTAATGTGAATGCGTCATCATCTGAAGCATTTTTAATTGGATGT | |
| | AATTATCTTGGCAAACCACGCGAACAAATAGATGGTTATGTCATGCATGCAAATT | |
| | ACATATTTTGGAGGAATACAAATCCAATTCAGTTGTCTTCCTATTCTTTATTTGA | |
| | CATGAGTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTATAATTA | |
| | GAGAAAACAACAGAGTTGTTATTTCTAGTGATGTTCTTGTTAACAACTAAACGAA | |
| | CAatgtttgtttttcttgttttattgccactagtctctagtcagtgtgttaatct | |
| | tacaaccagaactcaattaccccctgcatacactaattctttcacacgtggtgtt | |
| | tattaccctgacaaagttttcagatcctcagttttacattcaactcaggacttgt | |
| | tcttacctttcttttccaatgttacttggttccatgttatctctgggaccaatgg | |
| | tactaagaggtttgataaccctgtcctaccatttaatgatggtgtttattttgct | |
| | tccattgagaagtctaacataataagaggctggatttttggtactactttagatt | |
| | cgaagacccagtccctacttattgttaataacgctactaatgttgttattaaagt | |
| | ctgtgaatttcaattttgtaatgatccatttttggaccacaaaaacaacaaaagt | |
| | tggatggaaagtgagttcagagtttattctagtgcgaataattgcacttttgaat | |
| | atgtctctcagccttttcttatggaccttgaaggaaaacagggtaatttcaaaaa | |
| | tcttagggaatttgtgtttaagaatattgatggttattttaaaatatattctaag | |
| | cacacgcctattatagtgcgtgagccagaagatctccctcagggtttttcggctt | |
| | tagaaccattggtagatttgccaataggtattaacatcactaggtttcaaacttt | |
| | acttgctttacatagaagttatttgactcctggtgattcttcttcaggttggaca | |
| | gctggtgctgcagcttattatgtgggttatcttcaacctaggacttttctattaa | |
| | aatataatgaaaatggaaccattacagatgctgtagactgtgcacttgaccctct | |
| | ctcagaaacaaagtgtacgttgaaatccttcactgtagaaaaaggaatctatcaa | |
| | acttctaactttagagtccaaccaacagaatctattgttagatttcctaatatta | |
| | caaacttgtgcccttttgatgaagtttttaacgccaccagatttgcatctgttta | |
| | tgcttggaacaggaagagaatcagcaactgtgttgctgattattctgtcctatat | |
| | aatctcgcaccatttttcacttttaagtgttatggagtgtctcctactaaattaa | |
| | atgatctctgctttactaatgtctatgcagattcatttgtaattagaggtgatga | |
| | agtcagacaaatcgctccagggcaaactggaaatattgctgattataattataaa | |
| | ttaccagatgattttacaggctgcgttatagcttggaattctaacaagcttgatt | |
| | ctaaggttagtggtaattataattacctgtatagattgtttaggaagtctaatct | |
| | caaaccttttgagagagatatttcaactgaaatctatcaggccggtaacaaacct | |
| | tgtaatggtgttgcaggttttaattgttactttcctttacgatcatatagtttcc | |
| | gacccacttatggtgttggtcaccaaccatacagagtagtagtactttcttttga | |
| | acttctacatgcaccagcaactgtttgtggacctaaaaagtctactaatttggtt | |
| | aaaaacaaatgtgtcaatttcaacttcaatggtttaaaaggcacaggtgttctta | |
| | ctgagtctaacaaaaagtttctgcctttccaacaatttggcagagacattgctga | |
| | cactactgatgctgtccgtgatccacagacacttgagattcttgacattacacca | |
| | tgttcttttggtggtgtcagtgttataacaccaggaacaaatacttctaaccagg | |
| | ttgctgttctttatcagggtgttaactgcacagaagtccctgttgctattcatgc | |
| | agatcaacttactcctacttggcgtgtttattctacaggttctaatgtttttcaa | |
| | acacgtgcaggctgtttaataggggctgaatatgtcaacaactcatatgagtgtg | |
| | acatacccattggtgcaggtatatgcgctagttatcagactcagcaatccatcat | |
| | tgcctacactatgtcacttggtgcagaaaattcagttgcttactctaataactct | |
| | attgccatacccacaaattttactattagtgttaccacagaaattctaccagtgt | |
| | ctatgaccaagacatcagtagattgtacaatgtacatttgtggtgattcaactga | |
| | atgcagcaatcttttgttgcaatatggcagtttttgtacacaattaaaacgtgct | |
| | ttaactggaatagctgttgaacaagacaaaaacacccaagaagtttttgcacaag | |
| | tcaaacaaatttacaaaacaccaccaattaaatattttggtggttttaatttttc | |
| | acaaatattaccagatccatcaaaaccaagcaagaggtcatttattgaagatcta | |
| | cttttcaacaaagtgacacttgcagatgctggcttcatcaaacaatatggtgatt | |
| | gccttggtgatattgctgctagagatctcatttgcgctcaaaaatttaagggact | |
| | tacagttttaccacctttacttactgacgaaatgattgcgcaatatacatccgca | |
| | ttgttagccggaactattacatccggatggacttttggcgcaggcgcagcattac | |
| | agattccattcgctatgcaaatggcttataggtttaacggtataggcgttacgca | |
| | aaacgtactttatgagaatcaaaaacttatcgctaaccaatttaattccgctatc | |
| | ggtaagattcaggattcattgtctagtactgctagtgcactcggtaagttgcaag | |
| | acgtagtgaatcacaacgctcaagcacttaatacactcgttaaacagcttagttc | |
| | taagtttggcgcaatttctagtgtgcttaacgatatattttcgagactcgataaa | |
| | gtcgaagccgaagtgcaaatcgatagattgattaccggtaggttgcaatcattgc | |
| | aaacatacgttacacagcaattgattagggccgcagagatacgcgctagcgctaa | |
| | tctcgcagctactaaaatgtctgaatgcgtactcggacaatctaaacgtgtcgat | |
| | ttttgcggtaagggatatcatcttatgtcttttccacaatctgcacctcacggag | |
| | tcgtgtttttacacgttacttatgtgccagctcaagagaaaaattttacaaccgc | |
| | tcctgctatttgtcatgacggtaaggcacattttcctagagagggcgtattcgtt | |
| | tctaacggtacacattggttcgttacacaacgtaatttttacgaacctcaaatta | |
| | ttactactgataatacattcgtatcaggtaattgtgacgtagtgataggtatcgt | |
| | taataatacagtttacgatccacttcaacctgaactcgatagttttaaagaggaa | |
| | ctcgataagtattttaaaaatcatacatcacctgacgtcgacttaggcgatattt | |
| | caggtattaacgctagtgtcgttaacattcaaaaagagattgatagacttaacga | |
| | agtcgctaaaaatcttaacgaatcacttatcgatctgcaagagttaggtaagtat | |
| | gagcaatatattaaatggccttggtatatttggttaggctttatagccggattga | |
| | tcgcaatcgttatggttacaattatgttatgttgtatgacatcatgttgttcatg | |
| | tcttaagggatgttgttcatgcggatcatgttgtaaatttgacgaagacgattcc | |
| | gaaccagtgcttaaaggcgttaagttacattatacataaACGAACTTATGGATTT | |
| | GTTTATGAGAATCTTCACAATTGGAACTGTAACTTTGAAGCAAGGTGAAATCAAG | |
| | GATGCTACTCCTTCAGATTTTGTTCGCGCTACTGCAACGATACCGATACAAGCCT | |
| | CACTCCCTTTCGGATGGCTTATTGTTGGCGTTGCACTTCTTGCTGTTTTTCAGAG | |
| | CGCTTCCAAAATCATAACCCTCAAAAAGAGATGGCAACTAGCACTCTCCAAGGGT | |
| | GTTCACTTTGTTTGCAACTTGCTGTTGTTGTTTGTAACAGTTTACTCACACCTTT | |
| | TGCTCGTTGCTGCTGGCCTTGAAGCCCCTTTTCTCTATCTTTATGCTTTAGTCTA | |
| | CTTCTTGCAGAGTATAAACTTTGTAAGAATAATAATGAGGCTTTGGCTTTGCTGG | |
| | AAATGCCGTTCCAAAAACCCATTACTTTATGATGCCAACTATTTTCTTTGCTGGC | |
| | ATACTAATTGTTACGACTATTGTATACCTTACAATAGTGTAACTTCTTCAATTGT | |
| | CATTACTTCAGGTGATGGCACAACAAGTCCTATTTCTGAACATGACTACCAGATT | |
| | GGTGGTTATACTGAAAAATGGGAATCTGGAGTAAAAGACTGTGTTGTATTACACA | |
| | GTTACTTCACTTCAGACTATTACCAGCTGTACTCAACTCAATTGAGTACAGACAC | |
| | TGGTGTTGAACATGTTACCTTCTTCATCTACAATAAAATTGTTGATGAGCCTGAA | |
| | GAACATGTCCAAATTCACACAATCGACGGTTCATCCGGAGTTGTTAATCCAGTAA | |
| | TGGAACCAATTTATGATGAACCGACGACGACTACTAGCGTGCCTTTGTAAGCACA | |
| | AGCTGATGAGTACGAACTTATGTACTCATTCGGTTCGGAAGAGACAGGTACGTTA | |
| | ATAGTTAATAGCGTACTTCTTTTTCTTGCTTTCGTGGTATTCTTGCTAGTTACAC | |
| | TAGCCATCCTTACTGCGCTTCGATTGTGTGCGTACTGCTGCAATATTGTTAACGT | |
| | GAGTCTTGTAAAACCTTCTTTTTACGTTTACTCTCGTGTTAAAAATCTGAATTCT | |
| | TCTAGAGTTCCTGATCTTCTGGTCTAAACGAACTAAATATTATATTAGTTTTTCT | |
| | GTTTGGAACTTTAATTTTAGCCATGGCAGATTCCAACGGTACTATTACCGTTGAA | |
| | GAGCTTAAAAAGCTCCTTGAACAATGGAACCTAGTAATAGGTTTCCTATTCCTTA | |
| | CATGGATTTGTCTTCTACAATTTGCCTATGCCAACAGGAATAGGTTTTTGTATAT | |
| | AATTAAGTTAATTTTCCTCTGGCTGTTATGGCCAGTAACTTTAGCTTGTTTTGTG | |
| | CTTGCTGCTGTTTACAGAATAAATTGGATCACCGGTGGAATTGCTATCGCAATGG | |
| | CTTGTCTTGTAGGCTTGATGTGGCTCAGCTACTTCATTGCTTCTTTCAGACTGTT | |
| | TGCGCGTACGCGTTCCATGTGGTCATTCAATCCAGAAACTAACATTCTTCTCAAC | |
| | GTGCCACTCCATGGCACTATTCTGACCAGACCGCTTCTAGAAAGTGAACTCGTAA | |
| | TCGGAGCTGTGATCCTTCGTGGACATCTTCGTATTGCTGGACACCATCTAGGACG | |
| | CTGTGACATCAAGGACCTGCCTAAAGAAATCACTGTTGCTACATCACGAACGCTT | |
| | TCTTATTACAAATTGGGAGCTTCGCAGCGTGTAGCAGGTGACTCAGGTTTTGCTG | |
| | CATACAGTCGCTACAGGATTGGCAACTATAAATTAAACACAGACCATTCCAGTAG | |
| | CAGTGACAATATTGCTTTGCTTGTACAGTAAGTGACAACAGATGTTTCATCTCGT | |
| | TGACTTTCAGGTTACTATAGCAGAGATATTACTAATTATTATGAGGACTTTTAAA | |
| | GTTTCCATTTGGAATCTTGATTACATCATAAACCTCATAATTAAAAATTTATCTA | |
| | AGTCACTAACTGAGAATAAATATTCTCAATTAGATGAAGAGCAACCAATGGAGAT | |
| | TGATTAAACGAACATGAAAATTATTCTTTTCTTGGCACTGATAACACTCGCTACT | |
| | TGTGAGCTTTATCACTACCAAGAGTGTGTTAGAGGTACAACAGTACTTTTAAAAG | |
| | AACCTTGCTCTTCTGGAACATACGAGGGCAATTCACCATTTCATCCTCTAGCTGA | |
| | TAACAAATTTGCACTGACTTGCTTTAGCACTCAATTTGCTTTTGCTTGTCCTGAC | |
| | GGCGTAAAACACGTCTATCAGTTACGTGCCAGATCAGTTTCACCTAAACTGTTCA | |
| | TCAGACAAGAGGAAGTTCAAGAACTTTACTCTCCAATTTTTCTTATTGTTGCGGC | |
| | AATAGTGTTTATAACACTTTGCTTCACACTCAAAAGAAAGACAGAATGATTGAAC | |
| | TTTCATTAATTGACTTCTATTTGTGCTTTTTAGCCTTTCTGCTATTCCTTGTTTT | |
| | AATTATGCTTATTATCTTTTGGTTCTCACTTGAACTGCAAGATCATAATGAAACT | |
| | TGTCACGCCTAAACGAACATGAAATTTCTTGTTTTCTTAGGAATCATCACAACTG | |
| | TAGCTGCATTTCACCAAGAATGTAGTTTACAGTCATGTACTCAACATCAACCATA | |
| | TGTAGTTGATGACCCGTGTCCTATTCACTTCTATTCTAAATGGTATATTAGAGTA | |
| | GGAGCTAGAAAATCAGCACCTTTAATTGAATTGTGCGTGGATGAGGCTGGTTCTA | |
| | AATCACCCATTCAGTACATCGATATCGGTAATTATACAGTTTCCTGTTCACCTTT | |
| | TACAATTAATTGCCAGGAACCTAAATTGGGTAGTCTTGTAGTGCGTTGTTCGTTC | |
| | TATGAAGACTTTTTAGAGTATCATGACGTTCGTGTTGTTTTAGATTTCATCTAAA | |
| | CGAACAAACTAAAATGTCTGATAATGGACCCCAAAATCAGCGAAATGCACCCCGC | |
| | ATTACGTTTGGTGGACCCTCAGATTCAACTGGCAGTAACCAGAATGGAGAACGCA | |
| | GTGGGGCGCGATCAAAACAACGTCGGCCCCAAGGTTTACCCAATAATACTGCGTC | |
| | TTGGTTCACCGCTCTCACTCAACATGGCAAGGAAGACCTTAAATTCCCTCGAGGA | |
| | CAAGGCGTTCCAATTAACACCAATAGCAGTCCAGATGACCAAATTGGCTACTACC | |
| | GAAGAGCTACCAGACGAATTCGTGGTGGTGACGGTAAAATGAAAGATCTCAGTCC | |
| | AAGATGGTATTTCTACTACCTAGGAACTGGGCCAGAAGCTGGACTTCCCTATGGT | |
| | GCTAACAAAGACGGCATCATATGGGTTGCAACTGAGGGAGCCTTGAATACACCAA | |
| | AAGATCACATTGGCACCCGCAATCCTGCTAACAATGCTGCAATCGTGCTACAACT | |
| | TCCTCAAGGAACAACATTGCCAAAAGGCTTCTACGCAGAAGGGAGCAGAGGCGGC | |
| | AGTCAAGCCTCTTCTCGTTCCTCATCACGTAGTCGCAACAGTTCAAGAAATTCAA | |
| | CTCCAGGCAGCAGTAGGGGAACTTCTCCTGCTAGAATGGCTGGCAATGGCGGTGA | |
| | TGCTGCTCTTGCTTTGCTGCTGCTTGACAGATTGAACCAGCTTGAGAGCAAAATG | |
| | TCTGGTAAAGGCCAACAACAACAAGGCCAAACTGTCACTAAGAAATCTGCTGCTG | |
| | AGGCTTCTAAGAAGCCTCGGCAAAAACGTACTGCCACTAAAGCATACAATGTAAC | |
| | ACAAGCTTTCGGCAGACGTGGTCCAGAACAAACCCAAGGAAATTTTGGGGACCAG | |
| | GAACTAATCAGACAAGGAACTGATTACAAACATTGGCCGCAAATTGCACAATTTG | |
| | CCCCCAGCGCTTCAGCGTTCTTCGGAATGTCGCGCATTGGCATGGAAGTCACACC | |
| | TTCGGGAACGTGGTTGACCTACACAGGTGCCATCAAATTGGATGACAAAGATCCA | |
| | AATTTCAAAGATCAAGTCATTTTGCTGAATAAGCATATTGACGCATACAAAACAT | |
| | TCCCACCAACAGAGCCTAAAAAGGACAAAAAGAAGAAGGCTGATGAAACTCAAGC | |
| | CTTACCGCAGAGACAGAAGAAACAGCAAACTGTGACTCTTCTTCCTGCTGCAGAT | |
| | TTGGATGATTTCTCCAAACAATTGCAACAATCCATGAGCAGTGCTGACTCAACTC | |
| | AGGCCTAAACTCATGCAGACCACACAAGGCAGATGGGCTATATAAACGTTTTCGC | |
| | TTTTCCGTTTACGATATATAGTCTACTCTTGTGCAGAATGAATTCTCGTAACTAC | |
| | ATAGCACAAGTAGATGTAGTTAACTTTAATCTCACATAGCAATCTTTAATCAGTG | |
| | TGTAACATTAGGGAGGACTTGAAAGAGCCACCACATTTTCACCGAGGCCACGCGG | |
| | AGTACGATCGAGTGTACAGTGAACAATGCTAGGGAGAGCTGCCTATATGGAAGAG | |
| | CCCTAATGTGTAAAATTAATTTTAGTAGTGCTATCCCCATGTGATTTTAATAGCT | |
| | TCTTAGGAGAATGAC | |
| CoV 2- | ATTAAAGGTTTATACCTTCCCAGGTAACAAACCAACCAACTTTCGATCTCTTGTA | 11 |
| Omicron | GATCTGTTCTCTAAACGAACTTTAAAATCTGTGTGGCTGTCACTCGGCTGCATGC | |
| BA2- | TTAGTGCACTCACGCAGTATAATTAATAACTAATTACTGTCGTTGACAGGACACG | |
| WWD FL | AGTAACTCGTCTATCTTCTGCAGGCTGCTTACGGTTTCGTCCGTGTTGCAGCCGA | |
| genome | TCATCAGCACATCTAGGTTTCGTCCGGGTGTGACCGAAAGGTAAGATGGAGAGCC | |
| (w/o furin | TTGTCCCTGGTTTCAACGAGAAAACACACGTCCAACTCAGTTTGCCTGTTTTACA | |
| cleavage | GGTTCGCGACGTGCTCGTACGTGGCTTTGGAGACTCCGTGGAGGAGGTCTTATCA | |
| site and | GAGGCACGTCAACATCTTAAAGATGGCACTTGTGGCTTAGTAGAAGTTGAAAAAG | |
| poly(A)) | GCGTTTTGCCTCAACTTGAACAGCCCTATGTGTTCATCAAACGTTCGGATGCTCG | |
| Spike | AACTGCACCTCATGGTCATGTTATGGTTGAGCTGGTAGCAGAACTCGAAGGCATT | |
| sequence | CAGTACGGTCGTAGTGGTGAGACACTTGGTGTCCTTGTCCCTCATGTGGGCGAAA | |
| bolded | TACCAGTGGCTTACCGCAAGGTTCTTCTTCGTAAGAACGGTAATAAAGGAGCTGG | |
| | TGGCCATAGTTACGGCGCCGATCTAAAGTCATTTGACTTAGGCGACGAGCTTGGC | |
| | ACTGATCCTTATGAAGATTTTCAAGAAAACTGGAACACTAAACATAGCAGTGGTG | |
| | TTACCCGTGAACTCATGCGTGAGCTTAACGGAGGGGCATACACTCGCTATGTCGA | |
| | TAACAACTTCTGTGGCCCTGATGGCTACCCTCTTGAGTGCATTAAAGACCTTCTA | |
| | GCACGTGCTGGTAAAGCTTCATGCACTTTGTCCGAACAACTGGACTTTATTGACA | |
| | CTAAGAGGGGTGTATACTGCTGCCGTGAACATGAGCATGAAATTGCTTGGTACAC | |
| | GGAACGTTCTGAAAAGAGCTATGAATTGCAGACACCTTTTGAAATTAAATTGGCA | |
| | AAGAAATTTGACACCTTCAATGGGGAATGTCCAAATTTTGTATTTCCCTTAAATT | |
| | CCATAATCAAGACTATTCAACCAAGGGTTGAAAAGAAAAAGCTTGATGGCTTTAT | |
| | GGGTAGAATTCGATCTGTCTATCCAGTTGCGTCACCAAATGAATGCAACCAAATG | |
| | TGCCTTTCAACTCTCATGAAGTGTGATCATTGTGGTGAAACTTCATGGCAGACGG | |
| | GCGATTTTGTTAAAGCCACTTGCGAATTTTGTGGCACTGAGAATTTGACTAAAGA | |
| | AGGTGCCACTACTTGTGGTTACTTACCCCAAAATGCTGTTGTTAAAATTTATTGT | |
| | CCAGCATGTCACAATTCAGAAGTAGGACCTGAGCATAGTCTTGCCGAATACCATA | |
| | ATGAATCTGGCTTGAAAACCATTCTTCGTAAGGGTGGTCGCACTATTGCCTTTGG | |
| | AGGCTGTGTGTTCTCTTATGTTGGTTGCCATAACAAGTGTGCCTATTGGGTTCCA | |
| | CGTGCTAGCGCTAACATAGGTTGTAACCATACAGGTGTTGTTGGAGAAGGTTCCG | |
| | AAGGTCTTAATGACAACCTTCTTGAAATACTCCAAAAAGAGAAAGTCAACATCAA | |
| | TATTGTTGGTGACTTTAAACTTAATGAAGAGATCGCCATTATTTTGGCATCTTTT | |
| | TCTGCTTCCACAAGTGCTTTTGTGGAAACTGTGAAAGGTTTGGATTATAAAGCAT | |
| | TCAAACAAATTGTTGAATCCTGTGGTAATTTTAAAGTTACAAAAGGAAAAGCTAA | |
| | AAAAGGTGCCTGGAATATTGGTGAACAGAAATCAATACTGAGTCCTCTTTATGCA | |
| | TTTGCATCAGAGGCTGCTCGTGTTGTACGATCAATTTTCTCCCGCACTCTTGAAA | |
| | CTGCTCAAAATTCTGTGCGTGTTTTACAGAAGGCCGCTATAACAATACTAGATGG | |
| | AATTTCACAGTATTCACTGAGACTCATTGATGCTATGATGTTCACATCTGATTTG | |
| | GCTACTAACAATCTAGTTGTAATGGCCTACATTACAGGTGGTGTTGTTCAGTTGA | |
| | CTTCGCAGTGGCTAACTAACATCTTTGGCACTGTTTATGAAAAACTCAAACCCGT | |
| | CCTTGATTGGCTTGAAGAGAAGTTTAAGGAAGGTGTAGAGTTTCTTAGAGACGGT | |
| | TGGGAAATTGTTAAATTTATCTCAACCTGTGCTTGTGAAATTGTCGGTGGACAAA | |
| | TTGTCACCTGTGCAAAGGAAATTAAGGAGAGTGTTCAGACATTCTTTAAGCTTGT | |
| | AAATAAATTTTTGGCTTTGTGTGCTGACTCTATCATTATTGGTGGAGCTAAACTT | |
| | AAAGCCTTGAATTTAGGTGAAACATTTGTCACGCACTCAAAGGGATTGTACAGAA | |
| | AGTGTGTTAAATCCAGAGAAGAAACTGGCCTACTCATGCCTCTAAAAGCCCCAAA | |
| | AGAAATTATCTTCTTAGAGGGAGAAACACTTCCCACAGAAGTGTTAACAGAGGAA | |
| | GTTGTCTTGAAAACTGGTGATTTACAACCATTAGAACAACCTACTAGTGAAGCTG | |
| | TTGAAGCTCCATTGGTTGGTACACCAGTTTGTATTAACGGGCTTATGTTGCTCGA | |
| | AATCAAAGACACAGAAAAGTACTGTGCCCTTGCACCTAATATGATGGTAACAAAC | |
| | AATACCTTCACACTCAAAGGCGGTGCACCAACAAAGGTTACTTTTGGTGATGACA | |
| | CTGTGATAGAAGTGCAAGGTTACAAGAGTGTGAATATCACTTTTGAACTTGATGA | |
| | AAGGATTGATAAAGTACTTAATGAGAAGTGCTCTGCCTATACAGTTGAACTCGGT | |
| | ACAGAAGTAAATGAGTTCGCCTGTGTTGTGGCAGATGCTGTCATAAAAACTTTGC | |
| | AACCAGTATCTGAATTACTTACACCACTGGGCATTGATTTAGATGAGTGGAGTAT | |
| | GGCTACATACTACTTATTTGATGAGTCTGGTGAGTTTATATTGGCTTCACATATG | |
| | TATTGTTCTTTCTACCCTCCAGATGAGGATGAAGAAGAAGGTGATTGTGAAGAAG | |
| | AAGAGTTTGAGCCATCAACTCAATATGAGTATGGTACTGAAGATGATTACCAAGG | |
| | TAAACCTTTGGAATTTGGTGCCACTTCTGCTGCTCTTCAACCTGAAGAAGAGCAA | |
| | GAAGAAGATTGGTTAGATGATGATAGTCAACAAACTGTTGGTCAACAAGACGGCA | |
| | GTGAGGACAATCAGACAACTACTATTCAAACAATTGTTGAGGTTCAACCTCAATT | |
| | AGAGATGGAACTTACACCAGTTGTTCAGACTATTGAAGTGAATAGTTTTAGTGGT | |
| | TATTTAAAACTTACTGACAATGTATACATTAAAAATGCAGACATTGTGGAAGAAG | |
| | CTAAAAAGGTAAAACCAACAGTGGTTGTTAATGCAGCCAATGTTTACCTTAAACA | |
| | TGGAGGAGGTGTTGCAGGAGCCTTAAATAAGGCTACTAACAATGCCATGCAAGTT | |
| | GAATCTGATGATTACATAGCTACTAATGGACCACTTAAAGTGGGTGGTAGTTGTG | |
| | TTTTAAGCGGACACAATCTTGCTAAACACTGTCTTCATGTTGTCGGCCCAAATGT | |
| | TAACAAAGGTGAAGACATTCAACTTCTTAAGAGTGCTTATGAAAATTTTAATCAG | |
| | CACGAAGTTCTACTTGCACCATTATTATCAGCTGGTATTTTTGGTGCTGACCCTA | |
| | TACATTCTTTAAGAGTTTGTGTAGATACTGTTCGCACAAATGTCTACTTAGCTGT | |
| | CTTTGATAAAAATCTCTATGACAAACTTGTTTCAAGCTTTTTGGAAATGAAGAGT | |
| | GAAAAGCAAGTTGAACAAAAGATCGCTGAGATTCCTAAAGAGGAAGTTAAGCCAT | |
| | TTATAACTGAAAGTAAACCTTCAGTTGAACAGAGAAAACAAGATGATAAGAAAAT | |
| | CAAAGCTTGTGTTGAAGAAGTTACAACAACTCTGGAAGAAACTAAGTTCCTCACA | |
| | GAAAACTTGTTACTTTATATTGACATTAATGGCAATCTTCATCCAGATTCTGCCA | |
| | CTCTTGTTAGTGACATTGACATCACTTTCTTAAAGAAAGATGCTCCATATATAGT | |
| | GGGTGATGTTGTTCAAGAGGGTGTTTTAACTGCTGTGGTTATACCTACTAAAAAG | |
| | GCTGGTGGCACTACTGAAATGCTAGCGAAAGCTTTGAGAAAAGTGCCAACAGACA | |
| | ATTATATAACCACTTACCCGGGTCAGGGTTTAAATGGTTACACTGTAGAGGAGGC | |
| | AAAGACAGTGCTTAAAAAGTGTAAAAGTGCCTTTTACATTCTACCATCTATTATC | |
| | TCTAATGAGAAGCAAGAAATTCTTGGAACTGTTTCTTGGAATTTGCGAGAAATGC | |
| | TTGCACATGCAGAAGAAACACGCAAATTAATGCCTGTCTGTGTGGAAACTAAAGC | |
| | CATAGTTTCAACTATACAGCGTAAATATAAGGGTATTAAAATACAAGAGGGTGTG | |
| | GTTGATTATGGTGCTAGATTTTACTTTTACACCAGTAAAACAACTGTAGCGTCAC | |
| | TTATCAACACACTTAACGATCTAAATGAAACTCTTGTTACAATGCCACTTGGCTA | |
| | TGTAACACATGGCTTAAATTTGGAAGAAGCTGCTCGGTATATGAGATCTCTCAAA | |
| | GTGCCAGCTACAGTTTCTGTTTCTTCACCTGATGCTGTTACAGCGTATAATGGTT | |
| | ATCTTACTTCTTCTTCTAAAACACCTGAAGAACATTTTATTGAAACCATCTCACT | |
| | TGCTGGTTCCTATAAAGATTGGTCCTATTCTGGACAATCTACACAACTAGGTATA | |
| | GAATTTCTTAAGAGAGGTGATAAAAGTGTATATTACACTAGTAATCCTACCACAT | |
| | TCCACCTAGATGGTGAAGTTATCACCTTTGACAATCTTAAGACACTTCTTTCTTT | |
| | GAGAGAAGTGAGGACTATTAAGGTGTTTACAACAGTAGACAACATTAACCTCCAC | |
| | ACGCAAGTTGTGGACATGTCAATGACATATGGACAACAGTTTGGTCCAACTTATT | |
| | TGGATGGAGCTGATGTTACTAAAATAAAACCTCATAATTCACATGAAGGTAAAAC | |
| | ATTTTATGTTTTACCTAATGATGACACTCTACGTGTTGAGGCTTTTGAGTACTAC | |
| | CACACAACTGATCCTAGTTTTCTGGGTAGGTACATGTCAGCATTAAATCACACTA | |
| | AAAAGTGGAAATACCCACAAGTTAATGGTTTAACTTCTATTAAATGGGCAGATAA | |
| | CAACTGTTATCTTGCCACTGCATTGTTAACACTCCAACAAATAGAGTTGAAGTTT | |
| | AATCCACCTGCTCTACAAGATGCTTATTACAGAGCAAGGGCTGGTGAAGCTGCTA | |
| | ACTTTTGTGCACTTATCTTAGCCTACTGTAATAAGACAGTAGGTGAGTTAGGTGA | |
| | TGTTAGAGAAACAATGAGTTACTTGTTTCAACATGCCAATTTAGATTCTTGCAAA | |
| | AGAGTCTTGAACGTGGTGTGTAAAACTTGTGGACAACAGCAGACAACCCTTAAGG | |
| | GTGTAGAAGCTGTTATGTACATGGGCACACTTTCTTATGAACAATTTAAGAAAGG | |
| | TGTTCAGATACCTTGTACGTGTGGTAAACAAGCTACAAAATATCTAGTACAACAG | |
| | GAGTCACCTTTTGTTATGATGTCAGCACCACCTGCTCAGTATGAACTTAAGCATG | |
| | GTACATTTACTTGTGCTAGTGAGTACACTGGTAATTACCAGTGTGGTCACTATAA | |
| | ACATATAACTTCTAAAGAAACTTTGTATTGCATAGACGGTGCTTTACTTACAAAG | |
| | TCCTCAGAATACAAAGGTCCTATTACGGATGTTTTCTACAAAGAAAACAGTTACA | |
| | CAACAACCATAAAACCAGTTACTTATAAATTGGATGGTGTTGTTTGTACAGAAAT | |
| | TGACCCTAAGTTGGACAATTATTATAAGAAAGACAATTCTTATTTCACAGAGCAA | |
| | CCAATTGATCTTGTACCAAACCAACCATATCCAAACGCAAGCTTCGATAATTTTA | |
| | AGTTTGTATGTGATAATATCAAATTTGCTGATGATTTAAACCAGTTAACTGGTTA | |
| | TAAGAAACCTGCTTCAAGAGAGCTTAAAGTTACATTTTTCCCTGACTTAAATGGT | |
| | GATGTGGTGGCTATTGATTATAAACACTACACACCCTCTTTTAAGAAAGGAGCTA | |
| | AATTGTTACATAAACCTATTGTTTGGCATGTTAACAATGCAACTAATAAAGCCAC | |
| | GTATAAACCAAATACCTGGTGTATACGTTGTCTTTGGAGCACAAAACCAGTTGAA | |
| | ACATCAAATTCGTTTGATGTACTGAAGTCAGAGGACGCGCAGGGAATGGATAATC | |
| | TTGCCTGCGAAGATCTAAAACCAGTCTCTGAAGAAGTAGTGGAAAATCCTACCAT | |
| | ACAGAAAGACGTTCTTGAGTGTAATGTGAAAACTACCGAAGTTGTAGGAGACATT | |
| | ATACTTAAACCAGCAAATAATAGTTTAAAAATTACAGAAGAGGTTGGCCACACAG | |
| | ATCTAATGGCTGCTTATGTAGACAATTCTAGTCTTACTATTAAGAAACCTAATGA | |
| | ATTATCTAGAGTATTAGGTTTGAAAACCCTTGCTACTCATGGTTTAGCTGCTGTT | |
| | AATAGTGTCCCTTGGGATACTATAGCTAATTATGCTAAGCCTTTTCTTAACAAAG | |
| | TTGTTAGTACAACTACTAACATAGTTACACGGTGTTTAAACCGTGTTTGTACTAA | |
| | TTATATGCCTTATTTCTTTACTTTATTGCTACAATTGTGTACTTTTACTAGAAGT | |
| | ACAAATTCTAGAATTAAAGCATCTATGCCGACTACTATAGCAAAGAATACTGTTA | |
| | AGAGTGTCGGTAAATTTTGTCTAGAGGCTTCATTTAATTATTTGAAGTCACCTAA | |
| | TTTTTCTAAACTGATAAATATTATAATTTGGTTTTTACTATTAAGTGTTTGCCTA | |
| | GGTTCTTTAATCTACTCAACCGCTGCTTTAGGTGTTTTAATGTCTAATTTAGGCA | |
| | TGCCTTCTTACTGTACTGGTTACAGAGAAGGCTATTTGAACTCTACTAATGTCAC | |
| | TATTGCAACCTACTGTACTGGTTCTATACCTTGTAGTGTTTGTCTTAGTGGTTTA | |
| | GATTCTTTAGACACCTATCCTTCTTTAGAAACTATACAAATTACCATTTCATCTT | |
| | TTAAATGGGATTTAACTGCTTTTGGCTTAGTTGCAGAGTGGTTTTTGGCATATAT | |
| | TCTTTTCACTAGGTTTTTCTATGTACTTGGATTGGCTGCAATCATGCAATTGTTT | |
| | TTCAGCTATTTTGCAGTACATTTTATTAGTAATTCTTGGCTTATGTGGTTAATAA | |
| | TTAATCTTGTACAAATGGCCCCGATTTCAGCTATGGTTAGAATGTACATCTTCTT | |
| | TGCATCATTTTATTATGTATGGAAAAGTTATGTGCATGTTGTAGACGGTTGTAAT | |
| | TCATCAACTTGTATGATGTGTTACAAACGTAATAGAGCAACAAGAGTCGAATGTA | |
| | CAACTATTGTTAATGGTGTTAGAAGGTCCTTTTATGTCTATGCTAATGGAGGTAA | |
| | AGGCTTTTGCAAACTACACAATTGGAATTGTGTTAATTGTGATACATTCTGTGCT | |
| | GGTAGTACATTTATTAGTGATGAAGTTGCGAGAGACTTGTCACTACAGTTTAAAA | |
| | GACCAATAAATCCTACTGACCAGTCTTCTTACATCGTTGATAGTGTTACAGTGAA | |
| | GAATGGTTCCATCCATCTTTACTTTGATAAAGCTGGTCAAAAGACTTATGAAAGA | |
| | CATTCTCTCTCTCATTTTGTTAACTTAGACAACCTGAGAGCTAATAACACTAAAG | |
| | GTTCATTGCCTATTAATGTTATAGTTTTTGATGGTAAATCAAAATGTGAAGAATC | |
| | ATCTGCAAAATCAGCGTCTGTTTACTACAGTCAGCTTATGTGTCAACCTATACTG | |
| | TTACTAGATCAGGCATTAGTGTCTGATGTTGGTGATAGTGCGGAAGTTGCAGTTA | |
| | AAATGTTTGATGCTTACGTTAATACGTTTTCATCAACTTTTAACGTACCAATGGA | |
| | AAAACTCAAAACACTAGTTGCAACTGCAGAAGCTGAACTTGCAAAGAATGTGTCC | |
| | TTAGACAATGTCTTATCTACTTTTATTTCAGCAGCTCGGCAAGGGTTTGTTGATT | |
| | CAGATGTAGAAACTAAAGATGTTGTTGAATGTCTTAAATTGTCACATCAATCTGA | |
| | CATAGAAGTTACTGGCGATAGTTGTAATAACTATATGCTCACCTATAACAAAGTT | |
| | GAAAACATGACACCCCGTGACCTTGGTGCTTGTATTGACTGTAGTGCGCGTCATA | |
| | TTAATGCGCAGGTAGCAAAAAGTCACAACATTGCTTTGATATGGAACGTTAAAGA | |
| | TTTCATGTCATTGTCTGAACAACTACGAAAACAAATACGTAGTGCTGCTAAAAAG | |
| | AATAACTTACCTTTTAAGTTGACATGTGCAACTACTAGACAAGTTGTTAATGTTG | |
| | TAACAACAAAGATAGCACTTAAGGGTGGTAAAATTGTTAATAATTGGTTGAAGCA | |
| | GTTAATTAAAGTTACACTTGTGTTCCTTTTTGTTGCTGCTATTTTCTATTTAATA | |
| | ACACCTGTTCATGTCATGTCTAAACATACTGACTTTTCAAGTGAAATCATAGGAT | |
| | ACAAGGCTATTGATGGTGGTGTCACTCGTGACATAGCATCTACAGATACTTGTTT | |
| | TGCTAACAAACATGCTGATTTTGACACATGGTTTAGTCAGCGTGGTGGTAGTTAT | |
| | ACTAATGACAAAGCTTGCCCATTGATTGCTGCAGTCATAACAAGAGAAGTGGGTT | |
| | TTGTCGTGCCTGGTTTGCCTGGCACGATATTACGCACAACTAATGGTGACTTTTT | |
| | GCATTTCTTACCTAGAGTTTTTAGTGCAGTTGGTAACATCTGTTACACACCATCA | |
| | AAACTTATAGAGTACACTGACTTTGCAACATCAGCTTGTGTTTTGGCTGCTGAAT | |
| | GTACAATTTTTAAAGATGCTTCTGGTAAGCCAGTACCATATTGTTATGATACCAA | |
| | TGTACTAGAAGGTTCTGTTGCTTATGAAAGTTTACGCCCTGACACACGTTATGTG | |
| | CTCATGGATGGCTCTATTATTCAATTTCCTAACACCTACCTTGAAGGTTCTGTTA | |
| | GAGTGGTAACAACCTTTGATTCTGAGTACTGTAGGCACGGCACTTGTGAAAGATC | |
| | AGAAGCTGGTGTTTGTGTATCTACTAGTGGTAGATGGGTACTTAACAATGATTAT | |
| | TACAGATCTTTACCAGGAGTTTTCTGTGGTGTAGATGCTGTAAATTTACTTACTA | |
| | ATATGTTTACACCACTAATTCAACCTATTGGTGCTTTGGACATATCAGCATCTAT | |
| | AGTAGCTGGTGGTATTGTAGCTATCGTAGTAACATGCCTTGCCTACTATTTTATG | |
| | AGGTTTAGgAGAGCTTTTGGTGAATACAGTCATGTAGTTGCCTTTAATACTTTAC | |
| | TATTCCTTATGTCATTCACTGTACTCTGTTTAACACCAGTTTACTCATTCTTACC | |
| | TGGTGTTTATTCTGTTATTTACTTGTACTTGACATTTTATCTTACTAATGATGTT | |
| | TCTTTTTTAGCACATATTCAGTGGATGGTTATGTTCACACCTTTAGTACCTTTCT | |
| | GGATAACAATTGCTTATATCATTTGTATTTCCACAAAGCATTTCTATTGGTTCTT | |
| | TAGTAATTACCTAAAGAGACGTGTAGTCTTTAATGGTGTTTCCTTTAGTACTTTT | |
| | GAAGAAGCTGCGCTGTGCACCTTTTTGTTAAATAAAGAAATGTATCTAAAGTTGC | |
| | GTAGTGATGTGCTATTACCTCTTACGCAATATAATAGATACTTAGCTCTTTATAA | |
| | TAAGTACAAGTATTTTAGTGGAGCAATGGATACAACTAGCTACAGAGAAGCTGCT | |
| | TGTTGTCATCTCGCAAAGGCTCTCAATGACTTCAGTAACTCAGGTTCTGATGTTC | |
| | TTTACCAACCACCACAAACCTCTATCACCTCAGCTGTTTTGCAGAGTGGTTTTAG | |
| | AAAAATGGCATTCCCATCTGGTAAAGTTGAGGGTTGTATGGTACAAGTAACTTGT | |
| | GGTACAACTACACTTAACGGTCTTTGGCTTGATGACGTAGTTTACTGTCCAAGAC | |
| | ATGTGATCTGCACCTCTGAAGACATGCTTAACCCTAATTATGAAGATTTACTCAT | |
| | TCGTAAGTCTAATCATAATTTCTTGGTACAGGCTGGTAATGTTCAACTCAGGGTT | |
| | ATTGGACATTCTATGCAAAATTGTGTACTTAAGCTTAAGGTTGATACAGCCAATC | |
| | CTAAGACACCTAAGTATAAGTTTGTTCGCATTCAACCAGGACAGACTTTTTCAGT | |
| | GTTAGCTTGTTACAATGGTTCACCATCTGGTGTTTACCAATGTGCTATGAGGCCC | |
| | AATTTCACTATTAAGGGTTCATTCCTTAATGGTTCATGTGGTAGTGTTGGTTTTA | |
| | ACATAGATTATGACTGTGTCTCTTTTTGTTACATGCACCATATGGAATTACCAAC | |
| | TGGAGTTCATGCTGGCACAGACTTAGAAGGTAACTTTTATGGACCTTTTGTTGAC | |
| | AGGCAAACAGCACAAGCAGCTGGTACGGACACAACTATTACAGTTAATGTTTTAG | |
| | CTTGGTTGTACGCTGCTGTTATAAATGGAGACAGGTGGTTTCTCAATCGATTTAC | |
| | CACAACTCTTAATGACTTTAACCTTGTGGCTATGAAGTACAATTATGAACCTCTA | |
| | ACACAAGACCATGTTGACATACTAGGACCTCTTTCTGCTCAAACTGGAATTGCCG | |
| | TTTTAGATATGTGTGCTTCATTAAAAGAATTACTGCAAAATGGTATGAATGGACG | |
| | TACCATATTGGGTAGTGCTTTATTAGAAGATGAATTTACACCTTTTGATGTTGTT | |
| | AGACAATGCTCAGGTGTTACTTTCCAAAGTGCAGTGAAAAGAACAATCAAGGGTA | |
| | CACACCACTGGTTGTTACTCACAATTTTGACTTCACTTTTAGTTTTAGTCCAGAG | |
| | TACTCAATGGTCTTTGTTCTTTTTTTTGTATGAAAATGCCTTTTTACCTTTTGCT | |
| | ATGGGTATTATTGCTATGTCTGCTTTTGCAATGATGTTTGTCAAACATAAGCATG | |
| | CATTTCTCTGTTTGTTTTTGTTACCTTCTCTTGCCACTGTAGCTTATTTTAATAT | |
| | GGTCTATATGCCTGCTAGTTGGGTGATGCGTATTATGACATGGTTGGATATGGTT | |
| | GATACTAGTTTGTCTGGTTTTAAGCTAAAAGACTGTGTTATGTATGCATCAGCTG | |
| | TAGTGTTACTAATCCTTATGACAGCAAGAACTGTGTATGATGATGGTGCTAGGAG | |
| | AGTGTGGACACTTATGAATGTCTTGACACTCGTTTATAAAGTTTATTATGGTAAT | |
| | GCTTTAGATCAAGCCATTTCCATGTGGGCTCTTATAATCTCTGTTACTTCTAACT | |
| | ACTCAGGTGTAGTTACAACTGTCATGTTCTTGGCCAGAGGTATTGTTTTTATGTG | |
| | TGTTGAGTATTGCCCTATTTTCTTCATAACTGGTAATACACTTCAGTGTATAATG | |
| | CTAGTTTATTGTTTCTTAGGCTATTTTTGTACTTGTTACTTTGGCCTCTTTTGTT | |
| | TACTCAACCGCTACTTTAGACTGACTCTTGGTGTTTATGATTACTTAGTTTCTAC | |
| | ACAGGAGTTTAGATATATGAATTCACAGGGACTACTCCCACCCAAGAATAGCATA | |
| | GATGCCTTCAAACTCAACATTAAATTGTTGGGTGTTGGTGGCAAACCTTGTATCA | |
| | AAGTAGCCACTGTACAGTCTAAAATGTCAGATGTAAAGTGCACATCAGTAGTCTT | |
| | ACTCTCAGTTTTGCAACAACTCAGAGTAGAATCATCATCTAAATTGTGGGCTCAA | |
| | TGTGTCCAGTTACACAATGACATTCTCTTAGCTAAAGATACTACTGAAGCCTTTG | |
| | AAAAAATGGTTTCACTACTTTCTGTTTTGCTTTCCATGCAGGGTGCTGTAGACAT | |
| | AAACAAGCTTTGTGAAGAAATGCTGGACAACAGGGCAACCTTACAAGCTATAGCC | |
| | TCAGAGTTTAGTTCCCTTCCATCATATGCAGCTTTTGCTACTGCTCAAGAAGCTT | |
| | ATGAGCAGGCTGTTGCTAATGGTGATTCTGAAGTTGTTCTTAAAAAGTTGAAGAA | |
| | GTCTTTGAATGTGGCTAAATCTGAATTTGACCGTGATGCAGCCATGCAACGTAAG | |
| | TTGGAAAAGATGGCTGATCAAGCTATGACCCAAATGTATAAACAGGCTAGATCTG | |
| | AGGACAAGAGGGCAAAAGTTACTAGTGCTATGCAGACAATGCTTTTCACTATGCT | |
| | TAGAAAGTTGGATAATGATGCACTCAACAACATTATCAACAATGCAAGAGATGGT | |
| | TGTGTTCCCTTGAACATAATACCTCTTACAACAGCAGCCAAACTAATGGTTGTCA | |
| | TACCAGACTATAACACATATAAAAATACGTGTGATGGTACAACATTTACTTATGC | |
| | ATCAGCATTGTGGGAAATCCAACAGGTTGTAGATGCAGATAGTAAAATTGTTCAA | |
| | CTTAGTGAAATTAGTATGGACAATTCACCTAATTTAGCATGGCCTCTTATTGTAA | |
| | CAGCTTTAAGGGCCAATTCTGCTGTCAAATTACAGAATAATGAGCTTAGTCCTGT | |
| | TGCACTACGACAGATGTCTTGTGCTGCCGGTACTACACAAACTGCTTGCACTGAT | |
| | GACAATGCGTTAGCTTACTACAACACAACAAAGGGAGGTAGGTTTGTACTTGCAC | |
| | TGTTATCCGATTTACAGGATTTGAAATGGGCTAGATTCCCTAAGAGTGATGGAAC | |
| | TGGTACTATCTATACAGAACTGGAACCACCTTGTAGGTTTGTTACAGACACACCT | |
| | AAAGGTCCTAAAGTGAAGTATTTATACTTTATTAAAGGATTAAACAACCTAAATA | |
| | GAGGTATGGTACTTGGTAGTTTAGCTGCCACAGTACGTCTACAAGCTGGTAATGC | |
| | AACAGAAGTGCCTGCCAATTCAACTGTATTATCTTTCTGTGCTTTTGCTGTAGAT | |
| | GCTGCTAAAGCTTACAAAGATTATCTAGCTAGTGGGGGACAACCAATCACTAATT | |
| | GTGTTAAGATGTTGTGTACACACACTGGTACTGGTCAGGCAATAACAGTTACACC | |
| | GGAAGCCAATATGGATCAAGAATCCTTTGGTGGTGCATCGTGTTGTCTGTACTGC | |
| | CGTTGCCACATAGATCATCCAAATCCTAAAGGATTTTGTGACTTAAAAGGTAAGT | |
| | ATGTACAAATACCTACAACTTGTGCTAATGACCCTGTGGGTTTTACACTTAAAAA | |
| | CACAGTCTGTACCGTCTGCGGTATGTGGAAAGGTTATGGCTGTAGTTGTGATCAA | |
| | CTCCGCGAACCCATGCTTCAGTCAGCTGATGCACAATCGTTTTTAAACGGGTTTG | |
| | CGGTGTAAGTGCAGCCCGTCTTACACCGTGCGGCACAGGCACTAGTACTGATGTC | |
| | GTATACAGGGCTTTTGACATCTACAATGATAAAGTAGCTGGTTTTGCTAAATTCC | |
| | TAAAAACTAATTGTTGTCGCTTCCAAGAAAAGGACGAAGATGACAATTTAATTGA | |
| | TTCTTACTTTGTAGTTAAGAGACACACTTTCTCTAACTACCAACATGAAGAAACA | |
| | ATTTATAATTTACTTAAGGATTGTCCAGCTGTTGCTAAACATGACTTCTTTAAGT | |
| | TTAGAATAGACGGTGACATGGTACCACATATATCACGTCAACGTCTTACTAAATA | |
| | CACAATGGCAGACCTCGTCTATGCTTTAAGGCATTTTGATGAAGGTAATTGTGAC | |
| | ACATTAAAAGAAATACTTGTCACATACAATTGTTGTGATGATGATTATTTCAATA | |
| | AAAAGGACTGGTATGATTTTGTAGAAAACCCAGATATATTACGCGTATACGCCAA | |
| | CTTAGGTGAACGTGTACGCCAAGCTTTGTTAAAAACAGTACAATTCTGTGATGCC | |
| | ATGCGAAATGCTGGTATTGTTGGTGTACTGACATTAGATAATCAAGATCTCAATG | |
| | GTAACTGGTATGATTTCGGTGATTTCATACAAACCACGCCAGGTAGTGGAGTTCC | |
| | TGTTGTAGATTCTTATTATTCATTGTTAATGCCTATATTAACCTTGACCAGGGCT | |
| | TTAACTGCAGAGTCACATGTTGACACTGACTTAACAAAGCCTTACATTAAGTGGG | |
| | ATTTGTTAAAATATGACTTCACGGAAGAGAGGTTAAAACTCTTTGACCGTTATTT | |
| | TAAATATTGGGATCAGACATACCACCCAAATTGTGTTAACTGTTTGGATGACAGA | |
| | TGCATTCTGCATTGTGCAAACTTTAATGTTTTATTCTCTACAGTGTTCCCACCTA | |
| | CAAGTTTTGGACCACTAGTGAGAAAAATATTTGTTGATGGTGTTCCATTTGTAGT | |
| | TTCAACTGGATACCACTTCAGAGAGCTAGGTGTTGTACATAATCAGGATGTAAAC | |
| | TTACATAGCTCTAGACTTAGTTTTAAGGAATTACTTGTGTATGCTGCTGACCCTG | |
| | CTATGCACGCTGCTTCTGGTAATCTATTACTAGATAAACGCACTACGTGCTTTTC | |
| | AGTAGCTGCACTTACTAACAATGTTGCTTTTCAAACTGTCAAACCCGGTAATTTT | |
| | AACAAAGACTTCTATGACTTTGCTGTGTCTAAGGGTTTCTTTAAGGAAGGAAGTT | |
| | CTGTTGAATTAAAACACTTCTTCTTTGCTCAGGATGGTAATGCTGCTATCAGCGA | |
| | TTATGACTACTATCGTTATAATCTACCAACAATGTGTGATATCAGACAACTACTA | |
| | TTTGTAGTTGAAGTTGTTGATAAGTACTTTGATTGTTACGATGGTGGCTGTATTA | |
| | ATGCTAACCAAGTCATCGTCAACAACCTAGACAAATCAGCTGGTTTTCCATTTAA | |
| | TAAATGGGGTAAGGCTAGACTTTATTATGATTCAATGAGTTATGAGGATCAAGAT | |
| | GCACTTTTCGCATATACAAAACGTAATGTCATCCCTACTATAACTCAAATGAATC | |
| | TTAAGTATGCCATTAGTGCAAAGAATAGAGCTCGCACCGTAGCTGGTGTCTCTAT | |
| | CTGTAGTACTATGACCAATAGACAGTTTCATCAAAAATTATTGAAATCAATAGCC | |
| | GCCACTAGAGGAGCTACTGTAGTAATTGGAACAAGCAAATTCTATGGTGGTTGGC | |
| | ACAACATGTTAAAAACTGTTTATAGTGATGTAGAAAACCCTCACCTTATGGGTTG | |
| | GGATTATCCTAAATGTGATAGAGCCATGCCTAACATGCTTAGAATTATGGCCTCA | |
| | CTTGTTCTTGCTCGCAAACATACAACGTGTTGTAGCTTGTCACACCGTTTCTATA | |
| | GATTAGCTAATGAGTGTGCTCAAGTATTGAGTGAAATGGTCATGTGTGGCGGTTC | |
| | ACTATATGTTAAACCAGGTGGAACCTCATCAGGAGATGCCACAACTGCTTATGCT | |
| | AATAGTGTTTTTAACATTTGTCAAGCTGTCACGGCCAATGTTAATGCACTTTTAT | |
| | CTACTGATGGTAACAAAATTGCCGATAAGTATGTCCGCAATTTACAACACAGACT | |
| | TTATGAGTGTCTCTATAGAAATAGAGATGTTGACACAGACTTTGTGAATGAGTTT | |
| | TACGCATATTTGCGTAAACATTTCTCAATGATGATACTCTCTGACGATGCTGTTG | |
| | TGTGTTTCAATAGCACTTATGCATCTCAAGGTCTAGTGGCTAGCATAAAGAACTT | |
| | TAAGTCAGTTCTTTATTATCAAAACAATGTTTTTATGTCTGAAGCAAAATGTTGG | |
| | ACTGAGACTGACCTTACTAAAGGACCTCATGAATTTTGCTCTCAACATACAATGC | |
| | TAGTTAAACAGGGTGATGATTATGTGTACCTTCCTTACCCAGATCCATCAAGAAT | |
| | CCTAGGGGCCGGCTGTTTTGTAGATGATATCGTAAAAACAGATGGTACACTTATG | |
| | ATTGAACGGTTCGTGTCTTTAGCTATAGATGCTTACCCACTTACTAAACATCCTA | |
| | ATCAGGAGTATGCTGATGTCTTTCATTTGTACTTACAATACATAAGAAAGCTACA | |
| | TGATGAGTTAACAGGACACATGTTAGACATGTATTCTGTTATGCTTACTAATGAT | |
| | AACACTTCAAGGTATTGGGAACCTGAGTTTTATGAGGCTATGTACACACCGCATA | |
| | CAGTCTTACAGGCTGTTGGGGCTTGTGTTCTTTGCAATTCACAGACTTCATTAAG | |
| | ATGTGGTGCTTGCATACGTAGACCATTCTTATGTTGTAAATGCTGTTACGACCAT | |
| | GTCATATCAACATCACATAAATTAGTCTTGTCTGTTAATCCGTATGTTTGCAGTG | |
| | CTCCAGGTTGTGATGTCACAGATGTGACTCAACTTTACTTAGGAGGTATGAGCTA | |
| | TTATTGTAAATCACATAAACCACCCATTAGTTTTCCATTGTGTGCTAATGGACAA | |
| | GTTTTTGGTTTATATAAAAATACATGTGTTGGTAGCGATAATGTTACTGACTTTA | |
| | ATGCAATTGCAACATGTGACTGGACAAATGCTGGTGATTACATTTTAGCTAACAC | |
| | CTGTACTGAAAGACTCAAGCTTTTTGCAGCAGAAACGCTCAAAGCTACTGAGGAG | |
| | ACATTTAAACTGTCTTATGGTATTGCTACTGTACGTGAAGTGCTGTCTGACAGAG | |
| | AATTACATCTTTCATGGGAAGTTGGTAAACCTAGACCACCACTTAACCGAAATTA | |
| | TGTCTTTACTGGTTATCGTGTAACTAAAAACAGTAAAGTACAAATAGGAGAGTAC | |
| | ACCTTTGAAAAAGGTGACTATGGTGATGCTGTTGTTTACCGAGGTACAACAACTT | |
| | ACAAATTAAATGTTGGTGATTATTTTGTGCTGACATCACATACAGTAATGCCATT | |
| | AAGTGCACCTACACTAGTGCCACAAGAGCACTATGTTAGAATTACTGGCTTATAC | |
| | CCAACACTCAATATCTCAGATGAGTTTTCTAGCAATGTTGCAAATTATCAAAAGG | |
| | TTGGTATGCAAAAGTATTCTACACTCCAGGGACCACCTGGTACTGGTAAGAGTCA | |
| | TTTTGCTATTGGCCTAGCTCTCTACTACCCTTCTGCTCGCATAGTGTATACAGCT | |
| | TGCTCTCATGCCGCTGTTGATGCACTATGTGAGAAGGCATTAAAATATTTGCCTA | |
| | TAGATAAATGTAGTAGAATTATACCTGCACGTGCTCGTGTAGAGTGTTTTGATAA | |
| | ATTCAAAGTGAATTCAACATTAGAACAGTATGTCTTTTGTACTGTAAATGCATTG | |
| | CCTGAGACGACAGCAGATATAGTTGTCTTTGATGAAATTTCAATGGCCACAAATT | |
| | ATGATTTGAGTGTTGTCAATGCCAGATTACGTGCTAAGCACTATGTGTACATTGG | |
| | CGACCCTGCTCAATTACCTGCACCACGCACATTGCTAACTAAGGGCACACTAGAA | |
| | CCAGAATATTTCAATTCAGTGTGTAGACTTATGAAAACTATAGGTCCAGACATGT | |
| | TCCTCGGAACTTGTCGGCGTTGTCCTGCTGAAATTGTTGACACTGTGAGTGCTTT | |
| | GGTTTATGATAATAAGCTTAAAGCACATAAAGACAAATCAGCTCAATGCTTTAAA | |
| | ATGTTTTATAAGGGTGTTATCACGCATGATGTTTCATCTGCAATTAACAGGCCAC | |
| | AAATAGGCGTGGTAAGAGAATTCCTTACACGTAACCCTGCTTGGAGAAAAGCTGT | |
| | CTTTATTTCACCTTATAATTCACAGAATGCTGTAGCCTCAAAGATTTTGGGACTA | |
| | CCAACTCAAACTGTTGATTCATCACAGGGCTCAGAATATGACTATGTCATATTCA | |
| | CTCAAACCACTGAAACAGCTCACTCTTGTAATGTAAACAGATTTAATGTTGCTAT | |
| | TACCAGAGCAAAAGTAGGCATACTTTGCATAATGTCTGATAGAGACCTTTATGAC | |
| | AAGTTGCAATTTACAAGTCTTGAAATTCCACGTAGGAATGTGGCAACTTTACAAG | |
| | CTGAAAATGTAACAGGACTTTTTAAAGATTGTAGTAAGGTAATCACTGGGTTACA | |
| | TCCTACACAGGCACCTACACACCTCAGTGTTGACACTAAATTCAAAACTGAAGGT | |
| | TTATGTGTTGACATACCTGGCATACCTAAGGACATGACCTATAGAAGACTCATCT | |
| | CTATGATGGGTTTTAAAATGAATTATCAAGTTAATGGTTACCCTAACATGTTTAT | |
| | CACCCGCGAAGAAGCTATAAGACATGTACGTGCATGGATTGGCTTCGATGTCGAG | |
| | GGGTGTCATGCTACTAGAGAAGCTGTTGGTACCAATTTACCTTTACAGCTAGGTT | |
| | TTTCTACAGGTGTTAACCTAGTTGCTGTACCTACAGGTTATGTTGATACACCTAA | |
| | TAATACAGATTTTTCCAGAGTTAGTGCTAAACCACCGCCTGGAGATCAATTTAAA | |
| | CACCTCATACCACTTATGTACAAAGGACTTCCTTGGAATGTAGTGCGTATAAAGA | |
| | TTGTACAAATGTTAAGTGACACACTTAAAAATCTCTCTGACAGAGTCGTATTTGT | |
| | CTTATGGGCACATGGCTTTGAGTTGACATCTATGAAGTATTTTGTGAAAATAGGA | |
| | CCTGAGCGCACCTGTTGTCTATGTGATAGACGTGCCACATGCTTTTCCACTGCTT | |
| | CAGACACTTATGCCTGTTGGCATCATTCTATTGGATTTGATTACGTCTATAATCC | |
| | GTTTATGATTGATGTTCAACAATGGGGTTTTACAGGTAACCTACAAAGCAACCAT | |
| | GATCTGTATTGTCAAGTCCATGGTAATGCACATGTAGCTAGTTGTGATGCAATCA | |
| | TGACTAGGTGTCTAGCTGTCCACGAGTGCTTTGTTAAGCGTGTTGACTGGACTAT | |
| | TGAATATCCTATAATTGGTGATGAACTGAAGATTAATGCGGCTTGTAGAAAGGTT | |
| | CAACACATGGTTGTTAAAGCTGCATTATTAGCAGACAAATTCCCAGTTCTTCACG | |
| | ACATTGGTAACCCTAAAGCTATTAAGTGTGTACCTCAAGCTGATGTAGAATGGAA | |
| | GTTCTATGATGCACAGCCTTGTAGTGACAAAGCTTATAAAATAGAAGAATTATTC | |
| | TATTCTTATGCCACACATTCTGACAAATTCACAGATGGTGTATGCCTATTTTGGA | |
| | ATTGCAATGTCGATAGATATCCTGCTAATTCCATTGTTTGTAGATTTGACACTAG | |
| | AGTGCTATCTAACCTTAACTTGCCTGGTTGTGATGGTGGCAGTTTGTATGTAAAT | |
| | AAACATGCATTCCACACACCAGCTTTTGATAAAAGTGCTTTTGTTAATTTAAAAC | |
| | AATTACCATTTTTCTATTACTCTGACAGTCCATGTGAGTCTCATGGAAAACAAGT | |
| | AGTGTCAGATATAGATTATGTACCACTAAAGTCTGCTACGTGTATAACACGTTGC | |
| | AATTTAGGTGGTGCTGTCTGTAGACATCATGCTAATGAGTACAGATTGTATCTCG | |
| | ATGCTTATAACATGATGATCTCAGCTGGCTTTAGCTTGTGGGTTTACAAACAATT | |
| | TGATACTTATAACCTCTGGAACACTTTTACAAGACTTCAGAGTTTAGAAAATGTG | |
| | GCTTTTAATGTTGTAAATAAGGGACACTTTGATGGACAACAGGGTGAAGTACCAG | |
| | TTTCTATCATTAATAACACTGTTTACACAAAAGTTGATGGTGTTGATGTAGAATT | |
| | GTTTGAAAATAAAACAACATTACCTGTTAATGTAGCATTTGAGCTTTGGGCTAAG | |
| | CGCAACATTAAACCAGTACCAGAGGTGAAAATACTCAATAATTTGGGTGTGGACA | |
| | TTGCTGCTAATACTGTGATCTGGGACTACAAAAGAGATGCTCCAGCACATATATC | |
| | TACTATTGGTGTTTGTTCTATGACTGACATAGCCAAGAAACCAACTGAAACGATT | |
| | TGTGCACCACTCACTGTCTTTTTTGATGGTAGAGTTGATGGTCAAGTAGACTTAT | |
| | TTAGAAATGCCCGTAATGGTGTTCTTATTACAGAAGGTAGTGTTAAAGGTTTACA | |
| | ACCATCTGTAGGTCCCAAACAAGCTAGTCTTAATGGAGTCACATTAATTGGAGAA | |
| | GCCGTAAAAACACAGTTCAATTATTATAAGAAAGTTGATGGTGTTGTCCAACAAT | |
| | TACCTGAAACTTACTTTACTCAGAGTAGAAATTTACAAGAATTTAAACCCAGGAG | |
| | TCAAATGGAAATTGATTTCTTAGAATTAGCTATGGATGAATTCATTGAACGGTAT | |
| | AAATTAGAAGGCTATGCCTTCGAACATATCGTTTATGGAGATTTTAGTCATAGTC | |
| | AGTTAGGTGGTTTACATCTACTGATTGGACTAGCTAAACGTTTTAAGGAATCACC | |
| | TTTTGAATTAGAAGATTTTATTCCTATGGACAGTACAGTTAAAAACTATTTCATA | |
| | ACAGATGCGCAAACAGGTTCATCTAAGTGTGTGTGTTCTGTTATTGATTTATTAC | |
| | TTGATGATTTTGTTGAAATAATAAAATCCCAAGATTTATCTGTAGTTTCTAAGGT | |
| | TGTCAAAGTGACTATTGACTATACAGAAATTTCATTTATGCTTTGGTGTAAAGAT | |
| | GGCCATGTAGAAACATTTTACCCAAAATTACAATCTAGTCAAGCGTGGCAACCGG | |
| | GTGTTGCTATGCCTAATCTTTACAAAATGCAAAGAATGCTATTAGAAAAGTGTGA | |
| | CCTTCAAAATTATGGTGATAGTGCAACATTACCTAAAGGCATAATGATGAATGTC | |
| | GCAAAATATACTCAACTGTGTCAATATTTAAACACATTAACATTAGCTGTACCCT | |
| | ATAATATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATTCAGAT | |
| | CTTAATGACTTTGTCTCTGATGCAGATTCAACTTTGATTGGTGATTGTGCAACTG | |
| | TACATACAGCTAATAAATGGGATCTCATTATTAGTGATATGTACGACCCTAAGAC | |
| | TAAAAATGTTACAAAAGAAAATGACTCTAAAGAGGGTTTTTTCACTTACATTTGT | |
| | GGGTTTATACAACAAAAGCTAGCTCTTGGAGGTTCCGTGGCTATAAAGATAACAG | |
| | AACATTCTTGGAATGCTGATCTTTATAAGCTCATGGGACACTTCGCATGGTGGAC | |
| | AGCCTTTGTTACTAATGTGAATGCGTCATCATCTGAAGCATTTTTAATTGGATGT | |
| | AATTATCTTGGCAAACCACGCGAACAAATAGATGGTTATGTCATGCATGCAAATT | |
| | ACATATTTTGGAGGAATACAAATCCAATTCAGTTGTCTTCCTATTCTTTATTTGA | |
| | CATGAGTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTATAATTA | |
| | GAGAAAACAACAGAGTTGTTATTTCTAGTGATGTTCTTGTTAACAACTAAACGAA | |
| | CAatgtttgtttttcttgttttattgccactagtctctagtcagtgtgttaatct | |
| | tataaccagaactcaatcatacactaattctttcacacgtggtgtttattaccct | |
| | gacaaagttttcagatcctcagttttacattcaactcaggacttgttcttacctt | |
| | tcttttccaatgttacttggttccatgctatacatgtctctgggaccaatggtac | |
| | taagaggtttgataaccctgtcctaccatttaatgatggtgtttattttgcttcc | |
| | actgagaagtctaacataataagaggctggatttttggtactactttagattcga | |
| | agacccagtccctacttattgttaataacgctactaatgttgttattaaagtctg | |
| | tgaatttcaattttgtaatgatccatttttggatgtttattaccacaaaaacaac | |
| | aaaagttggatggaaagtgagttcagagtttattctagtgcgaataattgcactt | |
| | ttgaatatgtctctcagccttttcttatggaccttgaaggaaaacagggtaattt | |
| | caaaaatcttagggaatttgtgtttaagaatattgatggttattttaaaatatat | |
| | tctaagcacacgcctattaatttagggcgtgatctccctcagggtttttcggctt | |
| | tagaaccattggtagatttgccaataggtattaacatcactaggtttcaaacttt | |
| | acttgctttacatagaagttatttgactcctggtgattcttcttcaggttggaca | |
| | gctggtgctgcagcttattatgtgggttatcttcaacctaggacttttctattaa | |
| | aatataatgaaaatggaaccattacagatgctgtagactgtgcacttgaccctct | |
| | ctcagaaacaaagtgtacgttgaaatccttcactgtagaaaaaggaatctatcaa | |
| | acttctaactttagagtccaaccaacagaatctattgttagatttcctaatatta | |
| | caaacttgtgcccttttgatgaagtttttaacgccaccagatttgcatctgttta | |
| | tgcttggaacaggaagagaatcagcaactgtgttgctgattattctgtcctatat | |
| | aatttcgcaccatttttcgcttttaagtgttatggagtgtctcctactaaattaa | |
| | atgatctctgctttactaatgtctatgcagattcatttgtaattagaggtaatga | |
| | agtcagccaaatcgctccagggcaaactggaaatattgctgattataattataaa | |
| | ttaccagatgattttacaggctgcgttatagcttggaattctaacaagcttgatt | |
| | ctaaggttggtggtaattataattacctgtatagattgtttaggaagtctaatct | |
| | caaaccttttgagagagatatttcaactgaaatctatcaggccggtaacaaacct | |
| | tgtaatggtgttgcaggttttaattgttactttcctttacgatcatatggtttcc | |
| | gacccacttatggtgttggtcaccaaccatacagagtagtagtactttcttttga | |
| | acttctacatgcaccagcaactgtttgtggacctaaaaagtctactaatttggtt | |
| | aaaaacaaatgtgtcaatttcaacttcaatggtttaacaggcacaggtgttctta | |
| | ctgagtctaacaaaaagtttctgcctttccaacaatttggcagagacattgctga | |
| | cactactgatgctgtccgtgatccacagacacttgagattcttgacattacacca | |
| | tgttcttttggtggtgtcagtgttataacaccaggaacaaatacttctaaccagg | |
| | ttgctgttctttatcagggtgttaactgcacagaagtccctgttgctattcatgc | |
| | agatcaacttactcctacttggcgtgtttattctacaggttctaatgtttttcaa | |
| | acacgtgcaggctgtttaataggggctgaatatgtcaacaactcatatgagtgtg | |
| | acatacccattggtgcaggtatatgcgctagttatcagactcagcaatccatcat | |
| | tgcctacactatgtcacttggtgcagaaaattcagttgcttactctaataactct | |
| | attgccatacccacaaattttactattagtgttaccacagaaattctaccagtgt | |
| | ctatgaccaagacatcagtagattgtacaatgtacatttgtggtgattcaactga | |
| | atgcagcaatcttttgttgcaatatggcagtttttgtacacaattaaaacgtgct | |
| | ttaactggaatagctgttgaacaagacaaaaacacccaagaagtttttgcacaag | |
| | tcaaacaaatttacaaaacaccaccaattaaatattttggtggttttaatttttc | |
| | acaaatattaccagatccatcaaaaccaagcaagaggtcatttattgaagatcta | |
| | cttttcaacaaagtgacacttgcagatgctggcttcatcaaacaatatggtgatt | |
| | gccttggtgatattgctgctagagacctcatttgcgctcaaaaatttaacggact | |
| | tacagttttaccacctttacttactgacgaaatgattgcgcaatatacatccgca | |
| | ttgttagccggaactattacatccggatggacttttggcgcaggcgcagcattac | |
| | agattccattcgctatgcaaatggcttataggtttaacggtataggcgttacgca | |
| | aaacgtactttatgagaatcaaaaacttatcgctaaccaatttaattccgctatc | |
| | ggtaagattcaggattcattgtctagtactgctagtgcactcggtaagttgcaag | |
| | acgtagtgaatcataacgctcaagcacttaatacactcgttaaacagcttagttc | |
| | taaatttggcgcaatttctagtgtgcttaacgatatactatctagactcgataaa | |
| | gtcgaagccgaagtgcaaatcgatagattgattaccggtaggttgcaatcattgc | |
| | aaacatacgttacacagcaattgattagggccgcagagatacgcgctagcgctaa | |
| | tctcgcagctactaaaatgtctgaatgcgtactcggacaatctaaacgtgtcgat | |
| | ttttgcggtaagggatatcatcttatgtcttttccacaatctgcacctcacggag | |
| | tcgtgtttttacacgttacttatgtgccagctcaagagaaaaattttacaaccgc | |
| | tcctgctatttgtcatgacggtaaggcacattttcctagagagggcgtattcgtt | |
| | tctaacggtacacattggttcgttacacaacgtaatttttacgaacctcaaatta | |
| | ttactactgataatacattcgtatcaggtaattgtgacgtagtgataggtatcgt | |
| | taataatacagtttacgatccacttcaacctgaactcgatagttttaaagaggaa | |
| | ctcgataagtattttaaaaatcatacatcacctgacgtcgacttaggcgatattt | |
| | caggtattaacgctagtgtcgttaacattcaaaaagagattgatagacttaacga | |
| | agtcgctaaaaatcttaacgaatcacttatcgatctgcaagagttaggtaagtat | |
| | gagcaatatattaaatggccttggtatatttggttaggctttatagccggattga | |
| | tcgcaatcgttatggttacaattatgttatgttgtatgacatcatgttgttcatg | |
| | tcttaagggatgttgttcatgcggatcatgttgtaaatttgacgaagacgattcc | |
| | gaaccagtgcttaaaggcgttaagttacattatacataaACGAACTTATGGATTT | |
| | GTTTATGAGAATCTTCACAATTGGAACTGTAACTTTGAAGCAAGGTGAAATCAAG | |
| | GATGCTACTCCTTCAGATTTTGTTCGCGCTACTGCAACGATACCGATACAAGCCT | |
| | CACTCCCTTTCGGATGGCTTATTGTTGGCGTTGCACTTCTTGCTGTTTTTCAGAG | |
| | CGCTTCCAAAATCATAACCCTCAAAAAGAGATGGCAACTAGCACTCTCCAAGGGT | |
| | GTTCACTTTGTTTGCAACTTGCTGTTGTTGTTTGTAACAGTTTACTCACACCTTT | |
| | TGCTCGTTGCTGCTGGCCTTGAAGCCCCTTTTCTCTATCTTTATGCTTTAGTCTA | |
| | CTTCTTGCAGAGTATAAACTTTGTAAGAATAATAATGAGGCTTTGGCTTTGCTGG | |
| | AAATGCCGTTCCAAAAACCCATTACTTTATGATGCCAACTATTTTCTTTGCTGGC | |
| | ATACTAATTGTTACGACTATTGTATACCTTACAATAGTGTAACTTCTTCAATTGT | |
| | CATTACTTCAGGTGATGGCACAACAAGTCCTATTTCTGAACATGACTACCAGATT | |
| | GGTGGTTATACTGAAAAATGGGAATCTGGAGTAAAAGACTGTGTTGTATTACACA | |
| | GTTACTTCACTTCAGACTATTACCAGCTGTACTCAACTCAATTGAGTACAGACAC | |
| | TGGTGTTGAACATGTTACCTTCTTCATCTACAATAAAATTGTTGATGAGCCTGAA | |
| | GAACATGTCCAAATTCACACAATCGACGGTTCATCCGGAGTTGTTAATCCAGTAA | |
| | TGGAACCAATTTATGATGAACCGACGACGACTACTAGCGTGCCTTTGTAAGCACA | |
| | AGCTGATGAGTACGAACTTATGTACTCATTCGGTTCGGAAGAGACAGGTACGTTA | |
| | ATAGTTAATAGCGTACTTCTTTTTCTTGCTTTCGTGGTATTCTTGCTAGTTACAC | |
| | TAGCCATCCTTACTGCGCTTCGATTGTGTGCGTACTGCTGCAATATTGTTAACGT | |
| | GAGTCTTGTAAAACCTTCTTTTTACGTTTACTCTCGTGTTAAAAATCTGAATTCT | |
| | TCTAGAGTTCCTGATCTTCTGGTCTAAACGAACTAAATATTATATTAGTTTTTCT | |
| | GTTTGGAACTTTAATTTTAGCCATGGCAGATTCCAACGGTACTATTACCGTTGAA | |
| | GAGCTTAAAAAGCTCCTTGAACAATGGAACCTAGTAATAGGTTTCCTATTCCTTA | |
| | CATGGATTTGTCTTCTACAATTTGCCTATGCCAACAGGAATAGGTTTTTGTATAT | |
| | AATTAAGTTAATTTTCCTCTGGCTGTTATGGCCAGTAACTTTAGCTTGTTTTGTG | |
| | CTTGCTGCTGTTTACAGAATAAATTGGATCACCGGTGGAATTGCTATCGCAATGG | |
| | CTTGTCTTGTAGGCTTGATGTGGCTCAGCTACTTCATTGCTTCTTTCAGACTGTT | |
| | TGCGCGTACGCGTTCCATGTGGTCATTCAATCCAGAAACTAACATTCTTCTCAAC | |
| | GTGCCACTCCATGGCACTATTCTGACCAGACCGCTTCTAGAAAGTGAACTCGTAA | |
| | TCGGAGCTGTGATCCTTCGTGGACATCTTCGTATTGCTGGACACCATCTAGGACG | |
| | CTGTGACATCAAGGACCTGCCTAAAGAAATCACTGTTGCTACATCACGAACGCTT | |
| | TCTTATTACAAATTGGGAGCTTCGCAGCGTGTAGCAGGTGACTCAGGTTTTGCTG | |
| | CATACAGTCGCTACAGGATTGGCAACTATAAATTAAACACAGACCATTCCAGTAG | |
| | CAGTGACAATATTGCTTTGCTTGTACAGTAAGTGACAACAGATGTTTCATCTCGT | |
| | TGACTTTCAGGTTACTATAGCAGAGATATTACTAATTATTATGAGGACTTTTAAA | |
| | GTTTCCATTTGGAATCTTGATTACATCATAAACCTCATAATTAAAAATTTATCTA | |
| | AGTCACTAACTGAGAATAAATATTCTCAATTAGATGAAGAGCAACCAATGGAGAT | |
| | TGATTAAACGAACATGAAAATTATTCTTTTCTTGGCACTGATAACACTCGCTACT | |
| | TGTGAGCTTTATCACTACCAAGAGTGTGTTAGAGGTACAACAGTACTTTTAAAAG | |
| | AACCTTGCTCTTCTGGAACATACGAGGGCAATTCACCATTTCATCCTCTAGCTGA | |
| | TAACAAATTTGCACTGACTTGCTTTAGCACTCAATTTGCTTTTGCTTGTCCTGAC | |
| | GGCGTAAAACACGTCTATCAGTTACGTGCCAGATCAGTTTCACCTAAACTGTTCA | |
| | TCAGACAAGAGGAAGTTCAAGAACTTTACTCTCCAATTTTTCTTATTGTTGCGGC | |
| | AATAGTGTTTATAACACTTTGCTTCACACTCAAAAGAAAGACAGAATGATTGAAC | |
| | TTTCATTAATTGACTTCTATTTGTGCTTTTTAGCCTTTCTGCTATTCCTTGTTTT | |
| | AATTATGCTTATTATCTTTTGGTTCTCACTTGAACTGCAAGATCATAATGAAACT | |
| | TGTCACGCCTAAACGAACATGAAATTTCTTGTTTTCTTAGGAATCATCACAACTG | |
| | TAGCTGCATTTCACCAAGAATGTAGTTTACAGTCATGTACTCAACATCAACCATA | |
| | TGTAGTTGATGACCCGTGTCCTATTCACTTCTATTCTAAATGGTATATTAGAGTA | |
| | GGAGCTAGAAAATCAGCACCTTTAATTGAATTGTGCGTGGATGAGGCTGGTTCTA | |
| | AATCACCCATTCAGTACATCGATATCGGTAATTATACAGTTTCCTGTTCACCTTT | |
| | TACAATTAATTGCCAGGAACCTAAATTGGGTAGTCTTGTAGTGCGTTGTTCGTTC | |
| | TATGAAGACTTTTTAGAGTATCATGACGTTCGTGTTGTTTTAGATTTCATCTAAA | |
| | CGAACAAACTAAAATGTCTGATAATGGACCCCAAAATCAGCGAAATGCACCCCGC | |
| | ATTACGTTTGGTGGACCCTCAGATTCAACTGGCAGTAACCAGAATGGAGAACGCA | |
| | GTGGGGCGCGATCAAAACAACGTCGGCCCCAAGGTTTACCCAATAATACTGCGTC | |
| | TTGGTTCACCGCTCTCACTCAACATGGCAAGGAAGACCTTAAATTCCCTCGAGGA | |
| | CAAGGCGTTCCAATTAACACCAATAGCAGTCCAGATGACCAAATTGGCTACTACC | |
| | GAAGAGCTACCAGACGAATTCGTGGTGGTGACGGTAAAATGAAAGATCTCAGTCC | |
| | AAGATGGTATTTCTACTACCTAGGAACTGGGCCAGAAGCTGGACTTCCCTATGGT | |
| | GCTAACAAAGACGGCATCATATGGGTTGCAACTGAGGGAGCCTTGAATACACCAA | |
| | AAGATCACATTGGCACCCGCAATCCTGCTAACAATGCTGCAATCGTGCTACAACT | |
| | TCCTCAAGGAACAACATTGCCAAAAGGCTTCTACGCAGAAGGGAGCAGAGGCGGC | |
| | AGTCAAGCCTCTTCTCGTTCCTCATCACGTAGTCGCAACAGTTCAAGAAATTCAA | |
| | CTCCAGGCAGCAGTAGGGGAACTTCTCCTGCTAGAATGGCTGGCAATGGCGGTGA | |
| | TGCTGCTCTTGCTTTGCTGCTGCTTGACAGATTGAACCAGCTTGAGAGCAAAATG | |
| | TCTGGTAAAGGCCAACAACAACAAGGCCAAACTGTCACTAAGAAATCTGCTGCTG | |
| | AGGCTTCTAAGAAGCCTCGGCAAAAACGTACTGCCACTAAAGCATACAATGTAAC | |
| | ACAAGCTTTCGGCAGACGTGGTCCAGAACAAACCCAAGGAAATTTTGGGGACCAG | |
| | GAACTAATCAGACAAGGAACTGATTACAAACATTGGCCGCAAATTGCACAATTTG | |
| | CCCCCAGCGCTTCAGCGTTCTTCGGAATGTCGCGCATTGGCATGGAAGTCACACC | |
| | TTCGGGAACGTGGTTGACCTACACAGGTGCCATCAAATTGGATGACAAAGATCCA | |
| | AATTTCAAAGATCAAGTCATTTTGCTGAATAAGCATATTGACGCATACAAAACAT | |
| | TCCCACCAACAGAGCCTAAAAAGGACAAAAAGAAGAAGGCTGATGAAACTCAAGC | |
| | CTTACCGCAGAGACAGAAGAAACAGCAAACTGTGACTCTTCTTCCTGCTGCAGAT | |
| | TTGGATGATTTCTCCAAACAATTGCAACAATCCATGAGCAGTGCTGACTCAACTC | |
| | AGGCCTAAACTCATGCAGACCACACAAGGCAGATGGGCTATATAAACGTTTTCGC | |
| | TTTTCCGTTTACGATATATAGTCTACTCTTGTGCAGAATGAATTCTCGTAACTAC | |
| | ATAGCACAAGTAGATGTAGTTAACTTTAATCTCACATAGCAATCTTTAATCAGTG | |
| | TGTAACATTAGGGAGGACTTGAAAGAGCCACCACATTTTCACCGAGGCCACGCGG | |
| | AGTACGATCGAGTGTACAGTGAACAATGCTAGGGAGAGCTGCCTATATGGAAGAG | |
| | CCCTAATGTGTAAAATTAATTTTAGTAGTGCTATCCCCATGTGATTTTAATAGCT | |
| | TCTTAGGAGAATGAC | |
| CoV 2- | ATTAAAGGTTTATACCTTCCCAGGTAACAAACCAACCAACTTTCGATCTCTTGTA | 12 |
| Omicron | GATCTGTTCTCTAAACGAACTTTAAAATCTGTGTGGCTGTCACTCGGCTGCATGC | |
| BA4/5- | TTAGTGCACTCACGCAGTATAATTAATAACTAATTACTGTCGTTGACAGGACACG | |
| WWD FL | AGTAACTCGTCTATCTTCTGCAGGCTGCTTACGGTTTCGTCCGTGTTGCAGCCGA | |
| genome | TCATCAGCACATCTAGGTTTCGTCCGGGTGTGACCGAAAGGTAAGATGGAGAGCC | |
| (w/o furin | TTGTCCCTGGTTTCAACGAGAAAACACACGTCCAACTCAGTTTGCCTGTTTTACA | |
| cleavage | GGTTCGCGACGTGCTCGTACGTGGCTTTGGAGACTCCGTGGAGGAGGTCTTATCA | |
| site and | GAGGCACGTCAACATCTTAAAGATGGCACTTGTGGCTTAGTAGAAGTTGAAAAAG | |
| poly(A) | GCGTTTTGCCTCAACTTGAACAGCCCTATGTGTTCATCAAACGTTCGGATGCTCG | |
| Spike | AACTGCACCTCATGGTCATGTTATGGTTGAGCTGGTAGCAGAACTCGAAGGCATT | |
| sequence | CAGTACGGTCGTAGTGGTGAGACACTTGGTGTCCTTGTCCCTCATGTGGGCGAAA | |
| bolded. | TACCAGTGGCTTACCGCAAGGTTCTTCTTCGTAAGAACGGTAATAAAGGAGCTGG | |
| | TGGCCATAGTTACGGCGCCGATCTAAAGTCATTTGACTTAGGCGACGAGCTTGGC | |
| | ACTGATCCTTATGAAGATTTTCAAGAAAACTGGAACACTAAACATAGCAGTGGTG | |
| | TTACCCGTGAACTCATGCGTGAGCTTAACGGAGGGGCATACACTCGCTATGTCGA | |
| | TAACAACTTCTGTGGCCCTGATGGCTACCCTCTTGAGTGCATTAAAGACCTTCTA | |
| | GCACGTGCTGGTAAAGCTTCATGCACTTTGTCCGAACAACTGGACTTTATTGACA | |
| | CTAAGAGGGGTGTATACTGCTGCCGTGAACATGAGCATGAAATTGCTTGGTACAC | |
| | GGAACGTTCTGAAAAGAGCTATGAATTGCAGACACCTTTTGAAATTAAATTGGCA | |
| | AAGAAATTTGACACCTTCAATGGGGAATGTCCAAATTTTGTATTTCCCTTAAATT | |
| | CCATAATCAAGACTATTCAACCAAGGGTTGAAAAGAAAAAGCTTGATGGCTTTAT | |
| | GGGTAGAATTCGATCTGTCTATCCAGTTGCGTCACCAAATGAATGCAACCAAATG | |
| | TGCCTTTCAACTCTCATGAAGTGTGATCATTGTGGTGAAACTTCATGGCAGACGG | |
| | GCGATTTTGTTAAAGCCACTTGCGAATTTTGTGGCACTGAGAATTTGACTAAAGA | |
| | AGGTGCCACTACTTGTGGTTACTTACCCCAAAATGCTGTTGTTAAAATTTATTGT | |
| | CCAGCATGTCACAATTCAGAAGTAGGACCTGAGCATAGTCTTGCCGAATACCATA | |
| | ATGAATCTGGCTTGAAAACCATTCTTCGTAAGGGTGGTCGCACTATTGCCTTTGG | |
| | AGGCTGTGTGTTCTCTTATGTTGGTTGCCATAACAAGTGTGCCTATTGGGTTCCA | |
| | CGTGCTAGCGCTAACATAGGTTGTAACCATACAGGTGTTGTTGGAGAAGGTTCCG | |
| | AAGGTCTTAATGACAACCTTCTTGAAATACTCCAAAAAGAGAAAGTCAACATCAA | |
| | TATTGTTGGTGACTTTAAACTTAATGAAGAGATCGCCATTATTTTGGCATCTTTT | |
| | TCTGCTTCCACAAGTGCTTTTGTGGAAACTGTGAAAGGTTTGGATTATAAAGCAT | |
| | TCAAACAAATTGTTGAATCCTGTGGTAATTTTAAAGTTACAAAAGGAAAAGCTAA | |
| | AAAAGGTGCCTGGAATATTGGTGAACAGAAATCAATACTGAGTCCTCTTTATGCA | |
| | TTTGCATCAGAGGCTGCTCGTGTTGTACGATCAATTTTCTCCCGCACTCTTGAAA | |
| | CTGCTCAAAATTCTGTGCGTGTTTTACAGAAGGCCGCTATAACAATACTAGATGG | |
| | AATTTCACAGTATTCACTGAGACTCATTGATGCTATGATGTTCACATCTGATTTG | |
| | GCTACTAACAATCTAGTTGTAATGGCCTACATTACAGGTGGTGTTGTTCAGTTGA | |
| | CTTCGCAGTGGCTAACTAACATCTTTGGCACTGTTTATGAAAAACTCAAACCCGT | |
| | CCTTGATTGGCTTGAAGAGAAGTTTAAGGAAGGTGTAGAGTTTCTTAGAGACGGT | |
| | TGGGAAATTGTTAAATTTATCTCAACCTGTGCTTGTGAAATTGTCGGTGGACAAA | |
| | TTGTCACCTGTGCAAAGGAAATTAAGGAGAGTGTTCAGACATTCTTTAAGCTTGT | |
| | AAATAAATTTTTGGCTTTGTGTGCTGACTCTATCATTATTGGTGGAGCTAAACTT | |
| | AAAGCCTTGAATTTAGGTGAAACATTTGTCACGCACTCAAAGGGATTGTACAGAA | |
| | AGTGTGTTAAATCCAGAGAAGAAACTGGCCTACTCATGCCTCTAAAAGCCCCAAA | |
| | AGAAATTATCTTCTTAGAGGGAGAAACACTTCCCACAGAAGTGTTAACAGAGGAA | |
| | GTTGTCTTGAAAACTGGTGATTTACAACCATTAGAACAACCTACTAGTGAAGCTG | |
| | TTGAAGCTCCATTGGTTGGTACACCAGTTTGTATTAACGGGCTTATGTTGCTCGA | |
| | AATCAAAGACACAGAAAAGTACTGTGCCCTTGCACCTAATATGATGGTAACAAAC | |
| | AATACCTTCACACTCAAAGGCGGTGCACCAACAAAGGTTACTTTTGGTGATGACA | |
| | CTGTGATAGAAGTGCAAGGTTACAAGAGTGTGAATATCACTTTTGAACTTGATGA | |
| | AAGGATTGATAAAGTACTTAATGAGAAGTGCTCTGCCTATACAGTTGAACTCGGT | |
| | ACAGAAGTAAATGAGTTCGCCTGTGTTGTGGCAGATGCTGTCATAAAAACTTTGC | |
| | AACCAGTATCTGAATTACTTACACCACTGGGCATTGATTTAGATGAGTGGAGTAT | |
| | GGCTACATACTACTTATTTGATGAGTCTGGTGAGTTTATATTGGCTTCACATATG | |
| | TATTGTTCTTTCTACCCTCCAGATGAGGATGAAGAAGAAGGTGATTGTGAAGAAG | |
| | AAGAGTTTGAGCCATCAACTCAATATGAGTATGGTACTGAAGATGATTACCAAGG | |
| | TAAACCTTTGGAATTTGGTGCCACTTCTGCTGCTCTTCAACCTGAAGAAGAGCAA | |
| | GAAGAAGATTGGTTAGATGATGATAGTCAACAAACTGTTGGTCAACAAGACGGCA | |
| | GTGAGGACAATCAGACAACTACTATTCAAACAATTGTTGAGGTTCAACCTCAATT | |
| | AGAGATGGAACTTACACCAGTTGTTCAGACTATTGAAGTGAATAGTTTTAGTGGT | |
| | TATTTAAAACTTACTGACAATGTATACATTAAAAATGCAGACATTGTGGAAGAAG | |
| | CTAAAAAGGTAAAACCAACAGTGGTTGTTAATGCAGCCAATGTTTACCTTAAACA | |
| | TGGAGGAGGTGTTGCAGGAGCCTTAAATAAGGCTACTAACAATGCCATGCAAGTT | |
| | GAATCTGATGATTACATAGCTACTAATGGACCACTTAAAGTGGGTGGTAGTTGTG | |
| | TTTTAAGCGGACACAATCTTGCTAAACACTGTCTTCATGTTGTCGGCCCAAATGT | |
| | TAACAAAGGTGAAGACATTCAACTTCTTAAGAGTGCTTATGAAAATTTTAATCAG | |
| | CACGAAGTTCTACTTGCACCATTATTATCAGCTGGTATTTTTGGTGCTGACCCTA | |
| | TACATTCTTTAAGAGTTTGTGTAGATACTGTTCGCACAAATGTCTACTTAGCTGT | |
| | CTTTGATAAAAATCTCTATGACAAACTTGTTTCAAGCTTTTTGGAAATGAAGAGT | |
| | GAAAAGCAAGTTGAACAAAAGATCGCTGAGATTCCTAAAGAGGAAGTTAAGCCAT | |
| | TTATAACTGAAAGTAAACCTTCAGTTGAACAGAGAAAACAAGATGATAAGAAAAT | |
| | CAAAGCTTGTGTTGAAGAAGTTACAACAACTCTGGAAGAAACTAAGTTCCTCACA | |
| | GAAAACTTGTTACTTTATATTGACATTAATGGCAATCTTCATCCAGATTCTGCCA | |
| | CTCTTGTTAGTGACATTGACATCACTTTCTTAAAGAAAGATGCTCCATATATAGT | |
| | GGGTGATGTTGTTCAAGAGGGTGTTTTAACTGCTGTGGTTATACCTACTAAAAAG | |
| | GCTGGTGGCACTACTGAAATGCTAGCGAAAGCTTTGAGAAAAGTGCCAACAGACA | |
| | ATTATATAACCACTTACCCGGGTCAGGGTTTAAATGGTTACACTGTAGAGGAGGC | |
| | AAAGACAGTGCTTAAAAAGTGTAAAAGTGCCTTTTACATTCTACCATCTATTATC | |
| | TCTAATGAGAAGCAAGAAATTCTTGGAACTGTTTCTTGGAATTTGCGAGAAATGC | |
| | TTGCACATGCAGAAGAAACACGCAAATTAATGCCTGTCTGTGTGGAAACTAAAGC | |
| | CATAGTTTCAACTATACAGCGTAAATATAAGGGTATTAAAATACAAGAGGGTGTG | |
| | GTTGATTATGGTGCTAGATTTTACTTTTACACCAGTAAAACAACTGTAGCGTCAC | |
| | TTATCAACACACTTAACGATCTAAATGAAACTCTTGTTACAATGCCACTTGGCTA | |
| | TGTAACACATGGCTTAAATTTGGAAGAAGCTGCTCGGTATATGAGATCTCTCAAA | |
| | GTGCCAGCTACAGTTTCTGTTTCTTCACCTGATGCTGTTACAGCGTATAATGGTT | |
| | ATCTTACTTCTTCTTCTAAAACACCTGAAGAACATTTTATTGAAACCATCTCACT | |
| | TGCTGGTTCCTATAAAGATTGGTCCTATTCTGGACAATCTACACAACTAGGTATA | |
| | GAATTTCTTAAGAGAGGTGATAAAAGTGTATATTACACTAGTAATCCTACCACAT | |
| | TCCACCTAGATGGTGAAGTTATCACCTTTGACAATCTTAAGACACTTCTTTCTTT | |
| | GAGAGAAGTGAGGACTATTAAGGTGTTTACAACAGTAGACAACATTAACCTCCAC | |
| | ACGCAAGTTGTGGACATGTCAATGACATATGGACAACAGTTTGGTCCAACTTATT | |
| | TGGATGGAGCTGATGTTACTAAAATAAAACCTCATAATTCACATGAAGGTAAAAC | |
| | ATTTTATGTTTTACCTAATGATGACACTCTACGTGTTGAGGCTTTTGAGTACTAC | |
| | CACACAACTGATCCTAGTTTTCTGGGTAGGTACATGTCAGCATTAAATCACACTA | |
| | AAAAGTGGAAATACCCACAAGTTAATGGTTTAACTTCTATTAAATGGGCAGATAA | |
| | CAACTGTTATCTTGCCACTGCATTGTTAACACTCCAACAAATAGAGTTGAAGTTT | |
| | AATCCACCTGCTCTACAAGATGCTTATTACAGAGCAAGGGCTGGTGAAGCTGCTA | |
| | ACTTTTGTGCACTTATCTTAGCCTACTGTAATAAGACAGTAGGTGAGTTAGGTGA | |
| | TGTTAGAGAAACAATGAGTTACTTGTTTCAACATGCCAATTTAGATTCTTGCAAA | |
| | AGAGTCTTGAACGTGGTGTGTAAAACTTGTGGACAACAGCAGACAACCCTTAAGG | |
| | GTGTAGAAGCTGTTATGTACATGGGCACACTTTCTTATGAACAATTTAAGAAAGG | |
| | TGTTCAGATACCTTGTACGTGTGGTAAACAAGCTACAAAATATCTAGTACAACAG | |
| | GAGTCACCTTTTGTTATGATGTCAGCACCACCTGCTCAGTATGAACTTAAGCATG | |
| | GTACATTTACTTGTGCTAGTGAGTACACTGGTAATTACCAGTGTGGTCACTATAA | |
| | ACATATAACTTCTAAAGAAACTTTGTATTGCATAGACGGTGCTTTACTTACAAAG | |
| | TCCTCAGAATACAAAGGTCCTATTACGGATGTTTTCTACAAAGAAAACAGTTACA | |
| | CAACAACCATAAAACCAGTTACTTATAAATTGGATGGTGTTGTTTGTACAGAAAT | |
| | TGACCCTAAGTTGGACAATTATTATAAGAAAGACAATTCTTATTTCACAGAGCAA | |
| | CCAATTGATCTTGTACCAAACCAACCATATCCAAACGCAAGCTTCGATAATTTTA | |
| | AGTTTGTATGTGATAATATCAAATTTGCTGATGATTTAAACCAGTTAACTGGTTA | |
| | TAAGAAACCTGCTTCAAGAGAGCTTAAAGTTACATTTTTCCCTGACTTAAATGGT | |
| | GATGTGGTGGCTATTGATTATAAACACTACACACCCTCTTTTAAGAAAGGAGCTA | |
| | AATTGTTACATAAACCTATTGTTTGGCATGTTAACAATGCAACTAATAAAGCCAC | |
| | GTATAAACCAAATACCTGGTGTATACGTTGTCTTTGGAGCACAAAACCAGTTGAA | |
| | ACATCAAATTCGTTTGATGTACTGAAGTCAGAGGACGCGCAGGGAATGGATAATC | |
| | TTGCCTGCGAAGATCTAAAACCAGTCTCTGAAGAAGTAGTGGAAAATCCTACCAT | |
| | ACAGAAAGACGTTCTTGAGTGTAATGTGAAAACTACCGAAGTTGTAGGAGACATT | |
| | ATACTTAAACCAGCAAATAATAGTTTAAAAATTACAGAAGAGGTTGGCCACACAG | |
| | ATCTAATGGCTGCTTATGTAGACAATTCTAGTCTTACTATTAAGAAACCTAATGA | |
| | ATTATCTAGAGTATTAGGTTTGAAAACCCTTGCTACTCATGGTTTAGCTGCTGTT | |
| | AATAGTGTCCCTTGGGATACTATAGCTAATTATGCTAAGCCTTTTCTTAACAAAG | |
| | TTGTTAGTACAACTACTAACATAGTTACACGGTGTTTAAACCGTGTTTGTACTAA | |
| | TTATATGCCTTATTTCTTTACTTTATTGCTACAATTGTGTACTTTTACTAGAAGT | |
| | ACAAATTCTAGAATTAAAGCATCTATGCCGACTACTATAGCAAAGAATACTGTTA | |
| | AGAGTGTCGGTAAATTTTGTCTAGAGGCTTCATTTAATTATTTGAAGTCACCTAA | |
| | TTTTTCTAAACTGATAAATATTATAATTTGGTTTTTACTATTAAGTGTTTGCCTA | |
| | GGTTCTTTAATCTACTCAACCGCTGCTTTAGGTGTTTTAATGTCTAATTTAGGCA | |
| | TGCCTTCTTACTGTACTGGTTACAGAGAAGGCTATTTGAACTCTACTAATGTCAC | |
| | TATTGCAACCTACTGTACTGGTTCTATACCTTGTAGTGTTTGTCTTAGTGGTTTA | |
| | GATTCTTTAGACACCTATCCTTCTTTAGAAACTATACAAATTACCATTTCATCTT | |
| | TTAAATGGGATTTAACTGCTTTTGGCTTAGTTGCAGAGTGGTTTTTGGCATATAT | |
| | TCTTTTCACTAGGTTTTTCTATGTACTTGGATTGGCTGCAATCATGCAATTGTTT | |
| | TTCAGCTATTTTGCAGTACATTTTATTAGTAATTCTTGGCTTATGTGGTTAATAA | |
| | TTAATCTTGTACAAATGGCCCCGATTTCAGCTATGGTTAGAATGTACATCTTCTT | |
| | TGCATCATTTTATTATGTATGGAAAAGTTATGTGCATGTTGTAGACGGTTGTAAT | |
| | TCATCAACTTGTATGATGTGTTACAAACGTAATAGAGCAACAAGAGTCGAATGTA | |
| | CAACTATTGTTAATGGTGTTAGAAGGTCCTTTTATGTCTATGCTAATGGAGGTAA | |
| | AGGCTTTTGCAAACTACACAATTGGAATTGTGTTAATTGTGATACATTCTGTGCT | |
| | GGTAGTACATTTATTAGTGATGAAGTTGCGAGAGACTTGTCACTACAGTTTAAAA | |
| | GACCAATAAATCCTACTGACCAGTCTTCTTACATCGTTGATAGTGTTACAGTGAA | |
| | GAATGGTTCCATCCATCTTTACTTTGATAAAGCTGGTCAAAAGACTTATGAAAGA | |
| | CATTCTCTCTCTCATTTTGTTAACTTAGACAACCTGAGAGCTAATAACACTAAAG | |
| | GTTCATTGCCTATTAATGTTATAGTTTTTGATGGTAAATCAAAATGTGAAGAATC | |
| | ATCTGCAAAATCAGCGTCTGTTTACTACAGTCAGCTTATGTGTCAACCTATACTG | |
| | TTACTAGATCAGGCATTAGTGTCTGATGTTGGTGATAGTGCGGAAGTTGCAGTTA | |
| | AAATGTTTGATGCTTACGTTAATACGTTTTCATCAACTTTTAACGTACCAATGGA | |
| | AAAACTCAAAACACTAGTTGCAACTGCAGAAGCTGAACTTGCAAAGAATGTGTCC | |
| | TTAGACAATGTCTTATCTACTTTTATTTCAGCAGCTCGGCAAGGGTTTGTTGATT | |
| | CAGATGTAGAAACTAAAGATGTTGTTGAATGTCTTAAATTGTCACATCAATCTGA | |
| | CATAGAAGTTACTGGCGATAGTTGTAATAACTATATGCTCACCTATAACAAAGTT | |
| | GAAAACATGACACCCCGTGACCTTGGTGCTTGTATTGACTGTAGTGCGCGTCATA | |
| | TTAATGCGCAGGTAGCAAAAAGTCACAACATTGCTTTGATATGGAACGTTAAAGA | |
| | TTTCATGTCATTGTCTGAACAACTACGAAAACAAATACGTAGTGCTGCTAAAAAG | |
| | AATAACTTACCTTTTAAGTTGACATGTGCAACTACTAGACAAGTTGTTAATGTTG | |
| | TAACAACAAAGATAGCACTTAAGGGTGGTAAAATTGTTAATAATTGGTTGAAGCA | |
| | GTTAATTAAAGTTACACTTGTGTTCCTTTTTGTTGCTGCTATTTTCTATTTAATA | |
| | ACACCTGTTCATGTCATGTCTAAACATACTGACTTTTCAAGTGAAATCATAGGAT | |
| | ACAAGGCTATTGATGGTGGTGTCACTCGTGACATAGCATCTACAGATACTTGTTT | |
| | TGCTAACAAACATGCTGATTTTGACACATGGTTTAGTCAGCGTGGTGGTAGTTAT | |
| | ACTAATGACAAAGCTTGCCCATTGATTGCTGCAGTCATAACAAGAGAAGTGGGTT | |
| | TTGTCGTGCCTGGTTTGCCTGGCACGATATTACGCACAACTAATGGTGACTTTTT | |
| | GCATTTCTTACCTAGAGTTTTTAGTGCAGTTGGTAACATCTGTTACACACCATCA | |
| | AAACTTATAGAGTACACTGACTTTGCAACATCAGCTTGTGTTTTGGCTGCTGAAT | |
| | GTACAATTTTTAAAGATGCTTCTGGTAAGCCAGTACCATATTGTTATGATACCAA | |
| | TGTACTAGAAGGTTCTGTTGCTTATGAAAGTTTACGCCCTGACACACGTTATGTG | |
| | CTCATGGATGGCTCTATTATTCAATTTCCTAACACCTACCTTGAAGGTTCTGTTA | |
| | GAGTGGTAACAACCTTTGATTCTGAGTACTGTAGGCACGGCACTTGTGAAAGATC | |
| | AGAAGCTGGTGTTTGTGTATCTACTAGTGGTAGATGGGTACTTAACAATGATTAT | |
| | TACAGATCTTTACCAGGAGTTTTCTGTGGTGTAGATGCTGTAAATTTACTTACTA | |
| | ATATGTTTACACCACTAATTCAACCTATTGGTGCTTTGGACATATCAGCATCTAT | |
| | AGTAGCTGGTGGTATTGTAGCTATCGTAGTAACATGCCTTGCCTACTATTTTATG | |
| | AGGTTTAGgAGAGCTTTTGGTGAATACAGTCATGTAGTTGCCTTTAATACTTTAC | |
| | TATTCCTTATGTCATTCACTGTACTCTGTTTAACACCAGTTTACTCATTCTTACC | |
| | TGGTGTTTATTCTGTTATTTACTTGTACTTGACATTTTATCTTACTAATGATGTT | |
| | TCTTTTTTAGCACATATTCAGTGGATGGTTATGTTCACACCTTTAGTACCTTTCT | |
| | GGATAACAATTGCTTATATCATTTGTATTTCCACAAAGCATTTCTATTGGTTCTT | |
| | TAGTAATTACCTAAAGAGACGTGTAGTCTTTAATGGTGTTTCCTTTAGTACTTTT | |
| | GAAGAAGCTGCGCTGTGCACCTTTTTGTTAAATAAAGAAATGTATCTAAAGTTGC | |
| | GTAGTGATGTGCTATTACCTCTTACGCAATATAATAGATACTTAGCTCTTTATAA | |
| | TAAGTACAAGTATTTTAGTGGAGCAATGGATACAACTAGCTACAGAGAAGCTGCT | |
| | TGTTGTCATCTCGCAAAGGCTCTCAATGACTTCAGTAACTCAGGTTCTGATGTTC | |
| | TTTACCAACCACCACAAACCTCTATCACCTCAGCTGTTTTGCAGAGTGGTTTTAG | |
| | AAAAATGGCATTCCCATCTGGTAAAGTTGAGGGTTGTATGGTACAAGTAACTTGT | |
| | GGTACAACTACACTTAACGGTCTTTGGCTTGATGACGTAGTTTACTGTCCAAGAC | |
| | ATGTGATCTGCACCTCTGAAGACATGCTTAACCCTAATTATGAAGATTTACTCAT | |
| | TCGTAAGTCTAATCATAATTTCTTGGTACAGGCTGGTAATGTTCAACTCAGGGTT | |
| | ATTGGACATTCTATGCAAAATTGTGTACTTAAGCTTAAGGTTGATACAGCCAATC | |
| | CTAAGACACCTAAGTATAAGTTTGTTCGCATTCAACCAGGACAGACTTTTTCAGT | |
| | GTTAGCTTGTTACAATGGTTCACCATCTGGTGTTTACCAATGTGCTATGAGGCCC | |
| | AATTTCACTATTAAGGGTTCATTCCTTAATGGTTCATGTGGTAGTGTTGGTTTTA | |
| | ACATAGATTATGACTGTGTCTCTTTTTGTTACATGCACCATATGGAATTACCAAC | |
| | TGGAGTTCATGCTGGCACAGACTTAGAAGGTAACTTTTATGGACCTTTTGTTGAC | |
| | AGGCAAACAGCACAAGCAGCTGGTACGGACACAACTATTACAGTTAATGTTTTAG | |
| | CTTGGTTGTACGCTGCTGTTATAAATGGAGACAGGTGGTTTCTCAATCGATTTAC | |
| | CACAACTCTTAATGACTTTAACCTTGTGGCTATGAAGTACAATTATGAACCTCTA | |
| | ACACAAGACCATGTTGACATACTAGGACCTCTTTCTGCTCAAACTGGAATTGCCG | |
| | TTTTAGATATGTGTGCTTCATTAAAAGAATTACTGCAAAATGGTATGAATGGACG | |
| | TACCATATTGGGTAGTGCTTTATTAGAAGATGAATTTACACCTTTTGATGTTGTT | |
| | AGACAATGCTCAGGTGTTACTTTCCAAAGTGCAGTGAAAAGAACAATCAAGGGTA | |
| | CACACCACTGGTTGTTACTCACAATTTTGACTTCACTTTTAGTTTTAGTCCAGAG | |
| | TACTCAATGGTCTTTGTTCTTTTTTTTGTATGAAAATGCCTTTTTACCTTTTGCT | |
| | ATGGGTATTATTGCTATGTCTGCTTTTGCAATGATGTTTGTCAAACATAAGCATG | |
| | CATTTCTCTGTTTGTTTTTGTTACCTTCTCTTGCCACTGTAGCTTATTTTAATAT | |
| | GGTCTATATGCCTGCTAGTTGGGTGATGCGTATTATGACATGGTTGGATATGGTT | |
| | GATACTAGTTTGTCTGGTTTTAAGCTAAAAGACTGTGTTATGTATGCATCAGCTG | |
| | TAGTGTTACTAATCCTTATGACAGCAAGAACTGTGTATGATGATGGTGCTAGGAG | |
| | AGTGTGGACACTTATGAATGTCTTGACACTCGTTTATAAAGTTTATTATGGTAAT | |
| | GCTTTAGATCAAGCCATTTCCATGTGGGCTCTTATAATCTCTGTTACTTCTAACT | |
| | ACTCAGGTGTAGTTACAACTGTCATGTTCTTGGCCAGAGGTATTGTTTTTATGTG | |
| | TGTTGAGTATTGCCCTATTTTCTTCATAACTGGTAATACACTTCAGTGTATAATG | |
| | CTAGTTTATTGTTTCTTAGGCTATTTTTGTACTTGTTACTTTGGCCTCTTTTGTT | |
| | TACTCAACCGCTACTTTAGACTGACTCTTGGTGTTTATGATTACTTAGTTTCTAC | |
| | ACAGGAGTTTAGATATATGAATTCACAGGGACTACTCCCACCCAAGAATAGCATA | |
| | GATGCCTTCAAACTCAACATTAAATTGTTGGGTGTTGGTGGCAAACCTTGTATCA | |
| | AAGTAGCCACTGTACAGTCTAAAATGTCAGATGTAAAGTGCACATCAGTAGTCTT | |
| | ACTCTCAGTTTTGCAACAACTCAGAGTAGAATCATCATCTAAATTGTGGGCTCAA | |
| | TGTGTCCAGTTACACAATGACATTCTCTTAGCTAAAGATACTACTGAAGCCTTTG | |
| | AAAAAATGGTTTCACTACTTTCTGTTTTGCTTTCCATGCAGGGTGCTGTAGACAT | |
| | AAACAAGCTTTGTGAAGAAATGCTGGACAACAGGGCAACCTTACAAGCTATAGCC | |
| | TCAGAGTTTAGTTCCCTTCCATCATATGCAGCTTTTGCTACTGCTCAAGAAGCTT | |
| | ATGAGCAGGCTGTTGCTAATGGTGATTCTGAAGTTGTTCTTAAAAAGTTGAAGAA | |
| | GTCTTTGAATGTGGCTAAATCTGAATTTGACCGTGATGCAGCCATGCAACGTAAG | |
| | TTGGAAAAGATGGCTGATCAAGCTATGACCCAAATGTATAAACAGGCTAGATCTG | |
| | AGGACAAGAGGGCAAAAGTTACTAGTGCTATGCAGACAATGCTTTTCACTATGCT | |
| | TAGAAAGTTGGATAATGATGCACTCAACAACATTATCAACAATGCAAGAGATGGT | |
| | TGTGTTCCCTTGAACATAATACCTCTTACAACAGCAGCCAAACTAATGGTTGTCA | |
| | TACCAGACTATAACACATATAAAAATACGTGTGATGGTACAACATTTACTTATGC | |
| | ATCAGCATTGTGGGAAATCCAACAGGTTGTAGATGCAGATAGTAAAATTGTTCAA | |
| | CTTAGTGAAATTAGTATGGACAATTCACCTAATTTAGCATGGCCTCTTATTGTAA | |
| | CAGCTTTAAGGGCCAATTCTGCTGTCAAATTACAGAATAATGAGCTTAGTCCTGT | |
| | TGCACTACGACAGATGTCTTGTGCTGCCGGTACTACACAAACTGCTTGCACTGAT | |
| | GACAATGCGTTAGCTTACTACAACACAACAAAGGGAGGTAGGTTTGTACTTGCAC | |
| | TGTTATCCGATTTACAGGATTTGAAATGGGCTAGATTCCCTAAGAGTGATGGAAC | |
| | TGGTACTATCTATACAGAACTGGAACCACCTTGTAGGTTTGTTACAGACACACCT | |
| | AAAGGTCCTAAAGTGAAGTATTTATACTTTATTAAAGGATTAAACAACCTAAATA | |
| | GAGGTATGGTACTTGGTAGTTTAGCTGCCACAGTACGTCTACAAGCTGGTAATGC | |
| | AACAGAAGTGCCTGCCAATTCAACTGTATTATCTTTCTGTGCTTTTGCTGTAGAT | |
| | GCTGCTAAAGCTTACAAAGATTATCTAGCTAGTGGGGGACAACCAATCACTAATT | |
| | GTGTTAAGATGTTGTGTACACACACTGGTACTGGTCAGGCAATAACAGTTACACC | |
| | GGAAGCCAATATGGATCAAGAATCCTTTGGTGGTGCATCGTGTTGTCTGTACTGC | |
| | CGTTGCCACATAGATCATCCAAATCCTAAAGGATTTTGTGACTTAAAAGGTAAGT | |
| | ATGTACAAATACCTACAACTTGTGCTAATGACCCTGTGGGTTTTACACTTAAAAA | |
| | CACAGTCTGTACCGTCTGCGGTATGTGGAAAGGTTATGGCTGTAGTTGTGATCAA | |
| | CTCCGCGAACCCATGCTTCAGTCAGCTGATGCACAATCGTTTTTAAACGGGTTTG | |
| | CGGTGTAAGTGCAGCCCGTCTTACACCGTGCGGCACAGGCACTAGTACTGATGTC | |
| | GTATACAGGGCTTTTGACATCTACAATGATAAAGTAGCTGGTTTTGCTAAATTCC | |
| | TAAAAACTAATTGTTGTCGCTTCCAAGAAAAGGACGAAGATGACAATTTAATTGA | |
| | TTCTTACTTTGTAGTTAAGAGACACACTTTCTCTAACTACCAACATGAAGAAACA | |
| | ATTTATAATTTACTTAAGGATTGTCCAGCTGTTGCTAAACATGACTTCTTTAAGT | |
| | TTAGAATAGACGGTGACATGGTACCACATATATCACGTCAACGTCTTACTAAATA | |
| | CACAATGGCAGACCTCGTCTATGCTTTAAGGCATTTTGATGAAGGTAATTGTGAC | |
| | ACATTAAAAGAAATACTTGTCACATACAATTGTTGTGATGATGATTATTTCAATA | |
| | AAAAGGACTGGTATGATTTTGTAGAAAACCCAGATATATTACGCGTATACGCCAA | |
| | CTTAGGTGAACGTGTACGCCAAGCTTTGTTAAAAACAGTACAATTCTGTGATGCC | |
| | ATGCGAAATGCTGGTATTGTTGGTGTACTGACATTAGATAATCAAGATCTCAATG | |
| | GTAACTGGTATGATTTCGGTGATTTCATACAAACCACGCCAGGTAGTGGAGTTCC | |
| | TGTTGTAGATTCTTATTATTCATTGTTAATGCCTATATTAACCTTGACCAGGGCT | |
| | TTAACTGCAGAGTCACATGTTGACACTGACTTAACAAAGCCTTACATTAAGTGGG | |
| | ATTTGTTAAAATATGACTTCACGGAAGAGAGGTTAAAACTCTTTGACCGTTATTT | |
| | TAAATATTGGGATCAGACATACCACCCAAATTGTGTTAACTGTTTGGATGACAGA | |
| | TGCATTCTGCATTGTGCAAACTTTAATGTTTTATTCTCTACAGTGTTCCCACCTA | |
| | CAAGTTTTGGACCACTAGTGAGAAAAATATTTGTTGATGGTGTTCCATTTGTAGT | |
| | TTCAACTGGATACCACTTCAGAGAGCTAGGTGTTGTACATAATCAGGATGTAAAC | |
| | TTACATAGCTCTAGACTTAGTTTTAAGGAATTACTTGTGTATGCTGCTGACCCTG | |
| | CTATGCACGCTGCTTCTGGTAATCTATTACTAGATAAACGCACTACGTGCTTTTC | |
| | AGTAGCTGCACTTACTAACAATGTTGCTTTTCAAACTGTCAAACCCGGTAATTTT | |
| | AACAAAGACTTCTATGACTTTGCTGTGTCTAAGGGTTTCTTTAAGGAAGGAAGTT | |
| | CTGTTGAATTAAAACACTTCTTCTTTGCTCAGGATGGTAATGCTGCTATCAGCGA | |
| | TTATGACTACTATCGTTATAATCTACCAACAATGTGTGATATCAGACAACTACTA | |
| | TTTGTAGTTGAAGTTGTTGATAAGTACTTTGATTGTTACGATGGTGGCTGTATTA | |
| | ATGCTAACCAAGTCATCGTCAACAACCTAGACAAATCAGCTGGTTTTCCATTTAA | |
| | TAAATGGGGTAAGGCTAGACTTTATTATGATTCAATGAGTTATGAGGATCAAGAT | |
| | GCACTTTTCGCATATACAAAACGTAATGTCATCCCTACTATAACTCAAATGAATC | |
| | TTAAGTATGCCATTAGTGCAAAGAATAGAGCTCGCACCGTAGCTGGTGTCTCTAT | |
| | CTGTAGTACTATGACCAATAGACAGTTTCATCAAAAATTATTGAAATCAATAGCC | |
| | GCCACTAGAGGAGCTACTGTAGTAATTGGAACAAGCAAATTCTATGGTGGTTGGC | |
| | ACAACATGTTAAAAACTGTTTATAGTGATGTAGAAAACCCTCACCTTATGGGTTG | |
| | GGATTATCCTAAATGTGATAGAGCCATGCCTAACATGCTTAGAATTATGGCCTCA | |
| | CTTGTTCTTGCTCGCAAACATACAACGTGTTGTAGCTTGTCACACCGTTTCTATA | |
| | GATTAGCTAATGAGTGTGCTCAAGTATTGAGTGAAATGGTCATGTGTGGCGGTTC | |
| | ACTATATGTTAAACCAGGTGGAACCTCATCAGGAGATGCCACAACTGCTTATGCT | |
| | AATAGTGTTTTTAACATTTGTCAAGCTGTCACGGCCAATGTTAATGCACTTTTAT | |
| | CTACTGATGGTAACAAAATTGCCGATAAGTATGTCCGCAATTTACAACACAGACT | |
| | TTATGAGTGTCTCTATAGAAATAGAGATGTTGACACAGACTTTGTGAATGAGTTT | |
| | TACGCATATTTGCGTAAACATTTCTCAATGATGATACTCTCTGACGATGCTGTTG | |
| | TGTGTTTCAATAGCACTTATGCATCTCAAGGTCTAGTGGCTAGCATAAAGAACTT | |
| | TAAGTCAGTTCTTTATTATCAAAACAATGTTTTTATGTCTGAAGCAAAATGTTGG | |
| | ACTGAGACTGACCTTACTAAAGGACCTCATGAATTTTGCTCTCAACATACAATGC | |
| | TAGTTAAACAGGGTGATGATTATGTGTACCTTCCTTACCCAGATCCATCAAGAAT | |
| | CCTAGGGGCCGGCTGTTTTGTAGATGATATCGTAAAAACAGATGGTACACTTATG | |
| | ATTGAACGGTTCGTGTCTTTAGCTATAGATGCTTACCCACTTACTAAACATCCTA | |
| | ATCAGGAGTATGCTGATGTCTTTCATTTGTACTTACAATACATAAGAAAGCTACA | |
| | TGATGAGTTAACAGGACACATGTTAGACATGTATTCTGTTATGCTTACTAATGAT | |
| | AACACTTCAAGGTATTGGGAACCTGAGTTTTATGAGGCTATGTACACACCGCATA | |
| | CAGTCTTACAGGCTGTTGGGGCTTGTGTTCTTTGCAATTCACAGACTTCATTAAG | |
| | ATGTGGTGCTTGCATACGTAGACCATTCTTATGTTGTAAATGCTGTTACGACCAT | |
| | GTCATATCAACATCACATAAATTAGTCTTGTCTGTTAATCCGTATGTTTGCAGTG | |
| | CTCCAGGTTGTGATGTCACAGATGTGACTCAACTTTACTTAGGAGGTATGAGCTA | |
| | TTATTGTAAATCACATAAACCACCCATTAGTTTTCCATTGTGTGCTAATGGACAA | |
| | GTTTTTGGTTTATATAAAAATACATGTGTTGGTAGCGATAATGTTACTGACTTTA | |
| | ATGCAATTGCAACATGTGACTGGACAAATGCTGGTGATTACATTTTAGCTAACAC | |
| | CTGTACTGAAAGACTCAAGCTTTTTGCAGCAGAAACGCTCAAAGCTACTGAGGAG | |
| | ACATTTAAACTGTCTTATGGTATTGCTACTGTACGTGAAGTGCTGTCTGACAGAG | |
| | AATTACATCTTTCATGGGAAGTTGGTAAACCTAGACCACCACTTAACCGAAATTA | |
| | TGTCTTTACTGGTTATCGTGTAACTAAAAACAGTAAAGTACAAATAGGAGAGTAC | |
| | ACCTTTGAAAAAGGTGACTATGGTGATGCTGTTGTTTACCGAGGTACAACAACTT | |
| | ACAAATTAAATGTTGGTGATTATTTTGTGCTGACATCACATACAGTAATGCCATT | |
| | AAGTGCACCTACACTAGTGCCACAAGAGCACTATGTTAGAATTACTGGCTTATAC | |
| | CCAACACTCAATATCTCAGATGAGTTTTCTAGCAATGTTGCAAATTATCAAAAGG | |
| | TTGGTATGCAAAAGTATTCTACACTCCAGGGACCACCTGGTACTGGTAAGAGTCA | |
| | TTTTGCTATTGGCCTAGCTCTCTACTACCCTTCTGCTCGCATAGTGTATACAGCT | |
| | TGCTCTCATGCCGCTGTTGATGCACTATGTGAGAAGGCATTAAAATATTTGCCTA | |
| | TAGATAAATGTAGTAGAATTATACCTGCACGTGCTCGTGTAGAGTGTTTTGATAA | |
| | ATTCAAAGTGAATTCAACATTAGAACAGTATGTCTTTTGTACTGTAAATGCATTG | |
| | CCTGAGACGACAGCAGATATAGTTGTCTTTGATGAAATTTCAATGGCCACAAATT | |
| | ATGATTTGAGTGTTGTCAATGCCAGATTACGTGCTAAGCACTATGTGTACATTGG | |
| | CGACCCTGCTCAATTACCTGCACCACGCACATTGCTAACTAAGGGCACACTAGAA | |
| | CCAGAATATTTCAATTCAGTGTGTAGACTTATGAAAACTATAGGTCCAGACATGT | |
| | TCCTCGGAACTTGTCGGCGTTGTCCTGCTGAAATTGTTGACACTGTGAGTGCTTT | |
| | GGTTTATGATAATAAGCTTAAAGCACATAAAGACAAATCAGCTCAATGCTTTAAA | |
| | ATGTTTTATAAGGGTGTTATCACGCATGATGTTTCATCTGCAATTAACAGGCCAC | |
| | AAATAGGCGTGGTAAGAGAATTCCTTACACGTAACCCTGCTTGGAGAAAAGCTGT | |
| | CTTTATTTCACCTTATAATTCACAGAATGCTGTAGCCTCAAAGATTTTGGGACTA | |
| | CCAACTCAAACTGTTGATTCATCACAGGGCTCAGAATATGACTATGTCATATTCA | |
| | CTCAAACCACTGAAACAGCTCACTCTTGTAATGTAAACAGATTTAATGTTGCTAT | |
| | TACCAGAGCAAAAGTAGGCATACTTTGCATAATGTCTGATAGAGACCTTTATGAC | |
| | AAGTTGCAATTTACAAGTCTTGAAATTCCACGTAGGAATGTGGCAACTTTACAAG | |
| | CTGAAAATGTAACAGGACTTTTTAAAGATTGTAGTAAGGTAATCACTGGGTTACA | |
| | TCCTACACAGGCACCTACACACCTCAGTGTTGACACTAAATTCAAAACTGAAGGT | |
| | TTATGTGTTGACATACCTGGCATACCTAAGGACATGACCTATAGAAGACTCATCT | |
| | CTATGATGGGTTTTAAAATGAATTATCAAGTTAATGGTTACCCTAACATGTTTAT | |
| | CACCCGCGAAGAAGCTATAAGACATGTACGTGCATGGATTGGCTTCGATGTCGAG | |
| | GGGTGTCATGCTACTAGAGAAGCTGTTGGTACCAATTTACCTTTACAGCTAGGTT | |
| | TTTCTACAGGTGTTAACCTAGTTGCTGTACCTACAGGTTATGTTGATACACCTAA | |
| | TAATACAGATTTTTCCAGAGTTAGTGCTAAACCACCGCCTGGAGATCAATTTAAA | |
| | CACCTCATACCACTTATGTACAAAGGACTTCCTTGGAATGTAGTGCGTATAAAGA | |
| | TTGTACAAATGTTAAGTGACACACTTAAAAATCTCTCTGACAGAGTCGTATTTGT | |
| | CTTATGGGCACATGGCTTTGAGTTGACATCTATGAAGTATTTTGTGAAAATAGGA | |
| | CCTGAGCGCACCTGTTGTCTATGTGATAGACGTGCCACATGCTTTTCCACTGCTT | |
| | CAGACACTTATGCCTGTTGGCATCATTCTATTGGATTTGATTACGTCTATAATCC | |
| | GTTTATGATTGATGTTCAACAATGGGGTTTTACAGGTAACCTACAAAGCAACCAT | |
| | GATCTGTATTGTCAAGTCCATGGTAATGCACATGTAGCTAGTTGTGATGCAATCA | |
| | TGACTAGGTGTCTAGCTGTCCACGAGTGCTTTGTTAAGCGTGTTGACTGGACTAT | |
| | TGAATATCCTATAATTGGTGATGAACTGAAGATTAATGCGGCTTGTAGAAAGGTT | |
| | CAACACATGGTTGTTAAAGCTGCATTATTAGCAGACAAATTCCCAGTTCTTCACG | |
| | ACATTGGTAACCCTAAAGCTATTAAGTGTGTACCTCAAGCTGATGTAGAATGGAA | |
| | GTTCTATGATGCACAGCCTTGTAGTGACAAAGCTTATAAAATAGAAGAATTATTC | |
| | TATTCTTATGCCACACATTCTGACAAATTCACAGATGGTGTATGCCTATTTTGGA | |
| | ATTGCAATGTCGATAGATATCCTGCTAATTCCATTGTTTGTAGATTTGACACTAG | |
| | AGTGCTATCTAACCTTAACTTGCCTGGTTGTGATGGTGGCAGTTTGTATGTAAAT | |
| | AAACATGCATTCCACACACCAGCTTTTGATAAAAGTGCTTTTGTTAATTTAAAAC | |
| | AATTACCATTTTTCTATTACTCTGACAGTCCATGTGAGTCTCATGGAAAACAAGT | |
| | AGTGTCAGATATAGATTATGTACCACTAAAGTCTGCTACGTGTATAACACGTTGC | |
| | AATTTAGGTGGTGCTGTCTGTAGACATCATGCTAATGAGTACAGATTGTATCTCG | |
| | ATGCTTATAACATGATGATCTCAGCTGGCTTTAGCTTGTGGGTTTACAAACAATT | |
| | TGATACTTATAACCTCTGGAACACTTTTACAAGACTTCAGAGTTTAGAAAATGTG | |
| | GCTTTTAATGTTGTAAATAAGGGACACTTTGATGGACAACAGGGTGAAGTACCAG | |
| | TTTCTATCATTAATAACACTGTTTACACAAAAGTTGATGGTGTTGATGTAGAATT | |
| | GTTTGAAAATAAAACAACATTACCTGTTAATGTAGCATTTGAGCTTTGGGCTAAG | |
| | CGCAACATTAAACCAGTACCAGAGGTGAAAATACTCAATAATTTGGGTGTGGACA | |
| | TTGCTGCTAATACTGTGATCTGGGACTACAAAAGAGATGCTCCAGCACATATATC | |
| | TACTATTGGTGTTTGTTCTATGACTGACATAGCCAAGAAACCAACTGAAACGATT | |
| | TGTGCACCACTCACTGTCTTTTTTGATGGTAGAGTTGATGGTCAAGTAGACTTAT | |
| | TTAGAAATGCCCGTAATGGTGTTCTTATTACAGAAGGTAGTGTTAAAGGTTTACA | |
| | ACCATCTGTAGGTCCCAAACAAGCTAGTCTTAATGGAGTCACATTAATTGGAGAA | |
| | GCCGTAAAAACACAGTTCAATTATTATAAGAAAGTTGATGGTGTTGTCCAACAAT | |
| | TACCTGAAACTTACTTTACTCAGAGTAGAAATTTACAAGAATTTAAACCCAGGAG | |
| | TCAAATGGAAATTGATTTCTTAGAATTAGCTATGGATGAATTCATTGAACGGTAT | |
| | AAATTAGAAGGCTATGCCTTCGAACATATCGTTTATGGAGATTTTAGTCATAGTC | |
| | AGTTAGGTGGTTTACATCTACTGATTGGACTAGCTAAACGTTTTAAGGAATCACC | |
| | TTTTGAATTAGAAGATTTTATTCCTATGGACAGTACAGTTAAAAACTATTTCATA | |
| | ACAGATGCGCAAACAGGTTCATCTAAGTGTGTGTGTTCTGTTATTGATTTATTAC | |
| | TTGATGATTTTGTTGAAATAATAAAATCCCAAGATTTATCTGTAGTTTCTAAGGT | |
| | TGTCAAAGTGACTATTGACTATACAGAAATTTCATTTATGCTTTGGTGTAAAGAT | |
| | GGCCATGTAGAAACATTTTACCCAAAATTACAATCTAGTCAAGCGTGGCAACCGG | |
| | GTGTTGCTATGCCTAATCTTTACAAAATGCAAAGAATGCTATTAGAAAAGTGTGA | |
| | CCTTCAAAATTATGGTGATAGTGCAACATTACCTAAAGGCATAATGATGAATGTC | |
| | GCAAAATATACTCAACTGTGTCAATATTTAAACACATTAACATTAGCTGTACCCT | |
| | ATAATATGAGAGTTATACATTTTGGTGCTGGTTCTGATAAAGGAGTTGCACCAGG | |
| | TACAGCTGTTTTAAGACAGTGGTTGCCTACGGGTACGCTGCTTGTCGATTCAGAT | |
| | CTTAATGACTTTGTCTCTGATGCAGATTCAACTTTGATTGGTGATTGTGCAACTG | |
| | TACATACAGCTAATAAATGGGATCTCATTATTAGTGATATGTACGACCCTAAGAC | |
| | TAAAAATGTTACAAAAGAAAATGACTCTAAAGAGGGTTTTTTCACTTACATTTGT | |
| | GGGTTTATACAACAAAAGCTAGCTCTTGGAGGTTCCGTGGCTATAAAGATAACAG | |
| | AACATTCTTGGAATGCTGATCTTTATAAGCTCATGGGACACTTCGCATGGTGGAC | |
| | AGCCTTTGTTACTAATGTGAATGCGTCATCATCTGAAGCATTTTTAATTGGATGT | |
| | AATTATCTTGGCAAACCACGCGAACAAATAGATGGTTATGTCATGCATGCAAATT | |
| | ACATATTTTGGAGGAATACAAATCCAATTCAGTTGTCTTCCTATTCTTTATTTGA | |
| | CATGAGTAAATTTCCCCTTAAATTAAGGGGTACTGCTGTTATGTCTTTAAAAGAA | |
| | GGTCAAATCAATGATATGATTTTATCTCTTCTTAGTAAAGGTAGACTTATAATTA | |
| | GAGAAAACAACAGAGTTGTTATTTCTAGTGATGTTCTTGTTAACAACTAAACGAA | |
| | CAatgtttgtttttcttgttttattgccactagtctctagtcagtgtgttaatct | |
| | tataaccagaactcaatcatacactaattctttcacacgtggtgtttattaccct | |
| | gacaaagttttcagatcctcagttttacattcaactcaggacttgttcttacctt | |
| | tcttttccaatgttacttggttccatgctatctctgggaccaatggtactaagag | |
| | gtttgataaccctgtcctaccatttaatgatggtgtttattttgcttccactgag | |
| | aagtctaacataataagaggctggatttttggtactactttagattcgaagaccc | |
| | agtccctacttattgttaataacgctactaatgttgttattaaagtctgtgaatt | |
| | tcaattttgtaatgatccatttttggatgtttattaccacaaaaacaacaaaagt | |
| | tggatggaaagtgagttcagagtttattctagtgcgaataattgcacttttgaat | |
| | atgtctctcagccttttcttatggaccttgaaggaaaacagggtaatttcaaaaa | |
| | tcttagggaatttgtgtttaagaatattgatggttattttaaaatatattctaag | |
| | cacacgcctattaatttagggcgtgatctccctcagggtttttcggctttagaac | |
| | cattggtagatttgccaataggtattaacatcactaggtttcaaactttacttgc | |
| | tttacatagaagttatttgactcctggtgattcttcttcaggttggacagctggt | |
| | gctgcagcttattatgtgggttatcttcaacctaggacttttctattaaaatata | |
| | atgaaaatggaaccattacagatgctgtagactgtgcacttgaccctctctcaga | |
| | aacaaagtgtacgttgaaatccttcactgtagaaaaaggaatctatcaaacttct | |
| | aactttagagtccaaccaacagaatctattgttagatttcctaatattacaaact | |
| | tgtgcccttttgatgaagtttttaacgccaccagatttgcatctgtttatgcttg | |
| | gaacaggaagagaatcagcaactgtgttgctgattattctgtcctatataatttc | |
| | gcaccatttttcgcttttaagtgttatggagtgtctcctactaaattaaatgatc | |
| | tctgctttactaatgtctatgcagattcatttgtaattagaggtaatgaagtcag | |
| | ccaaatcgctccagggcaaactggaaatattgctgattataattataaattacca | |
| | gatgattttacaggctgcgttatagcttggaattctaacaagcttgattctaagg | |
| | ttggtggtaattataattaccggtatagattgtttaggaagtctaatctcaaacc | |
| | ttttgagagagatatttcaactgaaatctatcaggccggtaacaaaccttgtaat | |
| | ggtgttgcaggtgttaattgttactttcctttacaatcatatggtttccgaccca | |
| | cttatggtgttggtcaccaaccatacagagtagtagtactttcttttgaacttct | |
| | acatgcaccagcaactgtttgtggacctaaaaagtctactaatttggttaaaaac | |
| | aaatgtgtcaatttcaacttcaatggtttaacaggcacaggtgttcttactgagt | |
| | ctaacaaaaagtttctgcctttccaacaatttggcagagacattgctgacactac | |
| | tgatgctgtccgtgatccacagacacttgagattcttgacattacaccatgttct | |
| | tttggtggtgtcagtgttataacaccaggaacaaatacttctaaccaggttgctg | |
| | ttctttatcagggtgttaactgcacagaagtccctgttgctattcatgcagatca | |
| | acttactcctacttggcgtgtttattctacaggttctaatgtttttcaaacacgt | |
| | gcaggctgtttaataggggctgaatatgtcaacaactcatatgagtgtgacatac | |
| | ccattggtgcaggtatatgcgctagttatcagactcagcaatccatcattgccta | |
| | cactatgtcacttggtgcagaaaattcagttgcttactctaataactctattgcc | |
| | atacccacaaattttactattagtgttaccacagaaattctaccagtgtctatga | |
| | ccaagacatcagtagattgtacaatgtacatttgtggtgattcaactgaatgcag | |
| | caatcttttgttgcaatatggcagtttttgtacacaattaaaacgtgctttaact | |
| | ggaatagctgttgaacaagacaaaaacacccaagaagtttttgcacaagtcaaac | |
| | aaatttacaaaacaccaccaattaaatattttggtggttttaatttttcacaaat | |
| | attaccagatccatcaaaaccaagcaagaggtcatttattgaagatctacttttc | |
| | aacaaagtgacacttgcagatgctggcttcatcaaacaatatggtgattgccttg | |
| | gtgatattgctgctagagacctcatttgcgctcaaaaatttaacggacttacagt | |
| | tttaccacctttacttactgacgaaatgattgcgcaatatacatccgcattgtta | |
| | gccggaactattacatccggatggacttttggcgcaggcgcagcattacagattc | |
| | cattcgctatgcaaatggcttataggtttaacggtataggcgttacgcaaaacgt | |
| | actttatgagaatcaaaaacttatcgctaaccaatttaattccgctatcggtaag | |
| | attcaggattcattgtctagtactgctagtgcactcggtaagttgcaagacgtag | |
| | tgaatcaaaacgctcaagcacttaatacactcgttaaacagcttagttctaattt | |
| | tggcgcaatttctagtgtgcttaacgatatactatctagactcgataaagtcgaa | |
| | gccgaagtgcaaatcgatagattgattaccggtaggttgcaatcattgcaaacat | |
| | acgttacacagcaattgattagggccgcagagatacgcgctagcgctaatctcgc | |
| | agctactaaaatgtctgaatgcgtactcggacaatctaaacgtgtcgatttttgc | |
| | ggtaagggatatcatcttatgtcttttccacaatctgcacctcacggagtcgtgt | |
| | ttttacacgttacttatgtgccagctcaagagaaaaattttacaaccgctcctgc | |
| | tatttgtcatgacggtaaggcacattttcctagagagggcgtattcgtttctaac | |
| | ggtacacattggttcgttacacaacgtaatttttacgaacctcaaattattacta | |
| | ctgataatacattcgtatcaggtaattgtgacgtagtgataggtatcgttaataa | |
| | tacagtttacgatccacttcaacctgaactcgatagttttaaagaggaactcgat | |
| | aagtattttaaaaatcatacatcacctgacgtcgacttaggcgatatttcaggta | |
| | ttaacgctagtgtcgttaacattcaaaaagagattgatagacttaacgaagtcgc | |
| | taaaaatcttaacgaatcacttatcgatctgcaagagttaggtaagtatgagcaa | |
| | tatattaaatggccttggtatatttggttaggctttatagccggattgatcgcaa | |
| | tcgttatggttacaattatgttatgttgtatgacatcatgttgttcatgtcttaa | |
| | gggatgttgttcatgcggatcatgttgtaaatttgacgaagacgattccgaacca | |
| | gtgcttaaaggcgttaagttacattatacataaACGAACTTATGGATTTGTTTAT | |
| | GAGAATCTTCACAATTGGAACTGTAACTTTGAAGCAAGGTGAAATCAAGGATGCT | |
| | ACTCCTTCAGATTTTGTTCGCGCTACTGCAACGATACCGATACAAGCCTCACTCC | |
| | CTTTCGGATGGCTTATTGTTGGCGTTGCACTTCTTGCTGTTTTTCAGAGCGCTTC | |
| | CAAAATCATAACCCTCAAAAAGAGATGGCAACTAGCACTCTCCAAGGGTGTTCAC | |
| | TTTGTTTGCAACTTGCTGTTGTTGTTTGTAACAGTTTACTCACACCTTTTGCTCG | |
| | TTGCTGCTGGCCTTGAAGCCCCTTTTCTCTATCTTTATGCTTTAGTCTACTTCTT | |
| | GCAGAGTATAAACTTTGTAAGAATAATAATGAGGCTTTGGCTTTGCTGGAAATGC | |
| | CGTTCCAAAAACCCATTACTTTATGATGCCAACTATTTTCTTTGCTGGCATACTA | |
| | ATTGTTACGACTATTGTATACCTTACAATAGTGTAACTTCTTCAATTGTCATTAC | |
| | TTCAGGTGATGGCACAACAAGTCCTATTTCTGAACATGACTACCAGATTGGTGGT | |
| | TATACTGAAAAATGGGAATCTGGAGTAAAAGACTGTGTTGTATTACACAGTTACT | |
| | TCACTTCAGACTATTACCAGCTGTACTCAACTCAATTGAGTACAGACACTGGTGT | |
| | TGAACATGTTACCTTCTTCATCTACAATAAAATTGTTGATGAGCCTGAAGAACAT | |
| | GTCCAAATTCACACAATCGACGGTTCATCCGGAGTTGTTAATCCAGTAATGGAAC | |
| | CAATTTATGATGAACCGACGACGACTACTAGCGTGCCTTTGTAAGCACAAGCTGA | |
| | TGAGTACGAACTTATGTACTCATTCGGTTCGGAAGAGACAGGTACGTTAATAGTT | |
| | AATAGCGTACTTCTTTTTCTTGCTTTCGTGGTATTCTTGCTAGTTACACTAGCCA | |
| | TCCTTACTGCGCTTCGATTGTGTGCGTACTGCTGCAATATTGTTAACGTGAGTCT | |
| | TGTAAAACCTTCTTTTTACGTTTACTCTCGTGTTAAAAATCTGAATTCTTCTAGA | |
| | GTTCCTGATCTTCTGGTCTAAACGAACTAAATATTATATTAGTTTTTCTGTTTGG | |
| | AACTTTAATTTTAGCCATGGCAGATTCCAACGGTACTATTACCGTTGAAGAGCTT | |
| | AAAAAGCTCCTTGAACAATGGAACCTAGTAATAGGTTTCCTATTCCTTACATGGA | |
| | TTTGTCTTCTACAATTTGCCTATGCCAACAGGAATAGGTTTTTGTATATAATTAA | |
| | GTTAATTTTCCTCTGGCTGTTATGGCCAGTAACTTTAGCTTGTTTTGTGCTTGCT | |
| | GCTGTTTACAGAATAAATTGGATCACCGGTGGAATTGCTATCGCAATGGCTTGTC | |
| | TTGTAGGCTTGATGTGGCTCAGCTACTTCATTGCTTCTTTCAGACTGTTTGCGCG | |
| | TACGCGTTCCATGTGGTCATTCAATCCAGAAACTAACATTCTTCTCAACGTGCCA | |
| | CTCCATGGCACTATTCTGACCAGACCGCTTCTAGAAAGTGAACTCGTAATCGGAG | |
| | CTGTGATCCTTCGTGGACATCTTCGTATTGCTGGACACCATCTAGGACGCTGTGA | |
| | CATCAAGGACCTGCCTAAAGAAATCACTGTTGCTACATCACGAACGCTTTCTTAT | |
| | TACAAATTGGGAGCTTCGCAGCGTGTAGCAGGTGACTCAGGTTTTGCTGCATACA | |
| | GTCGCTACAGGATTGGCAACTATAAATTAAACACAGACCATTCCAGTAGCAGTGA | |
| | CAATATTGCTTTGCTTGTACAGTAAGTGACAACAGATGTTTCATCTCGTTGACTT | |
| | TCAGGTTACTATAGCAGAGATATTACTAATTATTATGAGGACTTTTAAAGTTTCC | |
| | ATTTGGAATCTTGATTACATCATAAACCTCATAATTAAAAATTTATCTAAGTCAC | |
| | TAACTGAGAATAAATATTCTCAATTAGATGAAGAGCAACCAATGGAGATTGATTA | |
| | AACGAACATGAAAATTATTCTTTTCTTGGCACTGATAACACTCGCTACTTGTGAG | |
| | CTTTATCACTACCAAGAGTGTGTTAGAGGTACAACAGTACTTTTAAAAGAACCTT | |
| | GCTCTTCTGGAACATACGAGGGCAATTCACCATTTCATCCTCTAGCTGATAACAA | |
| | ATTTGCACTGACTTGCTTTAGCACTCAATTTGCTTTTGCTTGTCCTGACGGCGTA | |
| | AAACACGTCTATCAGTTACGTGCCAGATCAGTTTCACCTAAACTGTTCATCAGAC | |
| | AAGAGGAAGTTCAAGAACTTTACTCTCCAATTTTTCTTATTGTTGCGGCAATAGT | |
| | GTTTATAACACTTTGCTTCACACTCAAAAGAAAGACAGAATGATTGAACTTTCAT | |
| | TAATTGACTTCTATTTGTGCTTTTTAGCCTTTCTGCTATTCCTTGTTTTAATTAT | |
| | GCTTATTATCTTTTGGTTCTCACTTGAACTGCAAGATCATAATGAAACTTGTCAC | |
| | GCCTAAACGAACATGAAATTTCTTGTTTTCTTAGGAATCATCACAACTGTAGCTG | |
| | CATTTCACCAAGAATGTAGTTTACAGTCATGTACTCAACATCAACCATATGTAGT | |
| | TGATGACCCGTGTCCTATTCACTTCTATTCTAAATGGTATATTAGAGTAGGAGCT | |
| | AGAAAATCAGCACCTTTAATTGAATTGTGCGTGGATGAGGCTGGTTCTAAATCAC | |
| | CCATTCAGTACATCGATATCGGTAATTATACAGTTTCCTGTTCACCTTTTACAAT | |
| | TAATTGCCAGGAACCTAAATTGGGTAGTCTTGTAGTGCGTTGTTCGTTCTATGAA | |
| | GACTTTTTAGAGTATCATGACGTTCGTGTTGTTTTAGATTTCATCTAAACGAACA | |
| | AACTAAAATGTCTGATAATGGACCCCAAAATCAGCGAAATGCACCCCGCATTACG | |
| | TTTGGTGGACCCTCAGATTCAACTGGCAGTAACCAGAATGGAGAACGCAGTGGGG | |
| | CGCGATCAAAACAACGTCGGCCCCAAGGTTTACCCAATAATACTGCGTCTTGGTT | |
| | CACCGCTCTCACTCAACATGGCAAGGAAGACCTTAAATTCCCTCGAGGACAAGGC | |
| | GTTCCAATTAACACCAATAGCAGTCCAGATGACCAAATTGGCTACTACCGAAGAG | |
| | CTACCAGACGAATTCGTGGTGGTGACGGTAAAATGAAAGATCTCAGTCCAAGATG | |
| | GTATTTCTACTACCTAGGAACTGGGCCAGAAGCTGGACTTCCCTATGGTGCTAAC | |
| | AAAGACGGCATCATATGGGTTGCAACTGAGGGAGCCTTGAATACACCAAAAGATC | |
| | ACATTGGCACCCGCAATCCTGCTAACAATGCTGCAATCGTGCTACAACTTCCTCA | |
| | AGGAACAACATTGCCAAAAGGCTTCTACGCAGAAGGGAGCAGAGGCGGCAGTCAA | |
| | GCCTCTTCTCGTTCCTCATCACGTAGTCGCAACAGTTCAAGAAATTCAACTCCAG | |
| | GCAGCAGTAGGGGAACTTCTCCTGCTAGAATGGCTGGCAATGGCGGTGATGCTGC | |
| | TCTTGCTTTGCTGCTGCTTGACAGATTGAACCAGCTTGAGAGCAAAATGTCTGGT | |
| | AAAGGCCAACAACAACAAGGCCAAACTGTCACTAAGAAATCTGCTGCTGAGGCTT | |
| | CTAAGAAGCCTCGGCAAAAACGTACTGCCACTAAAGCATACAATGTAACACAAGC | |
| | TTTCGGCAGACGTGGTCCAGAACAAACCCAAGGAAATTTTGGGGACCAGGAACTA | |
| | ATCAGACAAGGAACTGATTACAAACATTGGCCGCAAATTGCACAATTTGCCCCCA | |
| | GCGCTTCAGCGTTCTTCGGAATGTCGCGCATTGGCATGGAAGTCACACCTTCGGG | |
| | AACGTGGTTGACCTACACAGGTGCCATCAAATTGGATGACAAAGATCCAAATTTC | |
| | AAAGATCAAGTCATTTTGCTGAATAAGCATATTGACGCATACAAAACATTCCCAC | |
| | CAACAGAGCCTAAAAAGGACAAAAAGAAGAAGGCTGATGAAACTCAAGCCTTACC | |
| | GCAGAGACAGAAGAAACAGCAAACTGTGACTCTTCTTCCTGCTGCAGATTTGGAT | |
| | GATTTCTCCAAACAATTGCAACAATCCATGAGCAGTGCTGACTCAACTCAGGCCT | |
| | AAACTCATGCAGACCACACAAGGCAGATGGGCTATATAAACGTTTTCGCTTTTCC | |
| | GTTTACGATATATAGTCTACTCTTGTGCAGAATGAATTCTCGTAACTACATAGCA | |
| | CAAGTAGATGTAGTTAACTTTAATCTCACATAGCAATCTTTAATCAGTGTGTAAC | |
| | ATTAGGGAGGACTTGAAAGAGCCACCACATTTTCACCGAGGCCACGCGGAGTACG | |
| | ATCGAGTGTACAGTGAACAATGCTAGGGAGAGCTGCCTATATGGAAGAGCCCTAA | |
| | TGTGTAAAATTAATTTTAGTAGTGCTATCCCCATGTGATTTTAATAGCTTCTTAG | |
| | GAGAATGAC | |

In various embodiment, the polynucleotide comprises a SARS-CoV-2 variant sequence from a natural isolate, wherein the spike protein coding sequence in the SARS-CoV-2 variant sequence is replaced with a recoded spike protein coding sequence from the SARS-CoV-2 variant. In various embodiments, the SARS-CoV-2 variant is the Alpha variant. In various embodiments, the SARS-CoV-2 variant is the Beta variant. In various embodiments, the SARS-CoV-2 variant is the Delta variant. In various embodiments, the SARS-CoV-2 variant is the Gamma variant. In various embodiments, the SARS-CoV-2 variant is the Omicron variant. In various embodiments, the SARS-CoV-2 variant is the Omicron variant sub-lineage BA.1, BA.1.1, BA.2, BA.3, BA.4 or BA.5. In various embodiments, the SARS-CoV-2 variant is the Omicron variant sub-lineage BA.4 or BA.5. Example sequences of these variants are as provided herein.

In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO: 10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

| | | SEQ |
|---|---|---|
| | | ID |
| Variant | Recoded Spike Sequence | NO: |
| Alpha | ATGTTTGTTTTTCTTGTTTTATTGCCACTAGTCTCTAGTCAGTGTGTTAATCTT | 3 |
| (UK) | ACAACCAGAACTCAATTACCCCCTGCATACACTAATTCTTTCACACGTGGTGTT | |
| | TATTACCCTGACAAAGTTTTCAGATCCTCAGTTTTACATTCAACTCAGGACTTG | |
| | TTCTTACCTTTCTTTTCCAATGTTACTTGGTTCCATGCTATCTCTGGGACCAAT | |
| | GGTACTAAGAGGTTTGATAACCCTGTCCTACCATTTAATGATGGTGTTTATTTT | |
| | GCTTCCACTGAGAAGTCTAACATAATAAGAGGCTGGATTTTTGGTACTACTTTA | |
| | GATTCGAAGACCCAGTCCCTACTTATTGTTAATAACGCTACTAATGTTGTTATT | |
| | AAAGTCTGTGAATTTCAATTTTGTAATGATCCATTTTTGGGTGTTTACCACAAA | |
| | AACAACAAAAGTTGGATGGAAAGTGAGTTCAGAGTTTATTCTAGTGCGAATAAT | |
| | TGCACTTTTGAATATGTCTCTCAGCCTTTTCTTATGGACCTTGAAGGAAAACAG | |
| | GGTAATTTCAAAAATCTTAGGGAATTTGTGTTTAAGAATATTGATGGTTATTTT | |
| | AAAATATATTCTAAGCACACGCCTATTAATTTAGTGCGTGATCTCCCTCAGGGT | |
| | TTTTCGGCTTTAGAACCATTGGTAGATTTGCCAATAGGTATTAACATCACTAGG | |
| | TTTCAAACTTTACTTGCTTTACATAGAAGTTATTTGACTCCTGGTGATTCTTCT | |
| | TCAGGTTGGACAGCTGGTGCTGCAGCTTATTATGTGGGTTATCTTCAACCTAGG | |
| | ACTTTTCTATTAAAATATAATGAAAATGGAACCATTACAGATGCTGTAGACTGT | |
| | GCACTTGACCCTCTCTCAGAAACAAAGTGTACGTTGAAATCCTTCACTGTAGAA | |
| | AAAGGAATCTATCAAACTTCTAACTTTAGAGTCCAACCAACAGAATCTATTGTT | |
| | AGATTTCCTAATATTACAAACTTGTGCCCTTTTGGTGAAGTTTTTAACGCCACC | |
| | AGATTTGCATCTGTTTATGCTTGGAACAGGAAGAGAATCAGCAACTGTGTTGCT | |
| | GATTATTCTGTCCTATATAATTCCGCATCATTTTCCACTTTTAAGTGTTATGGA | |
| | GTGTCTCCTACTAAATTAAATGATCTCTGCTTTACTAATGTCTATGCAGATTCA | |
| | TTTGTAATTAGAGGTGATGAAGTCAGACAAATCGCTCCAGGGCAAACTGGAAAG | |
| | ATTGCTGATTATAATTATAAATTACCAGATGATTTTACAGGCTGCGTTATAGCT | |
| | TGGAATTCTAACAATCTTGATTCTAAGGTTGGTGGTAATTATAATTACCTGTAT | |
| | AGATTGTTTAGGAAGTCTAATCTCAAACCTTTTGAGAGAGATATTTCAACTGAA | |
| | ATCTATCAGGCCGGTAGCACACCTTGTAATGGTGTTGAAGGTTTTAATTGTTAC | |
| | TTTCCTTTACAATCATATGGTTTCCAACCCACTtATGGTGTTGGTTACCAACCA | |
| | TACAGAGTAGTAGTACTTTCTTTTGAACTTCTACATGCACCAGCAACTGTTTGT | |
| | GGACCTAAAAAGTCTACTAATTTGGTTAAAAACAAATGTGTCAATTTCAACTTC | |
| | AATGGTTTAACAGGCACAGGTGTTCTTACTGAGTCTAACAAAAAGTTTCTGCCT | |
| | TTCCAACAATTTGGCAGAGACATTGaTGACACTACTGATGCTGTCCGTGATCCA | |
| | CAGACACTTGAGATTCTTGACATTACACCATGTTCTTTTGGTGGTGTCAGTGTT | |
| | ATAACACCAGGAACAAATACTTCTAACCAGGTTGCTGTTCTTTATCAGGgTGTT | |
| | AACTGCACAGAAGTCCCTGTTGCTATTCATGCAGATCAACTTACTCCTACTTGG | |
| | CGTGTTTATTCTACAGGTTCTAATGTTTTTCAAACACGTGCAGGCTGTTTAATA | |
| | GGGGCTGAACATGTCAACAACTCATATGAGTGTGACATACCCATTGGTGCAGGT | |
| | ATATGCGCTAGTTATCAGACTCAGCAATCCATCATTGCCTACACTATGTCACTT | |
| | GGTGCAGAAAATTCAGTTGCTTACTCTAATAACTCTATTGCCATACCCAtAAAT | |
| | TTTACTATTAGTGTTACCACAGAAATTCTACCAGTGTCTATGACCAAGACATCA | |
| | GTAGATTGTACAATGTACATTTGTGGTGATTCAACTGAATGCAGCAATCTTTTG | |
| | TTGCAATATGGCAGTTTTTGTACACAATTAAACCGTGCTTTAACTGGAATAGCT | |
| | GTTGAACAAGACAAAAACACCCAAGAAGTTTTTGCACAAGTCAAACAAATTTAC | |
| | AAAACACCACCAATTAAAGATTTTGGTGGTTTTAATTTTTCACAAATATTACCA | |
| | GATCCATCAAAACCAAGCAAGAGGTCATTTATTGAAGATCTACTTTTCAACAAA | |
| | GTGACACTTGCAGATGCTGGCTTCATCAAACAATATGGTGATTGCCTTGGTGAT | |
| | ATTGCTGCTAGAGACCTCATTTGCGCTCAAAAATTTAACGGACTTACAGTTTTA | |
| | CCACCTTTACTTACTGACGAAATGATTGCGCAATATACATCCGCATTGTTAGCC | |
| | GGAACTATTACATCCGGATGGACTTTTGGCGCAGGCGCAGCATTACAGATTCCA | |
| | TTCGCTATGCAAATGGCTTATAGGTTTAACGGTATAGGCGTTACGCAAAACGTA | |
| | CTTTATGAGAATCAAAAACTTATCGCTAACCAATTTAATTCCGCTATCGGTAAG | |
| | ATTCAGGATTCATTGTCTAGTACTGCTAGTGCACTCGGTAAGTTGCAAGACGTA | |
| | GTGAATCAAAACGCTCAAGCACTTAATACACTCGTTAAACAGCTTAGTTCTAAT | |
| | TTTGGCGCAATTTCTAGTGTGCTTAACGATATACTAgCAAGACTCGATAAAGTC | |
| | GAAGCCGAAGTGCAAATCGATAGATTGATTACCGGTAGGTTGCAATCATTGCAA | |
| | ACATACGTTACACAGCAATTGATTAGGGCCGCAGAGATACGCGCTAGCGCTAAT | |
| | CTCGCAGCTACTAAAATGTCTGAATGCGTACTCGGACAATCTAAACGTGTCGAT | |
| | TTTTGCGGTAAGGGATATCATCTTATGTCTTTTCCACAATCTGCACCTCACGGA | |
| | GTCGTGTTTTTACACGTTACTTATGTGCCAGCTCAAGAGAAAAATTTTACAACC | |
| | GCTCCTGCTATTTGTCATGACGGTAAGGCACATTTTCCTAGAGAGGGCGTATTC | |
| | GTTTCTAACGGTACACATTGGTTCGTTACACAACGTAATTTTTACGAACCTCAA | |
| | ATTATTACTACTCACAATACATTCGTATCAGGTAATTGTGACGTAGTGATAGGT | |
| | ATCGTTAATAATACAGTTTACGATCCACTTCAACCTGAACTCGATAGTTTTAAA | |
| | GAGGAACTCGATAAGTATTTTAAAAATCATACATCACCTGACGTCGACTTAGGC | |
| | GATATTTCAGGTATTAACGCTAGTGTCGTTAACATTCAAAAAGAGATTGATAGA | |
| | CTTAACGAAGTCGCTAAAAATCTTAACGAATCACTTATCGATCTGCAAGAGTTA | |
| | GGTAAGTATGAGCAATATATTAAATGGCCTTGGTATATTTGGTTAGGCTTTATA | |
| | GCCGGATTGATCGCAATCGTTATGGTTACAATTATGTTATGTTGTATGACATCA | |
| | TGTTGTTCATGTCTTAAGGGATGTTGTTCATGCGGATCATGTTGTAAATTTGAC | |
| | GAAGACGATTCCGAACCAGTGCTTAAAGGCGTTAAGTTACATTATACATAA | |
| Beta | See sequence listing. | 4 |
| (South | | |
| Africa) | | |
| Delta | See sequence listing. | 5 |
| (B.1. | | |
| 617.2) | | |
| Beta | ATGTTTGTTTTTCTTGTTTTATTGCCACTAGTCTCTAGTCAGTGTGTTAATTTT | 6 |
| | ACAACCAGAACTCAATTACCCCCTGCATACACTAATTCTTTCACACGTGGTGTT | |
| | TATTACCCTGACAAAGTTTTCAGATCCTCAGTTTTACATTCAACTCAGGACTTG | |
| | TTCTTACCTTTCTTTTCCAATGTTACTTGGTTCCATGCTATACATGTCTCTGGG | |
| | ACCAATGGTACTAAGAGGTTTGCTAACCCTGTCCTACCATTTAATGATGGTGTT | |
| | TATTTTGCTTCCACTGAGAAGTCTAACATAATAAGAGGCTGGATTTTTGGTACT | |
| | ACTTTAGATTCGAAGACCCAGTCCCTACTTATTGTTAATAACGCTACTAATGTT | |
| | GTTATTAAAGTCTGTGAATTTCAATTTTGTAATGATCCATTTTTGGGTGTTTAT | |
| | TACCACAAAAACAACAAAAGTTGGATGGAAAGTGAGTTCAGAGTTTATTCTAGT | |
| | GCGAATAATTGCACTTTTGAATATGTCTCTCAGCCTTTTCTTATGGACCTTGAA | |
| | GGAAAACAGGGTAATTTCAAAAATCTTAGGGAATTTGTGTTTAAGAATATTGAT | |
| | GGTTATTTTAAAATATATTCTAAGCACACGCCTATTAATTTAGTGCGTGGTCTC | |
| | CCTCAGGGTTTTTCGGCTTTAGAACCATTGGTAGATTTGCCAATAGGTATTAAC | |
| | ATCACTAGGTTTCAAACTTTACATAGAAGTTATTTGACTCCTGGTGATTCTTCT | |
| | TCAGGTTGGACAGCTGGTGCTGCAGCTTATTATGTGGGTTATCTTCAACCTAGG | |
| | ACTTTTCTATTAAAATATAATGAAAATGGAACCATTACAGATGCTGTAGACTGT | |
| | GCACTTGACCCTCTCTCAGAAACAAAGTGTACGTTGAAATCCTTCACTGTAGAA | |
| | AAAGGAATCTATCAAACTTCTAACTTTAGAGTCCAACCAACAGAATCTATTGTT | |
| | AGATTTCCTAATATTACAAACTTGTGCCCTTTTGGTGAAGTTTTTAACGCCACC | |
| | AGATTTGCATCTGTTTATGCTTGGAACAGGAAGAGAATCAGCAACTGTGTTGCT | |
| | GATTATTCTGTCCTATATAATTCCGCATCATTTTCCACTTTTAAGTGTTATGGA | |
| | GTGTCTCCTACTAAATTAAATGATCTCTGCTTTACTAATGTCTATGCAGATTCA | |
| | TTTGTAATTAGAGGTGATGAAGTCAGACAAATCGCTCCAGGGCAAACTGGAAAT | |
| | ATTGCTGATTATAATTATAAATTACCAGATGATTTTACAGGCTGCGTTATAGCT | |
| | TGGAATTCTAACAATCTTGATTCTAAGGTTGGTGGTAATTATAATTACCTGTAT | |
| | AGATTGTTTAGGAAGTCTAATCTCAAACCTTTTGAGAGAGATATTTCAACTGAA | |
| | ATCTATCAGGCCGGTAGCACACCTTGTAATGGTGTTAAAGGTTTTAATTGTTAC | |
| | TTTCCTTTACAATCATATGGTTTCCAACCCACTTATGGTGTTGGTTACCAACCA | |
| | TACAGAGTAGTAGTACTTTCTTTTGAACTTCTACATGCACCAGCAACTGTTTGT | |
| | GGACCTAAAAAGTCTACTAATTTGGTTAAAAACAAATGTGTCAATTTCAACTTC | |
| | AATGGTTTAACAGGCACAGGTGTTCTTACTGAGTCTAACAAAAAGTTTCTGCCT | |
| | TTCCAACAATTTGGCAGAGACATTGCTGACACTACTGATGCTGTCCGTGATCCA | |
| | CAGACACTTGAGATTCTTGACATTACACCATGTTCTTTTGGTGGTGTCAGTGTT | |
| | ATAACACCAGGAACAAATACTTCTAACCAGGTTGCTGTTCTTTATCAGGGTGTT | |
| | AACTGCACAGAAGTCCCTGTTGCTATTCATGCAGATCAACTTACTCCTACTTGG | |
| | CGTGTTTATTCTACAGGTTCTAATGTTTTTCAAACACGTGCAGGCTGTTTAATA | |
| | GGGGCTGAACATGTCAACAACTCATATGAGTGTGACATACCCATTGGTGCAGGT | |
| | ATATGCGCTAGTTATCAGACTCAGCAATCCATCATTGCCTACACTATGTCACTT | |
| | GGTGTAGAAAATTCAGTTGCTTACTCTAATAACTCTATTGCCATACCCACAAAT | |
| | TTTACTATTAGTGTTACCACAGAAATTCTACCAGTGTCTATGACCAAGACATCA | |
| | GTAGATTGTACAATGTACATTTGTGGTGATTCAACTGAATGCAGCAATCTTTTG | |
| | TTGCAATATGGCAGTTTTTGTACACAATTAAACCGTGCTTTAACTGGAATAGCT | |
| | GTTGAACAAGACAAAAACACCCAAGAAGTTTTTGCACAAGTCAAACAAATTTAC | |
| | AAAACACCACCAATTAAAGATTTTGGTGGTTTTAATTTTTCACAAATATTACCA | |
| | GATCCATCAAAACCAAGCAAGAGGTCATTTATTGAAGATCTACTTTTCAACAAA | |
| | GTGACACTTGCAGATGCTGGCTTCATCAAACAATATGGTGATTGCCTTGGTGAT | |
| | ATTGCTGCTAGAGATCTCATTTGCGCTCAAAAATTTAACGGACTTACAGTTTTA | |
| | CCACCTTTACTTACTGACGAAATGATTGCGCAATATACATCCGCATTGTTAGCC | |
| | GGAACTATTACATCCGGATGGACTTTTGGCGCAGGCGTAGCATTACAGATTCCA | |
| | TTCGCTATGCAAATGGCTTATAGGTTTAACGGTATAGGCGTTACGCAAAACGTA | |
| | CTTTATGAGAATCAAAAACTTATCGCTAACCAATTTAATTCCGCTATCGGTAAG | |
| | ATTCAGGATTCATTGTCTAGTACTGCTAGTGCACTCGGTAAGTTGCAAGACGTA | |
| | GTGAATCAAAACGCTCAAGCACTTAATACACTCGTTAAACAGCTTAGTTCTAAT | |
| | TTTGGCGCAATTTCTAGTGTGCTTAACGATATACTATCTAGACTCGATAAAGTC | |
| | GAAGCCGAAGTGCAAATCGATAGATTGATTACCGGTAGGTTGCAATCATTGCAA | |
| | ACATACGTTACACAGCAATTGATTAGGGCCGCAGAGATACGCGCTAGCGCTAAT | |
| | CTCGCAGCTACTAAAATGTCTGAATGCGTACTCGGACAATCTAAACGTGTCGAT | |
| | TTTTGCGGTAAGGGATATCATCTTATGTCTTTTCCACAATCTGCACCTCACGGA | |
| | GTCGTGTTTTTACACGTTACTTATGTGCCAGCTCAAGAGAAAAATTTTACAACC | |
| | GCTCCTGCTATTTGTCATGACGGTAAGGCACATTTTCCTAGAGAGGGCGTATTC | |
| | GTTTCTAACGGTACACATTGGTTCGTTACACAACGTAATTTTTACGAACCTCAA | |
| | ATTATTACTACTGATAATACATTCGTATCAGGTAATTGTGACGTAGTGATAGGT | |
| | ATCGTTAATAATACAGTTTACGATCCACTTCAACCTGAACTCGATAGTTTTAAA | |
| | GAGGAACTCGATAAGTATTTTAAAAATCATACATCACCTGACGTCGACTTAGGC | |
| | GATATTTCAGGTATTAACGCTAGTGTCGTTAACATTCAAAAAGAGATTGATAGA | |
| | CTTAACGAAGTCGCTAAAAATCTTAACGAATCACTTATCGATCTGCAAGAGTTA | |
| | GGTAAGTATGAGCAATATATTAAATGGCCTTGGTATATTTGGTTAGGCTTTATA | |
| | GCCGGATTGATCGCAATCGTTATGGTTACAATTATGTTATGTTGTATGACATCA | |
| | TGTTGTTCATGTCTTAAGGGATGTTGTTCATGCGGATCATGTTGTAAATTTGAC | |
| | GAAGACGATTCCGAACCAGTGCTTAAAGGCGTTAAGTTACATTATACATAA | |
| delta | ATGTTTGTTTTTCTTGTTTTATTGCCACTAGTCTCTAGTCAGTGTGTTAATCTT | 7 |
| | AgAACCAGAACTCAATTACCCCCTGCATACACTAATTCTTTCACACGTGGTGTT | |
| | TATTACCCTGACAAAGTTTTCAGATCCTCAGTTTTACATTCAACTCAGGACTTG | |
| | TTCTTACCTTTCTTTTCCAATGTTACTTGGTTCCATGCTATACATGTCTCTGGG | |
| | ACCAATGGTACTAAGAGGTTTGATAACCCTGTCCTACCATTTAATGATGGTGTT | |
| | TATTTTGCTTCCACTGAGAAGTCTAACATAATAAGAGGCTGGATTTTTGGTACT | |
| | ACTTTAGATTCGAAGACCCAGTCCCTACTTATTGTTAATAACGCTACTAATGTT | |
| | GTTATTAAAGTCTGTGAATTTCAATTTTGTAATGATCCATTTTTGGaTGTTTAT | |
| | TACCACAAAAACAACAAAAGTTGGATGGAAAGTGGAGTTTATTCTAGTGCGAAT | |
| | AATTGCACTTTTGAATATGTCTCTCAGCCTTTTCTTATGGACCTTGAAGGAAAA | |
| | CAGGGTAATTTCAAAAATCTTAGGGAATTTGTGTTTAAGAATATTGATGGTTAT | |
| | TTTAAAATATATTCTAAGCACACGCCTATTAATTTAGTGCGTGATCTCCCTCAG | |
| | GGTTTTTCGGCTTTAGAACCATTGGTAGATTTGCCAATAGGTATTAACATCACT | |
| | AGGTTTCAAACTTTACTTGCTTTACATAGAAGTTATTTGACTCCTGGTGATTCT | |
| | TCTTCAGGTTGGACAGCTGGTGCTGCAGCTTATTATGTGGGTTATCTTCAACCT | |
| | AGGACTTTTCTATTAAAATATAATGAAAATGGAACCATTACAGATGCTGTAGAC | |
| | TGTGCACTTGACCCTCTCTCAGAAACAAAGTGTACGTTGAAATCCTTCACTGTA | |
| | GAAAAAGGAATCTATCAAACTTCTAACTTTAGAGTCCAACCAACAGAATCTATT | |
| | GTTAGATTTCCTAATATTACAAACTTGTGCCCTTTTGGTGAAGTTTTTAACGCC | |
| | ACCAGATTTGCATCTGTTTATGCTTGGAACAGGAAGAGAATCAGCAACTGTGTT | |
| | GCTGATTATTCTGTCCTATATAATTCCGCATCATTTTCCACTTTTAAGTGTTAT | |
| | GGAGTGTCTCCTACTAAATTAAATGATCTCTGCTTTACTAATGTCTATGCAGAT | |
| | TCATTTGTAATTAGAGGTGATGAAGTCAGACAAATCGCTCCAGGGCAAACTGGA | |
| | AAGATTGCTGATTATAATTATAAATTACCAGATGATTTTACAGGCTGCGTTATA | |
| | GCTTGGAATTCTAACAATCTTGATTCTAAGGTTGGTGGTAATTATAATTACCOG | |
| | TATAGATTGTTTAGGAAGTCTAATCTCAAACCTTTTGAGAGAGATATTTCAACT | |
| | GAAATCTATCAGGCCGGTAGCAaACCTTGTAATGGTGTTGAAGGTTTTAATTGT | |
| | TACTTTCCTTTACAATCATATGGTTTCCAACCCACTAATGGTGTTGGTTACCAA | |
| | CCATACAGAGTAGTAGTACTTTCTTTTGAACTTCTACATGCACCAGCAACTGTT | |
| | TGTGGACCTAAAAAGTCTACTAATTTGGTTAAAAACAAATGTGTCAATTTCAAC | |
| | TTCAATGGTTTAACAGGCACAGGTGTTCTTACTGAGTCTAACAAAAAGTTTCTG | |
| | CCTTTCCAACAATTTGGCAGAGACATTGCTGACACTACTGATGCTGTCCGTGAT | |
| | CCACAGACACTTGAGATTCTTGACATTACACCATGTTCTTTTGGTGGTGTCAGT | |
| | GTTATAACACCAGGAACAAATACTTCTAACCAGGTTGCTGTTCTTTATCAGGgT | |
| | GTTAACTGCACAGAAGTCCCTGTTGCTATTCATGCAGATCAACTTACTCCTACT | |
| | TGGCGTGTTTATTCTACAGGTTCTAATGTTTTTCAAACACGTGCAGGCTGTTTA | |
| | ATAGGGGCTGAACATGTCAACAACTCATATGAGTGTGACATACCCATTGGTGCA | |
| | GGTATATGCGCTAGTTATCAGACTCAGCAATCCATCATTGCCTACACTATGTCA | |
| | CTTGGTGCAGAAAATTCAGTTGCTTACTCTAATAACTCTATTGCCATACCCACA | |
| | AATTTTACTATTAGTGTTACCACAGAAATTCTACCAGTGTCTATGACCAAGACA | |
| | TCAGTAGATTGTACAATGTACATTTGTGGTGATTCAACTGAATGCAGCAATCTT | |
| | TTGTTGCAATATGGCAGTTTTTGTACACAATTAAACCGTGCTTTAACTGGAATA | |
| | GCTGTTGAACAAGACAAAAACACCCAAGAAGTTTTTGCACAAGTCAAACAAATT | |
| | TACAAAACACCACCAATTAAAGATTTTGGTGGTTTTAATTTTTCACAAATATTA | |
| | CCAGATCCATCAAAACCAAGCAAGAGGTCATTTATTGAAGATCTACTTTTCAAC | |
| | AAAGTGACACTTGCAGATGCTGGCTTCATCAAACAATATGGTGATTGCCTTGGT | |
| | GATATTGCTGCTAGAGATCTCATTTGCGCTCAAAAATTTAACGGACTTACAGTT | |
| | TTACCACCTTTACTTACTGACGAAATGATTGCGCAATATACATCCGCATTGTTA | |
| | GCCGGAACTATTACATCCGGATGGACTTTTGGCGCAGGCGCAGCATTACAGATT | |
| | CCATTCGCTATGCAAATGGCTTATAGGTTTAACGGTATAGGCGTTACGCAAAAC | |
| | GTACTTTATGAGAATCAAAAACTTATCGCTAACCAATTTAATTCCGCTATCGGT | |
| | AAGATTCAGGATTCATTGTCTAGTACTGCTAGTGCACTCGGTAAGTTGCAAaAt | |
| | GTAGTGAATCAAAACGCTCAAGCACTTAATACACTCGTTAAACAGCTTAGTTCT | |
| | AATTTTGGCGCAATTTCTAGTGTGCTTAACGATATACTATCTAGACTCGATAAA | |
| | GTCGAAGCCGAAGTGCAAATCGATAGATTGATTACCGGTAGGTTGCAATCATTG | |
| | CAAACATACGTTACACAGCAATTGATTAGGGCCGCAGAGATACGCGCTAGCGCT | |
| | AATCTCGCAGCTACTAAAATGTCTGAATGCGTACTCGGACAATCTAAACGTGTC | |
| | GATTTTTGCGGTAAGGGATATCATCTTATGTCTTTTCCACAATCTGCACCTCAC | |
| | GGAGTCGTGTTTTTACACGTTACTTATGTGCCAGCTCAAGAGAAAAATTTTACA | |
| | ACCGCTCCTGCTATTTGTCATGACGGTAAGGCACATTTTCCTAGAGAGGGCGTA | |
| | TTCGTTTCTAACGGTACACATTGGTTCGTTACACAACGTAATTTTTACGAACCT | |
| | CAAATTATTACTACTGATAATACATTCGTATCAGGTAATTGTGACGTAGTGATA | |
| | GGTATCGTTAATAATACAGTTTACGATCCACTTCAACCTGAACTCGATAGTTTT | |
| | AAAGAGGAACTCGATAAGTATTTTAAAAATCATACATCACCTGACGTCGACTTA | |
| | GGCGATATTTCAGGTATTAACGCTAGTGTCGTTAACATTCAAAAAGAGATTGAT | |
| | AGACTTAACGAAGTCGCTAAAAATCTTAACGAATCACTTATCGATCTGCAAGAG | |
| | TTAGGTAAGTATGAGCAATATATTAAATGGCCTTGGTATATTTGGTTAGGCTTT | |
| | ATAGCCGGATTGATCGCAATCGTTATGGTTACAATTATGTTATGTTGTATGACA | |
| | TCATGTTGTTCATGTCTTAAGGGATGTTGTTCATGCGGATCATGTTGTAAATTT | |
| | GACGAAGACGATTCCGAACCAGTGCTTAAAGGCGTTAAGTTACATTATACATAA | |

In various embodiments, the polynucleotide further comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end.

### Vectors, Cells, Polypeptides

Various embodiments provide for a vector comprising a polynucleotide of the present invention. The polynucleotides of the present invention are the recoded polypeptides as discussed herein.

Various embodiments provide for a cell comprising a vector comprising a polynucleotide of the present invention. The vectors comprising a polynucleotide of the present invention are those as discussed herein.

Various embodiments provide for a bacterial artificial chromosome (BAC) comprising a polynucleotide of the present invention. The polynucleotides of the present invention are the recoded polypeptides as discussed herein.

Various embodiments provide for a cell comprising a polynucleotide of the present invention.

Various embodiments provide for a cell comprising modified/deoptimized infectious SARS-CoV-2 variant RNA of the present invention.

In various embodiments, the cell is a Vero cell, HeLa Cell, baby hamster kidney (BHK) cell, MA104 cell, 293T Cell, BSR-T7 Cell, MRC-5 cell, CHO cell, or PER.C6 cell. In particular embodiments, the cell is Vero cell or baby hamster kidney (BHK) cell.

Various embodiments provide for a polypeptide encoded by a polynucleotide of the present invention. The polynucleotides of the present invention are the recoded polypeptides as discussed herein. The polypeptide exhibits properties that are different than a polypeptide encoded by the SARS-CoV-2 variant from a natural isolate. For example, the polypeptide encoded by recoded polynucleotides and deoptimized polynucleotides as discussed herein can exert attenuating properties to the virus.

### Modified Viruses

Various embodiments of the present invention provide for a modified SARS-CoV-2 variant comprising a polypeptide encoded by a polynucleotide of the present invention. The polynucleotides of the present invention are the recoded polypeptides as discussed herein.

Various embodiments of the present invention provide for a modified SARS-CoV-2 variant comprising a polynucleotide of the present invention. The polynucleotides of the present invention are any one of the recoded polypeptides discussed herein.

In various embodiments, the expression of one or more of its viral proteins is reduced compared to its parent SARS-CoV-2 variant.

In various embodiments, the parent SARS-CoV-2 coronavirus is a SARS-CoV-2 variant. In various embodiments, SARS-CoV-2 variant is the U.K. variant, South Africa variant, Brazil variant, Delta variant, or Omicron variant. In various embodiments, SARS-CoV-2 variant is an Omicron variant sub-lineage BA.1, BA.1.1, BA.2, BA.3, BA.4 or BA.5. In various embodiments, SARS-CoV-2 variant is an Omicron variant sub-lineage BA.4 or BA.5.

Examples of the U.K. variant, South Africa variant, Brazil variant, Delta variant and Omicron variant include but are not limited to those discussed herein.

In various embodiments, the parent SARS-CoV-2 variant is a previously modified viral nucleic acid, or a previously attenuated viral nucleic acid.

In various embodiments the reduction in the expression of one or more of its viral proteins is reduced as the result of recoding a spike protein.

In various embodiments, the polynucleotide encodes one or more viral proteins or one or more fragments thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide remains the same.

In various embodiments, the polynucleotide encodes a spike protein or a fragment thereof of a parent SARS-CoV-2 variant, wherein the polynucleotide is recoded compared to its parent SARS-CoV-2 variant polynucleotide, and wherein the amino acid sequence of the spike protein or a fragment thereof of the parent SARS-CoV-2 variant encoded by the polynucleotide comprises up to 15 amino acid substitutions, additions, or deletions. In various embodiments, the amino acid sequence comprises up to 2, 3, 4, 5, 6, 7, 8, 9, 10, 11, 12, 13, 14, or 15 amino acid substitutions, additions, or deletions. In various embodiments, the amino acid sequence comprises 12 amino acid deletions. In various embodiments, the amino acid sequence comprises 1-3, 4-6, 7-9, 10-12, or 13-15 amino acid deletions. The amino acid substitutions, additions, or deletions can be due to one or more point mutations in the recoded sequence. In various embodiments, the amino acid deletion, substitution, or addition results from nucleic acid deletion(s), substitution(s) or addition(s) before the polyA tail of the nucleic acid sequence of the parent SARS-CoV-2 variant sequence.

In various embodiments, the polynucleotide is recoded by reducing codon-pair bias (CPB) or reducing codon usage bias compared to its parent SARS-CoV-2 variant polynucleotide.

In various embodiments, the polynucleotide is recoded by increasing the number of CpG or UpA di-nucleotides compared to its parent SARS-CoV-2 variant polynucleotide.

In various embodiments, each of the recoded spike protein or a fragment thereof has a codon pair bias less than −0.05, less than −0.1, less than −0.2, less than −0.3, or less than −0.4.

In various embodiments, each of the recoded spike protein or a fragment thereof has a codon pair bias less than −0.01, less than −0.02, less than −0.03, or less than −0.04. In various embodiments, each of the recoded spike protein or a fragment thereof has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In various embodiments, the codon pair bias of each of the recoded spike protein or a fragment thereof is reduced by at least 0.01, or at least 0.02, or at least 0.03, or at least 0.04. In various embodiments, the codon pair bias of each of the recoded spike protein or a fragment thereof is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding nucleic acid encoding the spike protein or fragment thereof. In certain embodiments, it is in comparison corresponding sequence from which the calculation is to be made; for example, the corresponding sequence of the spike encoding nucleic acid of SARS-CoV-2 variant.

In various embodiments, the parent SARS-CoV-2 coronavirus is a SARS-CoV-2 variant. In various embodiments, SARS-CoV-2 variant is the U.K. variant, South Africa variant, Brazil variant, Delta variant, or Omicron variant. In various embodiments, SARS-CoV-2 variant is an Omicron variant sub-lineage BA.1, BA.1.1, BA.2, BA.3, BA.4 or BA.5. In various embodiments, SARS-CoV-2 variant is an Omicron variant sub-lineage BA.4 or BA.5.

Examples of the U.K. variant, South Africa variant, Brazil variant, Delta variant and Omicron variant include but are not limited to those discussed herein.

In various embodiments, the parent SARS-CoV-2 variant is a previously modified viral nucleic acid, or a previously attenuated viral nucleic acid.

In various embodiments, the polynucleotide is CPB deoptimized compared to its parent SARS-CoV-2 variant polynucleotide. In various embodiments, the polynucleotide is codon deoptimized compared to its parent SARS-CoV-2 variant polynucleotide.

In various embodiments, the CPB deoptimized is based on CPB in humans. In various embodiments, the CPB deoptimized is based on CPB in a coronavirus. In various embodiments, the CPB deoptimized is based on CPB in a SARS-CoV-2 coronavirus. In various embodiments, the CPB deoptimized is based on CPB in a wild-type SARS-CoV-2 coronavirus. The wild-type SARS-CoV-2 coronavirus may be a SARS-CoV-2 variant coronavirus in accordance with various embodiments discuss herein.

In various embodiments, the codon usage deoptimized is based on frequently used codons in humans. In various embodiments, the codon usage deoptimized is based on frequently used codons in a coronavirus. In various embodiments, the codon usage deoptimized is based on frequently used codons or a SARS-CoV-2 coronavirus. In various embodiments, the codon usage deoptimized is based on frequently used codons in a wild-type SARS-CoV-2 coronavirus. In some embodiments, the wild-type SARS-CoV-2 coronavirus may be a SARS-CoV-2 variant coronavirus in accordance with various embodiments discuss herein.

In various embodiments, the polynucleotide comprises the spike protein or a fragment thereof. In various embodiments, polynucleotide comprises a deletion of nucleotides that results in a deletion of amino acids in the spike protein that eliminates the furin cleavage site. While not wishing to be bound by any particular theory, the inventors believe that eliminating the furin cleavage site will be one of the drivers of safety of the vaccine and/or immune composition.

In various embodiment, the polynucleotide comprises SEQ ID NO:1, wherein the spike protein coding sequence in SEQ ID NO:1 is replaced with a recoded spike protein coding sequence from a SARS-CoV-2 variant.

In various embodiment, the polynucleotide comprises SEQ ID NO:1 wherein nt 9469 is changed from to A to G and nt 26222 changed from T to G, wherein the spike protein coding sequence in SEQ ID NO:1 is replaced with a recoded spike protein coding sequence from a SARS-CoV-2 variant.

In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is one or more mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is two or more mutations in SEQ ID NO:1. In various embodiment, the polynucleotide comprises SEQ ID NO: 1, wherein the spike protein coding sequence in SEQ ID NO:1 is replaced with a recoded spike protein coding sequence from a SARS-CoV-2 variant, and wherein there is up to 5 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 10 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 20 mutations in SEQ ID NO:1. In various embodiment, the polynucleotide comprises SEQ ID NO: 1, wherein the spike protein coding sequence in SEQ ID NO:1 is replaced with a recoded spike protein coding sequence from a SARS-CoV-2 variant, and wherein there is up to 30 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 40 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 50 mutations in SEQ ID NO:1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 60 mutations in SEQ ID NO: 1. In various embodiment, the polynucleotide comprises SEQ ID NO:1, wherein the spike protein coding sequence in SEQ ID NO:1 is replaced with a recoded spike protein coding sequence from a SARS-CoV-2 variant, and wherein there is up to 70 mutations in SEQ ID NO:1. In various embodiments, the mutations in SEQ ID NO:1 is not an Alpha variant, Beta variant, Delta variant, Gamma variant, or Omicron variant.

In various embodiments, the SARS-CoV-2 variant is the Alpha variant. In various embodiments, the SARS-CoV-2 variant is the Beta variant. In various embodiments, the SARS-CoV-2 variant is the Delta variant. In various embodiments, the SARS-CoV-2 variant is the Gamma variant. In various embodiments, the SARS-CoV-2 variant is the Omicron variant. In various embodiments, SARS-CoV-2 variant is an Omicron variant sub-lineage BA.1, BA.1.1, BA.2, BA.3, BA.4 or BA.5. In various embodiments, SARS-CoV-2 variant is an Omicron variant sub-lineage BA.4 or BA.5.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiment, the polynucleotide comprises a SARS-CoV-2 variant sequence from a natural isolate, wherein the spike protein coding sequence in the SARS-CoV-2 variant sequence is replaced with a recoded spike protein coding sequence from the SARS-CoV-2 variant. In various embodiments, the SARS-CoV-2 variant is the Alpha variant. In various embodiments, the SARS-CoV-2 variant is the Beta variant. In various embodiments, the SARS-CoV-2 variant is the Delta variant. In various embodiments, the SARS-CoV-2 variant is the Gamma variant. In various embodiments, the SARS-CoV-2 variant is the Omicron variant. In various embodiments, the SARS-CoV-2 variant is the Omicron variant sub-lineage. Example sequences of these variants are as provided herein.

In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO: 10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the polynucleotide further comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end.

In various embodiments, the polynucleotide encodes SEQ ID NO:2 (recoded spike protein). In various embodiments, the polynucleotide encodes SEQ ID NO:2 (recoded spike protein), with up to 10 mutations.

In various embodiments, the recoded spike protein encoding sequence with up to 10 mutations for these modified variants is not SEQ ID NO: 1's spike encoding sequence. In various embodiments, the recoded spike protein encoding sequence with up to 10 mutations for these modified variants is not the spike encoding sequence in SEQ ID NO:1 wherein nt 9469 is changed from to A to G and nt 26222 changed from T to G.

Immune and/or Vaccines Compositions

Various embodiments provide for an immune composition for inducing an immune response in a subject, comprising: a modified SARS-CoV-2 variant of the present invention. The modified SARS-CoV-2 variant is any one of the modified SARS-CoV-2 variant discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Omicron variant. In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO: 10. In various embodiments, the polynucleotide comprises a polynucleotide having at least 99% sequence identity to SEQ ID NO: 10. In various embodiments, the polynucleotide comprises a polynucleotide having at least 99.5% sequence identity to SEQ ID NO: 10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the modified SARS-CoV-2 variant of the present invention is a live-attenuated virus.

Various embodiments provide for a multivalent immune composition for inducing a protective an immune response in a subject, comprising: two or more modified SARS-CoV-2 variant of the present invention.

Various embodiments provide for a multivalent immune composition for inducing a protective an immune response in a subject, comprising: a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV-2 coronavirus is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In some embodiments the immune composition further comprises an acceptable excipient or carrier as described herein. In some embodiments, the immune composition further comprises a stabilizer as described herein. In some embodiments, the immune composition further comprise an adjuvant as described herein. In some embodiments, the immune composition further comprises sucrose, glycine or both. In various embodiments, the immune composition further comprises about sucrose (5%) and about glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the immune composition is provided at dosages of about 10³-10⁷ PFU. In various embodiments, the immune composition is provided at dosages of about 10⁴-10⁶ PFU. In various embodiments, the immune composition is provided at a dosage of about 10³ PFU. In various embodiments, the immune composition is provided at a dosage of about 10⁴ PFU. In various embodiments, the immune composition is provided at a dosage of about 10⁵ PFU. In various embodiments, the immune composition is provided at a dosage of about 10⁶ PFU. In various embodiments, the immune composition is provided at a dosage of about 10⁷ PFU. In various embodiments, the immune composition is provided at a dosage of about 10⁸ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU. In various embodiments, the immune composition is provided at a dosage of about 5×10⁸ PFU.

Various embodiments provide for a vaccine composition for inducing an immune response in a subject, comprising: a modified SARS-CoV-2 variant of the present invention. The modified SARS-CoV-2 variant is any one of the modified SARS-CoV-2 variant discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Omicron variant. In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO: 10. In various embodiments, the polynucleotide comprises a polynucleotide having at least 99% sequence identity to SEQ ID NO: 10. In various embodiments, the polynucleotide comprises a polynucleotide having at least 99.5% sequence identity to SEQ ID NO: 10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO: 11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the modified SARS-CoV-2 variant of the present invention is a live-attenuated virus.

Various embodiments provide for a multivalent vaccine composition for inducing an immune response in a subject, comprising: two or more modified SARS-CoV-2 variant of the present invention.

Various embodiments provide for a multivalent vaccine composition for inducing a protective an immune response in a subject, comprising: a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In some embodiments the vaccine composition further comprises an acceptable carrier or excipient as described herein. In some embodiments, the immune composition further comprises a stabilizer as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁷ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU. In various embodiments, the vaccine composition is provided at a dosage of about 10⁸ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU. In various embodiments, the immune composition is provided at a dosage of about 5×10⁸ PFU.

Various embodiments provide for a vaccine composition for inducing a protective immune response in a subject, comprising: a modified SARS-CoV-2 variant of the present invention. The modified SARS-CoV-2 variant is any one of the modified SARS-CoV-2 variant discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Omicron variant. In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO: 10. In various embodiments, the polynucleotide comprises a polynucleotide having at least 99% sequence identity to SEQ ID NO: 10. In various embodiments, the polynucleotide comprises a polynucleotide having at least 99.5% sequence identity to SEQ ID NO: 10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO: 11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the modified SARS-CoV-2 variant of the present invention is a live-attenuated virus.

Various embodiments provide for a multivalent vaccine composition for inducing a protective an immune response in a subject, comprising: two or more modified SARS-CoV-2 variant of the present invention.

Various embodiments provide for a multivalent vaccine composition for inducing a protective immune response in a subject, comprising: a modified SARS-CoV-2 coronavirus that is deoptimized in reference to original SARS-CoV-2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In some embodiments the vaccine composition further comprises an acceptable carrier or excipient as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁴ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU. In various embodiments, the vaccine composition is provided at a dosage of about 10⁸ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU. In various embodiments, the immune composition is provided at a dosage of about 5×10⁸ PFU.

It should be understood that an attenuated virus of the invention, where used to elicit an immune response in a subject (or protective immune response) or to prevent a subject from or reduce the likelihood of becoming afflicted with a virus-associated disease, can be administered to the subject in the form of a composition additionally comprising a pharmaceutically acceptable carrier or excipient. Pharmaceutically acceptable carriers and excipients are known to those skilled in the art and include, but are not limited to, one or more of 0.01-0.1M and preferably 0.05M phosphate buffer, phosphate-buffered saline (PBS), DMEM, L-15, a 10-25% sucrose solution in PBS, a 10-25% sucrose solution in DMEM, or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate, gelatin, recombinant human serum albumin, human serum albumin, and/or magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers or excipients can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments, the vaccine composition or immune composition is formulated for delivery intravenously, or intrathecally, subcutaneously, intramuscularly, intradermally or intranasally. In various embodiments, the vaccine composition or immune composition is formulated for delivery intranasally. In various embodiments, the vaccine composition or immune composition is formulated for delivery via a nasal drop or nasal spray.

### Methods of Using the Compositions of the Present Invention.

Various embodiments provide for a method of eliciting an immune response in a subject, comprising: administering to the subject a dose of an immune composition the present invention. The immune composition is any one of the immune composition discussed herein. In various embodiments, the dose is a prophylactically effective or therapeutically effective dose.

Various embodiments provide for a method of eliciting an immune response in a subject, comprising: administering to the subject a dose of a multivalent immune composition the present invention. The multivalent immune composition is any one of the immune composition discussed herein. In various embodiments, the dose is a prophylactically effective or therapeutically effective dose.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Omicron variant. In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO: 10. In various embodiments, the polynucleotide comprises a polynucleotide having at least 99% sequence identity to SEQ ID NO: 10. In various embodiments, the polynucleotide comprises a polynucleotide having at least 99.5% sequence identity to SEQ ID NO: 10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO: 11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the multivalent immune composition comprises: a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In various embodiments, the immune composition is administered intravenously, or intrathecally, subcutaneously, intramuscularly, intradermally or intranasally. In various embodiments, the immune composition is administered intranasally. In various embodiments, the immune composition is administered via a nasal drop or nasal spray.

Various embodiments provide for a method of eliciting an immune response in a subject, comprising: administering to the subject a dose of a vaccine composition the present invention. The vaccine composition is any one of the vaccine composition discussed herein.

Various embodiments provide for a method of eliciting an immune response in a subject, comprising: administering to the subject a dose of a multivalent vaccine composition the present invention. The multivalent vaccine composition is any one of the vaccine composition discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8 In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments the multivalent vaccine composition comprises a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In various embodiments, the immune response is a protective immune response. In various embodiments, the dose is a prophylactically effective or therapeutically effective dose.

In various embodiments, the vaccine composition is administered intravenously, or intrathecally, subcutaneously, intramuscularly, intradermally or intranasally. In various embodiments, the vaccine composition is administered intranasally. In various embodiments, the vaccine composition is administered via a nasal drop or nasal spray.

Various embodiments provide for a method of eliciting an immune response in a subject, comprising: administering to the subject a dose of a modified SARS-CoV-2 variant of the present invention. The modified SARS-CoV-2 variant is any one of the modified SARS-CoV-2 variant discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO: 9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the SARS-CoV-2 variant is the Omicron variant. In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO: 10. In various embodiments, the polynucleotide comprises a polynucleotide having at least 99% sequence identity to SEQ ID NO: 10. In various embodiments, the polynucleotide comprises a polynucleotide having at least 99.5% sequence identity to SEQ ID NO: 10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the immune response is a protective immune response. In various embodiments, the dose is a prophylactically effective or therapeutically effective dose.

In various embodiments, the dose is about 10³-10⁷ PFU. In various embodiments, the dose is about 10⁴-10⁶ PFU. In various embodiments, the dose is about 10³ PFU. In various embodiments, the dose is about 10⁴ PFU. In various embodiments, the dose is about 10⁵ PFU. In various embodiments, the dose is about 10⁶ PFU. In various embodiments, the dose is about 10⁷ PFU. In various embodiments, the dose is about 10⁸ PFU.

In various embodiments, the dose is about 5×10³ PFU. In various embodiments, the dose is about 5×10⁴ PFU. In various embodiments, the dose is about 5×10⁵ PFU. In various embodiments, the dose is about 5×10⁶ PFU. In various embodiments, the dose is about 5×10⁷ PFU. In various embodiments, the dose is about 5×10⁸ PFU.

In various embodiments, the modified SARS-CoV-2 variant is administered intravenously, or intrathecally, subcutaneously, intramuscularly, intradermally or intranasally. In various embodiments, the modified SARS-CoV-2 coronavirus is administered intranasally. In various embodiments, the modified SARS-CoV-2 coronavirus is administered via a nasal drop or nasal spray.

Various embodiments provide for a method of eliciting an immune response in a subject, comprising: administering to the subject a prime dose of a modified SARS-CoV-2 variant of the present invention; and administering to the subject one or more boost doses of a modified SARS-CoV-2 variant of the present invention. In various embodiments, the modified SARS-CoV-2 variant is any one of the modified SARS-CoV-2 variant discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the prime dose and the one or more boost doses utilizes the same modified SARS-CoV-2 variant. In various embodiments, the prime dose and the one or more boost doses utilizes a different modified SARS-CoV-2 variant. In various embodiments, the dose is a prophylactically effective or therapeutically effective dose.

In various embodiments, the prime dose and/or the one or more boost doses of the modified SARS-CoV-2 variant is administered intravenously, or intrathecally, subcutaneously, intramuscularly, intradermally or intranasally. In various embodiments, the prime dose and/or the one or more boost doses of the modified SARS-CoV-2 variant is administered intranasally. In various embodiments, the prime dose and/or the one or more boost doses of the modified SARS-CoV-2 variant is administered via a nasal drop or nasal spray.

Various embodiments provide for a method of eliciting an immune response in a subject, comprising: administering to the subject a prime dose of an immune composition of the present invention; and administering to the subject one or more boost doses of an immune composition of the present invention. In various embodiments, the immune composition is any one of the immune composition discussed herein.

Various embodiments provide for a method of eliciting an immune response in a subject, comprising: administering to the subject a prime dose of a multivalent immune composition of the present invention; and administering to the subject one or more boost doses of a multivalent immune composition of the present invention. In various embodiments, the multivalent immune composition is any one of the immune composition discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the multivalent immune composition comprises: a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In various embodiments, the prime dose and the one or more boost doses utilizes the same immune composition comprising the same modified SARS-CoV-2 variant. In various embodiments, the prime dose and the one or more boost doses utilizes a different immune composition comprising a different modified SARS-CoV-2 variant. In various embodiments, the dose is a prophylactically effective or therapeutically effective dose.

In various embodiments, the prime dose and/or the one or more boost doses of the immune composition is administered intravenously, or intrathecally, subcutaneously, intramuscularly, intradermally or intranasally. In various embodiments, the prime dose and/or the one or more boost doses of the immune composition is administered intranasally. In various embodiments, the prime dose and/or the one or more boost doses of the immune composition is administered via a nasal drop or nasal spray.

Various embodiments provide for a method of eliciting an immune response in a subject, comprising: administering to the subject a prime dose of a vaccine composition of the present invention; and administering to the subject one or more boost doses of a vaccine composition of the present invention. In various embodiments, the vaccine composition is any one of the vaccine composition discussed herein.

Various embodiments provide for a method of eliciting an immune response in a subject, comprising: administering to the subject a prime dose of a multivalent vaccine composition of the present invention; and administering to the subject one or more boost doses of a multivalent vaccine composition of the present invention. In various embodiments, the vaccine composition is any one of the vaccine composition discussed herein.

In various embodiments, the SARS-CoV-2 variant is the Beta variant. In various embodiments, the polynucleotide comprises SEQ ID NO:8. In various embodiments, the polynucleotide comprises SEQ ID NO:8, with up to 20 mutations in SEQ ID NO:8. In various embodiments, the polynucleotide comprises a polynucleotide having at least 95%, 96%, 98%, 99% sequence identity to SEQ ID NO:8. In various embodiments, the polynucleotide comprises a polynucleotide having at least 99% sequence identity to SEQ ID NO:8. In various embodiments, the polynucleotide comprises a polynucleotide having at least 99.5% sequence identity to SEQ ID NO:8.

In various embodiments, the SARS-CoV-2 variant is the Delta variant. In various embodiments, the polynucleotide comprises SEQ ID NO:9. In various embodiments, the polynucleotide comprises SEQ ID NO:9, with up to 20 mutations in SEQ ID NO:9. In various embodiments, the polynucleotide comprises a polynucleotide having at least 95%, 96%, 98%, 99% sequence identity to SEQ ID NO:9. In various embodiments, the polynucleotide comprises a polynucleotide having at least 99% sequence identity to SEQ ID NO:9. In various embodiments, the polynucleotide comprises a polynucleotide having at least 99.5% sequence identity to SEQ ID NO:9.

In various embodiments, the polynucleotide comprises SEQ ID NO:10. In various embodiments, the polynucleotide comprises SEQ ID NO:10, with up to 20 mutations in SEQ ID NO:10. In various embodiments, the polynucleotide comprises a polynucleotide having at least 95%, 96%, 98%, 99% sequence identity to SEQ ID NO:10. In various embodiments, the polynucleotide comprises a polynucleotide having at least 99% sequence identity to SEQ ID NO:10. In various embodiments, the polynucleotide comprises a polynucleotide having at least 99.5% sequence identity to SEQ ID NO:10.

In various embodiments, the SARS-CoV-2 variant is the Omicron variant. In various embodiments, the polynucleotide comprises SEQ ID NO:11. In various embodiments, the polynucleotide comprises SEQ ID NO:11, with up to 20 mutations in SEQ ID NO:10. In various embodiments, the polynucleotide comprises a polynucleotide having at least 95%, 96%, 98%, 99% sequence identity to SEQ ID NO: 11. In various embodiments, the polynucleotide comprises a polynucleotide having at least 99% sequence identity to SEQ ID NO: 11. In various embodiments, the polynucleotide comprises a polynucleotide having at least 99.5% sequence identity to SEQ ID NO: 11.

In various embodiments, the SARS-CoV-2 variant is the Omicron variant. In various embodiments, the polynucleotide comprises SEQ ID NO:12. In various embodiments, the polynucleotide comprises SEQ ID NO:10, with up to 20 mutations in SEQ ID NO:12. In various embodiments, the polynucleotide comprises a polynucleotide having at least 95%, 96%, 98%, 99% sequence identity to SEQ ID NO: 12. In various embodiments, the polynucleotide comprises a polynucleotide having at least 99% sequence identity to SEQ ID NO: 12. In various embodiments, the polynucleotide comprises a polynucleotide having at least 99.5% sequence identity to SEQ ID NO: 12.

In various embodiments, the modified SARS-CoV-2 variant comprises a recoded spike protein. In various embodiments, the recoded spike protein encoding sequence is SEQ ID NO:3. In various embodiments, the recoded spike protein encoding sequence is SEQ ID NO:4. In various embodiments, the recoded spike protein encoding sequence is SEQ ID NO:5. In various embodiments, the recoded spike protein encoding sequence is SEQ ID NO:6. In various embodiments, the recoded spike protein encoding sequence is SEQ ID NO:7. In various embodiments, the recoded spike protein encoding sequence is nucleotide 21,563 to 25,339 of SEQ ID NO:8. In various embodiments, the recoded spike protein encoding sequence is nucleotide 21,563 to 25,342 of SEQ ID NO:9. In various embodiments, the recoded spike protein encoding sequence is nucleotide 21,563 to 25,339 of SEQ ID NO:10. In various embodiments, the recoded spike protein encoding sequence is nucleotide 21,563 to 25,339 of SEQ ID NO:11. In various embodiments, the recoded spike protein encoding sequence is nucleotide 21,563 to 25,333 of SEQ ID NO: 12.

In various embodiments, the recoded spike protein encoding sequence is SEQ ID NO:3 with up to 10 mutations. In various embodiments, the recoded spike protein encoding sequence is SEQ ID NO:4 with up to 10 mutations. In various embodiments, the recoded spike protein encoding sequence is SEQ ID NO:5 with up to 10 mutations. In various embodiments, the recoded spike protein encoding sequence is SEQ ID NO:6 with up to 10 mutations. In various embodiments, the recoded spike protein encoding sequence is SEQ ID NO:7 with up to 10 mutations. In various embodiments, the recoded spike protein encoding sequence is nucleotide 21,563 to 25,339 of SEQ ID NO:10 with up to 10 mutations. In various embodiments, the recoded spike protein encoding sequence is nucleotide 21,563 to 25,339 of SEQ ID NO: 11 with up to 10 mutations. In various embodiments, the recoded spike protein encoding sequence is nucleotide 21,563 to 25,333 of SEQ ID NO:12 with up to 10 mutations.

In various embodiments, the recoded spike protein encoding sequence is at least 98%, or at least 99% identical to SEQ ID NO:3. In various embodiments, the recoded spike protein encoding sequence is at least 98%, or at least 99% identical to SEQ ID NO:4. In various embodiments, the recoded spike protein encoding sequence is at least 98%, or at least 99% identical to SEQ ID NO:5. In various embodiments, the recoded spike protein encoding sequence is at least 98%, or at least 99% identical to SEQ ID NO:6. In various embodiments, the recoded spike protein encoding sequence is at least 98%, or at least 99% identical to SEQ ID NO:7. In various embodiments, the recoded spike protein encoding sequence is at least 98%, or at least 99% identical to nucleotide 21,563 to 25,339 of SEQ ID NO:10. In various embodiments, the recoded spike protein encoding sequence is at least 98%, or at least 99% identical to nucleotide 21,563 to 25,339 of SEQ ID NO:11. In various embodiments, the recoded spike protein encoding sequence is at least 98%, or at least 99% identical to nucleotide 21,563 to 25,333 of SEQ ID NO: 12.

In various embodiments, the multivalent vaccine composition comprises: a modified SARS-CoV-2 coronavirus that is deoptimized in reference to original SARS-CoV2 and one or more modified SARS-CoV-2 variants of the present invention. That is, a modified SARS-CoV-2 coronavirus that is deoptimized in reference to original SARS-CoV2 is not a modified SARS-CoV-2 variant. Each modified SARS-CoV-2 variant is any one of the modified SARS-CoV-2 variant discussed herein.

An example of a modified SARS-CoV-2 coronavirus that is deoptimized in reference to original SARS-CoV2 is on that is deoptimized in reference to the Washington Isolate. In various embodiments, the modified SARS-CoV-2 coronavirus comprises a polynucleotide having SEQ ID NO: 1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant. In various embodiments, the modified SARS-CoV-2 coronavirus comprises a polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant.

In various embodiments, the prime dose and the one or more boost doses utilizes the same vaccine composition comprising the same modified SARS-CoV-2 variant. In various embodiments, the prime dose and the one or more boost doses utilizes a different vaccine composition comprising a different modified SARS-CoV-2 variant. In various embodiments, the dose is a prophylactically effective or therapeutically effective dose.

In various embodiments, the prime dose and/or the one or more boost doses of the vaccine composition is administered intravenously, or intrathecally, subcutaneously, intramuscularly, intradermally or intranasally. In various embodiments, the prime dose and/or the one or more boost doses of the vaccine composition is administered intranasally. In various embodiments, the prime dose and/or the one or more boost doses of the vaccine composition is administered via a nasal drop or nasal spray.

The timing between the prime and boost dosages can vary, for example, depending on the stage of infection or disease (e.g., non-infected, infected, number of days post infection), and the patient's health. In various embodiments, the one or more boost dose is administered about 2 weeks after the prime dose. That is, the prime dose is administered and about two weeks thereafter, a boost dose is administered. In various embodiments, the one or more boost dose is administered about 4 weeks after the prime dose. In various embodiments, the one or more boost dose is administered about 6 weeks after the prime dose. In various embodiments, the one or more boost dose is administered about 8 weeks after the prime dose. In various embodiments, the one or more boost dose is administered about 12 weeks after the prime dose. In various embodiments, the one or more boost dose is administered about 1-12 weeks after the prime dose.

In various embodiments, the one or more boost doses can be given as one boost dose. In other embodiments, the one or more boost doses can be given as a boost dose periodically. For example, it can be given quarterly, every 4 months, every 6 months, yearly, every 2 years, every 3 years, every 4 years, every 5 years, every 6 years, every 7 years, every 8 years, every 9 years, or every 10 years.

In various embodiments, the prime dose and boost does are each about 10³-10⁷ PFU. In various embodiments, the prime dose and boost does are each about 10⁴-10⁶ PFU. In various embodiments, the prime dose and boost does are each about 10³ PFU. In various embodiments, the prime dose and boost does are each about 10⁴ PFU. In various embodiments, the prime dose and boost does are each about 10⁵ PFU. In various embodiments, the prime dose and boost does are each about 10⁶ PFU. In various embodiments, the dose is about 10⁷ PFU. In various embodiments, the dose is about 10⁸ PFU.

In various embodiments, the prime dose and boost does are each about 5×10³ PFU. In various embodiments, the prime dose and boost does are each about 5×10⁴ PFU. In various embodiments, the prime dose and boost does are each about 5×10⁵ PFU. In various embodiments, the prime dose and boost does are each about 5×10⁶ PFU. In various embodiments, the prime dose and boost does are each about 5×10⁷ PFU. In various embodiments, the prime dose and boost does are each about 5×10⁸ PFU.

In various embodiments, the dosage for the prime dose and the boost dose is the same.

In various embodiments, the dosage amount can vary between the prime and boost dosages. As a non-limiting example, the prime dose can contain fewer copies of the virus compared to the boost dose. For example, the prime dose is about 10³ PFU and the boost dose is about 10⁴-10⁶ PFU, or, the prime dose is about 10⁴ and the boost dose is about 10⁵-10⁷ PFU.

In various embodiments, wherein the boost dose is administered periodically, the subsequent boost doses can be less than the first boost dose.

As another non-limiting example, the prime dose can contain more copies of the virus compared to the boost dose.

In various embodiments, the immune response is a protective immune response.

In various embodiments, the dose is a prophylactically effective or therapeutically effective dose.

In various embodiments, intranasal administration of a modified SARS-CoV-2 variant of the present invention, the immune composition of the present invention, the vaccine composition of the present invention, the multivalent immune composition of the present invention, or the multivalent vaccine composition of the present invention comprises: instructing the subject blow the nose and tilt the head back; optionally, instructing the subject reposition the head to avoid having composition dripping outside of the nose or down the throat; administering about 0.25 mL comprising the dosage into each nostril; instructing the subject to sniff gently; and instructing the subject to not blow the nose for a period of time; for example, about 60 minutes.

In some embodiments, the subject is not taking any immunosuppressive medications. In various embodiments, the subject is not taking any immunosuppressive medications about 180 days, 150 days, 120 days, 90 days, 75 days, 60 days, 45 days, 30 days, 15 days or 7 days before the administration of the modified SARS-CoV-2 variant of the present invention, the immune composition of the present invention or the vaccine composition of the present invention. In various embodiments, the subject does not take any immunosuppressive medications for about 1 day, 7 days, 14 days, 30 days, 45 days, 60 days, 75 days, 90 days, 120 days, 150 days, 180 days, 9 months, 12 months, 15 months, 18 months, 21 months, or 24 months after the administration of the modified SARS-CoV-2 variant of the present invention, the immune composition of the present invention or the vaccine composition of the present invention.

Immunosuppressive medications (including, but not limited to, the following: Corticosteroids (e.g., prednisone (Deltasone, Orasone), budesonide (Entocort EC), prednisolone (Millipred)), Calcineurin inhibitors (e.g., cyclosporine (Neoral, Sandimmune, SangCya), tacrolimus (Astagraf XL, Envarsus XR, Prograf), Mechanistic target of rapamycin (mTOR) inhibitors (e.g., sirolimus (Rapamune), everolimus (Afinitor, Zortress)), Inosine monophosphate dehydrogenase (IMDH) inhibitors, (e.g., azathioprine (Azasan, Imuran), leflunomide (Arava), mycophenolate (CellCept, Myfortic)), Biologics (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio)), Monoclonal antibodies (e.g., basiliximab (Simulect), daclizumab (Zinbryta), muromonab (Orthoclone OKT3)).

### Medical Uses

Various embodiments of the present invention provide for a modified SARS-CoV-2 variant of the present invention, a vaccine composition of the present invention, or an immune composition of the present invention for use in eliciting an immune response, or for therapeutic or prophylactic treatment of COVID-19.

Various embodiments of the present invention provide for a modified SARS-CoV-2 variant of the present invention, a vaccine composition of the present invention, or an immune composition of the present invention for use in eliciting an immune response, or for therapeutic or prophylactic treatment of COVID-19, wherein the use comprises a prime dose of the modified SARS-CoV-2 variant of the present invention, or the vaccine composition of the present invention, or the immune composition of the present invention, and one or more boost doses of the modified SARS-CoV-2 variant of the present invention, or the vaccine composition of the present invention, or the immune composition of the present invention.

Various embodiments of the present invention provide for a use of modified SARS-CoV-2 variant of the present invention, a vaccine composition of the present invention, or an immune composition of the present invention in the manufacture of a medicament for eliciting an immune response, or for therapeutic or prophylactic treatment of COVID-19.

Various embodiments of the present invention provide for a use of modified SARS-CoV-2 variant of the present invention, a vaccine composition of the present invention, or an immune composition of the present invention in the manufacture of a medicament for use in eliciting an immune response, or for therapeutic or prophylactic treatment of COVID-19, wherein the medicament comprises a prime dose of the modified SARS-CoV-2 variant of the present invention, or the vaccine composition of the present invention, or the immune composition of the present invention, and one or more boost doses of the modified SARS-CoV-2 variant of the present invention, or the vaccine composition of the present invention, or the immune composition of the present invention.

In various embodiments, the immune composition is a multivalent immune composition as described herein.

In various embodiments, the vaccine composition is a multivalent vaccine composition as described herein.

The modified SARS-CoV-2 variant of the present invention is any one of the modified SARS-CoV-2 coronavirus discussed herein. The vaccine composition of the present invention is any one of the vaccine compositions discussed herein. The immune composition of the present invention is any one of the immune compositions discussed herein.

In various embodiments, the immune response is a protective immune response.

### Methods of Making

Various embodiments provide for a method of making a modified SARS-CoV-2 variant, comprising: obtaining a nucleotide sequence encoding one or more proteins of a parent SARS-CoV-2 variant or one or more fragments thereof, recoding the nucleotide sequence to reduce protein expression of the one or more proteins, or the one or more fragments thereof, and substituting a nucleic acid having the recoded nucleotide sequence into the parent SARS-CoV-2 variant genome to make the modified SARS-CoV-2 variant genome, wherein expression of the recoded nucleotide sequence is reduced compared to the parent virus.

In various embodiment, making the modified SARS-CoV-2 variant genome comprises using a cloning host.

In various embodiment, making the modified SARS-CoV-2 variant genome comprises constructing an infectious cDNA clone, using BAC vector, using an overlap extension PCR strategy, or long PCR-based fusion strategy.

In various embodiment, the modified SARS-CoV-2 variant genome further comprises one or more mutations, including deletion, substitutions and additions. One or more can be 1-5, 6-10, 11-15, 16-20, 21-25, 26-30, 31-35, 36-40, 41-45, 46-50, 51-60, 61-70, 71-80, 81-90, or 91-100 mutations.

In various embodiments, recoding the nucleotide sequence to reduce protein expression of the one or more proteins, or the one or more fragments thereof is by way of reducing codon-pair bias (CPB) compared to its parent SARS-CoV-2 variant polynucleotide, reducing codon usage bias compared to its parent SARS-CoV-2 variant polynucleotide, or increasing the number of CpG or UpA di-nucleotides compared to its parent SARS-CoV-2 variant polynucleotide, as discuss herein.

Various embodiments of the present invention provide for a method of generating a modified viral genome, comprising performing reverse transcription polymerase chain reaction (“RT-PCR”) on a viral RNA from a SARS-CoV-2 variant to generate cDNA; performing polymerase chain reaction (“PCR”) to generate and amplify two or more overlapping cDNA fragments from the cDNA, wherein the two or more overlapping cDNA fragments collectively encode the SARS-CoV-2 variant; substituting one or more overlapping cDNA fragments comprising a modified sequence for one or more corresponding overlapping cDNA fragment generated from the viral RNA; and performing overlapping and amplifying PCR to construct the modified viral genome, wherein the modified viral genome comprises one or more modified sequences. In various embodiments, the method comprises performing at least 1 passage of SARS-CoV-2 variant RNA viral isolate on permissive cells before performing the RT-PCR on the viral RNA from the SARS-CoV-2 variant to generate the cDNA.

Various embodiments of the invention provide for a method of generating a modified viral genome, comprising performing polymerase chain reaction (“PCR”) to generate and amplify two or more overlapping cDNA fragments from cDNA encoding viral RNA from a SARS-CoV-2 variant, wherein the two or more overlapping cDNA fragments collectively encode the SARS-CoV-2 variant, wherein one or more overlapping cDNA fragments comprises a modified sequence; performing overlapping and amplifying PCR to construct the modified viral genome, wherein the modified viral genome comprises one or more modified sequences.

Various embodiments of the invention provide for a method of generating a modified viral genome, comprising performing polymerase chain reaction (“PCR”) to generate and amplify two or more overlapping cDNA fragments from cDNA encoding viral RNA from a SARS-CoV-2 variant, wherein the two or more overlapping cDNA fragments collectively encode the SARS-CoV-2 variant; substituting one or more overlapping cDNA fragments comprising a modified sequence for one or more corresponding overlapping cDNA fragment; performing overlapping and amplifying PCR to construct the modified viral genome, wherein the modified viral genome comprises one or more modified sequences.

In various embodiments, performing PCR to generate and amplify two or more overlapping cDNA fragments from the cDNA comprises using two or more primer pairs, each pair specific for each of the overlapping cDNA fragments. In various embodiments, performing PCR to generate and amplify two or more overlapping cDNA fragments from the cDNA comprises using two or more primer pairs selected from Table 4.

In various embodiments, performing PCR to generate and amplify two or more overlapping cDNA fragments from the cDNA comprises using 5 or more primer pairs, each pair specific for each of the overlapping cDNA fragments. In various embodiments, the two or more overlapping cDNA fragments from the cDNA is 5 or more overlapping cDNA fragments and the 5 or more overlapping cDNA fragments collectively encode the RNA virus. In various embodiments, performing PCR to generate and amplify 5 or more overlapping cDNA fragments from the cDNA comprises using 5 or more primer pairs selected from Table 4.

In various embodiments, performing PCR to generate and amplify two or more overlapping cDNA fragments from the cDNA comprises using 10 or more primer pairs, each pair specific for each of the overlapping cDNA fragments. In various embodiments, the two or more overlapping cDNA fragments from the cDNA is 10 or more overlapping cDNA fragments and the 10 or more overlapping cDNA fragments collectively encode the RNA virus. In various embodiments, performing PCR to generate and amplify 10 or more overlapping cDNA fragments from the cDNA comprises using 10 or more primer pairs selected from Table 4.

In various embodiments, performing PCR to generate and amplify two or more overlapping cDNA fragments from the cDNA comprises using 15 or more primer pairs, each pair specific for each of the overlapping cDNA fragments. In various embodiments, the two or more overlapping cDNA fragments from the cDNA is 15 or more overlapping cDNA fragments and the 15 or more overlapping cDNA fragments collectively encode the RNA virus. In various embodiments, performing PCR to generate and amplify 15 or more overlapping cDNA fragments from the cDNA comprises using 15 or more primer pairs selected from Table 4.

In various embodiments, the two or more overlapping cDNA fragments from the cDNA is 20 or more overlapping cDNA fragments and the 20 or more overlapping cDNA fragments collectively encode the RNA virus. In various embodiments, performing PCR to generate and amplify 20 or more overlapping cDNA fragments from the cDNA comprises using 20 or more primer pairs, each pair specific for each overlapping cDNA fragments.

In various embodiments, the two or more overlapping cDNA fragments from the cDNA is 25 or more overlapping cDNA fragments and the 25 or more overlapping cDNA fragments collectively encode the RNA virus. In various embodiments, performing PCR to generate and amplify 25 or more overlapping cDNA fragments from the cDNA comprises using 25 or more primer pairs, each pair specific for each overlapping cDNA fragments.

In various embodiments, the two or more overlapping cDNA fragments from the cDNA is 19 overlapping cDNA fragments and the 19 overlapping cDNA fragments collectively encode the SARS-CoV-2 variant; for example, Alpha, Beta, Delta, or Gamma as discussed herein. In various embodiments, performing PCR to generate and amplify 19 overlapping cDNA fragments from the first cDNA comprises using all 19 primer pairs from Table 4.

In various embodiments, the length of the overlap is about 40-400 bp. In various embodiments, the length of the overlap is about 200 bp. In various embodiments, the length of the overlap is about 40-100 bp. In various embodiments, the length of the overlap is about 100-200 bp. In various embodiments, the length of the overlap is about 100-150 bp. In various embodiments, the length of the overlap is about 150-200 bp. In various embodiments, the length of the overlap is about 200-250 bp. In various embodiments, the length of the overlap is about 200-300 bp. In various embodiments, the length of the overlap is about 300-400 bp.

In various embodiments, the length of the primers is about 15-55 base pairs (bp) in length. In various embodiments, the length of the primers is about 19-55 bp in length. In various embodiments, the length of the primers is about 10-65 bp in length. In various embodiments, the length of the primers is about 16-20, 21-25, 26-30, 31-35, 36-40, 41-45, 46-50, 51-55, 56-60, or 61-65 bp in length.

In various embodiments, performing overlapping PCR to construct the deoptimized viral genome is done on the two or more overlapping cDNA fragments at the same time. Thus, ifthere are 5 more overlapping cDNA fragments, overlapping PCR to construct the deoptimized viral genome is done on those 5 fragments at the same time. As further examples, if there are 8 more overlapping cDNA fragments, overlapping PCR to construct the deoptimized viral genome is done on those 8 fragments at the same time; if there are 10 more overlapping cDNA fragments, overlapping PCR to construct the deoptimized viral genome is done on those 10 fragments at the same time; if there are 15 more overlapping cDNA fragments, overlapping PCR to construct the deoptimized viral genome is done on those 15 fragments at the same time; if there are 19 more overlapping cDNA fragments, overlapping PCR to construct the deoptimized viral genome is done on those 19 fragments at the same time if there are 20 more overlapping cDNA fragments, overlapping PCR to construct the deoptimized viral genome is done on those 20 fragments at the same time; if there are 25 more overlapping cDNA fragments, overlapping PCR to construct the deoptimized viral genome is done on those 25 fragments at the same time; if there are 30 more overlapping cDNA fragments, overlapping PCR to construct the deoptimized viral genome is done on those 30 fragments at the same time.

In various embodiments, the methods do not use an intermediate DNA clone, such as a plasmid, BAC or YAC. In various embodiments, the methods do not use a cloning host. In various embodiments, the methods do not include an artificial intron in the sequences; for example, to disrupt an offending sequence locus.

### Attenuated Virus Generation

Various embodiments of the present invention provide for a method of generating a modified SARS-CoV-2 variant.

In various embodiments, the method comprises: transfection a population of cells with a vector comprising the viral genome of the present invention; passaging the population of cells in a cell culture at least one time; collecting supernatant from cell culture.

In various embodiments, the method comprises: transfecting a population of cells with a modified infectious SARS-CoV-2 variant RNA of the present invention; culturing the population of cells; and collecting infection medium comprising the modified SARS-CoV-2 variant. In various embodiments, culturing the population of cells comprising passaging the population of cells in a cell culture one or more times.

In various embodiments, the method further comprises concentrating the supernatant or the infection medium.

In various embodiments, the method comprises passaging the population of cells 2 to 15 times; and collecting supernatant from the cell culture of the population of cells. In various embodiments, the method comprises passaging the population of cells 2 to 10 times; and collecting supernatant from the cell culture of the population of cells. In various embodiments, the method comprises passaging the population of cells 2 to 7 times; and collecting supernatant from the cell culture of the population of cells. In various embodiments, the method comprises passaging the population of cells 2 to 5 times; and collecting supernatant from the cell culture of the population of cells. In various embodiments, the method comprises passaging the population of cells 2, 3, 4, 5, 6, 7, 8, or 10 times; and collecting supernatant from the cell culture of the population of cells. In various embodiments, collecting supernatant from the cell culture is done during each passage of the population of cells. In other embodiments, collecting supernatant from the cell culture is done during one or more passages of the population of cells. For example, it can be done every other passage; every two passage, every three passage, etc.

Various embodiments of the invention provide for a method of generating a deoptimized SARS-CoV-2 variant, comprising transfecting host cells with a quantity of a deoptimized infectious RNA; culturing the host cells; and collecting infection medium comprising the deoptimized virus.

In various embodiments, the method comprises performing reverse transcription polymerase chain reaction (“RT-PCR”) on a viral RNA from a SARS-CoV-2 variant to generate cDNA; performing polymerase chain reaction (“PCR”) to generate and amplify two or more overlapping cDNA fragments from the cDNA, wherein the two or more overlapping cDNA fragments collectively encode the SARS-CoV-2 variant; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; performing in vitro transcription of a deoptimized viral genome to generate a deoptimized RNA transcript; culturing the host cells; and collecting infection medium comprising the deoptimized virus.

In various embodiments, the method further comprises generating the quantity of deoptimized infectious RNA in accordance with various embodiments of the present invention before transfecting host cells with the quantity of the deoptimized infectious RNA. Thus, the invention comprises performing in vitro transcription of a deoptimized viral genome to generate a deoptimized RNA transcript; and transfecting host cells with a quantity of a deoptimized infectious RNA; culturing the host cells; and collecting infection medium comprising the deoptimized virus.

In other embodiments, the method comprises performing polymerase chain reaction (“PCR”) to generate and amplify two or more overlapping cDNA fragments from cDNA encoding viral RNA from a SARS-CoV-2 variant, wherein the two or more overlapping cDNA fragments collectively encode the SARS-CoV-2 variant, wherein one or more overlapping cDNA fragments comprises a deoptimized sequence; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript; culturing the host cells; and collecting infection medium comprising the deoptimized virus.

In other embodiments, the method comprises performing polymerase chain reaction (“PCR”) to generate and amplify two or more overlapping cDNA fragments from cDNA encoding viral RNA from a SARS-CoV-2 variant, wherein the two or more overlapping cDNA fragments collectively encode the SARS-CoV-2 variant; substituting one or more overlapping cDNA fragments comprising a deoptimized sequence for one or more corresponding overlapping cDNA fragment generated from the viral RNA; performing overlapping and amplifying PCR to construct the deoptimized viral genome, wherein the deoptimized viral genome comprises one or more deoptimized sequences; and performing in vitro transcription of a deoptimized viral genome to generate a deoptimized RNA transcript; culturing the host cells; and collecting infection medium comprising the deoptimized virus.

In various embodiments, the method comprises performing at least 1 passage of wild-type RNA viral isolate on permissive cells before performing the RT-PCR on the viral RNA from the SARS-CoV-2 variant to generate the cDNA.

In various embodiments, the method further comprising extracting the viral RNA from the SARS-CoV-2 variant prior to performing RT-PCR.

Specific embodiments of the modified viral genome and methods of generating the modified viral genome are as provided herein and are included in these embodiments of producing these modified SARS-CoV-2 variants.

### Kits

The present invention is also directed to a kit to vaccinate a subject, to elicit an immune response or to elicit a protective immune response in a subject. The kit is useful for practicing the inventive method of elicit an immune response or to elicit a protective immune response. The kit is an assemblage of materials or components, including at least one of the inventive compositions. Thus, in some embodiments the kit contains a composition including any one of the modified SARS-CoV-2 variant discussed herein, any one of the immune compositions discussed herein, or any one of the vaccine compositions discussed herein of the present invention. Thus, in some embodiments the kit contains unitized single dosages of the composition including the modified SARS-CoV-2 variant, the immune compositions, or the vaccine compositions of the present invention as described herein; for example, each vial contains enough for a dose of about 10³-10⁷ PFU of the modified SARS-CoV-2 variant, or more particularly, 10⁴-10⁶ PFU of the modified SARS-CoV-2 variant, 10⁴ PFU of the modified SARS-CoV-2 variant, 10⁵ PFU of the modified SARS-CoV-2 variant, or 10⁶ PFU of the modified SARS-CoV-2 variant; or more particularly, 5×10⁴-5×10⁶ PFU of the modified SARS-CoV-2 variant, 5×10⁴ PFU of the modified SARS-CoV-2 variant, 5×10⁵ PFU of the modified SARS-CoV-2 variant, or 5×10⁶ PFU of the modified SARS-CoV-2 variant, or 5×10⁷ PFU of the modified SARS-CoV-2 variant. In various embodiments, the kit contains multiple dosages of the composition including the modified SARS-CoV-2 variant, the immune compositions, the vaccine compositions, multivalent immune compositions, or multivalent vaccine compositions of the present invention as described herein; for example, if the kit contains 10 dosages per vial, each vial contains about 10×10³-10⁷ PFU of the modified SARS-CoV-2 variant, or more particularly, 10×10⁴-10⁶ PFU of the modified SARS-CoV-2 variant, 10×10⁴ PFU of the modified SARS-CoV-2 variant, 10×10⁵ PFU of the modified SARS-CoV-2 variant, or 10×10⁶ PFU of the modified SARS-CoV-2 variant, or more particularly, 50×10⁴-50×10⁶ PFU of the modified SARS-CoV-2 variant, 50×10⁴ PFU of the modified SARS-CoV-2 variant, 50×10⁵ PFU of the modified SARS-CoV-2 variant, or 50×10⁶ PFU of the modified SARS-CoV-2 variant, or 50×10⁷ PFU of the modified SARS-CoV-2 variant.

The exact nature of the components configured in the inventive kit depends on its intended purpose. For example, some embodiments are configured for the purpose of vaccinating a subject, for eliciting an immune response or for eliciting a protective immune response in a subject. In one embodiment, the kit is configured particularly for the purpose of prophylactically treating mammalian subjects. In another embodiment, the kit is configured particularly for the purpose of prophylactically treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to vaccinate a subject, to elicit an immune response or to elicit a protective immune response in a subject. For example, for nasal administration, instructions for use can include but are not limited to instructions for the subject to blow the nose and tilt the head back, instructions for the subject reposition the head to avoid having composition dripping outside of the nose or down the throat, instructions for administering about 0.25 mL comprising the dosage into each nostril; instructions for the subject to sniff gently, and/or instructions for the subject to not blow the nose for a period of time; for example, about 60 minutes. Further instructions can include instruction for the subject to not take any immunosuppressive medications

Optionally, the kit also contains other useful components, such as, diluents, buffers, pharmaceutically acceptable carriers, syringes, droppers, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by known methods, preferably to provide a sterile, contaminant-free environment. The packaging materials employed in the kit are those customarily utilized in vaccines. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing modified SARS-CoV-2 variant, the immune compositions, or the vaccine compositions of the present invention as described herein. The packaging material generally has an external label which indicates the contents and/or purpose of the kit and/or its components.

Sequences
(deoptimized in reference to Washington isolate (GenBank: MN985325.1), with
a 36 nucleotide deletion in the spike protein and without a polyA tail).
SEQ ID NO: 1
attaaaggtttataccttcccaggtaacaaaccaaccaactttcgatctcttgtagatctgttctctaaacgaactttaaaatctgtgtggctgtcactcggct
gcatgcttagtgcactcacgcagtataattaataactaattactgtcgttgacaggacacgagtaactcgtctatcttctgcaggctgcttacggtttcgtccg
tgttgcagccgatcatcagcacatctaggtttcgtccgggtgtgaccgaaaggtaagatggagagccttgtccctggtttcaacgagaaaacacacgtccaact
cagtttgcctgttttacaggttcgcgacgtgctcgtacgtggctttggagactccgtggaggaggtcttatcagaggcacgtcaacatcttaaagatggcactt
gtggcttagtagaagttgaaaaaggcgttttgcctcaacttgaacagccctatgtgttcatcaaacgttcggatgctcgaactgcacctcatggtcatgttatg
gttgagctggtagcagaactcgaaggcattcagtacggtcgtagtggtgagacacttggtgtccttgtccctcatgtgggcgaaataccagtggcttaccgcaa
ggttcttcttcgtaagaacggtaataaaggagctggtggccatagttacggcgccgatctaaagtcatttgacttaggcgacgagcttggcactgatccttatg
aagattttcaagaaaactggaacactaaacatagcagtggtgttacccgtgaactcatgcgtgagcttaacggaggggcatacactcgctatgtcgataacaac
ttctgtggccctgatggctaccctcttgagtgcattaaagaccttctagcacgtgctggtaaagcttcatgcactttgtccgaacaactggactttattgacac
taagaggggtgtatactgctgccgtgaacatgagcatgaaattgcttggtacacggaacgttctgaaaagagctatgaattgcagacaccttttgaaattaaat
tggcaaagaaatttgacaccttcaatggggaatgtccaaattttgtatttcccttaaattccataatcaagactattcaaccaagggttgaaaagaaaaagctt
gatggctttatgggtagaattcgatctgtctatccagttgcgtcaccaaatgaatgcaaccaaatgtgcctttcaactctcatgaagtgtgatcattgtggtga
aacttcatggcagacgggcgattttgttaaagccacttgcgaattttgtggcactgagaatttgactaaagaaggtgccactacttgtggttacttaccccaaa
atgctgttgttaaaatttattgtccagcatgtcacaattcagaagtaggacctgagcatagtcttgccgaataccataatgaatctggcttgaaaaccattctt
cgtaagggtggtcgcactattgcctttggaggctgtgtgttctcttatgttggttgccataacaagtgtgcctattgggttccacgtgctagcgctaacatagg
ttgtaaccatacaggtgttgttggagaaggttccgaaggtcttaatgacaaccttcttgaaatactccaaaaagagaaagtcaacatcaatattgttggtgact
ttaaacttaatgaagagatcgccattattttggcatctttttctgcttccacaagtgcttttgtggaaactgtgaaaggtttggattataaagcattcaaacaa
attgttgaatcctgtggtaattttaaagttacaaaaggaaaagctaaaaaaggtgcctggaatattggtgaacagaaatcaatactgagtcctctttatgcatt
tgcatcagaggctgctcgtgttgtacgatcaattttctcccgcactcttgaaactgctcaaaattctgtgcgtgttttacagaaggccgctataacaatactag
atggaatttcacagtattcactgagactcattgatgctatgatgttcacatctgatttggctactaacaatctagttgtaatggcctacattacaggtggtgtt
gttcagttgacttcgcagtggctaactaacatctttggcactgtttatgaaaaactcaaacccgtccttgattggcttgaagagaagtttaaggaaggtgtaga
gtttcttagagacggttgggaaattgttaaatttatctcaacctgtgcttgtgaaattgtcggtggacaaattgtcacctgtgcaaaggaaattaaggagagtg
ttcagacattctttaagcttgtaaataaatttttggctttgtgtgctgactctatcattattggtggagctaaacttaaagccttgaatttaggtgaaacattt
gtcacgcactcaaagggattgtacagaaagtgtgttaaatccagagaagaaactggcctactcatgcctctaaaagccccaaaagaaattatcttcttagaggg
agaaacacttcccacagaagtgttaacagaggaagttgtcttgaaaactggtgatttacaaccattagaacaacctactagtgaagctgttgaagctccattgg
ttggtacaccagtttgtattaacgggcttatgttgctcgaaatcaaagacacagaaaagtactgtgcccttgcacctaatatgatggtaacaaacaataccttc
acactcaaaggcggtgcaccaacaaaggttacttttggtgatgacactgtgatagaagtgcaaggttacaagagtgtgaatatcacttttgaacttgatgaaag
gattgataaagtacttaatgagaagtgctctgcctatacagttgaactcggtacagaagtaaatgagttcgcctgtgttgtggcagatgctgtcataaaaactt
tgcaaccagtatctgaattacttacaccactgggcattgatttagatgagtggagtatggctacatactacttatttgatgagtctggtgagtttaTattggct
tcacatatgtattgttctttctaccctccagatgaggatgaagaagaaggtgattgtgaagaagaagagtttgagccatcaactcaatatgagtatggtactga
agatgattaccaaggtaaacctttggaatttggtgccacttctgctgctcttcaacctgaagaagagcaagaagaagattggttagatgatgatagtcaacaaa
ctgttggtcaacaagacggcagtgaggacaatcagacaactactattcaaacaattgttgaggttcaacctcaattagagatggaacttacaccagttgttcag
actattgaagtgaatagttttagtggttatttaaaacttactgacaatgtatacattaaaaatgcagacattgtggaagaagctaaaaaggtaaaaccaacagt
ggttgttaatgcagccaatgtttaccttaaacatggaggaggtgttgcaggagccttaaataaggctactaacaatgccatgcaagttgaatctgatgattaca
tagctactaatggaccacttaaagtgggtggtagttgtgttttaagcggacacaatcttgctaaacactgtcttcatgttgtcggcccaaatgttaacaaaggt
gaagacattcaacttcttaagagtgcttatgaaaattttaatcagcacgaagttctacttgcaccattattatcagctggtatttttggtgctgaccctataca
ttctttaagagtttgtgtagatactgttcgcacaaatgtctacttagctgtctttgataaaaatctctatgacaaacttgtttcaagctttttggaaatgaaga
gtgaaaagcaagttgaacaaaagatcgctgagattcctaaagaggaagttaagccatttataactgaaagtaaaccttcagttgaacagagaaaacaagatgat
aagaaaatcaaagcttgtgttgaagaagttacaacaactctggaagaaactaagttcctcacagaaaacttgttactttatattgacattaatggcaatcttca
tccagattctgccactcttgttagtgacattgacatcactttcttaaagaaagatgctccatatatagtgggtgatgttgttcaagagggtgttttaactgctg
tggttatacctactaaaaaggctggtggcactactgaaatgctagcgaaagctttgagaaaagtgccaacagacaattatataaccacttacccgggtcagggt
ttaaatggttacactgtagaggaggcaaagacagtgcttaaaaagtgtaaaagtgccttttacattctaccatctattatctctaatgagaagcaagaaattct
tggaactgtttcttggaatttgcgagaaatgcttgcacatgcagaagaaacacgcaaattaatgcctgtctgtgtggaaactaaagccatagtttcaactatac
agcgtaaatataagggtattaaaatacaagagggtgtggttgattatggtgctagattttacttttacaccagtaaaacaactgtagcgtcacttatcaacaca
cttaacgatctaaatgaaactcttgttacaatgccacttggctatgtaacacatggcttaaatttggaagaagctgctcggtatatgagatctctcaaagtgcc
agctacagtttctgtttcttcacctgatgctgttacagcgtataatggttatcttacttcttcttctaaaacacctgaagaacattttattgaaaccatctcac
ttgctggttcctataaagattggtcctattctggacaatctacacaactaggtatagaatttcttaagagaggtgataaaagtgtatattacactagtaatcct
accacattccacctagatggtgaagttatcacctttgacaatcttaagacacttctttctttgagagaagtgaggactattaaggtgtttacaacagtagacaa
cattaacctccacacgcaagttgtggacatgtcaatgacatatggacaacagtttggtccaacttatttggatggagctgatgttactaaaataaaacctcata
attcacatgaaggtaaaacattttatgttttacctaatgatgacactctacgtgttgaggcttttgagtactaccacacaactgatcctagttttctgggtagg
tacatgtcagcattaaatcacactaaaaagtggaaatacccacaagttaatggtttaacttctattaaatgggcagataacaactgttatcttgccactgcatt
gttaacactccaacaaatagagttgaagtttaatccacctgctctacaagatgcttattacagagcaagggctggtgaagctgctaacttttgtgcacttatct
tagcctactgtaataagacagtaggtgagttaggtgatgttagagaaacaatgagttacttgtttcaacatgccaatttagattcttgcaaaagagtcttgaac
gtggtgtgtaaaacttgtggacaacagcagacaacccttaagggtgtagaagctgttatgtacatgggcacactttcttatgaacaatttaagaaaggtgttca
gataccttgtacgtgtggtaaacaagctacaaaatatctagtacaacaggagtcaccttttgttatgatgtcagcaccacctgctcagtatgaacttaagcatg
gtacatttacttgtgctagtgagtacactggtaattaccagtgtggtcactataaacatataacttctaaagaaactttgtattgcatagacggtgctttactt
acaaagtcctcagaatacaaaggtcctattacggatgttttctacaaagaaaacagttacacaacaaccataaaaccagttacttataaattggatggtgttgt
ttgtacagaaattgaccctaagttggacaattattataagaaagacaattcttatttcacagagcaaccaattgatcttgtaccaaaccaaccatatccaaacg
caagcttcgataattttaagtttgtatgtgataatatcaaatttgctgatgatttaaaccagttaactggttataagaaacctgcttcaagagagcttaaagtt
acatttttccctgacttaaatggtgatgtggtggctattgattataaacactacacaccctcttttaagaaaggagctaaattgttacataaacctattgtttg
gcatgttaacaatgcaactaataaagccacgtataaaccaaatacctggtgtatacgttgtctttggagcacaaaaccagttgaaacatcaaattcgtttgatg
tactgaagtcagaggacgcgcagggaatggataatcttgcctgcgaagatctaaaaccagtctctgaagaagtagtggaaaatcctaccatacagaaagacgtt
cttgagtgtaatgtgaaaactaccgaagttgtaggagacattatacttaaaccagcaaataatagtttaaaaattacagaagaggttggccacacagatctaat
ggctgcttatgtagacaattctagtcttactattaagaaacctaatgaattatctagagtattaggtttgaaaacccttgctactcatggtttagctgctgtta
atagtgtcccttgggatactatagctaattatgctaagccttttcttaacaaagttgttagtacaactactaacatagttacacggtgtttaaaccgtgtttgt
actaattatatgccttatttctttactttattgctacaattgtgtacttttactagaagtacaaattctagaattaaagcatctatgccgactactatagcaaa
gaatactgttaagagtgtcggtaaattttgtctagaggcttcatttaattatttgaagtcacctaatttttctaaactgataaatattataatttggtttttac
tattaagtgtttgcctaggttctttaatctactcaaccgctgctttaggtgttttaatgtctaatttaggcatgccttcttactgtactggttacagagaaggc
tatttgaactctactaatgtcactattgcaacctactgtactggttctataccttgtagtgtttgtcttagtggtttagattctttagacacctatccttcttt
agaaactatacaaattaccatttcatcttttaaatgggatttaactgcttttggcttagttgcagagtggtttttggcatatattcttttcactaggtttttct
atgtacttggattggctgcaatcatgcaattgtttttcagctattttgcagtacattttattagtaattcttggcttatgtggttaataattaatcttgtacaa
atggccccgatttcagctatggttagaatgtacatcttctttgcatcattttattatgtatggaaaagttatgtgcatgttgtagacggttgtaattcatcaac
ttgtatgatgtgttacaaacgtaatagagcaacaagagtcgaatgtacaactattgttaatggtgttagaaggtccttttatgtctatgctaatggaggtaaag
gcttttgcaaactacacaattggaattgtgttaattgtgatacattctgtgctggtagtacatttattagtgatgaagttgcgagagacttgtcactacagttt
aaaagaccaataaatcctactgaccagtcttcttacatcgttgatagtgttacagtgaagaatggttccatccatctttactttgataaagctggtcaaaagac
ttatgaaagacattctctctctcattttgttaacttagacaacctgagagctaataacactaaaggttcattgcctattaatgttatagtttttgatggtaaat
caaaatgtgaagaatcatctgcaaaatcagcgtctgtttactacagtcagcttatgtgtcaacctatactgttactagatcaggcattagtgtctgatgttggt
gatagtgcggaagttgcagttaaaatgtttgatgcttacgttaatacgttttcatcaacttttaacgtaccaatggaaaaactcaaaacactagttgcaactgc
agaagctgaacttgcaaagaatgtgtccttagacaatgtcttatctacttttatttcagcagctcggcaagggtttgttgattcagatgtagaaactaaagatg
ttgttgaatgtcttaaattgtcacatcaatctgacatagaagttactggcgatagttgtaataactatatgctcacctataacaaagttgaaaacatgacaccc
cgtgaccttggtgcttgtattgactgtagtgcgcgtcatattaatgcgcaggtagcaaaaagtcacaacattgctttgatatggaacgttaaagatttcatgtc
attgtctgaacaactacgaaaacaaatacgtagtgctgctaaaaagaataacttaccttttaagttgacatgtgcaactactagacaagttgttaatgttgtaa
caacaaagatagcacttaagggtggtaaaattgttaataattggttgaagcagttaattaaagttacacttgtgttcctttttgttgctgctattttctattta
ataacacctgttcatgtcatgtctaaacatactgacttttcaagtgaaatcataggatacaaggctattgatggtggtgtcactcgtgacatagcatctacaga
tacttgttttgctaacaaacatgctgattttgacacatggtttagtcagcgtggtggtagttatactaatgacaaagcttgcccattgattgctgcagtcataa
caagagaagtgggttttgtcgtgcctggtttgcctggcacgatattacgcacaactaatggtgactttttgcatttcttacctagagtttttagtgcagttggt
aacatctgttacacaccatcaaaacttatagagtacactgactttgcaacatcagcttgtgttttggctgctgaatgtacaatttttaaagatgcttctggtaa
gccagtaccatattgttatgataccaatgtactagaaggttctgttgcttatgaaagtttacgccctgacacacgttatgtgctcatggatggctctattattc
aatttcctaacacctaccttgaaggttctgttagagtggtaacaacCtttgattctgagtactgtaggcacggcacttgtgaaagatcagaagctggtgtttgt
gtatctactagtggtagatgggtacttaacaatgattattacagatctttaccaggagttttctgtggtgtagatgctgtaaatttacttactaatatgtttac
accactaattcaacctattggtgctttggacatatcagcatctatagtagctggtggtattgtagctatcgtagtaacatgccttgcctactattttatgaggt
ttagaagagcttttggtgaatacagtcatgtagttgcctttaatactttactattccttatgtcattcactgtactctgtttaacaccagtttactcattctta
cctggtgtttattctgttatttacttgtacttgacattttatcttactaatgatgtttcttttttagcacatattcagtggatggttatgttcacacctttagt
acctttctggataacaattgcttatatcatttgtatttccacaaagcatttctattggttctttagtaattacctaaagagacgtgtagtctttaatggtgttt
cctttagtacttttgaagaagctgcgctgtgcacctttttgttaaataaagaaatgtatctaaagttgcgtagtgatgtgctattacctcttacgcaatataat
agatacttagctctttataataagtacaagtattttagtggagcaatggatacaactagctacagagaagctgcttgttgtcatctcgcaaaggctctcaatga
cttcagtaactcaggttctgatgttctttaccaaccaccacaaacctctatcacctcagctgttttgcagagtggttttagaaaaatggcattcccatctggta
aagttgagggttgtatggtacaagtaacttgtggtacaactacacttaacggtctttggcttgatgacgtagtttactgtccaagacatgtgatctgcacctct
gaagacatgcttaaccctaattatgaagatttactcattcgtaagtctaatcataatttcttggtacaggctggtaatgttcaactcagggttattggacattc
tatgcaaaattgtgtacttaagcttaaggttgatacagccaatcctaagacacctaagtataagtttgttcgcattcaaccaggacagactttttcagtgttag
cttgttacaatggttcaccatctggtgtttaccaatgtgctatgaggcccaatttcactattaagggttcattccttaatggttcatgtggtagtgttggtttt
aacatagattatgactgtgtctctttttgttacatgcaccatatggaattaccaactggagttcatgctggcacagacttagaaggtaacttttatggaccttt
tgttgacaggcaaacagcacaagcagctggtacggacacaactattacagttaatgttttagcttggttgtacgctgctgttataaatggagacaggtggtttc
tcaatcgatttaccacaactcttaatgactttaaccttgtggctatgaagtacaattatgaacctctaacacaagaccatgttgacatactaggacctctttct
gctcaaactggaattgccgttttagatatgtgtgcttcattaaaagaattactgcaaaatggtatgaatggacgtaccatattgggtagtgctttattagaaga
tgaatttacaccttttgatgttgttagacaatgctcaggtgttactttccaaagtgcagtgaaaagaacaatcaagggtacacaccactggttgttactcacaa
ttttgacttcacttttagttttagtccagagtactcaatggtctttgttcttttttttgtatgaaaatgcctttttaccttttgctatgggtattattgctatg
tctgcttttgcaatgatgtttgtcaaacataagcatgcatttctctgtttgtttttgttaccttctcttgccactgtagcttattttaatatggtctatatgcc
tgctagttgggtgatgcgtattatgacatggttggatatggttgatactagtttgtctggttttaagctaaaagactgtgttatgtatgcatcagctgtagtgt
tactaatccttatgacagcaagaactgtgtatgatgatggtgctaggagagtgtggacacttatgaatgtcttgacactcgtttataaagtttattatggtaat
gctttagatcaagccatttccatgtgggctcttataatctctgttacttctaactactcaggtgtagttacaactgtcatgttCttggccagaggtattgtttt
tatgtgtgttgagtattgccctattttcttcataactggtaatacacttcagtgtataatgctagtttattgtttcttaggctatttttgtacttgttactttg
gcctcttttgtttactcaaccgctactttagactgactcttggtgtttatgattacttagtttctacacaggagtttagatatatgaattcacagggactactc
ccacccaagaatagcatagatgccttcaaactcaacattaaattgttgggtgttggtggcaaaccttgtatcaaagtagccactgtacagtctaaaatgtcaga
tgtaaagtgcacatcagtagtcttactctcagttttgcaacaactcagagtagaatcatcatctaaattgtgggctcaatgtgtccagttacacaatgacattc
tcttagctaaagatactactgaagcctttgaaaaaatggtttcactactttctgttttgctttccatgcagggtgctgtagacataaacaagctttgtgaagaa
atgctggacaacagggcaaccttacaagctatagcctcagagtttagttcccttccatcatatgcagcttttgctactgctcaagaagcttatgagcaggctgt
tgctaatggtgattctgaagttgttcttaaaaagttgaagaagtctttgaatgtggctaaatctgaatttgaccgtgatgcagccatgcaacgtaagttggaaa
agatggctgatcaagctatgacccaaatgtataaacaggctagatctgaggacaagagggcaaaagttactagtgctatgcagacaatgcttttcactatgctt
agaaagttggataatgatgcactcaacaacattatcaacaatgcaagagatggttgtgttcccttgaacataatacctcttacaacagcagccaaactaatggt
tgtcataccagactataacacatataaaaatacgtgtgatggtacaacatttacttatgcatcagcattgtgggaaatccaacaggttgtagatgcagatagta
aaattgttcaacttagtgaaattagtatggacaattcacctaatttagcatggcctcttattgtaacagctttaagggccaattctgctgtcaaattacagaat
aatgagcttagtcctgttgcactacgacagatgtcttgtgctgccggtactacacaaactgcttgcactgatgacaatgcgttagcttactacaacacaacaaa
gggaggtaggtttgtacttgcactgttatccgatttacaggatttgaaatgggctagattccctaagagtgatggaactggtactatctatacagaactggaac
caccttgtaggtttgttacagacacacctaaaggtcctaaagtgaagtatttatactttattaaaggattaaacaacctaaatagaggtatggtacttggtagt
ttagctgccacagtacgtctacaagctggtaatgcaacagaagtgcctgccaattcaactgtattatctttctgtgcttttgctgtagatgctgctaaagctta
caaagattatctagctagtgggggacaaccaatcactaattgtgttaagatgttgtgtacacacactggtactggtcaggcaataacagttacaccggaagcca
atatggatcaagaatcctttggtggtgcatcgtgttgtctgtactgccgttgccacatagatcatccaaatcctaaaggattttgtgacttaaaaggtaagtat
gtacaaatacctacaacttgtgctaatgaccctgtgggttttacacttaaaaacacagtctgtaccgtctgcggtatgtggaaaggttatggctgtagttgtga
tcaactccgcgaacccatgcttcagtcagctgatgcacaatcgtttttaaacgggtttgcggtgtaagtgcagcccgtcttacaccgtgcggcacaggcactag
tactgatgtcgtatacagggcttttgacatctacaatgataaagtagctggttttgctaaattcctaaaaactaattgttgtcgcttccaagaaaaggacgaag
atgacaatttaattgattcttactttgtagttaagagacacactttctctaactaccaacatgaagaaacaatttataatttacttaaggattgtccagctgtt
gctaaacatgacttctttaagtttagaatagacggtgacatggtaccacatatatcacgtcaacgtcttactaaatacacaatggcagacctcgtctatgcttt
aaggcattttgatgaaggtaattgtgacacattaaaagaaatacttgtcacatacaattgttgtgatgatgattatttcaataaaaaggactggtatgattttg
tagaaaacccagatatattacgcgtatacgccaacttaggtgaacgtgtacgccaagctttgttaaaaacagtacaattctgtgatgccatgcgaaatgctggt
attgttggtgtactgacattagataatcaagatctcaatggtaactggtatgatttcggtgatttcatacaaaccacgccaggtagtggagttcctgttgtaga
ttcttattattcattgttaatgcctatattaaccttgaccagggctttaactgcagagtcacatgttgacactgacttaacaaagccttacattaagtgggatt
tgttaaaatatgacttcacggaagagaggttaaaactctttgaccgttattttaaatattgggatcagacataccacccaaattgtgttaactgtttggatgac
agatgcattctgcattgtgcaaactttaatgttttattctctacagtgttcccacctacaagttttggaccactagtgagaaaaatatttgttgatggtgttcc
atttgtagtttcaactggataccacttcagagagctaggtgttgtacataatcaggatgtaaacttacatagctctagacttagttttaaggaattacttgtgt
atgctgctgaccctgctatgcacgctgcttctggtaatctattactagataaacgcactacgtgcttttcagtagctgcacttactaacaatgttgcttttcaa
actgtcaaacccggtaattttaacaaagacttctatgactttgctgtgtctaagggtttctttaaggaaggaagttctgttgaattaaaacacttcttctttgc
tcaggatggtaatgctgctatcagcgattatgactactatcgttataatctaccaacaatgtgtgatatcagacaactactatttgtagttgaagttgttgata
agtactttgattgttacgatggtggctgtattaatgctaaccaagtcatcgtcaacaacctagacaaatcagctggttttccatttaataaatggggtaaggct
agactttattatgattcaatgagttatgaggatcaagatgcacttttcgcatatacaaaacgtaatgtcatccctactataactcaaatgaatcttaagtatgc
cattagtgcaaagaatagagctcgcaccgtagctggtgtctctatctgtagtactatgaccaatagacagtttcatcaaaaattattgaaatcaatagccgcca
ctagaggagctactgtagtaattggaacaagcaaattctatggtggttggcacaacatgttaaaaactgtttatagtgatgtagaaaaccctcaccttatgggt
tgggattatcctaaatgtgatagagccatgcctaacatgcttagaattatggcctcacttgttcttgctcgcaaacatacaacgtgttgtagcttgtcacaccg
tttctatagattagctaatgagtgtgctcaagtattgagtgaaatggtcatgtgtggcggttcactatatgttaaaccaggtggaacctcatcaggagatgcca
caactgcttatgctaatagtgtttttaacatttgtcaagctgtcacggccaatgttaatgcacttttatctactgatggtaacaaaattgccgataagtatgtc
cgcaatttacaacacagactttatgagtgtctctatagaaatagagatgttgacacagactttgtgaatgagttttacgcatatttgcgtaaacatttctcaat
gatgatactctctgacgatgctgttgtgtgtttcaatagcacttatgcatctcaaggtctagtggctagcataaagaactttaagtcagttctttattatcaaa
acaatgtttttatgtctgaagcaaaatgttggactgagactgaccttactaaaggacctcatgaattttgctctcaacatacaatgctagttaaacagggtgat
gattatgtgtaccttccttacccagatccatcaagaatcctaggggccggctgttttgtagatgatatcgtaaaaacagatggtacacttatgattgaacggtt
cgtgtctttagctatagatgcttacccacttactaaacatcctaatcaggagtatgctgatgtctttcatttgtacttacaatacataagaaagctacatgatg
agttaacaggacacatgttagacatgtattctgttatgcttactaatgataacacttcaaggtattgggaacctgagttttatgaggctatgtacacaccgcat
acagtcttacaggctgttggggcttgtgttctttgcaattcacagacttcattaagatgtggtgcttgcatacgtagaccattcttatgttgtaaatgctgtta
cgaccatgtcatatcaacatcacataaattagtcttgtctgttaatccgtatgtttgcaGtgctccaggttgtgatgtcacagatgtgactcaactttacttag
gaggtatgagctattattgtaaatcacataaaccacccattagttttccattgtgtgctaatggacaagtttttggtttatataaaaatacatgtgttggtagc
gataatgttactgactttaatgcaattgcaacatgtgactggacaaatgctggtgattacattttagctaacacctgtactgaaagactcaagctttttgcagc
agaaacgctcaaagctactgaggagacatttaaactgtcttatggtattgctactgtacgtgaagtgctgtctgacagagaattacatctttcatgggaagttg
gtaaacctagaccaccacttaaccgaaattatgtctttactggttatcgtgtaactaaaaacagtaaagtacaaataggagagtacacctttgaaaaaggtgac
tatggtgatgctgttgtttaccgaggtacaacaacttacaaattaaatgttggtgattattttgtgctgacatcacatacagtaatgccattaagtgcacctac
actagtgccacaagagcactatgttagaattactggcttatacccaacactcaatatctcagatgagttttctagcaatgttgcaaattatcaaaaggttggta
tgcaaaagtattctacactccagggaccacctggtactggtaagagtcattttgctattggcctagctctctactacccttctgctcgcatagtgtatacagct
tgctctcatgccgctgttgatgcactatgtgagaaggcattaaaatatttgcctatagataaatgtagtagaattatacctgcacgtgctcgtgtagagtgttt
tgataaattcaaagtgaattcaacattagaacagtatgtcttttgtactgtaaatgcattgcctgagacgacagcagatatagttgtctttgatgaaatttcaa
tggccacaaattatgatttgagtgttgtcaatgccagattacgtgctaagcactatgtgtacattggcgaccctgctcaattacctgcaccacgcacattgcta
actaagggcacactagaaccagaatatttcaattcagtgtgtagacttatgaaaactataggtccagacatgttcctcggaacttgtcggcgttgtcctgctga
aattgttgacactgtgagtgctttggtttatgataataagcttaaagcacataaagacaaatcagctcaatgctttaaaatgttttataagggtgttatcacgc
atgatgtttcatctgcaattaacaggccacaaataggcgtggtaagagaattccttacacgtaaccctgcttggagaaaagctgtctttatttcaccttataat
tcacagaatgctgtagcctcaaagattttgggactaccaactcaaactgttgattcatcacagggctcagaatatgactatgtcatattcactcaaaccactga
aacagctcactcttgtaatgtaaacagatttaatgttgctattaccagagcaaaagtaggcatactttgcataatgtctgatagagacctttatgacaagttgc
aatttacaagtcttgaaattccacgtaggaatgtggcaactttacaagctgaaaatgtaacaggactttttaaagattgtagtaaggtaatcactgggttacat
cctacacaggcacctacacacctcagtgttgacactaaattcaaaactgaaggtttatgtgttgacatacctggcatacctaaggacatgacctatagaagact
catctctatgatgggttttaaaatgaattatcaagttaatggttaccctaacatgtttatcacccgcgaagaagctataagacatgtacgtgcatggattggct
tcgatgtcgaggggtgtcatgctactagagaagctgttggtaccaatttacctttacagctaggtttttctacaggtgttaacctagttgctgtacctacaggt
tatgttgatacacctaataatacagatttttccagagttagtgctaaaccaccgcctggagatcaatttaaacacctcataccacttatgtacaaaggacttcc
ttggaatgtagtgcgtataaagattgtacaaatgttaagtgacacacttaaaaatctctctgacagagtcgtatttgtcttatgggcacatggctttgagttga
catctatgaagtattttgtgaaaataggacctgagcgcacctgttgtctatgtgatagacgtgccacatgcttttccactgcttcagacacttatgcctgttgg
catcattctattggatttgattacgtctataatccgtttatgattgatgttcaacaatggggttttacaggtaacctacaaagcaaccatgatctgtattgtca
agtccatggtaatgcacatgtagctagttgtgatgcaatcatgactaggtgtctagctgtccacgagtgctttgttaagcgtgttgactggactattgaatatc
ctataattggtgatgaactgaagattaatgcggcttgtagaaaggttcaacacatggttgttaaagctgcattattagcagacaaattcccagttcttcacgac
attggtaaccctaaagctattaagtgtgtacctcaagctgatgtagaatggaagttctatgatgcacagccttgtagtgacaaagcttataaaatagaagaatt
attctattcttatgccacacattctgacaaattcacagatggtgtatgcctattttggaattgcaatgtcgatagatatcctgctaattccattgtttgtagat
ttgacactagagtgctatctaaccttaacttgcctggttgtgatggtggcagtttgtatgtaaataaacatgcattccacacaccagcttttgataaaagtgct
tttgttaatttaaaacaattaccatttttctattactctgacagtccatgtgagtctcatggaaaacaagtagtgtcagatatagattatgtaccactaaagtc
tgctacgtgtataacacgttgcaatttaggtggtgctgtctgtagacatcatgctaatgagtacagattgtatctcgatgcttataacatgatgatctcagctg
gctttagcttgtgggtttacaaacaatttgatacttataacctctggaacacttttacaagacttcagagtttagaaaatgtggcttttaatgttgtaaataag
ggacactttgatggacaacagggtgaagtaccagtttctatcattaataacactgtttacacaaaagttgatggtgttgatgtagaattgtttgaaaataaaac
aacattacctgttaatgtagcatttgagctttgggctaagcgcaacattaaaccagtaccagaggtgaaaatactcaataatttgggtgtggacattgctgcta
atactgtgatctgggactacaaaagagatgctccagcacatatatctactattggtgtttgttctatgactgacatagccaagaaaccaactgaaacgatttgt
gcaccactcactgtcttttttgatggtagagttgatggtcaagtagacttatttagaaatgcccgtaatggtgttcttattacagaaggtagtgttaaaggttt
acaaccatctgtaggtcccaaacaagctagtcttaatggagtcacattaattggagaagccgtaaaaacacagttcaattattataagaaagttgatggtgttg
tccaacaattacctgaaacttactttactcagagtagaaatttacaagaatttaaacccaggagtcaaatggaaattgatttcttagaattagctatggatgaa
ttcattgaacggtataaattagaaggctatgccttcgaacatatcgtttatggagattttagtcatagtcagttaggtggtttacatctactgattggactagc
taaacgttttaaggaatcaccttttgaattagaagattttattcctatggacagtacagttaaaaactatttcataacagatgcgcaaacaggttcatctaagt
gtgtgtgttctgttattgatttattacttgatgattttgttgaaataataaaatcccaagatttatctgtagtttctaaggttgtcaaagtgactattgactat
acagaaatttcatttatgctttggtgtaaagatggccatgtagaaacattttacccaaaattacaatctagtcaagcgtggcaaccgggtgttgctatgcctaa
tctttacaaaatgcaaagaatgctattagaaaagtgtgaccttcaaaattatggtgatagtgcaacattacctaaaggcataatgatgaatgtcgcaaaatata
ctcaactgtgtcaatatttaaacacattaacattagctgtaccctataatatgagagttatacattttggtgctggttctgataaaggagttgcaccaggtaca
gctgttttaagacagtggttgcctacgggtacgctgcttgtcgattcagatcttaatgactttgtctctgatgcagattcaactttgattggtgattgtgcaac
tgtacatacagctaataaatgggatctcattattagtgatatgtacgaccctaagactaaaaatgttacaaaagaaaatgactctaaagagggttttttcactt
acatttgtgggtttatacaacaaaagctagctcttggaggttccgtggctataaagataacagaacattcttggaatgctgatctttataagctcatgggacac
ttcgcatggtggacagcctttgttactaatgtgaatgcgtcatcatctgaagcatttttaattggatgtaattatcttggcaaaccacgcgaacaaatagatgg
ttatgtcatgcatgcaaattacatattttggaggaatacaaatccaattcagttgtcttcctattctttatttgacatgagtaaatttccccttaaattaaggg
gtactgctgttatgtctttaaaagaaggtcaaatcaatgatatgattttatctcttcttagtaaaggtagacttataattagagaaaacaacagagttgttatt
tctagtgatgttcttgttaacaactaaacgaacaatgtttgtttttcttgttttattgccactagtctctagtcagtgtgttaatcttacaaccagaactcaat
taccccctgcatacactaattctttcacacgtggtgtttattaccctgacaaagttttcagatcctcagttttacattcaactcaggacttgttcttacctttc
ttttccaatgttacttggttccatgctatacatgtctctgggaccaatggtactaagaggtttgataaccctgtcctaccatttaatgatggtgtttattttgc
ttccaTtgagaagtctaacataataagaggctggatttttggtactactttagattcgaagacccagtccctacttattgttaataacgctactaatgttgtta
ttaaagtctgtgaatttcaattttgtaatgatccatttttgggtgtttattaccacaaaaacaacaaaagttggatggaaagtgagttcagagtttattctagt
gcgaataattgcacttttgaatatgtctctcagccttttcttatggaccttgaaggaaaacagggtaatttcaaaaatcttagggaatttgtgtttaagaatat
tgatggttattttaaaatatattctaagcacacgcctattaatttagtgcgtgatctccctcagggtttttcggctttagaaccattggtagatttgccaatag
gtattaacatcactaggtttcaaactttacttgctttacGtagaagttatttgactcctggtgattcttcttcaggttggacagctggtgctgcagcttattat
gtgggttatcttcaacctaggacttttctattaaaatataatgaaaatggaaccattacagatgctgtagactgtgcacttgaccctctctcagaaacaaagtg
tacgttgaaatccttcactgtagaaaaaggaatctatcaaacttctaactttagagtccaaccaacagaatctattgttagatttcctaatattacaaacttgt
gcccttttggtgaagtttttaacgccaccagatttgcatctgtttatgcttggaacaggaagagaatcagcaactgtgttgctgattattctgtcctatataat
tccgcatcattttccacttttaagtgttatggagtgtctcctactaaattaaatgatctctgctttactaatgtctatgcagattcatttgtaattagaggtga
tgaagtcagacaaatcgctccagggcaaactggaaagattgctgattataattataaattaccagatgattttacaggctgcgttatagcttggaattctaaca
atcttgattctaaggttggtggtaattataattacctgtatagattgtttaggaagtctaatctcaaaccttttgagagagatatttcaactgaaatctatcag
gccggtagcacaccttgtaatggtgttgaaggttttaattgttactttcctttacaatcatatggtttccaacccactaatggtgttggttaccaaccatacag
agtagtagtactttcttttgaacttctacatgcaccagcaactgtttgtggacctaaaaagtctactaatttggttaaaaacaaatgtgtcaatttcaacttca
atggtttaacaggcacaggtgttcttactgagtctaacaaaaagtttctgcctttccaacaatttggcagagacattgctgacactactgatgctgtccgtgat
ccacagacacttgagattcttgacattacaccatgttcttttggtggtgtcagtgttataacaccaggaacaaatacttctaaccaggttgctgttctttatca
ggatgttaactgcacagaagtccctgttgctattcatgcagatcaacttactcctacttggcgtgtttattctacaggttctaatgtttttcaaacacgtgcag
gctgtttaataggggctgaacatgtcaacaactcatatgagtgtgacatacccattggtgcaggtatatgcgctagttatcagactcagcaatccatcattgcc
tacactatgtcacttggtgcagaaaattcagttgcttactctaataactctattgccatacccacaaattttactattagtgttaccacagaaattctaccagt
gtctatgaccaagacatcagtagattgtacaatgtacatttgtggtgattcaactgaatgcagcaatcttttgttgcaatatggcagtttttgtacacaattaa
accgtgctttaactggaatagctgttgaacaagacaaaaacacccaagaagtttttgcacaagtcaaacaaatttacaaaacaccaccaattaaagattttggt
ggttttaatttttcacaaatattaccagatccatcaaaaccaagcaagaggtcatttattgaagatctacttttcaacaaagtgacacttgcagatgctggctt
catcaaacaatatggtgattgccttggtgatattgctgctagagatctcatttgcgctcaaaaatttaacggacttacagttttaccacctttacttactgacg
aaatgattgcgcaatatacatccgcattgttagccggaactattacatccggatggacttttggcgcaggcgTagcattacagattccattcgctatgcaaatg
gcttataggtttaacggtataggcgttacgcaaaacgtactttatgagaatcaaaaacttatcgctaaccaatttaattccgctatcggtaagattcaggattc
attgtctagtactgctagtgcactcggtaagttgcaagacgtagtgaatcaaaacgctcaagcacttaatacactcgttaaacagcttagttctaattttggcg
caatttctagtgtgcttaacgatatactatctagactcgataaagtcgaagccgaagtgcaaatcgatagattgattaccggtaggttgcaatcattgcaaaca
tacgttacacagcaattgattagggccgcagagatacgcgctagcgctaatctcgcagctactaaaatgtctgaatgcgtactcggacaatctaaacgtgtcga
tttttgcggtaagggatatcatcttatgtcttttccacaatctgcacctcacggagtcgtgtttttacacgttacttatgtgccagctcaagagaaaaatttta
caaccgctcctgctatttgtcatgacggtaaggcacattttcctagagagggcgtattcgtttctaacggtacacattggttcgttacacaacgtaatttttac
gaacctcaaattattactactgataatacattcgtatcaggtaattgtgacgtagtgataggtatcgttaataatacagtttacgatccacttcaacctgaact
cgatagttttaaagaggaactcgataagtattttaaaaatcatacatcacctgacgtcgacttaggcgatatttcaggtattaacgctagtgtcgttaacattc
aaaaagagattgatagacttaacgaagtcgctaaaaatcttaacgaatcacttatcgatctgcaagagttaggtaagtatgagcaatatattaaatggccttgg
tatatttggttaggctttatagccggattgatcgcaatcgttatggttacaattatgttatgttgtatgacatcatgttgttcatgtcttaagggatgttgttc
atgcggatcatgttgtaaatttgacgaagacgattccgaaccagtgcttaaaggcgttaagttacattatacataaacgaacttatggatttgtttatgagaat
cttcacaattggaactgtaactttgaagcaaggtgaaatcaaggatgctactccttcagattttgttcgcgctactgcaacgataccgatacaagcctcactcc
ctttcggatggcttattgttggcgttgcacttcttgctgtttttcagagcgcttccaaaatcataaccctcaaaaagagatggcaactagcactctccaagggt
gttcactttgtttgcaacttgctgttgttgtttgtaacagtttactcacaccttttgctcgttgctgctggccttgaagccccttttctctatctttatgcttt
agtctacttcttgcagagtataaactttgtaagaataataatgaggctttggctttgctggaaatgccgttccaaaaacccattactttatgatgccaactatt
ttctttgctggcatactaattgttacgactattgtataccttacaatagtgtaacttcttcaattgtcattacttcaggtgatggcacaacaagtcctatttct
gaacatgactaccagattggtggttatactgaaaaatgggaatctggagtaaaagactgtgttgtattacacagttacttcacttcagactattaccagctgta
ctcaactcaattgagtacagacactggtgttgaacatgttaccttcttcatctacaataaaattgttgatgagcctgaagaacatgtccaaattcacacaatcg
acggttcatccggagttgttaatccagtaatggaaccaatttatgatgaaccgacgacgactactagcgtgcctttgtaagcacaagctgatgagtacgaactt
atgtactcattcgtttcggaagagacaggtacgttaatagttaatagcgtacttctttttcttgctttcgtggtattcttgctagttacactagccatccttac
tgcgcttcgattgtgtgcgtactgctgcaatattgttaacgtgagtcttgtaaaaccttctttttacgtttactctcgtgttaaaaatctgaattcttctagag
ttcctgatcttctggtctaaacgaactaaatattatattagtttttctgtttggaactttaattttagccatggcagattccaacggtactattaccgttgaag
agcttaaaaagctccttgaacaatggaacctagtaataggtttcctattccttacatggatttgtcttctacaatttgcctatgccaacaggaataggtttttg
tatataattaagttaattttcctctggctgttatggccagtaactttagcttgttttgtgcttgctgctgtttacagaataaattggatcaccggtggaattgc
tatcgcaatggcttgtcttgtaggcttgatgtggctcagctacttcattgcttctttcagactgtttgcgcgtacgcgttccatgtggtcattcaatccagaaa
ctaacattcttctcaacgtgccactccatggcactattctgaccagaccgcttctagaaagtgaactcgtaatcggagctgtgatccttcgtggacatcttcgt
attgctggacaccatctaggacgctgtgacatcaaggacctgcctaaagaaatcactgttgctacatcacgaacgctttcttattacaaattgggagcttcgca
gcgtgtagcaggtgactcaggttttgctgcatacagtcgctacaggattggcaactataaattaaacacagaccattccagtagcagtgacaatattgctttgc
ttgtacagtaagtgacaacagatgtttcatctcgttgactttcaggttactatagcagagatattactaattattatgaggacttttaaagtttccatttggaa
tcttgattacatcataaacctcataattaaaaatttatctaagtcactaactgagaataaatattctcaattagatgaagagcaaccaatggagattgattaaa
cgaacatgaaaattattcttttcttggcactgataacactcgctacttgtgagctttatcactaccaagagtgtgttagaggtacaacagtacttttaaaagaa
ccttgctcttctggaacatacgagggcaattcaccatttcatcctctagctgataacaaatttgcactgacttgctttagcactcaatttgcttttgcttgtcc
tgacggcgtaaaacacgtctatcagttacgtgccagatcagtttcacctaaactgttcatcagacaagaggaagttcaagaactttactctccaatttttctta
ttgttgcggcaatagtgtttataacactttgcttcacactcaaaagaaagacagaatgattgaactttcattaattgacttctatttgtgctttttagcctttc
tgctattccttgttttaattatgcttattatcttttggttctcacttgaactgcaagatcataatgaaacttgtcacgcctaaacgaacatgaaatttcttgtt
ttcttaggaatcatcacaactgtagctgcatttcaccaagaatgtagtttacagtcatgtactcaacatcaaccatatgtagttgatgacccgtgtcctattca
cttctattctaaatggtatattagagtaggagctagaaaatcagcacctttaattgaattgtgcgtggatgaggctggttctaaatcacccattcagtacatcg
atatcggtaattatacagtttcctgttcaccttttacaattaattgccaggaacctaaattgggtagtcttgtagtgcgttgttcgttctatgaagacttttta
gagtatcatgacgttcgtgttgttttagatttcatctaaacgaacaaactaaaatgtctgataatggaccccaaaatcagcgaaatgcaccccgcattacgttt
ggtggaccctcagattcaactggcagtaaccagaatggagaacgcagtggggcgcgatcaaaacaacgtcggccccaaggtttacccaataatactgcgtcttg
gttcaccgctctcactcaacatggcaaggaagaccttaaattccctcgaggacaaggcgttccaattaacaccaatagcagtccagatgaccaaattggctact
accgaagagctaccagacgaattcgtggtggtgacggtaaaatgaaagatctcagtccaagatggtatttctactacctaggaactgggccagaagctggactt
ccctatggtgctaacaaagacggcatcatatgggttgcaactgagggagccttgaatacaccaaaagatcacattggcacccgcaatcctgctaacaatgctgc
aatcgtgctacaacttcctcaaggaacaacattgccaaaaggcttctacgcagaagggagcagaggcggcagtcaagcctcttctcgttcctcatcacgtagtc
gcaacagttcaagaaattcaactccaggcagcagtaggggaacttctcctgctagaatggctggcaatggcggtgatgctgctcttgctttgctgctgcttgac
agattgaaccagcttgagagcaaaatgtctggtaaaggccaacaacaacaaggccaaactgtcactaagaaatctgctgctgaggcttctaagaagcctcggca
aaaacgtactgccactaaagcatacaatgtaacacaagctttcggcagacgtggtccagaacaaacccaaggaaattttggggaccaggaactaatcagacaag
gaactgattacaaacattggccgcaaattgcacaatttgcccccagcgcttcagcgttcttcggaatgtcgcgcattggcatggaagtcacaccttcgggaacg
tggttgacctacacaggtgccatcaaattggatgacaaagatccaaatttcaaagatcaagtcattttgctgaataagcatattgacgcatacaaaacattccc
accaacagagcctaaaaaggacaaaaagaagaaggctgatgaaactcaagccttaccgcagagacagaagaaacagcaaactgtgactcttcttcctgctgcag
atttggatgatttctccaaacaattgcaacaatccatgagcagtgctgactcaactcaggcctaaactcatgcagaccacacaaggcagatgggctatataaac
gttttcgcttttccgtttacgatatatagtctactcttgtgcagaatgaattctcgtaactacatagcacaagtagatgtagttaactttaatctcacatagca
atctttaatcagtgtgtaacattagggaggacttgaaagagccaccacattttcaccgaggccacgcggagtacgatcgagtgtacagtgaacaatgctaggga
gagctgcctatatggaagagccctaatgtgtaaaattaattttagtagtgctatccccatgtgattttaatagcttcttaggagaatgac
(recoded spike protein in comparison to USA/WA1/2020 wild-type spike).
SEQ ID NO: 2
MFVFLVLLPLVSSQCVNLTTRTQLPPAYTNSFTRGVYYPDKVFRSSVLHSTQDLFLPFFSNVTWFH
AIHVSGTNGTKRFDNPVLPFNDGVYFASIEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALRRSYLTPGDSSSGWTAGAAA
YYVGYLQPRTFLLKYNENGTITDAVDCALDPLSETKCTLKSFTVEKGIYQTSNFRVQPTESIVRFPN
ITNLCPFGEVFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELLHAPATVCG
PKKSTNLVKNKCVNFNFNGLTGTGVLTESNKKFLPFQQFGRDIADTTDAVRDPQTLEILDITPCSF
GGVSVITPGTNTSNQVAVLYQDVNCTEVPVAIHADQLTPTWRVYSTGSNVFQTRAGCLIGAEHV
NNSYECDIPIGAGICASYQTQQSIIAYTMSLGAENSVAYSNNSIAIPTNFTISVTTEILPVSMTKTSVD
CTMYICGDSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLPPLLTDEMI
AQYTSALLAGTITSGWTFGAGVALQIPFAMQMAYRFNGIGVTQNVLYENQKLIANQFNSAIGKIQ
DSLSSTASALGKLQDVVNQNAQALNTLVKQLSSNFGAISSVLNDILSRLDKVEAEVQIDRLITGRL
QSLQTYVTQQLIRAAEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNLNESLI
DLQELGKYEQYIKWPWYIWLGFIAGLIAIVMVTIMLCCMTSCCSCLKGCCSCGSCCKFDEDDSEP
VLKGVKLHYT

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Synthesis of SARS-CoV-2 Alpha, Variant, Beta Variant and Delta Variant

Synthesis of the Alpha variant, Beta variant and the Delta variant is similar as described for the deoptimized SARS-CoV-2, Coronavirus strain 2019-nCoV USA-WA1/2020 described herein, with exception that the fragments carrying the mutations of each variant were used.

Key mutations for each variant within the Spike gene were identified. About 6-10 sequences of the variant were selected from GISAID and a multi-alignment using BLASTn comparing to our original WT design or CDX-005 (with deoptimization in Spike).

Once the nucleotide mutations were identified, the codons of the Deoptimized Coronavirus strain 2019-nCoV/USA-WA1/2020 design (described herein) were replaced with the codons from the variants. If the mutation resulted in a deletion, the same deletion was made for the deoptimized sequence of the variant.

Thereafter, the DNA fragments carrying these mutations were synthesized. The Spike gene was separated into 3 fragments, herein referred to as F14, F15, and F16. F16 contained the deoptimized regions. Based on the location of the mutations, either 2 or all 3 of these fragments were synthesized.

Briefly, after all 19 fragments were obtained by PCR/RT-PCR process, overlapping PCR was performed to construct the viral genome, followed by in vitro transcription and Vero E6 transfection. The same primer pairs used in the synthesis of the deoptimized SARS-CoV-2 were used in the synthesis of the deoptimized SARS-CoV-2 variants.

### Example 2

### Synthesis of SARS-CoV-2

### Procedures

### RT-PCR

Coronavirus strain 2019-nCoV/USA-WA1/2020 (“WA1”) (BEI Resources NR-52281, Lot 70034262) was distributed by BEI Resources after 3 passages on Vero (CCL81) at CDC, and one passage on Vero E6 at BEI Resources. The full virus genome sequence after 4 passages was determined by CDC and found to contain no nucleotide differences (Harcourt et al., 2020) compared to the clinical specimen from which it was derived (GenBank Accession MN985325) Upon receipt, WA1 and was amplified by a further two passages on Vero E6 cells in DMEM containing 2% FBS at 37° C.

Passage 6 WA1 virus was used to purify viral genome RNA by extraction with Trizol reagent (Thermo Fisher) according to standard protocols. Briefly, 0.5 ml virus sample with a titer of 1×10∂PFU/ml was extracted with an equal volume of Trizol. The procedure had previously been validated in four separate experiment to completely inactivate SARS-CoV2 virus infectivity. After phase separation by addition of 0.1 ml chloroform, the RNA in aqueous phase was precipitated with an equal volume of isopropanol. The precipitated RNA was washed in 70% ethanol, dried, and resuspended in 20 ul RNAse-free water.

Viral cDNA Generation

Wild-type cDNA were synthesized using SuperScript IV First Strand Synthesis system. In each reaction, a total reaction volume of 13 μl for Tube #1 was set up as follows:
1. 50 μM Oligo d(T)20: 1 ul (Alternatively, primer #1822 (10 μM): 1 μl)
2. 50 ng/μl Random Hexamer: 1 μl
3. 10 mM dNTP: 1 μl
4. WT RNA: 2-10 μl
5. H2O: add to 13 μl

The sample was mixed and incubated at 65° C. for 5 minutes, then immediately put on ice for 1 minute. Another tube (Tube #2) was prepared with a total reaction volume of 7 μl:
1. 5× Buffer: 4 μl
2. 100 mM DTT: 1 μl
3. Rnase Inhibitor (40 U/μl): 1 μl (optional)
4. SuperScript IV enzyme: 1 μl

We mixed Tube #1 and Tube #2, for a total reaction volume of 20 μl, and incubated at 23° C. for 10 minutes, followed by 50° C. for 50 minutes, and 80° C. for 10 minutes to generate cDNA.

### Overlapping Polymerase Chain Reaction

Q5 High-Fidelity 2× Master Mixture (NEB, Ipswich, Massachusetts) were used to amplify genome fragments from cDNA.

The 20 μl reaction containing 1 μl fresh-made cDNA, 1 μl of forward and reverse primers (detailed in Table 4) at 0.5 μM concentration, 10 μl of the 2×Q5 master mixture and H₂O. Reaction parameters were as follows: 98° C. 30 sec to initiate the reaction, followed by 30 cycles of 98° C. for 10 sec, 60° C. for 30 seconds, and 65° C. for 1 min and a final extension at 65° C. for 5 min. Totally 19 genome fragments, all about 1.8 Kb except fragment 19 (about 1.2 Kb) were obtained, which cover the whole viral genome with 200 bp overlapping region between any two of them using specific primers (Table 4). Amplicons were verified by agarose gel electrophoresis and purified using the QIAquick PCR Purification Kit (Qiagen). Elutions were quantified by Nanodrop.

Q5® High-Fidelity DNA Polymerase (NEB, Ipswich, Massachusetts) were used to re-construct the whole COVID-19 genome.

First, all 19 genome fragments were used in an overlapping reaction to reconstruct the full genome. Briefly, a mixture with 30-40 ng of each DNA fragment (the molar ratio among all pieces are at 1:1), 10 μl 5× reaction buffer, 1 μl 10 mM dNTP, 0.5 μl Q5 polymerase and H₂O to a final volume of 50 μl was made. The reaction was carried out under following condition: 98° C. for 30 sec, and 72° C. for 16 min 30 sec for 10 cycles.

Next, 2 μl overlapping reaction product were mixed with 4 μl 5× reaction buffer, 1 μl 10 mM dNTP, 1 μl of each flanking primers at 0.5 μM, 0.241 Q5 polymerase and H₂O to a final volume of 20 μl and PCR was carried out as follows: 98° C. 30 sec to initiate the reaction, followed by 15 cycles of 98° C. for 10 sec, 60° C. for 45 sec, and 72° C. for 16 minutes 30 seconds, and a final extension at 65° C. for 5 min. To check the results, 5 μl PCR product was visualized on 0.4% agarose gel.

TABLE 4
Primers for RT-PCR

| SEQ | | | |
|---|---|---|---|
| ID | | | |
| NO: | No. | Name | oligo sequence 5′-3′ |
| 15 | 2312 | 2312-Fr1- | GAtaatacgactcactatagATTAAAGG |
| | | T7G-F3 | TTTATACCTTCCCAGGTAAC |
| 16 | 1786 | 1786-COV-2 | GATGCCAAAATAATGGCGATCTC |
| 17 | 1787 | 1787-COV-3 | GTTGGTTGCCATAACAAGTGTG |
| 18 | 1788 | 1788-COV-4 | CTAATTGAGGTTGAACCTCAACAATTG |
| 19 | 1789 | 1789-COV-5 | GAGTATGGTACTGAAGATGATTACCAAG |
| 20 | 1790 | 1790-COV-6 | CTAGGTGGAATGTGGTAGGATTAC |
| 21 | 1791 | 1791-COV-7 | GCTGTTACAGCGTATAATGGTTATCTTA |
| | | | C |
| 22 | 1792 | 1792-COV-8 | GCTGGTTTAAGTATAATGTCTCCTACAA |
| | | | C |
| 23 | 1793 | 1793-COV-9 | GCACAAAACCAGTTGAAACATCAAATTC |
| 24 | 1794 | 1794-COV-10 | GCAACTAGTGTTTTGAGTTTTTCCATTG |
| 25 | 1795 | 1795-COV-11 | GTGAAGAATCATCTGCAAAATCAGC |
| 26 | 1796 | 1796-COV-12 | CAAATGATATAAGCAATTGTTATCCAGA |
| | | | AAGG |
| 27 | 1797 | 1797-COV-13 | GCCTTTAATACTTTACTATTCCTTATGT |
| | | | CATTCAC |
| 28 | 1798 | 1798-COV-14 | CCAGACAAACTAGTATCAACCATATCC |
| 29 | 1799 | 1799-COV-15 | GCTATGGGTATTATTGCTATGTCTG |
| 30 | 1800 | 1800-COV-16 | CCTACAAGGTGGTTCCAGTTC |
| 31 | 1801 | 1801-COV-17 | CGACAGATGTCTTGTGCTG |
| 32 | 1802 | 1802-COV-18 | GGTATCCAGTTGAAACTACAAATGG |
| 33 | 1803 | 1803-COV-19 | GATCAGACATACCACCCAAATTG |
| 34 | 1804 | 1804-COV-20 | CTTATGTATTGTAAGTACAAATGAAAGA |
| | | | CATCAG |
| 35 | 1805 | 1805-COV-21 | GGTGATGATTATGTGTACCTTCCTTAC |
| 36 | 1806 | 1806-COV-22 | CTGTTAATTGCAGATGAAACATCATGC |
| 37 | 1807 | 1807-COV-23 | GTGTGTAGACTTATGAAAACTATAGGTC |
| | | | C |
| 38 | 1808 | 1808-COV-24 | CATACAAACTGCCACCATCAC |
| 39 | 1809 | 1809-COV-25 | CCTTGTAGTGACAAAGCTTATAAAATAG |
| | | | AAG |
| 40 | 1810 | 1810-COV-26 | CTGGTGCAACTCCTTTATCAG |
| 41 | 1811 | 1811-COV-27 | GCAAAGAATGCTATTAGAAAAGTGTGAC |
| 42 | 1812 | 1812-COV-28 | GATAGATTCCTTTTTCTACAGTGAAGGA |
| | | | TTTC |
| 43 | 1813 | 1813-COV-29 | GACTCCTGGTGATTCTTCTTCAG |
| 44 | 1814 | 1814-COV-30 | CTCTAGCAGCAATATCACCAAGG |
| 45 | 1815 | 1815-COV-31 | GCACAAGTCAAACAAATTTACAAAACAC |
| 46 | 1816 | 1816-COV-32 | CAAAAGGTGTGAGTAAACTGTTACAAAC |
| 47 | 1817 | 1817-COV-33 | CTCACTCCCTTTCGGATGG |
| 48 | 1818 | 1818-COV-34 | GAGGTTTATGATGTAATCAAGATTCCAA |
| | | | ATGG |
| 49 | 1819 | 1819-COV-35 | GCTACAGGATTGGCAACTATAAATTAAA |
| | | | C |
| 50 | 1820 | 1820-COV-36 | CCATTCTAGCAGGAGAAGTTCC |
| 51 | 1821 | 1821-COV-37 | GCAATCCTGCTAACAATGCTG |
| 52 | 1822 | 1822-COV-38 | ttttTTTTTTTTTTTTTTTTTTTTTGTC |
| | | | ATTCTCCTAAGAAGCTATTAAAATC |

### In Vitro Transcription

DNA templates amplified from full-length PCR were purified using conventional phenol/chloroform extraction followed by Ethanol precipitation in the presence of 3M Sodium Acetate prior to RNA work. RNA transcripts was in vitro synthesized using the HiScribe T7 Transcription Kit (New England Biolabs) according to the manufacturer's instruction with some modifications. A 20 μl reaction was set up by adding 500 ng DNA template and 2.4 μl 50 mM GTP (cap analog-to-GTP ratio is 1:1). The reaction was incubated at 37° C. for 3 hr. Then RNA was precipitated and purified by Lithium Chloride precipitation and washed once with 70% Ethanol. The N gene DNA template was also prepared by PCR from cDNA using specific forward primer (2320-N-F: GAAtaatacgactcactataggGACGTTCGTGTTGTTTTAGATTTCATCTAAACG (SEQ ID NO:53), the lowercase sequence represents T7 promoter; the underlined sequence represents the 5′ NTR upstream of the N gene ORF) and reverse primer (2130-N-R, tttttttGTCATTCTCCTAAGAAGCTATTAAAATCACATGG (SEQ ID NO:54)).

### Transfection of Vero E6 Cells by RNA Electroporation

Vero E6 cells were obtained from ATCC (CRL-1586) and maintained in DMEM high glucose supplemented with 10% FBS. To transfect viral RNA, 10 μg of purified full length genome RNA transcripts, together with 5 ug of capped WA1-N mRNA, were electroporated into Vero E6 cells using the Maxcyte ATX system according manufacturer's instructions. Briefly, 3-4×10⁶ Vero E6 cells were once washed in Maxcyte electroporation buffer and resuspended in 100 μl of the same. The cell suspension was mixed gently with the RNA sample, and the RNA/cell mixture transferred to Maxcyte OC-100 processing assemblies. Electroporation was performed using the pre-programmed Vero cell electroporation protocol. After 30 minutes recovery of the transfected cells at 37° C./5% CO₂, cells were resuspended in warm DMEM/10% FBS and distributed among three T25 flasks at various seeding densities (1/2, 1/3, 1/6 of the total cells). Transfected cells were incubated at 37° C./5% CO₂ for 6 days or until CPE appeared. Infection medium was collected on days 2, 4, and 6, with completely media change at day 2 and day 4 (DMEM/5% FBS). The generated viruses were detectable by plaque assay as early as 2 days post transfection, with peak virus generation between days 4-6.

### Passaging of Stock Virus and Plaque Titration of SARS-CoV-2 in Vero E6 Cells

Serial 10-fold dilutions were prepared in DMEM/2% FBS. 0.5 ml of each dilution were added to 12-wells of Vero E6 cells that were 80% confluent. After 1 hour incubation at 37° C., the inoculum was removed, and 2 ml of semisolid overlay was added per well, containing 1×DMEM, 0.3% Gum Tragacanth, 2% FBS and 1× Penicillin/Streptomycin. After 3 or 4 day incubation at 37° C./5% CO₂ the overlay was removed, wells were rinsed gently with PBS, followed by fixation and staining with Crystal Violet.

RNA obtained from in vitro transcription was used to transfect Vero E6 cells with wt WA1 and CDX-005 and recover live virus that was titrated in Vero E6 cells. After incubation for 3 days, plaque assays were stained.

### Multistep Virus Growth Kinetics

Vero cells (WHO 10-87) were grown for 3 days in 12 well plates containing 1 ml DMEM with 5% fetal bovine serum (FBS) until they reached near confluency. Prior to infection, spent cell culture medium was replaced with 0.5 ml fresh DMEM containing 1% FBS and 30 PFU of the indicated viruses (0.0001 MOI). After a 1-hour incubation at 33° C. or 37° C./5% CO2, inoculum was discarded, cell monolayers were washed once with 1 ml Dulbecco's PBS, followed by addition of 1 ml DMEM containing 1% FBS. Infected cells were incubated at 33° C. or 37° C. for 0, 6, 24, 48, or 72 hrs. At the indicated timepoints, cells and supernatants were collected (one well per time point), frozen once at −80 C and thawed. Infectious virus titers in the lysates were determined by plaque assay on Vero E6 at 37° C.

### Results

Generation of individual genome fragments 1-19 and the whole genomic DNA generated by overlapping PCR went well, with clear bands visible on 0.4% agarose gels.

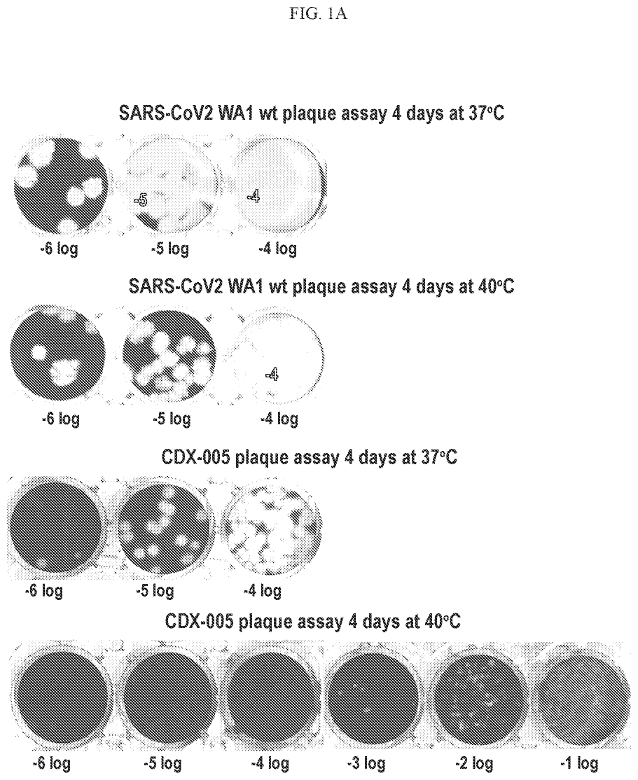

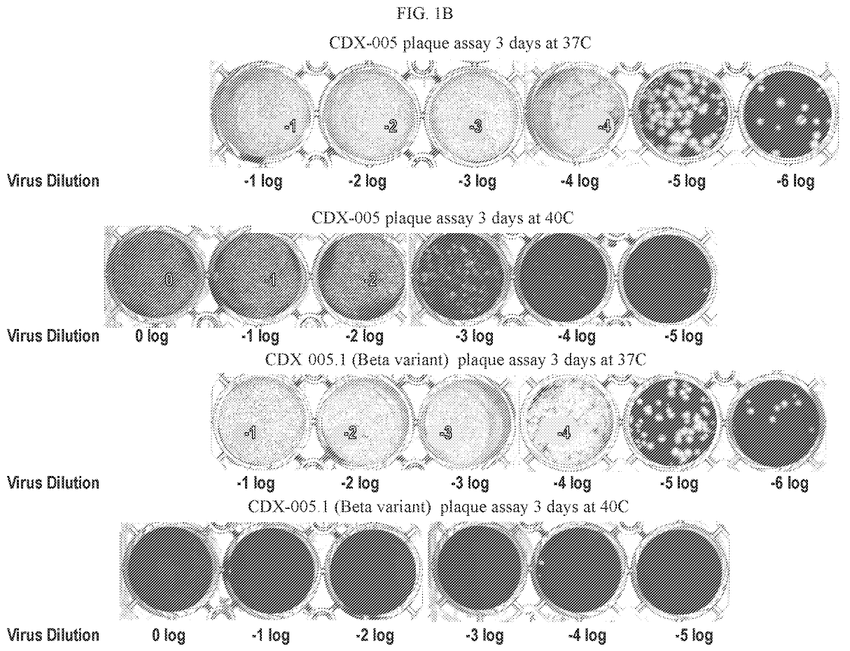

In vitro transcription produced RNA used to transfect Vero E6 cells with S-WWW (WT) and S-WWD and recover live virus that was titrated in Vero E6 cells. After incubation for 3 days, the plaque assays were stained and we observed smaller plaques observed in the partially spike-deoptimized S-WWD candidate (FIG. 1) and a 40% reduced final titer.

### Example 3

The CDX-005 pre-master virus seed (preMVS) was developed as follows: RNA of SARS-COV-2 BetaCoV/USA/WA1/2020 (GenBank: MN985325.1) was extracted from infected, characterized Vero E6 cells (ATCC CRL-1586 Lot #70010177) and converted to 19 overlapping DNA fragments by RT-PCR using commercially available reagents and kits. Overlapping PCR was used to stitch together 19 1.8 kb wt genome fragments along with one deoptimized Spike gene cassette. Specifically, 1,272 nucleotides of the Spike ORF were human codon pair deoptimized from genome position 24115-25387 resulting in 283 silent mutations changes relative to parental WA1/2020 virus. The resulting full-length cDNA was transcribed in vitro to make full-length viral RNA. Viral recovery was conducted in a new BSL-3 laboratory at Stony Brook University (NY) that was commissioned for the first time in April 2020, with our project being the only project ever to occur in the lab. This viral RNA was then electroporated in characterized Vero E6 cells (Lot #70010177). This yielded CDX-005 virus (FIG. 1) that was subsequently passaged an additional time on Vero E6 cells to yield passage 1, P1 (Lot #1-060820-9-1). P1 material was used in the hamster study described below.

### Example 4

### Hamster Studies

The WHO ad hoc Expert Working Group on COVID-19 modelling concluded that both rhesus macaques and ferrets appear to reproduce mild to moderate human disease, but more recent work suggests that Syrian Golden Hamsters may be a more useful model to replicate more severe pulmonary manifestations of this infection.

Thirty-six male Syrian hamsters (Charles Rivers) 5-6 weeks old were utilized on study. For challenge, hamsters were anaesthetized with ketamine (100 mg/kg) and xylazine (10 mg/kg) via intraperitoneal injection and inoculated intranasally on Day 0 (12 per group) with 0.05 ml of either nominal doses of 5×10⁴ PFU/ml or 5×10³ PFU/ml of wt WA1 SARS-CoV-2 or 5×10⁴ PFU/ml CDX-005 Animals were observed twice daily and body weights collected daily through Day 8 and then daily from Day 16-Day 18. On Day 16, three CDX-005 inoculated animals were challenged intranasally with 5×104 PFU/ml wt WA1. Six naïve hamsters inoculated with either 5×104 PFU/ml (N=3) or 5×103 PFU/ml (N=3) of wt WA1 served as controls. We combined these two groups as titers overlapped at the two inoculation doses.

### Weight

These 36 hamsters and an additional 58 (half female/half male) 5-6 weeks old Syrian Golden hamsters (Charles Rivers) were used to study the effects of CDX-005 and wt WA1inoculation on hamster health as assessed by weight loss. (These additional hamsters are currently being evaluated for other CDX-005 and wt WA1 mediated effects.) In total forty 5×104 PFU CDX-005, forty 5×104 PFU wt WA1, and twelve 5×103 PFU wt WA1 were weighed daily for up to nine days. The N decreased over time for each group as animals were sacrificed for other endpoints on various days PI. The minimum N for 5×10⁴ PFU CDX-005 and 5×10⁴ PFU wt WA1 was 10 and 3 for 5×10³ PFU wt WA1.

### Tissue Harvesting

On days 2, 4, 6 post inoculation three hamsters from each group and on three hamsters on Day 18 from animals challenged on Day 16 were euthanized by intravenous injection of Beuthanasia at 150 mg/kg. The left lung was collected for viral load determination. To measure viral load, lung was homogenized in a 10% w/v in DMEM with antibiotics using a tissue homogenizer (Omni homogenizer) on Day 18 in animals challenged on Day 16 We attempted to perform nasal washes but were unsuccessful in obtaining reproducible washes in these small animals.

### Histopathology

Histopathology was performed by a blinded licensed veterinary pathologist. The lungs, brains, and kidneys were formalin fixed, dehydrated, embedded in paraffin, and stained with hematoxylin and eosin. Light microscopic evaluation was conducted by a blinded board-certified veterinary pathologist. Each tissue was graded on multiple pathological parameters and sections scored as 0=Normal, 1=Minimal, 2=Mild, 3=Moderate, 4=Marked, or 5=Severe. Evaluation of all tissues included assessment of cellular infiltration. At least five sections were examined for each organ and scores averaged.

### Viral Load

Viral load was measured by qPCR and TCID50 in harvested tissue. To measure viral load, tissue was homogenized in a 10% w/v in DMEM with antibiotics using a bead mill homogenizer (Omni). Infectious virus titers were determined by 50% tissue culture infectious dose (TCID50) assay titrating 10-fold serial dilutions of the lung homogenate on Vero E6 cells and are expressed in log 10 TCID50 units per ml. RNA was extracted from 100 μl of brain homogenate using the Quick-RNA Viral Kit (Zymo Research) according to the manufacturer's protocol. qRT-PCR was performed using the iTaq 1—step universal probe kit (Bio-Rad) using the following PCR cycling conditions: 40 cycles of 15 s at 95° C., 15 s at 60° C. and 20 s at 72° C.

### Antibodies—Plaque Reduction Neutralization Titer

Hamster sera collected at Day 16 PI were heat inactivated for 30′ at 56 C. 50 ul two-fold serial dilutions were performed in DMEM/1% FBS in 96-well U-bottom plates, starting with an initial dilution of 1:5. Approximately 30 PFU of SARS-CoV-2 Washington/1/2020 in 50 ul DMEM/1% FBS was added to the serum dilutions and mixed, bringing the final volume in the neutralization wells to 100 ul, and the total initial serum dilution to 1:10. Dilution plates were incubated for one hour at 37° C./5% CO2.

The cell growth medium on 24-well plates containing confluent monolayers of Vero E6 cells (seeded one day prior in DMEM/5% FBS), was removed, and 150 ul fresh DMEM/1% FBS was added, followed by 100 ul of each neutralization reaction. After one hour virus adsorption at 37° C./5% CO2, 0.75 ml semisolid overlay was added to the 24 well plates for a final concentration of 1×DMEM, 1.75% FBS, 0.3% Gum Tragacanth, 1× Penicillin+Streptomycin, in a total volume of 1 ml. 24 well plates were incubated 48 hours at 37° C. to allow for plaque formation. Plaques were visualized by fixing and staining the cell monolayers with 1% Crystal Violet in 50% Methanol/4% Formaldehyde. The plaque reduction neutralization titer (PRNT)50, 80, 90 was determined as the reciprocal of the last serum dilution that reduced plaque numbers by the pre-defined cutoff (50%, 80%, 90%) relative to the plaque numbers in non-neutralized wells (containing naïve hamster serum). Sera that failed to neutralize at the lowest dilution (1:10) was assigned a titer of 5, and sera that neutralized at the highest tested serum dilution (1:1280) were assigned a titer of ≥1280.

### Antibodies—IgG ELISA

Ninety-six well plates were coated with SARS-CoV-2(2019-nCoV) Spike S1-His (Sino Biological) at 30 ng/well in 50 ng/ml BSA/0.05M Carbonate/Bicarbonate Buffer pH9.6 overnight at 4° C. Plates were blocked with 10% goat serum in PBS 2 hr at 37° C., washed four times with washing buffer (0.1% Tween 20 in PBS) then incubated with a serially diluted serum (1:10 starting dilution and two folds thereafter) in 10% Goat serum/0.05% Tween-20 in PBS and incubated 1 hr at 37° C. Plates were washed four times with washing buffer then incubated with 1:10,000 horseradish peroxidase (HRP) conjugated affinity pure goat anti-Syrian hamster IgG (H & L) (Jackson ImmunoResearch Laboratories, Inc.) for 1 hr at 37° C. After the incubation, the plates were washed four times with washing buffer and Thermo Scientific OPD (o-phenylenediamine dihydrochloride) was added for colorimetric reaction. Following 10 min of incubation in the dark at 25° C., the reaction was stopped by adding 50 ml 2.5M sulfuric acid solution and the resultant absorbance was read on a microplate reader at 490 nm. Relative IgG levels among different groups were reported and compared as the log of the dilution at which the intensity of OPD colorimetric reaction product reached five time above the background (no serum) control intensity.

### Example 5

### CDX-005 Characteristics

CDX-005 contains 283 silent mutations in the Spike gene relative to wt WA1 virus. The resulting full-length wt WA1 and deoptimized cDNAs were transcribed in vitro to make full-length viral RNA that was electroporated into Vero E6 cells. Transfected cells were incubated for 6 days or until CPE appeared. Infection medium was collected on Days 2, 4, and 6. Virus titer was determined by plaque assay on Vero E6 cells. Plaques were visible as early as Day 2 post transfection, with peak virus generation on Days 4-6. Though plaques formed by CDX-005 and CDX-007 are smaller than wt, both grow robustly in Vero E6 cells, indicating their suitability for scale-up manufacturing. Thus, as with our other SAVE vaccines, we were able to rapidly generate multiple vaccine candidates with different degrees of attenuation.

In CDX-005, 1,272 nucleotides of the Spike ORF were codon pair deoptimized for human cells, yielding 283 silent mutations. The polybasic furin cleavage site was removed from the Spike protein for added attenuation and safety.

We performed growth optimization studies for CDX-005 in our GMP characterized animal origin-free (AOF) Vero (WHO-10-87) cells so that we can begin large scale vaccine production by Q4 2020. Growth at 33° C. results in higher titers for both CDX-005 and wt WA1 than at 37° C. Virus peaks before the cytopathic effect (CPE) is observed, with 80-90% of virus being cell associated at the peak. Though the kinetics differ, similar virus titers can be achieved at 0.01 MOI and 0.0001 MOI.

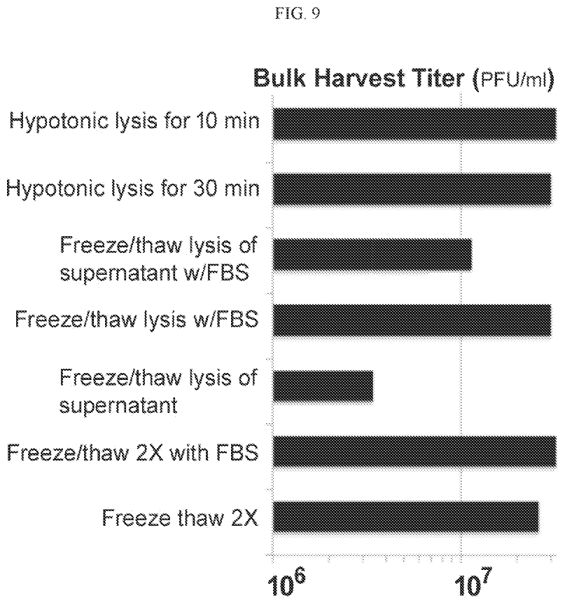

We also investigated optimal conditions for virus harvest. Vero WHO 10-87 cells were grown DMEM with 5% fetal bovine serum (FBS) at 37° C./5% CO₂. At 48 h post-infection with CDX-005 in culture at 33° C., cells and supernatant were harvested using the schemes described in FIG. 9.

The data demonstrate that 48 hr after 0.01 MOI infection at 33° C. most CDX-005 is cell-associated (˜80-90%) but that virus recovery from Vero cells is straight-forward. Hypotonic lysis is an effective means to harvest CDX-005, and the broad lysis window suggests this method will be feasible in scaled batches where some flexibility may be beneficial.

Freeze/thaw lysis is also effective and FBS is neither necessary nor beneficial. This is desirable both because FBS during infection can lead to Vero cell overgrowth, reducing virus yield, and the FDA prefers serum-free production. Also of note, CDX-005 appears to be stable when frozen in plain DMEM as FBS provided little or no stabilization at least after two freeze/thaw cycles. Thus, with optimal timing of harvest, whether grown at 33° C. or 37° C., crude bulk titers of 2-3×10⁷ PFU/ml of CDX-005 are routinely observed, or about 10⁶ PFU/cm² growth surface area.

Based on these studies we are currently growing CDX-005 by inoculating Vero (WHO-10-87) cells with 0.01 MOI at 33° C. We have selected and tested a vaccine formulation of DMEM with 5% sucrose and 5% glycine for our first-in-human studies in the UK. In this formulation, CDX-005 is stable for at least three freeze-thaw cycles and one month at −80° C. (the longest tested storage duration thus far).

Finally, as a first step in assessing the genomic stability of CDX-005, we have sequenced viral passages 1-6 after propagating the virus on Vero (WHO 10-87) cells. The data indicate that the virus is extremely stable. Sequencing of passage 6 revealed no subpopulations. We have grown and harvested nine passages.

### Example 6

As a prelude to moving CDX-005 to first-in-human clinical trials, we examined response of non-human primates to the vaccine. We have inoculated, intranasally, fifteen African green monkeys, six with 10⁶ PFU wt WA1, six with 10⁶ PFU CDX-005, and three with Dulbecco's PBS. Our findings show that while viral titers in lavage fluid of wt WA1 and CDX-005 inoculated animals were similar at Day 4 PI, viral titers remained high in wt WA1 but plummeted to undetectable in CDX-005 inoculated monkeys. These data further demonstrate CDX-005's potential as a SARS-CoV-2 vaccine.

### Example 7

### Recovery of SARS-CoV2 Beta Virus CDX.005.1

CDX-005.1 is based on the backbone of clinical stage CDX-005 (Wuhan lineage) that was recovered previously. The CDX-005 spike gene contains a codon-pair deoptimized cassette of 283 synonymous mutations, designed by the Codagenix Synthetic Attenuated Virus Engineering (SAVE) platform. The spike gene was further modified by a deletion of the furin cleavage site (36 nucleotide deletion). While not wishing to be bound by any particular theory, we believe that the absence of the furin cleavage site may contribute to attenuation in the human host of a SARS-CoV-2 carrying such mutation. We therefore decided to use CDX-005 as the backbone of our CDX-005.1. The furin cleavage site deletion is located in genome fragment F15.

To define a sequence for SARS-CoV-2 Beta vaccine candidate (CDX-005.1), various Beta variants on GISAID were selected and compared to CDX-005 by NCBI Blastn multiple sequence alignment. Nine key mutations, relative to the CDX-005 spike, were present in the spike genes of the majority of the Beta sequences we assessed (Table 5).

TABLE 5
Summary of genetic modifications to the
CDX-005 spike to generate CDX-005.1 spike

| Position in | Original | Mutated | Resulting in |
|---|---|---|---|
| spike gene (nt) | nucleotide | nucleotide | amino acid change |
| 21614 | C | T | L18 (CTT) to F (TTT) |
| 21801 | A | C | D80 (GAT) to A (GCT) |
| 22206 | A | G | D215 (GAT) to G (GGT) |
| 22286-22294 | CTTGCTTTA | Deletion | Del242-244 |
| 22813 | G | T | K417 (AAG) to N (AAT) |
| 23012 | G | A | E484 (GAA) to K (AAA) |
| 23063 | A | T | N501 (AAT) to Y (TAT) |
| 23403 | A | G | D614 (GAT) to G (GGT) |
| 23628 | C | T | A701 (GCA) to V (GTA) |

To construct the deoptimized CDX-005.1 genome, we de novo synthesized new fragments F14 and F15 containing the nine identified SARS-CoV-2 Beta mutations. The remaining 17 fragments (F1-F13 and F16-19) were recovered from CDX-005 Phase 1 clinical trial material. To assemble a full-length synthetic cDNA genome of CDX-005.1, the 19 overlapping PCR fragments were combined in a single overlapping PCR reaction.

| | | | SEQ | Primer |
|---|---|---|---|---|
| No. | Name | Description | ID NO | sequence 5′-3′ |
| 2312 | 2312-Fr1- | Forward primer | 15 | GAtaatacgactca |
| | T7G-F3 | for fragment 1 | | ctatagATTAAAGG |
| | | (T7 promoter | | TTTATACCTTCCCA |
| | | sequence were | | GGTAAC |
| | | add prior to | | |
| | | the viral | | |
| | | genome sequence | | |
| | | To facilitate | | |
| | | in vitro | | |
| | | transcription) | | |
| 1811 | 1811-COV- | Forward primer | 41 | GCAAAGAATGCTAT |
| | 27 | for fragment 14 | | TAGAAAAGTGTGAC |
| 1812 | 1812-COV- | Reverse primer | 42 | GATAGATTCCTTTT |
| | 28 | for fragment 14 | | TCTACAGTGAAGGA |
| | | | | TTTC |
| 1813 | 1813-COV- | Forward primer | 43 | GACTCCTGGTGATT |
| | 29 | for fragment 15 | | CTTCTTCAG |
| 1814 | 1814-COV- | Reverse primer | 44 | CTCTAGCAGCAATA |
| | 30 | for fragment 15 | | TCACCAAGG |

The resulting full-length PCR-assembled cDNA genome was used as template for in vitro transcription with T7 RNA polymerase driven by an added T7 promoter at the 5′ terminus of F1. The in vitro transcribed full length genome RNA together with in vitro transcribed nucleoprotein (NP) helper mRNA was co-transfected into Vero WHO 10-87 cells by electroporation. The virus resulting from this transfection is named CDX-005.1.

The viral genome of CDX-005 (SIIPL Vaccine Batch 403002) was converted to cDNA by reverse transcription and PCR-amplified as 17 overlapping sub-genomic DNA fragments. In addition, we de novo synthesized two new Beta-specific fragments 14 and 15. Each fragment overlapped with its neighboring fragment(s) by about 200 bp. The purified individual CDX-005.1 fragments F14, F15 and CDX-005 genome fragments F1-F13 and F16-F19 were pooled in a single tube overlap PCR reaction with two primers flanking the viral genome. The forward primer (2312) corresponding to the 5′ end of the virus genome included an upstream T7 RNA polymerase promoter. The 19-fragment overlap PCR produced a DNA amplicon of approximately 30 kb, suggesting that whole genomic cDNA was successfully generated, with clear bands visible on 0.5% agarose gels. After purification, the PCR-assembled full-length cDNA genomes were used as template for synthesis of infectious viral RNA by in vitro transcription in the presence of G cap-analog. The resulting transcript RNA appeared as a smear ranging from 8 kb to 1 kb relative to a DNA ladder run in parallel.

To test the integrity of the PCR-assembled full length cDNA genomes, a digest with restriction endonuclease Nhe I was used. CDX-005.1 genome cDNAs produced a unique and distinct fragment pattern owing to an additional Nhe I site that was designed in the deoptimized region of spike.

The fragment patterns of the Nhe I digested cDNA genomes corresponded to the in silico-predicted DNA fragment sizes indicating the viral cDNA genomes were assembled correctly. Of note, the portion of PCR product that did not migrate into the agarose gel disappeared after Nhe I digest and was converted into Nhe I RFLP fragments of expected size, suggesting that this material, too, corresponded to correctly formed full-length genome cDNA.

Reverse genetics-derived synthetic CDX-005.1 viruses were rescued, by co-electroporation into Vero 10-87 cells of in vitro transcribed genome RNA along with nucleoprotein mRNA as helper. Virus recovery of the CDX-005.1 vaccine strain was performed under biosafety level 2 enhanced (BSL2+) conditions, following approved Institutional Biosafety Committee guidelines. Infectious CDX-005.1 virus was detectable in the culture supernatant by plaque assay at 3 days after electroporation (4.6×10⁵ PFU/ml), and steadily increased to about 10⁷ PFU/ml by 6 days (FIG. 9).

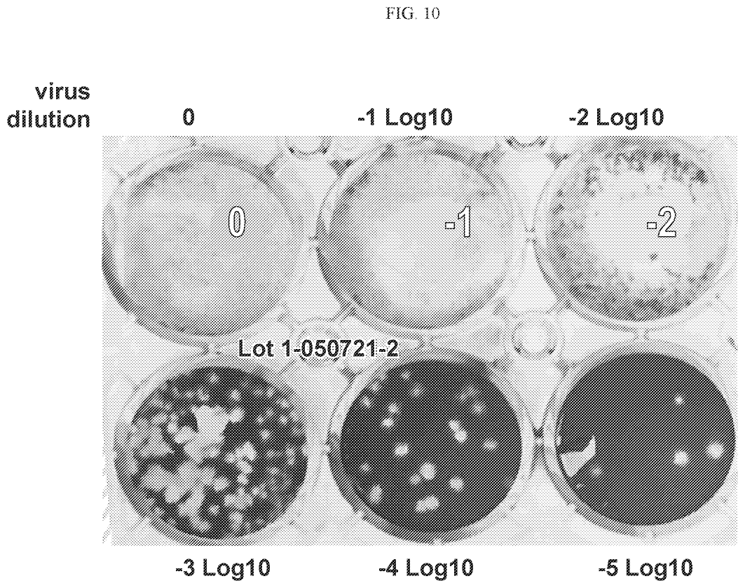

We have previously observed that the original CDX-005 vaccine strain was temperature sensitive for plaque formation at 40° C., a desirable safety feature for live attenuated vaccines, as it may plausibly predict virus shutoff at a temperature equivalent to human fever. To test if the ts phenotype extends CDX-005.1, we performed side-by-side plaque assays of CDX-005 and CDX-005.1 at the permissive temperature (37° C.) and the restrictive temperature (40° C.). Indeed, we observed a significant temperature sensitive phenotype of both viruses, with a reduction of plaque formation of approximately 1,000-fold for CDX-005 (confirming previous observations), whereas CDX-005.1 was unable to form any detectable plaques at any dilution (FIG. 10).

Using our established method of coronavirus genome assembly by overlapping PCR, Codagenix recovered live vaccine candidate against SARS-CoV-2 variant Beta (CDX-005.1) in 3 weeks from receipt of synthetic sequences.

CDX-005.1 grows to similar titers and displays similar plaque morphology as CDX-005. CDX-005 (1-5×10⁷ PFU/mL) at the permissive temperatures (33° C.-37° C.). CDX-005.1 severely temperature restricted for growth at 40° C., a feature previously observed for parental CDX-005 (Wuhan lineage).

### Example 8

### Sequencing of Beta (CDX-005.1) at Passage 1

To define a sequence for SARS-CoV-2 Beta vaccine candidate, 10-20 Beta variants on GISAID were selected and compared to CDX-005 by NCBI Blastn multiple sequence alignment. Ten (10) key mutations were present in the spike gene of every assessed Beta sequence and nine (9) nucleotides were deleted. Ten (10) nucleotides in the original CDX-005 spike gene were then substituted with these selected mutations to obtain the Beta variant spike sequence. The viral backbone is CDX-005 which is deleted for the furin cleavage site (36-nt deletion).

The newly constructed full-length Beta viral genome was in vitro transcribed followed by RNA purification. The purified genomic RNA was then transfected into WHO 10-87 Vero cells. The recovered virus from passage 1 was harvested and the viral RNA was extracted by Trizol protocol. Standard RT-PCR was performed, and 19 PCR fragments were PCR-amplified and subjected to Sanger sequencing to check the virus identity and to identify any spurious mutations. Sequencing reactions were mixed at Codagenix under BSL2 containment and submitted to Genewiz for sequencing. The resulting sequence was aligned with the designed sequence of the COVID-Beta variant on the backbone of the vaccine strain CDX-005.

10 nucleotide mutations and 9 deletions were listed as follows:

| Position | Original | Mutated | Resulting in |
|---|---|---|---|
| on Spike | nucleotide | nucleotide | amino acid change |
| 21614 | C | U | L(CUU)→F(UUU) |
| 21801 | A | C | D(GAU)→A(GCU) |
| 21846 | U | C | I(AUU)→T(ACU) |
| 22206 | A | G | D(GAU)→G(GGU) |
| 22287 | U | A | L(CUU)→H(CAU) |
| 22289- | GCTTT | deleted | A, L, and R |
| 22297 | ACGT | | deleted |
| 22813 | G | U | K(AAG)→N(AAU) |
| 23012 | G | A | E(GAA)→K(AAA) |
| 23063 | A | U | N(AAU)→Y(UAU) |
| 23403 | A | G | D(GAU)→G(GGU) |
| 23628 | C | U | A(GCA)→V(GUA) |

Compared with the designed sequence CDX.005.1 of Beta vaccine candidate, the obtained sequences from passage 1 had three point-mutations: A1870G, A7917U, and G14540U. The sequencing traces demonstrated that genomes with mutated nucleotides outcompete their original counterparts during viral replication. The mutated nucleotides were already the dominant species at passage 1, indicating that they are cell-adapted mutations.

Two mutations resulted in amino acid changes except for A1870G. Mutations that differ from the designed sequence are listed below:

| Mutation | Original | Mutated | |
|---|---|---|---|
| site | nt | nt | Amino Acid Change |
| 1870 | A | G | Ala(GCA)→(GCG), |
| | | | no change |
| 7917 | A | U | Glu(GAA)→Val(GUA) |
| 14540 | G | U | Ser(AGU)→Ile(AUU) |

### Example 9

### Recovery of CDX-005.2, Delta

Genetically modified, live-attenuated SARS-CoV-2 delta variant vaccine candidates CDX-005.2 was generated by a reverse genetics approach for coronaviruses (CoV) developed at Codagenix. Our approach is entirely “test tube-based; and eliminates the need of an intermediate cloning host (such as E. coli or yeast) to genetically manipulate CoV genomes. This allowed us to sidestep the genetic instability/toxicity problems of CoV genomes commonly encountered in traditional bacteria- or yeast-based reverse genetics systems.

CDX-005.2 is based on the backbone of clinical stage CDX-005 (Wuhan lineage) that was recovered previously at Codagenix. The CDX-005 spike contains a codon-pair deoptimized cassette of 283 synonymous mutations, according to our Synthetic Attenuated Virus Engineering platform (SAVE). In addition, the spike protein was modified by a 12 amino acid (36 nucleotides) deletion of the furin cleavage site (36 nucleotide deletion). While not wishing to be bound by any particular theory, we believe that the absence of the furin cleavage site may contribute to attenuation in the human host of a SARS-CoV-2 carrying such mutation. We therefore decided to use CDX-005 as the backbone of our CDX-005.2. The furin cleavage site deletion is located in genome fragment F15.

To define a sequence for SARS-CoV-2 Delta vaccine candidate (CDX-005.2), various delta variants on GISAID were selected and compared to CDX-005 by NCBI Blastn multiple sequence alignment. Eight key mutations were present in the spike gene of every assessed Delta sequence relative to the CDX-005 spike (Table 6).

TABLE 6
Summary of genetic modifications to the
CDX-005 spike to generate CDX-005.2 spike

| Position | | | Resulting in |
|---|---|---|---|
| in spike | Original | Mutated | amino acid |
| gene (nt) | nucleotide | nucleotide | change |
| 21618 | C | G | T(ACA)→R(AGA) |
| 21986 | G | A | G(GGU)→D(GAU) |
| 22029 | A | G | E(GAG)→G(GGA) |
| 22030 | G | A | |
| 22908 | U | G | L(CUG)→R(CGG) |
| 22959 | C | A | T(ACA)→K(AAA) |
| 24368 | G | A | D(GAC)→N(AAU) |
| 24370 | C | U | |

To construct the deoptimized CDX-005.2 genome, we de novo synthesized new fragments F14, F15 and F16 containing the 8 identified SARS-CoV-2 Delta mutations. The remaining 16 fragments (F1-F13 and F17-19) were recovered form CDX-005 Phase 1 clinical trial material. To assemble a full-length synthetic cDNA genome of CDX-005.2, the 19 overlapping PCR fragments thus recovered were combined in a single overlapping PCR reaction. F16-Min DNA template was designed to contain a codon pair-deoptimized region of 1213 nucleotides, analogous to that present in the original CDX-005. The resulting full-length PCR-assembled cDNA genome was used as template for in vitro transcription with T7 RNA polymerase driven by an added T7 promoter at the 5′ terminus of F1. The in vitro transcribed full length genome RNA together with in vitro transcribed nucleoprotein (NP) helper mRNA was co-transfected into Vero WHO 10-87 cells by electroporation. The virus resulting from this transfection is named CDX-005.2.

| | | | SEQ | |
|---|---|---|---|---|
| | | | ID | Primer |
| No. | Name | Description | NO: | sequence 5′-3′ |
| 2312 | 2312-Fr1- | Forward primer | 15 | GAtaatacgactca |
| | T7G-F3 | for fragment 1 | | ctatagATTAAAGG |
| | | (T7 promoter | | TTTATACCTTCCCA |
| | | sequence added | | GGTAAC |
| | | prior to the | | |
| | | viral genome | | |
| | | sequence to | | |
| | | facilitate | | |
| | | in vitro | | |
| | | transcription) | | |
| 1811 | 1811-COV- | Forward primer | 41 | GCAAAGAATGCTAT |
| | 27 | for fragment 14 | | TAGAAAAGTGTGAC |
| 1812 | 1812-COV- | Reverse primer | 42 | GATAGATTCCTTTT |
| | 28 | for fragment 14 | | TCTACAGTGAAGGA |
| | | | | TTTC |
| 1813 | 1813-COV- | Forward primer | 43 | GACTCCTGGTGATT |
| | 29 | for fragment 15 | | CTTCTTCAG |
| 1814 | 1814-COV- | Reverse primer | 44 | CTCTAGCAGCAATA |
| | 30 | for fragment 15 | | TCACCAAGG |
| 1815 | 1815-COV- | Forward primer | 45 | GCACAAGTCAAACA |
| | 31 | for fragment 16 | | AATTTACAAAACAC |
| 1816 | 1816-COV- | Reverse primer | 46 | CAAAAGGTGTGAGT |
| | 32 | for fragment 16 | | AAACTGTTACAAAC |

The viral genome of CDX-005 (SIIPL Vaccine Batch 403002) was converted to cDNA by reverse transcription and PCR-amplified as 16 overlapping sub-genomic DNA fragments. In addition, we de novo synthesized three new delta-specific fragments 14, 15, and 16. Each fragment overlapped with its neighboring fragment(s) by about 200 bp. The purified individual CDX-005.2 fragments 14-16 and CDX-005 genome fragments 1-13 and 17-19 were pooled in a single tube overlap PCR reaction with two primers flanking the viral genome. The forward primer (2312) corresponding to the 5′ end of the virus genome included an upstream T7 RNA polymerase promoter. The 19-fragment overlap PCR produced a DNA amplicon of approximately 30 kb, suggesting that whole genomic cDNA was successfully generated, with clear bands visible on 0.4% agarose gels. After purification the PCR-assembled full-length cDNA genomes were used as template for synthesis of infectious viral RNA by in vitro transcription in the presence of G cap-analog. The resulting transcript RNA appeared as a smear ranging from 8 kb to 1 kb relative to a DNA ladder run in parallel.

To test the integrity of the PCR-assembled full length cDNA genomes, a digest with restriction endonuclease Nhe I was used. CDX-005.2 genome cDNAs produced a unique and distinct fragment pattern owing to an additional Nhe I site that was designed in the deoptimized region of Spike. The fragment patterns of the Nhe I digested cDNA genomes corresponded to the in silico-predicted DNA fragment sizes indicating the viral cDNA genomes were assembled correctly. Of note, the portion of PCR product that did not migrate into the agarose gel disappeared after Nhe I digest and was converted into Nhe I RFLP fragments of expected size, suggesting that this material, too, corresponded to correctly formed full-length genome cDNA.

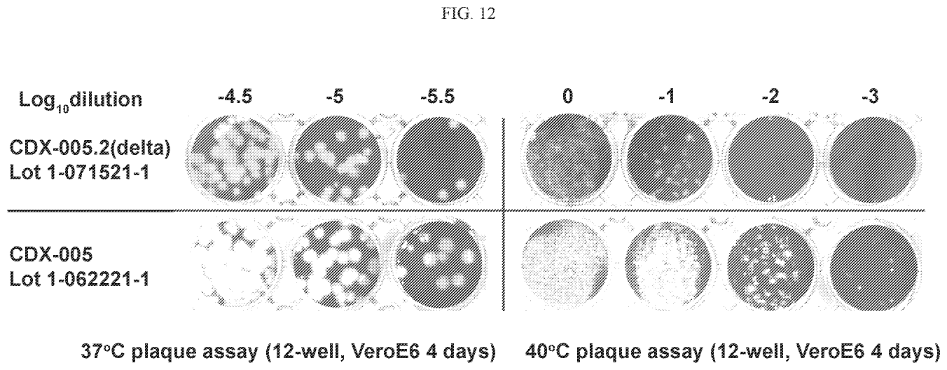

Reverse genetics-derived synthetic CDX-005.2 viruses was rescued, by co-electroporation into Vero 10-87 cells of in vitro transcribed genome RNA along with nucleoprotein mRNA as helper. Virus recovery of the CDX-005.2 vaccine strain was performed under biosafety level 2 enhanced (BSL2+) conditions, following approved Institutional Biosafety Committee guidelines. Infectious CDX-005.2 virus was detectable in the culture supernatant by plaque assay at 4 days after electroporation (2.5×10⁵ PFU/ml), and steadily increased to about 10⁷ PFU/ml by 7 days (FIG. 12).

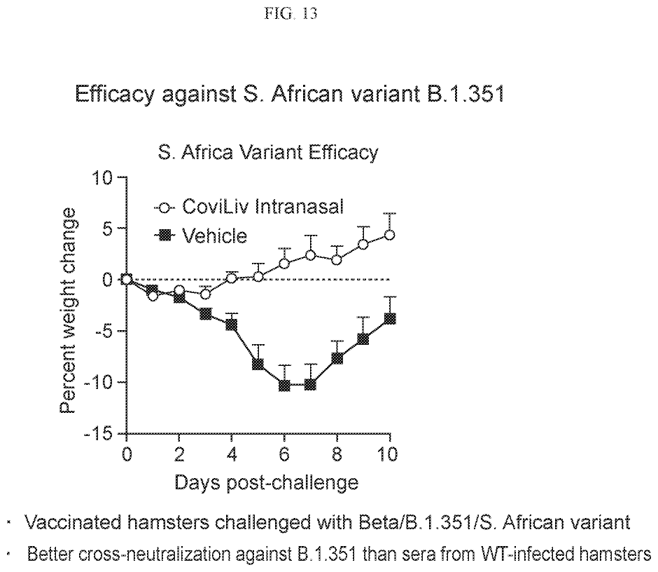

We have previously observed that the original CDX-005 vaccine strain was temperature sensitive for plaque formation at 40° C., a desirable safety feature for live attenuated vaccines, as it may plausibly predict virus shutoff at a temperature equivalent to human fever. To test if the ts phenotype extends CDX-005.2, we performed side-by-side plaque assays of CDX-005 and CDX-005.2 at the permissive temperature (37° C.) and the restrictive temperature (40° C.). Indeed, we observed a significant temperature sensitive phenotype of both viruses, with a reduction of plaque formation of approximately 1,000-fold, and 10,000-fold for CDX-005 and CDX-005.2, respectively (FIG. 13).

Recovered CDX-005.2 is temperature sensitive for growth at 40° C., similar to parental CDX-005 and has a similar plaque morphology to CDX-005.

### Example 10

### Sequencing Report of SARS-CoV-2 Delta Vaccine Candidate (CDX-005.2) at Passages 1 and 2

To define a sequence for SARS-CoV-2 Delta vaccine candidate (CDX-005.2), 10-20 Delta variants on GISAID were selected and compared to CDX-005 by NCBI Blastn multiple sequence alignment. Eight key mutations were present in the spike gene of every assessed Delta variant sequence. Eight nucleotides in original CDX-005 spike gene were then substituted with these selected mutations to obtain the Delta variant spike sequence.

Where a Delta mutation was in the deoptimized region, the deoptimized codon was replaced by the wild-type codon. The viral backbone is CDX-005, which contains a 36-nucleotide deletion of the furin cleavage site. The newly constructed full-length Delta viral genome was transcribed, followed by RNA purification.

The purified genomic RNA was then transfected into WHO 10-87 Vero cells. The recovered viruses from passage 1 and passage 2 were harvested and the viral RNA was extracted with TRIzol™ (for passage 1) and QiaAmp viral kit (for passage 2) via the manufacturer's protocols. Standard RT-PCR was performed, and 19 PCR fragments were further amplified and analyzed by Sanger sequencing to confirm the virus identity and to identify any spurious mutations.

Sequencing reactions were set up at Codagenix under BSL2 containment and submitted to Genewiz Inc (South Plainfield, NJ) and Eurofins Genomics (Louisville, KY) for sequencing. The resulting sequence was aligned with the designed sequence of the COVID Delta variant on the backbone of the CDX-005 vaccine.

11 single nucleotide changes and one 6 nucleotide deletion was introduced into the spike gene of CDX-005 in order to generate CDX-005.2 with a matching amino acid sequence to that of the SARS-CoV-2 delta variants prevailing at the time of design (Table 7). All delta-specific sequence edits were confirmed in CDX.005.2 virus at both Passage 1 (Lot 1-071521-1) and Passage 2 (Lot 1-073121-1). The same 12 amino acid furin cleavage site deletion as is present in CDX-005 was implemented in CDX-005.2, and was herein verified. The genome sequences of CDX-005.2 at Passage 1 and Passage 2 were identical. Five spontaneous point-mutations were detected in CDX-005.2 at both Passage 1 and Passage 2: G1013A, C10833A, A11089G, A12557U, and G21668A. The sequencing traces at each position with a mutation show some level of genetic heterogeneity, with some portion of the population still carrying the original nucleotide. The mutated nucleotides were the dominant species at passage 1, but four of them outcompeted their original counterparts upon an additional passage (passage 2), indicating that they are likely cell culture adaptation mutations. C10833A remained a mixed species, with adenine remaining dominant at passage 2. Four of the mutations resulted in amino acid changes, while A1 1098G maintained the same amino acid. All amino acid differences from the design sequence were outside of the deoptimized region and could be a result of normal virus adaptation to cell culture.

One further sequence variation between CDX-005 (Codagenix Passage 2) and CDX-005.2 (Codagenix Passage 2) was detected at genome position 28818. Whereas this position is cytidine in the CDX-005 reference sequence (Codagenix Passage 2), it is uracil in CDX-005.2, resulting in a Ser to Leu amino acid change in N nucleoprotein.

TABLE 7
Sequence Comparison between CDX-005
(P2, Lot 1-061920-1) and CDX-005.2-Delta
(P2, Lot 1-073121-1)

| | | Nucleotide | | |
|---|---|---|---|---|
| Genome | Nucleotide | in CDX- | | |
| position | in CDX-005 | 005.2 | Resulting amino | |
| (Gene) | (P2) | (P2) | acid change | Comment |
| 1013 (nsp2) | G | A | Glu (GAA)→Lys (AAA) | spontaneous |
| 10833 (nsp5) | C | A | Ala (GCC)→Asp (GAC) | spontaneous |
| 11089 (nsp6) | A | G | Glu (GAA→GAG), no | spontaneous |
| | | | change | |
| 12557 (nsp8) | A | U | Ile (AUC)→Phe (UUC) | spontaneous |
| 21618 (spike) | C | G | Thr (ACA)→Arg (AGA) | Delta specific |
| 21668 (spike) | G | A | Val (GUU)→Ile (AUU) | spontaneous |
| 21846 (spike) | U* | C | Ile (AUU)→Thr (ACU) | Delta specific |
| 21987 (spike) | G | A | Gly (GGU)→Asp (GAU) | Delta specific |
| 22029 (spike) | A (del) | G | Glu (GAG)→Gly (GGA) | Delta specific |
| 22030 (spike) | G (del) | A | | Delta specific |
| 22296 (spike) | G* | A (22290) | Arg (CGU)→His (CAU) | Delta specific |
| 22917 (spike) | U | G (22911) | Leu (CUG)→Arg (CGG) | Delta specific |
| 22995 (spike) | C | A (22989) | Thr (ACA)→Lys (AAA) | Delta specific |
| 23403 (spike) | A | G (23397) | His (CAU)→Arg (CGU) | Delta specific |
| 24201 (spike) | U* | C (24195) | Val (GUA)→Ala (GCA) | Delta specific |
| 24374 (spike) | G | A (24368) | Asp (GAC)→Asn (AAU) | Delta specific |
| 24376 (spike) | C | U (24370) | | Delta specific |
| 28818 (N) | C | U (22812) | Ser (UCA)→Leu (UUA) | U in SIIPL |
| | | | | Vaccine Lot |

*Spontaneous mutations in CDX-005 that were restored to the wt SARS-CoV-2(delta)-specific amino acid

### Example 11

Hamsters were vaccinated IN with deoptimized SARS-CoV2 (CDX.005) or wildtype SARS-CoV2 WA/i. Day 27 post-vaccination, hamsters were challenged IN SARS-CoV2 variant Beta. Neutralizing antibody titers were assessed via MN assay against SARS-CoV2 variant Beta. FIG. 14 shows that there was better cross-neutralization against B.1.351 than sera from WT-infected hamsters.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A polynucleotide, comprising: a polynucleotide encoding one or more viral proteins or one or more fragments thereof of a parent SARS-CoV-2 variant,
wherein the polynucleotide is recoded compared to its parent SARS-CoV-2 variant polynucleotide, and
wherein the amino acid sequence of the one or more viral proteins, or one or more fragments thereof of the parent SARS-CoV-2 variant encoded by the polynucleotide remains the same, or
wherein the amino acid sequence of the one or more viral proteins or one or more fragments thereof of the parent SARS-CoV-2 variant encoded by the polynucleotide comprises up to 20 amino acid substitutions, additions, or deletions,
wherein the one or more viral proteins or one or more fragments thereof comprises spike protein or a fragment thereof.

2. A polynucleotide of claim 1, wherein
the parent SARS-CoV-2 variant comprises SEQ ID NO:1, or
the parent SARS-CoV-2 variant comprises SEQ ID NO:1 wherein nt 9469 is changed from to A to G and nt 26222 changed from T to G, or
the parent SARS-CoV-2 variant comprises SEQ ID NO:1 wherein there is one or more mutations in SEQ ID NO:1; and
wherein a spike protein coding sequence in SEQ ID NO:1, SEQ ID NO:1 wherein nt 9469 is changed from to A to G and nt 26222 changed from T to G, or SEQ ID NO:1 wherein there is one or more mutations, is replaced with a recoded spike protein coding sequence from a SARS-CoV-2 variant.

3. A polynucleotide of claim 1, wherein the SARS-CoV-2 variant selected from the group consisting of U.K. variant, South Africa variant, Brazil variant, Delta variant, and Omicron variant.

4. A polynucleotide of claim 1,
wherein the polynucleotide is recoded by reducing codon-pair bias (CPB) or reducing codon usage bias compared to its parent SARS-CoV-2 variant polynucleotide, or
wherein the polynucleotide is recoded by increasing the number of CpG or UpA di-nucleotides compared to its parent SARS-CoV-2 variant polynucleotide.

5. (canceled)

6. A polynucleotide of claim 1, wherein each of the recoded one or more viral proteins, or each of the recoded one or more fragments thereof has a codon pair bias less than, −0.05, less than −0.1, less than −0.2, less than −0.3, or less than −0.4.

7. A polynucleotide of claim 1, wherein the polynucleotide is CPB deoptimized compared to its parent SARS-CoV-2 variant polynucleotide.

8. A polynucleotide of claim 1, wherein the polynucleotide is codon deoptimized compared to its parent SARS-CoV-2 variant polynucleotide.

9. A polynucleotide of claim 7, wherein the CPB deoptimized is based on CPB inhumans.

10. A polynucleotide of claim 7, wherein the CPB deoptimized is based on CPB in a coronavirus, or CPB in a wild-type SARS-CoV-2 coronavirus.

11. (canceled)

12. A polynucleotide of claim 1, wherein a furin cleavage site is eliminated.

13. A vector comprising a polynucleotide of claim 1.

14. A cell comprising a polynucleotide of claim 1, or a vector comprising a polynucleotide of claim 1.

15. The cell of claim 14, wherein the cell is Vero cell or baby hamster kidney (BHK) cell.

16. A polypeptide encoded by a polynucleotide of claim 1.

17. A modified SARS-CoV-2 variant comprising a polynucleotide of claim 1.

18. A modified SARS-CoV-2 variant comprising a polypeptide encoded by a polynucleotide of claim 1.

19. A modified SARS-CoV-2 variant of claim 17, wherein expression of one or more of its viral proteins is reduced compared to its parent SARS-CoV-2 variant.

20. A modified SARS-CoV-2 variant of claim 17, wherein the reduction in the expression of one or more of its viral proteins is reduced as the result of recoding a spike protein or a fragment thereof.

21. An immune composition or vaccine composition for inducing an immune response in a subject, comprising:
one or more modified SARS-CoV-2 variant of claim 17.

22. The immune composition or vaccine composition of claim 21, further comprising a modified SARS-CoV-2 coronavirus comprising
a polynucleotide having SEQ ID NO:1, SEQ ID NO:1 wherein nt 9469 is changed from to A to G and nt 26222 changed from T to G, or SEQ ID NO:1 with up to 20 mutations, wherein the polynucleotide having SEQ ID NO:1 with up to 20 mutations is not the same as the polynucleotide in the modified SARS-CoV-2 variant, or
polypeptide encoded by the polynucleotide having SEQ ID NO:1, SEQ ID NO:1 wherein nt 9469 is changed from to A to G and nt 26222 changed from T to G, or SEQ ID NO:1 with up to 20 mutations, wherein the polypeptide encoded by a polynucleotide having SEQ ID NO:1 with up to 20 mutations is not the same as the polypeptide in the modified SARS-CoV-2 variant,
wherein the immune composition or vaccine composition is a multivalent immune composition or vaccine composition.

23. The immune composition or vaccine composition of claim 21, further comprising a pharmaceutically acceptable carrier or excipient.

24. A method of eliciting an immune response in a subject, comprising: administering to the subject a dose of:
a modified SARS-CoV-2 variant of claim 17, or an immune composition or vaccine composition comprising one or more modified SARS-CoV-2 variant of claim 17.

25. A method of eliciting an immune response in a subject, comprising:
administering to the subject a prime dose of a modified SARS-CoV-2 coronavirus of claim 17, or an immune composition or vaccine composition of comprising one or more modified SARS-CoV-2 variant of claim 17; and
administering to the subject one or more boost doses of a modified SARS-CoV-2 coronavirus of claim 17, or an immune composition or vaccine composition of comprising one or more modified SARS-CoV-2 variant of claim 17.

26. A method of claim 24, wherein the immune response is a protective immune response.

27. A method of claim 24, wherein the dose is a prophylactically effective or therapeutically effective dose.

28. A method of claim 24, wherein administering is via a nasal route.

29. A method of claim 24, wherein administering is via nasal drop or via nasal spray.

30. (canceled)

31. A method of claim 24, wherein the dose is about 10⁴-10⁶ PFU.

32. A method of making adeoptimized SARS-CoV-2 variant, comprising:
obtaining a nucleotide sequence encoding one or more proteins of a parent SARS-CoV-2 variant or one or more fragments thereof,
recoding the nucleotide sequence to reduce protein expression of the one or more proteins, or the one or more fragments thereof; and
substituting a nucleic acid having the recoded nucleotide sequence into the parent SARS-CoV-2 variant genome to make the deoptimized SARS-CoV-2 variant genome,
wherein expression of the recoded nucleotide sequence is reduced compared to the parent virus.

33. (canceled)


SEQUENCE LISTING (54 sequences)

SEQ ID NO: 1 - DNA, 29834 bp, Artificial Sequence
  synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacgtagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgtagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 2 - protein, 1261 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALRRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGVALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3777 bp, Artificial
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccataaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactagcaagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactcacaatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataa

SEQ ID NO: 4 - DNA, 3777 bp, Artificial
  Synthetic construct
atgtttgtttttcttgttttattgccactagtctctagtcagtgtgttaattttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgtagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataa

SEQ ID NO: 5 - DNA, 3780 bp, Artificial
  Synthetic construct
atgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaaaatgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataa

SEQ ID NO: 6 - DNA, 3777 bp, Artificial
  Synthetic construct
atgtttgtttttcttgttttattgccactagtctctagtcagtgtgttaattttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgtagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgtagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataa

SEQ ID NO: 7 - DNA, 3780 bp, Artificial
  Synthetic construct
atgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaaaatgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataa

SEQ ID NO: 8 - DNA, 29825 bp, Artificial
  Synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcgtttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagtatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttatttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaattttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgtagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgtagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 9 - DNA, 29828 bp, Artificial
  Synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctataaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgacgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgagaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaattccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttagaaccagaactcaattaccccctgcatacactaattctttcacacgtggtatttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaaaatgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagtttaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 10 - DNA, 29825 bp, Artificial
  Synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttaggagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaagggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcacaacgctcaagcacttaatacactcgttaaacagcttagttctaagtttggcgcaatttctagtgtgcttaacgatatattttcgagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcggttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 11 - DNA, 29825 bp, Artificial
  Synthetic Construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttaggagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttataaccagaactcaatcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagggcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatttcgcaccatttttcgcttttaagtgttatggagtgtctcctactaaattaaatgatctctgctttactaatgtctatgcagattcatttgtaattagaggtaatgaagtcagccaaatcgctccagggcaaactggaaatattgctgattataattataaattaccagatgattttacaggctgcgttatagcttggaattctaacaagcttgattctaaggttggtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatggtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcataacgctcaagcacttaatacactcgttaaacagcttagttctaaatttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcggttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 12 - DNA, 29819 bp, Artificial
  Synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttaggagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttataaccagaactcaatcatacactaattctttcacacgtggtgtttattaccctgacaaagttttcagatcctcagttttacattcaactcaggacttgttcttacctttcttttccaatgttacttggttccatgctatctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttggatgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagggcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatttcgcaccatttttcgcttttaagtgttatggagtgtctcctactaaattaaatgatctctgctttactaatgtctatgcagattcatttgtaattagaggtaatgaagtcagccaaatcgctccagggcaaactggaaatattgctgattataattataaattaccagatgattttacaggctgcgttatagcttggaattctaacaagcttgattctaaggttggtggtaattataattaccggtatagattgtttaggaagtctaatctcaaaccttttgagagagatatttcaactgaaatctatcaggccggtaacaaaccttgtaatggtgttgcaggtgttaattgttactttcctttacaatcatatggtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcggttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 13 - protein, 12 aa, Artificial
  Synthetic construct
TNSPRRARSVAS

SEQ ID NO: 14 - DNA, 36 bp, Artificial
  Synthetic construct
actaattctcctcggcgggcacgtagtgtagctagt

SEQ ID NO: 15 - DNA, 48 bp, Artificial
  Synthetic construct
gataatacgactcactatagattaaaggtttataccttcccaggtaac

SEQ ID NO: 16 - DNA, 23 bp, Artificial
  Synthetic construct
gatgccaaaataatggcgatctc

SEQ ID NO: 17 - DNA, 22 bp, Artificial
  Synthetic Construct
gttggttgccataacaagtgtg

SEQ ID NO: 18 - DNA, 27 bp, Artificial
  Synthetic construct
ctaattgaggttgaacctcaacaattg

SEQ ID NO: 19 - DNA, 28 bp, Artificial
  Synthetic construct
gagtatggtactgaagatgattaccaag

SEQ ID NO: 20 - DNA, 24 bp, Artificial
  Synthetic construct
ctaggtggaatgtggtaggattac

SEQ ID NO: 21 - DNA, 29 bp, Artificial
  Synthetic Construct
gctgttacagcgtataatggttatcttac

SEQ ID NO: 22 - DNA, 29 bp, Artificial
  Synthetic construct
gctggtttaagtataatgtctcctacaac

SEQ ID NO: 23 - DNA, 28 bp, Artificial
  Synthetic Construct
gcacaaaaccagttgaaacatcaaattc

SEQ ID NO: 24 - DNA, 28 bp, Artificial
  Synthetic construct
gcaactagtgttttgagtttttccattg

SEQ ID NO: 25 - DNA, 25 bp, Artificial
  Synthetic construct
gtgaagaatcatctgcaaaatcagc

SEQ ID NO: 26 - DNA, 32 bp, Artificial
  Synthetic construct
caaatgatataagcaattgttatccagaaagg

SEQ ID NO: 27 - DNA, 35 bp, Artificial
  Synthetic construct
gcctttaatactttactattccttatgtcattcac

SEQ ID NO: 28 - DNA, 27 bp, Artificial
  Synthetic Construct
ccagacaaactagtatcaaccatatcc

SEQ ID NO: 29 - DNA, 25 bp, Artificial
  Synthetic construct
gctatgggtattattgctatgtctg

SEQ ID NO: 30 - DNA, 21 bp, Artificial
  Synthetic construct
cctacaaggtggttccagttc

SEQ ID NO: 31 - DNA, 19 bp, Artificial
  Synthetic construct
cgacagatgtcttgtgctg

SEQ ID NO: 32 - DNA, 25 bp, Artificial
  Synthetic construct
ggtatccagttgaaactacaaatgg

SEQ ID NO: 33 - DNA, 23 bp, Artificial
  Synthetic construct
gatcagacataccacccaaattg

SEQ ID NO: 34 - DNA, 34 bp, Artificial
  Synthetic construct
cttatgtattgtaagtacaaatgaaagacatcag

SEQ ID NO: 35 - DNA, 27 bp, Artificial
  Synthetic construct
ggtgatgattatgtgtaccttccttac

SEQ ID NO: 36 - DNA, 27 bp, Artificial
  Synthetic construct
ctgttaattgcagatgaaacatcatgc

SEQ ID NO: 37 - DNA, 29 bp, Artificial
  Synthetic construct
gtgtgtagacttatgaaaactataggtcc

SEQ ID NO: 38 - DNA, 21 bp, Artificial
  Synthetic construct
catacaaactgccaccatcac

SEQ ID NO: 39 - DNA, 31 bp, Artificial
  Synthetic construct
ccttgtagtgacaaagcttataaaatagaag

SEQ ID NO: 40 - DNA, 21 bp, Artificial
  Synthetic construct
ctggtgcaactcctttatcag

SEQ ID NO: 41 - DNA, 28 bp, Artificial
  Synthetic construct
gcaaagaatgctattagaaaagtgtgac

SEQ ID NO: 42 - DNA, 32 bp, Artificial
  Synthetic construct
gatagattcctttttctacagtgaaggatttc

SEQ ID NO: 43 - DNA, 23 bp, Artificial
  Synthetic construct
gactcctggtgattcttcttcag

SEQ ID NO: 44 - DNA, 23 bp, Artificial
  Synthetic Construct
ctctagcagcaatatcaccaagg

SEQ ID NO: 45 - DNA, 28 bp, Artificial
  Synthetic construct
gcacaagtcaaacaaatttacaaaacac

SEQ ID NO: 46 - DNA, 28 bp, Artificial
  Synthetic construct
caaaaggtgtgagtaaactgttacaaac

SEQ ID NO: 47 - DNA, 19 bp, Artificial
  Synthetic construct
ctcactccctttcggatgg

SEQ ID NO: 48 - DNA, 32 bp, Artificial
  Synthetic construct
gaggtttatgatgtaatcaagattccaaatgg

SEQ ID NO: 49 - DNA, 29 bp, Artificial
  Synthetic construct
gctacaggattggcaactataaattaaac

SEQ ID NO: 50 - DNA, 22 bp, Artificial
  Synthetic construct
ccattctagcaggagaagttcc

SEQ ID NO: 51 - DNA, 21 bp, Artificial
  Synthetic construct
gcaatcctgctaacaatgctg

SEQ ID NO: 52 - DNA, 53 bp, Artificial
  Synthetic construct
tttttttttttttttttttttttttgtcattctcctaagaagctattaaaatc

SEQ ID NO: 53 - DNA, 55 bp, Artificial
  Synthetic construct
gaataatacgactcactatagggacgttcgtgttgttttagatttcatctaaacg

SEQ ID NO: 54 - DNA, 55 bp, Artificial
  Synthetic construct
tttttttttttttttttttttgtcattctcctaagaagctattaaaatcacatgg